(19) United States
(12) Patent Application Publication
(10) Pub. No.: US 2023/0144548 A1
(43) Pub. Date: May 11, 2023

(54) RECOMBINANT AAV VECTORS WITH ALTERED IMMUNOGENCITY AND METHODS OF MAKING THE SAME

(71) Applicant: John Fraser Wright, Redwood City, CA (US)

(72) Inventor: John Fraser Wright, Redwood City, CA (US)

(21) Appl. No.: 17/624,200

(22) PCT Filed: Jun. 30, 2020
(86) PCT No.: PCT/US2020/040370

§ 371 Date: Dec. 30, 2021

Related U.S. Application Data
(60) Provisional application No. 62/956,898, filed on Jan. 3, 2020.
(60) Provisional application No. 62/868,983, filed on Jun. 30, 2019.

(51) Int. Cl. C12N 15/86; C12N 9/10; A61K 35/76; C12N 15/64

(52) U.S. Cl. CPC C12N 15/86; C12N 9/1007; A61K 35/76; C12N 15/64; C12N 2750/14133; C12N 2750/14143; C12N 2750/14152; C12N 2330/51

(57) ABSTRACT

The present invention provides methods of generating a recombinant AAV vector with reduced immunogenicity, comprising: providing eukaryotic cells with a nucleic acid comprising a sequence of interest that is flanked by AAV inverted terminal repeats, wherein the nucleic acid comprises CpG dinucleotide sites, wherein at least a portion of the CpG dinucleotide sites are methylated, wherein the eukaryotic cell expresses one or more other components necessary to achieve recombinant AAV biosynthesis, whereby the recombinant AAV vector is generated by the eukaryotic cell, wherein the generated recombinant AAV vector comprises nucleic acid wherein at least a portion of the CpG dinucleotide sites are methylated.

### CROSS-REFERENCE TO RELATED APPLICATIONS

This application claims the benefit of U.S. Provisional Appl. No. 62/868,983, filed on Jun. 30, 2019, and U.S. Provisional Appl. No. 62/956,898, filed Jan. 3, 2020, the contents of which are hereby incorporated by reference in their entirety.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 349,353 Byte ASCII (Text) file named “Sequence_listing_ST25.txt,” created on Jun. 30, 2020.

### FIELD OF THE INVENTION

The technical field of the invention relates to recombinant adeno associated virus-based vectors for human gene therapy, and in particular, novel manufacturing methods to reduce Pathogen Associated Molecular Patterns (PAMP's) to reduce recipient immune recognition of AAV vectors and vector-transduced cells, including prevention of formation of cytotoxic T lymphocyte (CTL's), and thereby increase the durability of therapeutic gene expression; or alternatively methods to increase PAMPs to increase recipient immune recognition of AAV vectors and vector-transduced cells, including amplification of formation of CTL's and thereby increase infectious agent or cancer immunotherapy.

### BACKGROUND OF THE INVENTION

The use of recombinant adeno associated virus (rAAV)-based gene transfer vectors for human gene therapy has been in development for over 20 years, and despite a series of suboptimal, sub-therapeutic and in several cases promising but only short-term therapeutics effects in clinical trials, has in the last 10 years demonstrated progressively improving clinical results. One important milestone on this trajectory was the FDA approval of the first gene therapy product for a genetic disease, Luxturna® (voretigene neparvovec-rzyl) developed by the Children's Hospital of Philadelphia, University of Pennsylvania and Spark Therapeutics, for RPE65 deficiency, in December 2017 by the U.S. Food and Drug Administration (FDA), with subsequent approval in Europe. Another important milestone was achieved with the FDA approval of Zolgensma® (onasemnogene abeparvovec-xioi) developed by Nationwide Children's Hospital and AveXis, for the treatment of pediatric patients less than 2 years of age with spinal muscular atrophy (SMA) with bi-allelic mutations in the survival motor neuron 1 (SMN1) gene, in May 2019. In addition, promising rAAV-based gene therapies for several other genetic diseases including hemophilia (types A and B) and Duchennes muscular dystrophy (DMD) are advancing in clinical trials. While promising efficacy is being reported in many studies, a feature of most trials that utilize systemic rAAV vector administration, including those for SMA, hemophilia A and B, and DMD, is the occurrence of elevated liver enzymes (ALT and AST) following vector administration in human clinical trials, corresponding to vector induced immune responses caused by vector administration, leading to suboptimal therapeutic gene expression levels and durability of expression.

The parent virus from which rAAV vectors are developed, adeno associated virus (AAV), is a small, stable, common virus in humans and the mammalian population, and considered innocuous because it is not believed to be pathogenic. The genome of the wild type AAV is composed of two genes, Rep and Cap, flanked by inverted terminal repeats (ITR's). Through a series of promoters, Rep encodes a series of gene products required for replication and packaging of new AAV virus particles, and Cap encodes three capsid proteins, VP1, VP2, and VP3, that form the protein capsid containing the vector genome. AAV is a member of the parvovirus family. In natural infection, AAV is dependent on co-infection with a ‘helper’ virus such as Adenoviruses or Herpesviruses, which provide ‘helper virus functions’ to AAV viruses that enable replication of the AAV, if present. A more detailed description of AAV is provided in Field's Virology, which is known, and readily available, to one of skill in the art.

A recombinant AAV (rAAV) vector is composed of an AAV capsid protein shell composed of VP1, VP2, and VP3 as in the wild type virus, but in which the genome of the wild type virus has been replaced with a transgene. To make a rAAV vector, the wild type AAV Rep and Cap genes encoded by the virus genome are removed and replaced with a transgene. Also typically included in the vector genome are regulatory elements such as a promoter to drive transgene expression, and a polyadenylation sequence. Techniques in virology, molecular biology, and cell biology have been developed to enable the construction and manufacture of rAAV for basic and translational research, and clinical development of rAAV based gene therapy investigational products.

Manufacturing of a rAAV gene therapy product can be divided into the ‘upstream’ vector production or vector generation phase, in which rAAV is generated in a cell line in which all genes required for formation and assembly of rAAV particles are provided, and ‘downstream’ vector purification, in which the rAAV particles generated in the upstream process are separated away from the complex mixture of other components of the upstream production cell culture system. Generally two systems have been developed for rAAV generation in cell culture: namely, 1) ‘helper virus free’ systems in which all components required for the biosynthesis/generation of rAAV vectors are provided on recombinant bacterial plasmid DNA introduced into mammalian cells grown in cell culture by transient transfection; and 2) cell culture systems in which some of the components required for the biosynthesis/generation of rAAV vectors are provided by wild-type or recombinant viruses/helper viruses, including adenoviruses, herpesviruses, baculoviruses, in cell culture by infection.

An advantage of the ‘helper virus free’ systems is elimination of safety concerns related to contamination of the therapeutic product with replication competent viruses, and an advantage of the helper virus systems is ease of scalability because the infection processes are easier to perform at large scale compared to transient transfection processes. Transient versus stable transfection is used because many of the components required for the biosynthesis/generation of rAAV vectors are toxic or cytostatic to production cells.

A major benefit to using rAAV and other recombinant viruses for therapeutic gene transfers is the high efficiency of gene delivery to cells. Viruses have evolved over long periods of time to efficiently enter and deliver their own (wild type) genomes to target cells (such as human cells), hence the use of a viral vector containing a therapeutic expression cassette takes advantage of this highly efficient gene delivery mechanism to achieve a therapeutic and beneficial effect in the recipient.

The objective and therapeutic benefit of gene therapy is the transfer and expression of therapeutic genes, for example delivery of therapeutic gene expressions cassettes using rAAV vectors, to persons with genetic disease who are missing normal, functional versions of specific genes. To be effective and useful, the gene transfer procedure and dose must achieve expression at levels sufficiently high to mitigate the disease process, and achieve long-term, durable expression, for example years to decades. Such expressions level and durability must generally be achieved by a single administration (‘once and done’) because adaptive immune responses mounted by the human subject after a first exposure generally preclude second and subsequent serial administration of the same rAAV product in the same human subject.

However, a major challenge to using rAAV and other recombinant viruses for therapeutic gene transfer is the barrier of the human immune response, which is highly evolved to protect humans from virus infection, because in nature wild-type viruses are generally deleterious to the health of persons that are infected by them. The human immune response to viruses and other biological pathogens including disease causing microbes is multi-factorial, and includes but is not limited to the following features and defense mechanisms: 1) innate immune sensors and mechanisms to immediately detect host ‘non-self’ biochemical features and patterns present in the structure of invading microbes such as viruses or bacteria that are not found in humans, that are termed Pathogen Associated Molecular Patterns (PAMP's); and 2) adaptive immune responses, which are in part activated by PAMP's, including host-generated viral antigen-recognizing antibodies and virus-infected cell recognizing cytotoxic T lymphocytes (CTL's) that are subsequently generated to remove and provide ongoing, longer-term protection against microbes, including viruses, virus infected human cells, and bacteria.

What are needed are new compositions and methods that reduce and, if possible eliminate the recognition of recombinant AAV vectors by the complex and efficient human immune response that has evolved to recognize and eliminate viruses in nature.

### SUMMARY OF THE INVENTION

It is to be understood that both the foregoing general description of the embodiments and the following detailed description are exemplary, and thus do not restrict the scope of the embodiments.

The inventions describe herein provide novel insights into the cause of unwanted immune responses in recombinant AAV vector administration and describe methods to decrease or eliminate them to achieve improvement in clinical gene therapy outcomes. The novel insights provided also describe methods to increase immune responses when desired, which will contribute to improved immunotherapeutics for cancer and vaccines for infectious disease.

In one aspect, the invention provides a method of generating a recombinant AAV vector with reduced immunogenicity, comprising:

providing eukaryotic cells with a nucleic acid comprising a sequence of interest that is flanked by AAV inverted terminal repeats,

wherein the nucleic acid comprises CpG dinucleotide sites, wherein at least a portion of the CpG dinucleotide sites are methylated, wherein the eukaryotic cell expresses one or more other components necessary to achieve recombinant AAV biosynthesis,

whereby the recombinant AAV vector is generated by the eukaryotic cell, wherein the generated recombinant AAV vector comprises nucleic acid wherein at least a portion of the CpG dinucleotide sites are methylated.

In another aspect, the invention provides a method of generating a recombinant AAV vector with reduced immunogenicity, comprising:

providing a eukaryotic cell line comprising a stably integrated nucleic acid comprising a sequence of interest that is flanked by AAV inverted terminal repeats,

wherein the nucleic acid comprises CpG dinucleotide sites, wherein the eukaryotic cell expresses one or more other components necessary to achieve recombinant AAV biosynthesis,

whereby the recombinant AAV vector is generated by the eukaryotic cell, wherein the generated recombinant AAV vector comprises nucleic acid wherein at least a portion of the CpG dinucleotide sites are methylated.

In another aspect, the invention provides a method of generating a recombinant AAV vector with reduced immunogenicity, comprising:

providing eukaryotic cells with a nucleic acid comprising a sequence of interest that is flanked by AAV inverted terminal repeats,

wherein the nucleic acid comprises CpG dinucleotide sites, wherein the eukaryotic cells have been modified to express increased levels of a polypeptide capable of methylating CpG dinucleotide sites,

wherein the eukaryotic cells express one or more other components necessary to achieve recombinant AAV biosynthesis,

whereby the recombinant AAV vector is generated by the eukaryotic cells, wherein the generated recombinant AAV vector comprises nucleic acid wherein at least a portion of the CpG dinucleotide sites are methylated.

In another aspect, the invention provides a method of generating a recombinant AAV vector with enhanced immunogenicity, comprising:

providing eukaryotic cells with a nucleic acid comprising a sequence of interest that is flanked by AAV inverted terminal repeats,

wherein the nucleic acid has been engineered to be enriched in immunogenic CpG containing motifs,

wherein the eukaryotic cell expresses one or more other components necessary to achieve recombinant AAV biosynthesis,

wherein the eukaryotic cell optionally has a reduced capability of methylating CpG dinucleotide sites,

whereby the recombinant AAV vector is generated by the eukaryotic cell, wherein the generated recombinant AAV vector comprises nucleic acid wherein at least a portion of the CpG dinucleotide sites are unmethylated.

In another aspect, the invention provides a method of generating a recombinant AAV vector with enhanced immunogenicity, comprising:

providing eukaryotic cells with a nucleic acid comprising a sequence of interest that is flanked by AAV inverted terminal repeats, wherein the nucleic acid comprises CpG dinucleotide sites,

wherein the eukaryotic cell expresses one or more other components necessary to achieve recombinant AAV biosynthesis,

wherein the eukaryotic cell has a reduced capability of methylating CpG dinucleotide sites,

whereby the recombinant AAV vector is generated by the eukaryotic cell, wherein the generated recombinant AAV vector comprises nucleic acid wherein at least a portion of the CpG dinucleotide sites are unmethylated.

In another aspect, the invention provides a method of making a plasmid DNA in bacterial cells, wherein the plasmid DNA comprises nucleic acid comprising a sequence of interest that is flanked by AAV inverted terminal repeats, wherein the nucleic acid comprises CpG dinucleotide sites,

comprising transforming the bacterial cells with the nucleic acid, wherein the bacterial cells are modified to express a polypeptide capable of methylating CpG dinucleotide sites, whereby the bacterial cells produce plasmid DNA comprising the nucleic acid, wherein at least a portion of the CpG dinucleotide sites in the nucleic acid are methylated.

In another aspect, the invention provides a method of making CpG methylated nucleic acid in vitro, wherein the nucleic acid comprises a sequence of interest that is flanked by AAV inverted terminal repeats,

comprising contacting the nucleic acid with a polypeptide capable of methylating CpG dinucleotide sites, whereby at least a portion of the CpG dinucleotide sites in the nucleic acid are methylated.

In another aspect, the invention provides a recombinant AAV vector generated according to the methods of the invention.

In another aspect, the invention provides a method of treating a disease or condition in a subject, comprising administering to the subject an effective amount of the recombinant AAV vector of the invention.

In another aspect, the invention provides an isolated eukaryotic cell useful for making a recombinant AAV according to the invention.

In another aspect, the invention relates to methods to generate recombinant AAV vectors in which the vector genome DNA contains CpG dinucleotides that occur at a frequency and are methylated at a frequency that is consistent with the frequency of occurrence and methylation corresponding to those found in human DNA so that said vector DNA is not recognized as a PAMP. In some embodiments, the methods comprise using a mammalian cell line stably transfected with therapeutic expression cassette composed of a gene of interest, a promoter, a-polyadenylation sequence, flanked by AAV inverted terminal repeats, which is expanded and then transiently transfected with other components required to achieve rAAV biosynthesis/generation, namely plasmid DNA expressing AAV Rep and Cap, and a plasmid DNA expressing helper virus genes Adenovirus E2, E4, and VA RNA.

In some embodiments, the methods use mammalian cell lines that are stably transfected with therapeutic expression cassette and also stably transfected with one or more supplemental methyl transferase genes, said cell line which is expanded and then transiently transfected with other components required to achieve rAAV biosynthesis/generation as described above.

In some embodiments the methods comprise transient transfection of mammalian cells such as HEK293 cells that are expanded and then transiently transfected with plasmid DNA, for example ‘triple transfection’ containing the above mentioned genes but in which the vector plasmid has been prepared in E. coli containing methyl transferases such that the vector plasmid has methylated CpG dinucleotides. In some embodiments, the methods comprise transient transfection of mammalian cells such as HEK293 cells that are expanded and then transiently transfected with synthetic DNA, such as Doggybone DNA, for example by ‘triple transfection’ containing the above mentioned genes but in which the synthetic DNA that contains the vector expression cassette has been prepared using methyl transferase so that it contains CpG dinucleotides that are methylated.

In some embodiments, the methods comprise using an insect cell line stably transfected with the therapeutic expression cassette and infected with recombinant baculoviruses that provide the other genes required to achieve rAAV biosynthesis/generation as described above.

In some embodiments, the methods comprise using an insect cell line stably transfected with the therapeutic expression cassette and also stably transfected with one or more supplemental methyl transferase genes, said cell line which is expanded and then infected with the other genes required to achieve rAAV biosynthesis/generation as described above.

The resulting vector can be purified from a crude cell harvest using methods and conditions consistent with current Good Manufacturing Practices for use in human gene therapy. In some embodiments of the methods herein, the methods provide the key benefit of prevention of packaging of unmethylated CpG sequences at levels that may bind TLR9 and activate detrimental immune responses.

The methods can include additional embodiments such as one or more of the following features. For example, in some embodiments, the plasmid DNA expressing helper virus genes may express Adenovirus or Herpesvirus or Baculovirus genes known to support rAAV generation in cell culture. In some embodiments, non-modified or stably transfected cell lines may be HEK293, HEK293T, BHK, CHO, HeLa, Vero, or any other mammalian cell lines know to support rAAV generation in cell culture. In some embodiments, non-modified or stably transfected cell lines may include SF9 or another other insect cell line known to support rAAV generation in cell culture. In some embodiments, the therapeutic expression cassette may contain one of the following genes of interest: coagulation factor IX, coagulation factor VIII, dystrophin, microdystrophin, alpha1 antitrypsin, and any other gene known to be missing and contributes to genetic disease. The DNA sequences for all of said genes may correspond to the cDNA sequence, or to any modification to said sequences arising due to truncations (for example ‘mini dystrophin,’ B domain deleted FVIII′, etc), enhanced potency (e.g. ‘Padua’ variant FIX), or codon modification (for example ‘codon optimization’) of any type. The therapeutic expression cassette may contain genes encoding a monoclonal antibody, a fragment of a monoclonal antibody, and any sequence variants of monoclonal antibody genes as mentioned above.

In some embodiments, the therapeutic expression cassette that when expressed following administration to subjects (such as humans) may provide a therapeutic benefit for genetic diseases including, hemophilia B, hemophilia A, Duchenne's Muscular Dystrophy, alpha1 antitrypsin deficiency, and any other genetic disease, for example those that can be treated using the corresponding recombinant proteins. In some embodiments, the therapeutic expression of the cassette may correspond to any gene that may have a therapeutic benefit for any genetic disease. In some embodiments, the therapeutic expression cassette may correspond to any gene that has a therapeutic benefit for diseases without clear genetic etiology, including cancer, autoimmune diseases, infectious diseases, Parkinson's Disease, Alzheimer's Disease, macular degeneration, and diabetes, for example those that can be treated using monoclonal antibodies. In some embodiments, the resulting vector, upon suitable purification and testing, may be administered to human subjects through the following routes of administration; intravenous, systemic, intramuscular, intracranial, intraocular, intraparenchymal, direct administration to a tissue or organ, etc. In other embodiments, the resulting vector may be used to modify human cells ex vivo, for example for gene transfer or gene editing of human stem cells prior to their readministration to a human subject.

In another aspect, the invention relates to a method to generate recombinant AAV vectors that increase immune responses using a mammalian cell line transiently transfected with therapeutic expression cassette composed of a gene of interest enriched in CpG motifs, a promoter, a-polyadenylation sequence, flanked by AAV inverted terminal repeats, which is expanded and then transiently transfected with other components required to achieve rAAV biosynthesis/generation, namely plasmid DNA expressing AAV Rep and Cap, and a plasmid DNA expressing helper virus genes Adenovirus E2, E4, and VA RNA, said method expressly intended to increase the packaging of unmethylated CpG sequences that are immune-stimulatory and contribute to desirable immune responses for elimination of vector transduced cells.

Modification of the commonly used transient transfection procedures for rAAV vector generation and production such that the plasmid DNA that is used as a component in the cell culture based production of rAAV vectors itself is methylated at the CpG motifs is an approach which is not currently practiced with plasmid DNA prepared as a raw material for rAAV vector production. It should be understood that when stating herein that the plasmid DNA is methylated, a range of methylation efficiencies that are practically achievable is envisaged and beneficial, in addition to complete (100%) methylation in every instance. The range can be very high, such as greater than 90% methylation; high, such as 60-90%; or moderate, such as 40-60%.

In some embodiments of the method where the therapeutic expression cassette is composed of a gene of interest enriched in CpG motifs, the methods can include one or more of the following features. For example, in some embodiments, the plasmid DNA expressing helper virus genes may express Herpes or Adenovirus virus genes known to support rAAV generation in cell culture. In some embodiments, the stably transfected cell line may be HEK293, HEK293T. In some embodiments, the stably transfected cell line may be BHK, CHO, HeLa, Vero, or any other mammalian cell lines known to support rAAV generation in cell culture. In some embodiments, the therapeutic expression cassette may contain any non-human gene that provides a target for immune recognition by the human immune response. In some embodiments, the therapeutic expression cassette may contain a non-human gene that encodes a protein that is presented at the surface of the transduced cell, thereby providing a recognition target for effector functions of the human immune responses. In some embodiments, the resulting rAAV vector may be targeted to cancer cells in a human subject, such that said cancer cells are specifically rendered targets for destruction by effector functions of the human immune response.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1. A plasmid expressing CpG methyltransferase M.SssI will be provided by transient transfection to increase CpG methylation of vector genomes during replication in HEK293 cells (A); vector plasmid DNA will be treated with M.SssI in vitro (B) prior to use for AAV vector production in HEK293 cells. Clinically relevant CpG densities and AAV serotypes will provide 12 unique vector constructs. Vector CpG methylation and TLR9-MyD88 pathway activation in vitro will be quantified using AAV batches generated by control and experimental protocols.

FIG. 2. Eukaryotic and prokaryotic provenance of AAV vector genome DNA. AAV vector plasmid encoding GFP was prepared by growing E. coli in the presence of dense nucleotide analog BrdU, purified and used for transfection production of recombinant AAV2 vector. Following purification (A), high resolution CsCl ultracentrifugation showed two AAV2-GFP activity at two densities; 1.39 mg/mL (expected peak, E) and 1.44 mg/mL (denser peak, P) (B).

FIG. 3. Model for AAV vector genome rescue, replication and packaging by transient transfection. The AAV genome in vector plasmid DNA is rescued by Holliday junction resolution leading to a closed circular AAV genome (top). Resolution at trs sites, and ITR repair leads to paired complementary (+/−) strands. Either strand can be packaged directly (left) to give AAV with a prokaryotic DNA vector genome or provide a template for de novo synthesis of a genome that is then packaged directly (right) providing a eukaryotic DNA genome. ‘a’ and ‘b’ are proposed points for introduction of methyl transferase to increase methylation of CpG dinucleotides.

FIG. 4. Research strategy graphic. Three parameters predicted to affect the Me^{neg}CpG-PAMP danger signal in AAV vectors are i) CpG methylation efficiency (Me)'s CpG/total CpG), ii) CpG density, and iii) AAV serotype. The predicted relationship between CpG density and methylation is shown the color scheme.

FIG. 6. Vector genome PAMP CpG content determines fate of AAV-transduced hepatocytes. Panel A: After systemic administration, AAV vectors containing DNA with high PAMP CpG content (a, red genome) transduce hepatocytes, leading to expression of the therapeutic (tx) protein but also cell surface presentation of capsid-derived peptides (red triangles) by MHC Class 1 molecules (b). A fraction of the vector dose enters proximal lymph nodes and is taken up by plasmacytoid dendritic cells (pDCs) (c) where vector DNA is processed in the lysosome and activates the TLR9-MyD88 pathway, and by conventional dendritic cells (cDCs) (d) where vector capsid-derived peptides (red ovals) are presented by MHC Class 2 molecules, recruiting capsid-specific CD4⁺ T cell help. These events lead to cDC licensing and maturation, and activation of capsid-specific CD8⁺ CTLs (e) that proliferate, migrate to the liver, and eliminate transduced hepatocytes (f). Panel B: AAV vectors containing DNA with low PAMP CpG content (g, green genome) similarly transduce hepatocytes but do not activate the TLR9-MyD88 pathway. CTLs are not formed, transduced hepatocytes are not eliminated, and cell-surface capsid peptide presentation wanes (h). In both cases AAV vectors activate the humoral arm of the immune response (i) leading to capsid antibodies.

### DETAILED DESCRIPTION

The present invention provides methods of making recombinant adeno associated viral vectors with altered immunogenicity profiles. This is advantageous when the intent is to reduce the immunogenicity of the recombinant vector to enable durable gene expression of a therapeutic polypeptide, or when the intent is to enhance the immunogenicity of the recombinant vector, for example, in vaccination or immunotherapy applications.

A novel approach to reduce immunogenicity is to increase CpG methylation by development of improved production technologies. Immunostimulatory CpG containing motifs are unmethylated CpG dinucleotides flanked by nucleotide sequences that enhance their ability to bind and activate Toll-like receptor 9 (TLR9). In some embodiments, the immunostimulatory CpG containing motifs are unmethylated CpG dinucleotides flanked by two 5′ purines and two 3′ pyrimidines. “Immunogenic” as used herein is interchangeable with “immunostimulatory.

Correction of CpG hypomethylation would allow the use of wild-type ORFs, and reduce immunostimulatory CpG motifs outside of the ORF. Improved production strategies vary depending on production cell line type (e.g. mammalian or insect) and mode of introduction (e.g. transfection or infection) of the genes required for vector generation. In some embodiments, the methods provide enough targeted methyl transferase during vector genome replication and packaging in production cells to achieve human physiological (˜75%) CpG methylation in the AAV vector product. The frequency of unmethylated CpG motifs in the genomes of AAV vectors prepared for human gene therapy is proposed to be a critical quality attribute for AAV-based investigation products for in vivo gene therapy, and specifications to ensure adequate innate-immune histocompatibility are provided herein.

In some embodiments, the invention disclosed herein is directed to making cell lines containing one or more copies of the vector genome expression cassette of interest using stable transfection, in certain embodiments including supplemental methyl transferases genes, an approach that can be performed because most therapeutic expression cassettes are not expected to be cytostatic or cytotoxic. This would require generation of a cell line for each transgene expression cassette of interest. To initiate rAAV vector production, helper virus-free transient transfection is performed to introduce the remaining components (gene products) required to achieve biosynthesis/generation of rAAV vectors, some of which are known to be cytostatic or cytotoxic. In contrast to currently described vector production methods (helper virus free transient transfection and helper virus infection methods), this novel method provides the safety of a helper virus-free method, and also significantly reduces or potentially eliminates the formation of unmethylated CpG motifs in the rAAV product, a feature implicated in the failure of gene therapy trials to date, addressing a key shortcoming of current transient transfection methods. The invention is also directed towards using existing cells lines (such as HEK293 or HEK293T) but in which the method to make the vector plasmid has been modified so that the CpG dinucleotides therein are methylated. The invention is also directed toward analogous strategies using recombinant baculovirus infection of insect cells to achieve methylation of the CpG dinucleotides in the AAV vectors prepared using these methods. It should be understood that there are likely to be some CpG motifs that are unmethylated but the majority of the CpG motifs will be methylated and only a minority of the CpG motifs will be unmethylated. This is a reasonable requirement because in humans, the extent of methylation is between 70-80% and that range is acceptable to avoid or reduce activation of deleterious immune responses. Achieving 100% methylation of CpG motifs should be understood as an unnecessary outcome for purposes of the invention. This approach is predicted to result in rAAV with reduced and/or minimal and/or no immune-stimulatory features, useful for durable therapeutic gene therapy in humans.

Based on CpG methylation mechanisms described and the insight provided in this application as approaches to reduce the immunogenicity of rAAV vectors, an alternative utility is an ability to design vectors and production systems to increase the immunostimulatory nature of rAAV vectors. Understanding the mechanism of packaging of vector genome DNA during vector generation in cell culture reveals a strategy to make highly immune-stimulatory rAAV vector by design. Specifically, codon modification to increase CpG motifs and CpG islands combined with: 1) the use of transient transfection with DNA lacking methylation on CpG motifs e.g. standard bacterial plasmid DNA or synthetic DNA such as ‘doggybone’ DNA; 2) use of mammalian cell lines for vector generation and production in which methyl transferase activity has been reduced or eliminated; or 3) use of insect cell lines for vector generation and production in which methyl transferase activity has been reduced or eliminated. More generally, any methods that reduces or prevents the expression of, or reduces or blocks the activity of enzymes that result in CpG methylation, such as methyl transferases, during the generation and production of rAAV vectors can result in rAAV vectors with strong immune-stimulatory features. Such vectors may be useful as a cancer immunotherapeutic, for example a rAAV with a vector genome that expresses a tumor antigen and containing a high number of unmethylated CpG motifs would provide stronger anti-tumor antigen CTL's. As another example, a rAAV with a vector genome that expresses a microbial antigen, including viral or bacterial antigens, and containing a high number of unmethylated CpG motifs would provide stronger immune responses to the corresponding microbial antigen. Targeted administration of such a vector to tumor cells in a human subject is predicted to cause a robust host immune response, resulting in efficient elimination of the targeted tumor cells by host immune effector functions such as cytotoxic T lymphocytes.

The viral vectors can be used for delivering heterologous nucleic acid sequences into a broad range of host cells, including both dividing and non-dividing cells. The vectors and other reagents, methods and pharmaceutical formulations of the present invention are additionally useful in a method of administering a protein or peptide to a subject in need thereof, as a method of treatment or otherwise. In this manner, the protein or peptide may thus be produced in vivo in the subject. The subject may be in need of the protein or peptide because the subject has a deficiency of the protein or peptide, or because the production of the protein or peptide in the subject may impart some therapeutic effect, as a method of treatment or otherwise, and as explained further below. The vectors and other reagents can also be administered directly to cells, for example, in ex vivo applications.

In general, the present invention may be employed to deliver any foreign nucleic acid with a biological effect to treat or ameliorate the symptoms associated with any disorder related to gene expression. In addition, the vectors can be administered to generate an immune response in a subject for therapeutic or prophylactic applications. Illustrative disease states include, but are not limited to: cystic fibrosis (and other diseases of the lung), hemophilia A, hemophilia B, thalassemia, anemia and other blood coagulation disorders, AIDs, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Gaucher's disease, Hurler's disease, adenosine deaminase deficiency, glycogen storage diseases and other metabolic defects, retinal degenerative diseases (and other diseases of the eye), diseases of solid organs (e.g., brain, liver, kidney, heart), and the like.

In addition, the present invention may be employed to deliver nucleic acids encoding monoclonal antibodies or fragments thereof that are known to provide beneficial biological effects to treat or ameliorate the symptoms associated with cancers, infectious diseases, and autoimmune diseases such as rheumatoid arthritis.

Gene therapy has substantial potential use in understanding and providing therapy for disease states. There are a number of inherited diseases in which defective genes are known and have been cloned. In some cases, the function of these cloned genes is known. In general, the above disease states fall into two classes: deficiency states, usually of enzymes, which are generally inherited in a recessive manner, and unbalanced states, at least sometimes involving regulatory or structural proteins, which are inherited in a dominant manner. For deficiency state diseases, gene transfer could be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. Thus the methods of the present invention permit the treatment of genetic diseases. As used herein, a disease state is treated by partially or wholly remedying the deficiency or imbalance that causes the disease or makes it more severe. The use of site-specific integration of nucleic sequences to cause mutations or to correct defects is also possible.

Finally, the instant invention finds further use in diagnostic and screening methods, whereby a gene of interest is transiently or stably expressed in a cell culture system, or alternatively, a transgenic animal model.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)). Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

Methods of Generating rAAV Vectors

In one embodiment, the invention provides a method of generating a recombinant adeno-associated viral (AAV) vector with reduced immunogenicity, comprising:

providing eukaryotic cells with a nucleic acid comprising a sequence of interest that is flanked by AAV inverted terminal repeats,

wherein the nucleic acid comprises CpG dinucleotide sites, wherein at least a portion of the CpG dinucleotide sites are methylated, wherein the eukaryotic cell expresses one or more other components necessary to achieve recombinant AAV biosynthesis,

whereby the recombinant AAV vector is generated by the eukaryotic cell, wherein the generated recombinant AAV vector comprises nucleic acid wherein at least a portion of the CpG dinucleotide sites are methylated.

In another embodiment, the invention provides a method of generating a recombinant AAV vector with reduced immunogenicity, comprising:

providing a eukaryotic cell line comprising a stably integrated nucleic acid comprising a sequence of interest that is flanked by AAV inverted terminal repeats,

wherein the nucleic acid comprises CpG dinucleotide sites, wherein the eukaryotic cell expresses one or more other components necessary to achieve recombinant AAV biosynthesis,

whereby the recombinant AAV vector is generated by the eukaryotic cell, wherein the generated recombinant AAV vector comprises nucleic acid wherein at least a portion of the CpG dinucleotide sites are methylated.

In another embodiment, the invention provides a method of generating a recombinant AAV vector with reduced immunogenicity, comprising:

providing eukaryotic cells with a nucleic acid comprising a sequence of interest that is flanked by AAV inverted terminal repeats,

wherein the nucleic acid comprises CpG dinucleotide sites, wherein the eukaryotic cells have been modified to express increased levels of a polypeptide capable of methylating CpG dinucleotide sites,

wherein the eukaryotic cells express one or more other components necessary to achieve recombinant AAV biosynthesis,

whereby the recombinant AAV vector is generated by the eukaryotic cells, wherein the generated recombinant AAV vector comprises nucleic acid wherein at least a portion of the CpG dinucleotide sites are methylated.

In another embodiment, the invention provides a method of generating a recombinant AAV vector with enhanced immunogenicity, comprising:

providing eukaryotic cells with a nucleic acid comprising a sequence of interest that is flanked by AAV inverted terminal repeats,

wherein the nucleic acid has been engineered to be enriched in immunogenic CpG containing motifs,

wherein the eukaryotic cell expresses one or more other components necessary to achieve recombinant AAV biosynthesis,

wherein the eukaryotic cell optionally has a reduced capability of methylating CpG dinucleotide sites,

whereby the recombinant AAV vector is generated by the eukaryotic cell, wherein the generated recombinant AAV vector comprises nucleic acid wherein at least a portion of the CpG dinucleotide sites are unmethylated.

In another embodiment, the invention provides a method of generating a recombinant AAV vector with enhanced immunogenicity, comprising:

providing eukaryotic cells with a nucleic acid comprising a sequence of interest that is flanked by AAV inverted terminal repeats, wherein the nucleic acid comprises CpG dinucleotide sites,

wherein the eukaryotic cell expresses one or more other components necessary to achieve recombinant AAV biosynthesis,

wherein the eukaryotic cell has a reduced capability of methylating CpG dinucleotide sites,

whereby the recombinant AAV vector is generated by the eukaryotic cell, wherein the generated recombinant AAV vector comprises nucleic acid wherein at least a portion of the CpG dinucleotide sites are unmethylated.

In some embodiments, the methods are useful for generating AAV vectors for gene therapy applications. “Gene therapy” is the insertion of genes into an individual's cells and/or tissues to treat a disease, commonly hereditary diseases wherein a defective mutant allele is replaced or supplemented with a functional one.

A “vector” is a replicon, such as a plasmid, cosmid, bacmid, phage or virus, to which another genetic sequence or element (either DNA or RNA) may be attached so as to bring about the replication of the attached sequence or element.

“Adeno-associated viruses,” from the parvovirus family, are small viruses with a genome of single stranded DNA. These viruses can insert genetic material at a specific site on chromosome 19 and are preferred because they are not associated with pathogenic disease in humans. The adeno-associated viral vector that can be used is not particularly limiting.

AAV vectors do not typically include viral genes associated with pathogenesis. Such vectors typically have one or more of the wild type AAV genes deleted in whole or in part, for example, rep and/or cap genes, but retain at least one functional flanking ITR sequence, as necessary for the rescue, replication, and packaging of the recombinant vector into an AAV vector particle. For example, in some embodiments, only the essential parts of vector e.g., the ITR and LTR elements, respectively are included. An AAV vector genome would therefore include sequences required in cis for replication and packaging (e.g., functional ITR sequences).

Recombinant AAV vector, as well as methods and uses thereof, can include any viral strain or serotype. As a non-limiting example, a recombinant AAV vector can be based upon any AAV genome, such as AAV-1, -2, -3, -4, -5, -6, -7, -8, -9, -10, -11, -12, or AAV-2i8, for example. Such vectors can be based on the same strain or serotype (or subgroup or variant), or be different from each other. As a non-limiting example, a recombinant AAV vector based upon one serotype genome can be identical to one or more of the capsid proteins that package the vector. In addition, a recombinant AAV vector genome can be based upon an AAV (e.g., AAV2) serotype genome distinct from one or more of the AAV capsid proteins that package the vector. For example, the AAV vector genome can be based upon AAV2, whereas at least one of the three capsid proteins could be a AAV1, AAV3, AAV4, AAV5, AAV6, AAV7, AAV8, AAV9, AAV10, AAV11, AAV12, or AAV-2i8 or variant thereof, for example. AAV variants include variants and chimeras of AAV1, AAV2, AAV3, AAV4, AAV5, AAV6, AAV7, AAV8, AAV9, AAV10, AAV11, AAV12 and AAV-2i8 capsids.

In some embodiments, adeno-associated virus (AAV) vectors include AAV1, AAV2, AAV3, AAV4, AAV5, AAV6, AAV7, AAV8, AAV9, AAV10, AAV11, AAV12, and AAV-2i8, as well as variants (e.g., capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. application Ser. No. 13/594,773, discloses LK01, LK02, LK03, etc.).

AAV and AAV variants (e.g., capsid variants) serotypes (e.g., VP1, VP2, and/or VP3 sequences) may or may not be distinct from other AAV serotypes, including, for example, AAV1-AAV12 (e.g., distinct from VP1, VP2, and/or VP3 sequences of any of AAV1-AAV12 serotypes).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

In various exemplary embodiments, an AAV vector related to a reference serotype has a polynucleotide, polypeptide or subsequence thereof that includes or consists of a sequence at least 80% or more (e.g., 85%, 90%, 95%, 96%, 97%, 98%, 99%, 99.1%, 99.2%, 99.3%, 99.4%, 99.5%, etc.) identical to one or more AAV1, AAV2, AAV3, AAV4, AAV5, AAV6, AAV7, AAV8, AAV9, AAV10, AAV11, AAV12, or AAV-2i8 (e.g., such as an ITR, or a VP1, VP2, and/or VP3 sequences).

In some embodiments, compositions, methods and uses of the invention include AAV sequences (polypeptides and nucleotides), and subsequences thereof that exhibit less than 100% sequence identity to a reference AAV serotype such as AAV1, AAV2, AAV3, AAV4, AAV5, AAV6, AAV7, AAV8, AAV9, AAV10, AAV11, AAV12, or AAV-2i8, but are distinct from and not identical to known AAV genes or proteins, such as AAV1, AAV2, AAV3, AAV4, AAV5, AAV6, AAV7, AAV8, AAV9, AAV10, AAV11, AAV12, or AAV-2i8, genes or proteins, etc. In one embodiment, an AAV polypeptide or subsequence thereof includes or consists of a sequence at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to any reference AAV sequence or subsequence thereof, such as AAV1, AAV2, AAV3, AAV4, AAV5, AAV6, AAV7, AAV8, AAV9, AAV10, AAV11, AAV12, or AAV-2i8 (e.g., VP1, VP2 and/or VP3 capsid or ITR). In particular aspects, an AAV variant has 1, 2, 3, 4, 5, 5-10, 10-15, 15-20 or more amino acid substitutions.

Recombinant AAV vectors, including AAV1, AAV2, AAV3, AAV4, AAV5, AAV6, AAV7, AAV8, AAV9, AAV10, AAV11, AA112, or AAV-2i8 and variant, related, hybrid and chimeric sequences, can be constructed using recombinant techniques that are known to the skilled artisan, to include one or more nucleic acid sequences (transgenes) flanked with one or more functional AAV ITR sequences.

As provided herein, the cells are provided with a nucleic acid comprising a sequence of interest that is flanked by AAV inverted terminal repeats.

In some embodiments, the nucleic acid has been engineered to decrease the frequency of CpG dinucleotide sites. In some embodiments, the CpG dinucleotide sites comprise immunogenic CpG containing motifs. In some embodiments, the frequency of CpG dinucleotide sites is decreased by at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99%.

In some embodiments, the nucleic acid has been engineered to be enriched in immunogenic CpG containing motifs. In some embodiments, the nucleic acid is engineered to have a frequency of CpG dinucleotide sites in the nucleic acid of about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% or more.

The nucleic acid can be provided to the cells in a number of ways and is not limiting. In some embodiments, the nucleic acid is provided to the eukaryotic cells by transient transfection. The terms “transform,” “transfect,” “transduce,” shall refer to any method or means by which a nucleic acid is introduced into a cell or host organism and may be used interchangeably to convey the same meaning. Such methods include, but are not limited to, transfection, electroporation, microinjection, infection, PEG-fusion and the like.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids and polynucleotides include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids and polynucleotides include naturally occurring, synthetic, and intentionally modified or altered sequences (e.g., variant nucleic acid).

A nucleic acid can also refer to a sequence which encodes a protein. Such proteins can be wild-type or a variant, modified or chimeric protein. A “variant protein” can mean a modified protein such that the modified protein has an amino acid alteration compared to wild-type protein. A “human protein” for use in the vectors of the invention is preferably a highly conserved protein which would not be recognized as a foreign or non-self antigen by the human immune system. Proteins encoded by a nucleic acid include therapeutic proteins.

A nucleic acid can also refer to a sequence which produces a transcript when transcribed. Such transcripts can be RNA, such as inhibitory RNA (RNAi, e.g., small or short hairpin (sh)RNA, microRNA (miRNA), small or short interfering (si)RNA, trans-splicing RNA, or antisense RNA).

Nucleic acids can be single, double, or triplex, linear or circular, and can be of any length. In discussing nucleic acids, a sequence or structure of a particular polynucleotide may be described herein according to the convention of providing the sequence in the 5′ to 3′ direction.

The nucleic acid can comprise an “expression operon” possessing operably linked transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism. The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

In some embodiments, the nucleic acid comprises a promoter, and a polyadenylation sequence. The term “promoters” or “promoter” as used herein can refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism can be utilized to enhance recombinant product expression from a DNA sequence that originates from another organism. For example, a vertebrate promoter may be used for the expression of jellyfish GFP in vertebrates. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art.

In one embodiment, high-level constitutive expression will be desired. Examples of such promoters include, without limitation, the retroviral Rous sarcoma virus (RSV) LTR promoter/enhancer, the cytomegalovirus (CMV) immediate early promoter/enhancer (see, e.g., Boshart et al, Cell, 41:521-530 (1985)), the SV40 promoter, the dihydrofolate reductase promoter, the cytoplasmic .beta.-actin promoter and the phosphoglycerol kinase (PGK) promoter.

In another embodiment, inducible promoters may be desired. Inducible promoters are those which are regulated by exogenously supplied compounds, either in cis or in trans, including without limitation, the zinc-inducible sheep metallothionine (MT) promoter; the dexamethasone (Dex)-inducible mouse mammary tumor virus (MMTV) promoter; the T7 polymerase promoter system (WO 98/10088); the tetracycline-repressible system (Gossen et al, Proc. Natl. Acad. Sci. USA, 89:5547-5551 (1992)); the tetracycline-inducible system (Gossen et al., Science, 268:1766-1769 (1995); see also Harvey et al., Curr. Opin. Chem. Biol., 2:512-518 (1998)); the RU486-inducible system (Wang et al., Nat. Biotech., 15:239-243 (1997) and Wang et al., Gene Ther., 4:432-441 (1997)]; and the rapamycin-inducible system (Magari et al., J. Clin. Invest., 100:2865-2872 (1997); Rivera et al., Nat. Medicine. 2:1028-1032 (1996)). Other types of inducible promoters which may be useful in this context are those which are regulated by a specific physiological state, e.g., temperature, acute phase, or in replicating cells only.

In another embodiment, the native promoter for the transgene or nucleic acid sequence of interest will be used. The native promoter may be preferred when it is desired that expression of the transgene or the nucleic acid sequence should mimic the native expression. The native promoter may be used when expression of the transgene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In one embodiment, the recombinant AAV vector comprises a transgene operably linked to a tissue-specific promoter. For instance, if expression in skeletal muscle is desired, a promoter active in muscle may be used. These include the promoters from genes encoding skeletal α-actin, myosin light chain 2A, dystrophin, muscle creatine kinase, as well as synthetic muscle promoters with activities higher than naturally-occurring promoters. See Li et al., Nat. Biotech., 17:241-245 (1999). Examples of promoters that are tissue-specific are known for liver albumin, Miyatake et al. J. Virol., 71:5124-32 (1997); hepatitis B virus core promoter, Sandig et al., Gene Ther. 3:1002-9 (1996); alphafetoprotein (AFP), Arbuthnot et al., Hum. Gene Ther., 7:1503-14 (1996)], bone (osteocalcin, Stein et al., Mol. Biol. Rep., 24:185-96 (1997); bone sialoprotein, Chen et al., J. Bone Miner. Res. 11:654-64 (1996)), lymphocytes (CD2, Hansal et al., J. Immunol., 161:1063-8 (1998); immunoglobulin heavy chain; T cell receptor a chain), neuronal (neuron-specific enolase (NSE) promoter, Andersen et al. Cell. Mol. Neurobiol., 13:503-15 (1993); neurofilament light-chain gene, Piccioli et al., Proc. Natl. Acad. Sci. USA, 88:5611-5 (1991); the neuron-specific vgf gene, Piccioli et al., Neuron, 15:373-84 (1995)]; among others.

The term “enhancers” or “enhancer” as used herein can refer to a DNA sequence that is located adjacent to the DNA sequence that encodes a recombinant product. Enhancer elements are typically located upstream of a promoter element or can be located downstream of or within a coding DNA sequence (e.g., a DNA sequence transcribed or translated into a recombinant product or products). Hence, an enhancer element can be located 100 base pairs, 200 base pairs, or 300 or more base pairs upstream or downstream of a DNA sequence that encodes recombinant product. Enhancer elements can increase an amount of recombinant product expressed from a DNA sequence above increased expression afforded by a promoter element. Multiple enhancer elements are readily available to persons of ordinary skill in the art.

In some embodiments, the nucleic acid provided to the eukaryotic cells is synthesized in vitro. In some embodiments, the nucleic acid is methylated in vitro with a methyltransferase protein capable of methylating CpG dinucleotide sites. In some embodiments, the nucleic acid is methylated in bacterial cells modified to express a methyltransferase protein capable of methylating CpG dinucleotide sites.

The methyltransferase protein for use in the present invention is not limiting. DNA methyltransferases (DNMTs) are a highly conserved family of proteins. In mammals, there are three major DNMTs: DNMT1, DNMT3a and DNMT3b. DNMT1 is known as a maintenance DNMT, while DNMT3a and 3b are de novo DNMTs. The common feature of these DNMTs is ten conserved amino acids in their C-terminal catalytic domain. They also have ten conserved motifs that fulfill catalytic, cofactor binding, and DNA targeting functions. DNMT1 binds to hemi-methylated DNA (one strand methylated) at CpG sties. After DNA replication, while the parent strand remains methylated the newly synthesized strand is not. DNMT1 binds to these hemi-methylated CpG sites and methylates the cytosine on the newly synthesized strand. The DNMT3a and DNMT3b methyltransferases do not require hemi-methylated DNA to bind, and are known to show equal affinity for hemi-methylated and non-methylated DNA. There is another DNMT in vertebrates, DNMT2. DNMT2 shares strong sequence homology to the other DNMTs, but is reported to show almost no detectable DNA-cysteine methylation activity with its role in DNA methylation unclear.

In some embodiments, the methyltransferase protein can be any of DNMT1, DNMT3a, DNMT3b and/or M.SssI.

Articles discussing DNA methyltransferases are listed and are incorporated herein by reference for their teaching of various DNA methyltransferases and their uses, in particular the Bestor review: (a) Alexandrov, A., Chernyakov, I., Gu, W., Hiley, S. L., Hughes, T. R., Grayhack, E. J., and Phizicky, E. M. (2006). Rapid tRNA decay can result from lack of nonessential modifications. Mol. Cell 21, 87-96; (b) Bestor, T. H. (2000). The DNA methyltransferases of mammals. Hum. Mol. Genet. 9, 2395-2402; (c) Chen, C. C., Wang, K. Y., and Shen, C. K. (2013). DNA 5-methylcytosine demethylation activities of the mammalian DNA methyltransferases. J. Biol. Chem. 288, 9084-9091; (d) Goll, M. G., Kirpekar, F., Maggert, K. A., Yoder, J. A., Hsieh, C. L., Zhang, X., Golic, K. G., Jacobsen, S. E., and Bestor, T. H. (2006). Methylation of tRNAAsp by the DNA methyltransferase homolog Dnmt2. Science 311, 395-398; (e) Gowher, H., Liebert, K., Hermann, A., Xu, G., and Jeltsch, A. (2005). Mechanism of stimulation of catalytic activity of Dnmt3A and Dnmt3B DNA-(cytosine-05)-methyltransferases by Dnmt3L. J. Biol. Chem. 280, 13341-13348; (f) Jeltsch, A., Nellen, W., and Lyko, F. (2006). Two substrates are better than one: dual specificities for Dnmt2 methyltransferases. Trends Biochem. Sci. 31, 306-308; (g) Okano, M., Bell, D. W., Haber, D. A., and Li, E. (1999). DNA methyltransferases Dnmt3a and Dnmt3b are essential for de novo methylation and mammalian development. Cell 99, 247-257; (h) Okano, M., Xie, S., and Li, E. (1998). Cloning and characterization of a family of novel mammalian DNA (cytosine-5) methyltransferases. Nat. Genet. 19, 219-220; (i) Rhee, I., Jair, K. W., Yen, R. W., Lengauer, C., Herman, J. G., Kinzler, K. W., Vogelstein, B., Baylin, S. B., and Schuebel, K. E. (2000). CpG methylation is maintained in human cancer cells lacking DNMT1. Nature 404, 1003-1007; (j) Robertson, K. D., Uzvolgyi, E., Liang, G., Talmadge, C., Sumegi, J., Gonzales, F. A., and Jones, P. A. (1999). The human DNA methyltransferases (DNMTs) 1, 3a and 3b: coordinate mRNA expression in normal tissues and overexpression in tumors. Nucleic Acids Res. 27, 2291-2298; (k) Sardet, C., Roegiers, F., Dumollard, R., Rouviere, C., and McDougall, A. (1998). Calcium waves and oscillations in eggs. Biophys. Chem. 72, 131-140; (1) Schaefer, M., Pollex, T., Hanna, K., Tuorto, F., Meusburger, M., Helm, M., and Lyko, F. (2010). RNA methylation by Dnmt2 protects transfer RNAs against stress-induced cleavage. Genes Dev. 24, 1590-1595; (m) Vertino, P. M., Yen, R. W., Gao, J., and Baylin, S. B. (1996). De novo methylation of CpG island sequences in human fibroblasts overexpressing DNA (cytosine-5-)-methyltransferase. Mol. Cell. Biol. 16, 4555-4565.

The eukaryotic cells for making the recombinant AAV vector are not limiting. In some embodiments, the eukaryotic cells are mammalian cells. In some embodiments, the eukaryotic cells are selected from the group consisting of HEK293, HEK293T, BHK, CHO, HeLa, and Vero cells. In some embodiments, the eukaryotic cells are insect cells.

In some embodiments, particularly in cases where it is desirable to reduce immunogenicity of the recombinant AAV vector, the eukaryotic cell has been modified to express a polypeptide capable of methylating CpG dinucleotide sites.

In some embodiments, the polypeptide is a methyltransferase protein selected from the group consisting of DNMT1, DNMT3a, DNMT3b and/or M.SssI.

In some embodiments where it is desirable to enhance the immunogenicity of the recombinant AAV vector, the eukaryotic cells are treated with one or more agents that inhibit the activity of one or more endogenous DNA methyltransferase proteins. In some embodiments, the DNA methyltransferase protein is selected from the group consisting of DNMT1, DNMT3a, DNMT3b and combinations thereof. For example, in some embodiments, the eukaryotic cells are treated with one or more agents that reduce the expression of one or more endogenous DNA methyltransferase proteins. In some embodiments, the eukaryotic cells have been modified to reduce expression of one or more endogenous DNA methyltransferase proteins. In some embodiments, agents that can be used to reduce expression include RNA, such as inhibitory RNA (RNAi, e.g., small or short hairpin (sh)RNA, microRNA (miRNA), small or short interfering (si)RNA, trans-splicing RNA, or antisense RNA). The eukaryotic cells can also be modified to express such agents.

In some embodiments, the eukaryotic cell is provided with nucleic acids capable of reducing the expression of one or more methyltransferase proteins. In some embodiments, the nucleic acid molecule comprises a nucleotide sequence that binds to at least a portion of a nucleotide sequence of a methyltransferase protein. The nucleic acid molecule can be of any length, so long as at least part of the molecule hybridizes sufficiently and specifically to a methyltransferase protein mRNA. The nucleic acid molecule can bind to any region of the mRNA. In some embodiments, the nucleic acid molecule can bind to and hybridize with an intronic sequence in the nucleic acid. In some embodiments, the nucleic acid molecule can bind to and hybridize with an exonic sequence in the nucleic acid.

In some embodiments, the genomic nucleotide sequence of DNMT1 is shown in SEQ ID NO: 1 (NCBI Reference Sequence: HGNC:2976). In some embodiments, the genomic nucleotide sequence of DNMT3a is shown in SEQ ID NO: 2 (NCBI Reference Sequence: HGNC:2978). In some embodiments, the genomic nucleotide sequence of DNMT3b is shown in SEQ ID NO: 3 (NCBI Reference Sequence: HGNC:2979). In some embodiments, methyltransferase M.SssI can be used. In some embodiments, the nucleotide sequence of M.SssI is SEQ ID NO:4 (NCBI accession no.: X17195).

In some embodiments, the mRNA sequence corresponds to the joined nucleotides present in exons at the following positions in SEQ ID NO:1: 41208 . . . 41467, 54210 . . . 54246, 55402 . . . 55509, 55718 . . . 55937, 56053 . . . 56100, 58920 . . . 58995, 60669 . . . 60747, 62382 . . . 62416, 63113 . . . 63197, 67923 . . . 67957, 69602 . . . 69689, 72927 . . . 72961, 73539 . . . 73620, 75869 . . . 75903, 76224 . . . 76269, 76365 . . . 76445, 76520 . . . 76629, 79778 . . . 79896, 80342 . . . 80434, 81231 . . . 81382, 81514 . . . 81701, 81808 . . . 81994, 84440 . . . 84537, 84742 . . . 84889, 86319 . . . 86434, 86630 . . . 86834, 87270 . . . 87403, 89763 . . . 89936, 92300 . . . 92521, 94067 . . . 94259, 95098 . . . 95182, 95378 . . . 95506, 95959 . . . 96241, 96470 . . . 96611, 97682 . . . 97848, 98278 . . . 98455, 99007 . . . 99202, 100000 . . . 100166, 100435 . . . 100551, 101980 . . . 102070, and 102586 . . . 102941.

In some embodiments, the mRNA sequence corresponds to the joined nucleotides present in exons at the following positions in SEQ ID NO:1: 41388 . . . 41467, 54210 . . . 54246, 55402 . . . 55509, 55718 . . . 55937, 56053 . . . 56100, 58920 . . . 58995, 60669 . . . 60747, 62382 . . . 62416, 63113 . . . 63197, 67923 . . . 67957, 69602 . . . 69689, 72927 . . . 72961, 73539 . . . 73620, 75869 . . . 75903, 76224 . . . 76269, 76365 . . . 76445, 76520 . . . 76629, 79778 . . . 79896, 80342 . . . 80434, 81231 . . . 81382, 81514 . . . 81701, 81808 . . . 81994, 84440 . . . 84537, 84742 . . . 84889, 86319 . . . 86434, 86630 . . . 86834, 87270 . . . 87403, 89763 . . . 89936, 92300 . . . 92521, 94067 . . . 94259, 95098 . . . 95182, 95378 . . . 95506, 95959 . . . 96241, 96470 . . . 96611, 97682 . . . 97848, 98278 . . . 98455, 99007 . . . 99202, 100000 . . . 100166, 100435 . . . 100551, 101980 . . . 102070, and 102586 . . . 102620.

In some embodiments, the mRNA sequence corresponds to the joined nucleotides present in exons at the following positions in SEQ ID NO:2: 5001 . . . 5161, 33430 . . . 33678, 47348 . . . 47452, 64880 . . . 65150, 72048 . . . 72091, 72504 . . . 72650, 99339 . . . 99554, 99842 . . . 100000, 100433 . . . 100540, 100815 . . . 100971, 101282 . . . 101431, 101527 . . . 101571, 102259 . . . 102338, 102939 . . . 103051, 103253 . . . 103436, 103609 . . . 103693, 105884 . . . 106029, 106861 . . . 106951, 107141 . . . 107289, 108376 . . . 108461, 110586 . . . 110655, 111766 . . . 111884, and

In some embodiments, the mRNA sequence corresponds to the joined nucleotides present in exons at the following positions in SEQ ID NO:2: 5001 . . . 5161, 33430 . . . 33678, 47348 . . . 47452, and 64880 . . . 66139.

In some embodiments, the mRNA sequence corresponds to the joined nucleotides present in exons at the following positions in SEQ ID NO:2: 5676 . . . 5765, 33430 . . . 33678, 47348 . . . 47452, 64880 . . . 65150, 72048 . . . 72091, 72504 . . . 72650, 99339 . . . 99554, 99842 . . . 100000, 100433 . . . 100540, 100815 . . . 100971, 101282 . . . 101431, 101527 . . . 101571, 102259 . . . 102338, 102939 . . . 103051, 103253 . . . 103436, 103609 . . . 103693, 105884 . . . 106029, 106861 . . . 106951, 107141 . . . 107289, 108376 . . . 108461, 110586 . . . 110655, 111766 . . . 111884, and 113171 . . . 114630. In some embodiments, the mRNA sequence corresponds to the joined nucleotides present in exons at the following positions in SEQ ID NO:2: 33607 . . . 33678, 47348 . . . 47452, 64880 . . . 65150, 72048 . . . 72091, 72504 . . . 72650, 99339 . . . 99554, 99842 . . . 100000, 100433 . . . 100540, 100815 . . . 100971, 101282 . . . 101431, 101527 . . . 101571, 102259 . . . 102338, 102939 . . . 103051, 103253 . . . 103436, 103609 . . . 103693, 105884 . . . 106029, 106861 . . . 106951, 107141 . . . 107289, 108376 . . . 108461, 110586 . . . 110655, 111766 . . . 111884, and 113171 . . . 113312.

In some embodiments, the mRNA sequence corresponds to the joined nucleotides present in exons at the following positions in SEQ ID NO:2: 33607 . . . 33678, 47348 . . . 47452, 64880 . . . 65150, 72048 . . . 72091, 72504 . . . 72650, 99339 . . . 99554, 99842 . . . 100000, 100433 . . . 100540, 100815 . . . 100971, 101282 . . . 101431, 101527 . . . 101571, 102259 . . . 102338, 102939 . . . 103051, 103253 . . . 103436, 103609 . . . 103693, 105884 . . . 106029, 106861 . . . 106951, 107141 . . . 107289, 108376 . . . 108461, 110586 . . . 110655, 111766 . . . 111884, and 113171 . . . 113312.

In some embodiments, the mRNA sequence corresponds to the joined nucleotides present in exons at the following positions in SEQ ID NO:2: 33607 . . . 33678, 47348 . . . 47452, and 64880 . . . 65203.

In some embodiments, the mRNA sequence corresponds to the joined nucleotides present in exons at the following positions in SEQ ID NO:2: 95276 . . . 95397, 97867 . . . 97934, 99339 . . . 99554, 99842 . . . 100000, 100433 . . . 100540, 100815 . . . 100971, 101282 . . . 101431, 101527 . . . 101571, 102259 . . . 102338, 102939 . . . 103051, 103253 . . . 103436, 103609 . . . 103693, 105884 . . . 106029, 106861 . . . 106951, 107141 . . . 107289, 108376 . . . 108461, 110586 . . . 110655, 111766 . . . 111884, and 113171 . . . 114630.

In some embodiments, the mRNA sequence corresponds to the joined nucleotides present in exons at the following positions in SEQ ID NO:2: 95394 . . . 95397, 97867 . . . 97934, 99339 . . . 99554, 99842 . . . 100000, 100433 . . . 100540, 100815 . . . 100971, 101282 . . . 101431, 101527 . . . 101571, 102259 . . . 102338, 102939 . . . 103051, 103253 . . . 103436, 103609 . . . 103693, 105884 . . . 106029, 106861 . . . 106951, 107141 . . . 107289, 108376 . . . 108461, 110586 . . . 110655, 111766 . . . 111884, and 113171 . . . 113312.

In some embodiments, the mRNA sequence corresponds to the joined nucleotides present in exons at the following positions in SEQ ID NO:3: 1 . . . 315, 17934 . . . 18081, 18969 . . . 19030, 22374 . . . 22475, 24118 . . . 24243, 24846 . . . 25067, 26470 . . . 26628, 29217 . . . 29324, 30242 . . . 30386, 31152 . . . 31211, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, 42954 . . . 43023, 43825 . . . 43943, and 45378 . . . 46972.

In some embodiments, the mRNA sequence corresponds to the joined nucleotides present in exons at the following positions in SEQ ID NO:3: 1 . . . 315, 17934 . . . 18081, 18969 . . . 19030, 22374 . . . 22475, 24118 . . . 24243, 24846 . . . 25067, 26470 . . . 26628, 29217 . . . 29324, 30242 . . . 30386, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, 42954 . . . 43023, 43825 . . . 43943, and 45378 . . . 46972.

In some embodiments, the mRNA sequence corresponds to the joined nucleotides present in exons at the following positions in SEQ ID NO:3: 1 . . . 315, 17934 . . . 18081, 18969 . . . 19030, 22374 . . . 22475, 24118 . . . 24243, 24846 . . . 25067, 26470 . . . 26628, 29217 . . . 29324, 30242 . . . 30386, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086 and 45378 . . . 46972.

In some embodiments, the mRNA sequence corresponds to the joined nucleotides present in exons at the following positions in SEQ ID NO:3: 1 . . . 315, 17934 . . . 18081, 18969 . . . 19030, 22374 . . . 22475, 24846 . . . 25067, 26470 . . . 26628, 29217 . . . 29324, 30242 . . . 30386, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, and 45378 . . . 46972.

In some embodiments, the mRNA sequence corresponds to the joined nucleotides present in exons at the following positions in SEQ ID NO:3: 1 . . . 315, 17934 . . . 18081, 18969 . . . 19030, 24846 . . . 25067, 26470 . . . 26628, 29217 . . . 29324, 30242 . . . 30386, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, and 45378 . . . 46972.

In some embodiments, the mRNA sequence corresponds to the joined nucleotides present in exons at the following positions in SEQ ID NO:3: 17468 . . . 17761, 17934 . . . 18081, 18969 . . . 19030, 22374 . . . 22475, 24118 . . . 24243, 24846 . . . 25067, 26470 . . . 26628, 29217 . . . 29324, 30242 . . . 30386, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, 42954 . . . 43023, 43825 . . . 43943, and 45378 . . . 46972.

In some embodiments, the mRNA sequence corresponds to the joined nucleotides present in exons at the following positions in SEQ ID NO:3: 17583 . . . 17761, 17934 . . . 18081, 18969 . . . 19030, 22374 . . . 22475, 24846 . . . 25067, 26470 . . . 26628, 29217 . . . 29324, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, 42954 . . . 43023, and 43825 . . . 43940.

In some embodiments, the mRNA sequence corresponds to the joined nucleotides present in exons at the following positions in SEQ ID NO:3: 17585 . . . 17761, 17934 . . . 18081, 18969 . . . 19030, 22374 . . . 22475, 24118 . . . 24243, 24846 . . . 25067, 26470 . . . 26628, 29217 . . . 29324, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, and 45378 . . . 46972.

In some embodiments, the mRNA sequence corresponds to the joined nucleotides present in exons at the following positions in SEQ ID NO:3: 17585 . . . 17761, 17934 . . . 18081, 18969 . . . 19030, and 22374 . . . 22475, 24118 . . . 24243, 24846 . . . 25067, 26470 . . . 26628, 29217 . . . 29324, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, 42954 . . . 43023, and 43825 . . . 43940.

In some embodiments, the mRNA sequence corresponds to the joined nucleotides present in exons at the following positions in SEQ ID NO:3: 17594 . . . 17761, 17934 . . . 18081, 18969 . . . 19030, 22374 . . . 22475, 24846 . . . 25067, 26470 . . . 26628, 29217 . . . 29324, 30242 . . . 30386, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, and 45378 . . . 46868.

In some embodiments, the mRNA sequence corresponds to the joined nucleotides present in exons at the following positions in SEQ ID NO:3: 17595 . . . 17761, 17934 . . . 18081, 18969 . . . 19030, 22374 . . . 22475, 24118 . . . 24243, 24846 . . . 25067, 26470 . . . 26628, 29217 . . . 29324, 30242 . . . 30386, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, and 45378 . . . 46972.

In some embodiments, the mRNA sequence corresponds to the joined nucleotides present in exons at the following positions in SEQ ID NO:3: 17732 . . . 17761, 17934 . . . 18081, 18969 . . . 19030, 22374 . . . 22475, 24118 . . . 24243, 24846 . . . 25067, 26470 . . . 26628, 29217 . . . 29324, 30242 . . . 30386, 33025 . . . 33150, 33266 . . . 33310, 34406 . . . 34485, 34803 . . . 34915, 36076 . . . 36259, 36860 . . . 36944, 37769 . . . 37914, 38451 . . . 38541, 38894 . . . 39042, 40001 . . . 40086, 42954 . . . 43023, 43825 . . . 43943, and 45378 . . . 45519.

In some embodiments, a region of the nucleic acid molecule is at least 70%, 75%, 80%, 85%, 90%, 91%, 92%, 93%, 94%, 95%, 96%, 97%, 98% or 99% complementary to at least a portion of SEQ ID NOS:1-4.

In some embodiments, the nucleic acids can comprise a DNA molecule, such as an antisense DNA molecule. In some embodiments, the composition can comprise an RNA molecule, such as an anti-sense RNA molecule, a small interfering RNA (siRNA) molecule, or small hairpin RNA (shRNA) molecule. In some embodiments, the expression of the DNA or RNA molecule may be regulated by a regulatory region present in the cells.

The nucleic acid that reduces the expression of one or more methyltransferase proteins can be an RNA interference molecule, the RNA interference molecule may be a shRNA, siRNA, miRNA, or guide RNA to CRISPR/CAS9 CRISPRi, etc. Combinations of shRNAs can also be used in accordance with the present invention.

In some embodiments, the nucleic acid that reduces the expression of one or more methyltransferase proteins comprises an siRNA.

A target sequence on a target mRNA can be selected from a given cDNA sequence corresponding to DNMT1, DNMT3a, or DNMT3b, in some embodiments, beginning 50 to 100 nt downstream (i.e., in the 3′ direction) from the start codon. In some embodiments, the target sequence can, however, be located in the 5′ or 3′ untranslated regions, or in the region nearby the start codon.

In one embodiment, the nucleic acid comprises a nucleotide sequence that binds to at least a portion of a nucleotide sequence of DNMT1, DNMT3a, or DNMT3b mRNA. In some embodiments, the nucleic acid molecule is a DNA. In some embodiments, the nucleic acid molecule is an RNA.

In some embodiments, the nucleic acid comprises an anti-sense DNA. Anti-sense DNA binds with mRNA and prevents translation of the mRNA. The anti-sense DNA can be complementary to a portion of DNMT1, DNMT3a, or DNMT3b mRNA. In some embodiments, the anti-sense DNA is complementary to the entire reading frame. In some embodiments, the anti-sense DNA is complementary to the entire reading frame of SEQ ID NOS:1, 2, 3 or 4. In some embodiments, the antisense DNA is complementary to a portion of SEQ ID NO:1, 2, 3, or 4. In some embodiments, the antisense DNA is at least 15 nucleotides, at least 20 nucleotides, at least 25 nucleotides, at least 30 nucleotides, at least 35 nucleotides, at least 40 nucleotides, at least 50 nucleotides, at least 75 nucleotides, at least 100 nucleotides, at least 150 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, or at least 4500 nucleotides.

In some embodiments, the nucleic acid comprises an anti-sense RNA. Anti-sense RNA binds with mRNA and prevents translation of the mRNA. The anti-sense RNA can be complementary to a portion of DNMT1, DNMT3a, or DNMT3b mRNA. In some embodiments, the anti-sense RNA is complementary to the entire reading frame of DNMT1, DNMT3a, or DNMT3b. In some embodiments, the anti-sense RNA is complementary to the entire reading frame of SEQ ID NO: 1, 2, 3, or 4. In some embodiments, the antisense RNA is complementary to a portion of SEQ ID NO: 1, 2, 3, or 4. In some embodiments, the antisense RNA is at least 15 nucleotides, at least 20 nucleotides, at least 25 nucleotides, at least 30 nucleotides, at least 35 nucleotides, at least 40 nucleotides, at least 50 nucleotides, at least 75 nucleotides, at least 100 nucleotides, at least 150 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, or at least 4500 nucleotides.

In some embodiments, the composition is an siRNA targeting DNMT1, DNMT3a, or DNMT3b. SiRNAs are small single or dsRNAs that do not significantly induce the antiviral response common among vertebrate cells but that do induce target mRNA degradation via the RNAi pathway. The term siRNA refers to RNA molecules that have either at least one double stranded region or at least one single stranded region and possess the ability to effect RNA interference (RNAi). It is specifically contemplated that siRNA can refer to RNA molecules that have at least one double stranded region and possess the ability to effect RNAi. The dsRNAs (siRNAs) may be generated by various methods including chemical synthesis, enzymatic synthesis of multiple templates, digestion of long dsRNAs by a nuclease with RNAse III domains, and the like. An “siRNA directed to” at least a particular region of DNMT1, DNMT3a, or DNMT3b means that a particular DNMT1, DNMT3a, or DNMT3b siRNA includes sequences that result in the reduction or elimination of expression of the target gene, i.e., the siRNA is targeted to the region or gene.

The nucleotide sequence of the siRNA is defined by the nucleotide sequence of its target gene. The DNMT1, DNMT3a, or DNMT3b siRNA contains a nucleotide sequence that is essentially identical to at least a portion of the target gene. In some embodiments, the siRNA contains a nucleotide sequence that is completely identical to at least a portion of the DNMT1, DNMT3a, or DNMT3b gene. Of course, when comparing an RNA sequence to a DNA sequence, an “identical” RNA sequence will contain ribonucleotides where the DNA sequence contains deoxyribonucleotides, and further that the RNA sequence will typically contain a uracil at positions where the DNA sequence contains thymidine.

In some embodiments, a DNMT1, DNMT3a, or DNMT3b siRNA comprises a double stranded structure, the sequence of which is “substantially identical” to at least a portion of the target gene. “Identity,” as known in the art, is the relationship between two or more polynucleotide (or polypeptide) sequences, as determined by comparing the sequences. In the art, identity also means the degree of sequence relatedness between polynucleotide sequences, as determined by the match of the order of nucleotides or amino acids between such sequences. Unless otherwise stated, sequence identity/similarity values provided herein refer to the value obtained using the BLAST 2.0 suite of programs using default parameters (Altschul, et al., (1997) Nucleic Acids Res. 25:3389-402).

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. In some embodiments, there is 100% sequence identity between the dsRNA for use as siRNA and at least 15 contiguous nucleotides of the target gene, although a dsRNA having 70%, 75%, 80%, 85%, 90%, or 95% or greater may also be used in the present invention. A siRNA that is essentially identical to a least a portion of the target gene may also be a dsRNA wherein one of the two complementary strands (or, in the case of a self-complementary RNA, one of the two self-complementary portions) is either identical to the sequence of that portion or the target gene or contains one or more insertions, deletions or single point mutations relative to the nucleotide sequence of that portion of the target gene. siRNA technology thus has the property of being able to tolerate sequence variations that might be expected to result from genetic mutation, strain polymorphism, or evolutionary divergence.

In some embodiments, the invention provides an DNMT1, DNMT3a, or DNMT3b siRNA that is capable of triggering RNA interference, a process by which a particular RNA sequence is destroyed (also referred to as gene silencing). In specific embodiments, DNMT1, DNMT3a, or DNMT3b siRNA are dsRNA molecules that are 100 bases or fewer in length (or have 100 base pairs or fewer in its complementarity region). In some embodiments, a dsRNA may be 15, 16, 17, 18, 19, 20, 21, 22, 23, 24, 25, 26, 27, 28, 29, 30, 40, 50, 60, 70, 80, 90, 100 110, 120, 130, 140, 150, 160, 170, 180, 190, 200, 225, 250, 275, 300, 325, 350, 375, 400, 450, 500, 550, 600, 650, 700, 750, 800, 850, 900, 950, or 1000 nucleotides or more in length. In certain embodiments, DNMT1, DNMT3a, or DNMT3b siRNA may be approximately 21 to 25 nucleotides in length. In some cases, it has a two nucleotide 3′ overhang and a 5′ phosphate. The particular DNMT1, DNMT3a, or DNMT3b RNA sequence is targeted as a result of the complementarity between the dsRNA and the particular DNMT1, DNMT3a, or DNMT3b RNA sequence. It will be understood that dsRNA or siRNA of the disclosure can effect at least a 20, 30, 40, 50, 60, 70, 80, 90 percent or more reduction of expression of a targeted DNMT1, DNMT3a, or DNMT3b RNA in target cell. dsRNA of the invention (the term “dsRNA” will be understood to include “siRNA” and/or “candidate siRNA”) is distinct and distinguishable from antisense and ribozyme molecules by virtue of the ability to trigger RNAi. Structurally, dsRNA molecules for RNAi differ from antisense and ribozyme molecules in that dsRNA has at least one region of complementarity within the RNA molecule. In some embodiments, the complementary (also referred to as “complementarity”) region comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 contiguous bases. In some embodiments, long dsRNA are employed in which “long” refers to dsRNA that are 1000 bases or longer (or 1000 base pairs or longer in complementarity region). The term “dsRNA” includes “long dsRNA”, “intermediate dsRNA” or “small dsRNA” (lengths of 2 to 100 bases or base pairs in complementarity region) unless otherwise indicated. In some embodiments, dsRNA can exclude the use of siRNA, long dsRNA, and/or “intermediate” dsRNA (lengths of 100 to 1000 bases or base pairs in complementarity region).

It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (such as when a single strand with a hairpin loop is used as a dsRNA for RNAi). For convenience, lengths of dsRNA may be referred to in terms of bases, which simply refers to the length of a single strand or in terms of base pairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted mRNA is referred to as the “antisense strand” and the strand with a sequence identical to the targeted mRNA is referred to as the “sense strand.” Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

Strands or regions that are complementary may or may not be 100% complementary (“completely or fully complementary”). It is contemplated that sequences that are “complementary” include sequences that are at least 50% complementary, and may be at least 50%, 60%, 70%, 80%, or 90% complementary. In some embodiments, siRNA generated from sequence based on one organism may be used in a different organism to achieve RNAi of the cognate target gene. In other words, siRNA generated from a dsRNA that corresponds to a human gene may be used in a mouse cell if there is the requisite complementarity, as described above. Ultimately, the requisite threshold level of complementarity to achieve RNAi is dictated by functional capability. It is specifically contemplated that there may be mismatches in the complementary strands or regions. Mismatches may number at most or at least 1, 2, 3, 4, 5, 6, 7, 8, 9, 10, 11, 12, 13, 14, 15, 16, 17, 18, 19, 20, 21, 22, 23, 24, 25 residues or more, depending on the length of the complementarity region.

In some embodiments, the single RNA strand or each of two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more (including the full-length DNMT1, DNMT3a, or DNMT3b mRNA without the poly-A tail) bases or base pairs. If the dsRNA is composed of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

In some embodiments, the strand or strands of dsRNA are 100 bases (or base pairs) or less. In specific embodiments the strand or strands of the dsRNA are less than 70 bases in length. With respect to those embodiments, the dsRNA strand or strands may be from 5-70, 10-65, 20-60, 30-55, 40-50 bases or base pairs in length. A dsRNA that has a complementarity region equal to or less than 30 base pairs (such as a single stranded hairpin RNA in which the stem or complementary portion is less than or equal to 30 base pairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 base pairs or fewer. In some cases, a dsRNA may be processed in the cell into siRNA.

The siRNA can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA of the disclosure can comprise a 3′ overhang. As used herein, a “3′ overhang” refers to at least one unpaired nucleotide extending from the 3′-end of a duplexed RNA strand.

Thus in some embodiments, the DNMT1, DNMT3a, or DNMT3b siRNA comprises at least one 3′ overhang of from 1 to about 6 nucleotides (which includes ribonucleotides or deoxynucleotides) in length, from 1 to about 5 nucleotides in length, from 1 to about 4 nucleotides in length, or from about 2 to about 4 nucleotides in length.

In some embodiments in which both strands of the DNMT1, DNMT3a, or DNMT3b siRNA molecule comprise a 3′ overhang, the length of the overhangs can be the same or different for each strand. In some embodiments, the 3′ overhang is present on both strands of the siRNA, and is 2 nucleotides in length. For example, each strand of the DNMT1, DNMT3a, or DNMT3b siRNA can comprise 3′ overhangs of dithymidylic acid (“TT”) or diuridylic acid (“UU”).

In order to enhance the stability of the present DNMT1, DNMT3a, or DNMT3b siRNA, the 3′ overhangs can be also stabilized against degradation. In some embodiments, the overhangs are stabilized by including purine nucleotides, such as adenosine or guanosine nucleotides. In some embodiments, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine can significantly enhance the nuclease resistance of the 3′ overhang in tissue culture medium.

In some embodiments, the DNMT1, DNMT3a, or DNMT3b siRNA can be targeted to any stretch of approximately 19-25 contiguous nucleotides in any of the target mRNA sequences (the “target sequence”). Techniques for selecting target sequences for siRNA are given, for example, in Tuschl T et al., “The siRNA User Guide,” revised Oct. 11, 2002, the entire disclosure of which is herein incorporated by reference. “The siRNA User Guide” is available on the world wide web at a website maintained by Dr. Thomas Tuschl, Department of Cellular Biochemistry, AG 105, Max-Planck-Institute for Biophysical Chemistry, 37077 Gottingen, Germany, and can be found by accessing the website of the Max Planck Institute and searching with the keyword “siRNA.” Thus, in some embodiments, the sense strand of the present siRNA comprises a nucleotide sequence identical to any contiguous stretch of about 19 to about 25 nucleotides in the target mRNA.

In some embodiments, the siRNA comprises a 21 nucleotide double stranded sequence. In some embodiments, the siRNA comprises a two-TT overhang (Yang et al., Nucleic Acid Research, 34(4), 1224-1236, 2006).

In some embodiments, the composition useful in the methods of the invention comprises an shRNA molecule that targets DNMT1, DNMT3a, or DNMT3b mRNA (DNMT1, DNMT3a, or DNMT3b shRNA). shRNA is an artificial RNA molecule with a tight hairpin turn that can be used to silence target gene expression via RNA interference (RNAi). In certain cases, expression of DNMT1, DNMT3a, or DNMT3b shRNA in cells is achieved through delivery of non-viral vectors (such as plasmids or bacterial vectors) or through viral vectors. shRNA is useful because it has a relatively low rate of degradation and turnover.

In order to obtain long-term gene silencing, expression vectors that continually express siRNAs in stably transfected mammalian cells can be used (Brummelkamp et al., Science 296: 550-553, 2002; Lee et al., Nature Biotechnol. 20:500-505, 2002; Miyagishi, M, and Taira, K. Nature Biotechnol. 20:497-500, 2002; Paddison, et al., Genes & Dev. 16:948-958, 2002; Paul et al., Nature Biotechnol. 20:505-508, 2002; Sui, Proc. Natl. Acad. Sci. USA 99(6):5515-5520, et al., 2002; Yu et al., Proc. Natl. Acad. Sci. USA 99(9):6047-6052, 2002). Many of these plasmids have been engineered to express shRNAs lacking poly (A) tails. Transcription of shRNAs is initiated at a polymerase III (pol III) promoter and is believed to be terminated at position 2 of a 4-5-thymine transcription termination site. Upon expression, shRNAs are thought to fold into a stem-loop structure with 3′ UU-overhangs. Subsequently, the ends of these shRNAs are processed, converting the shRNAs into ^{˜}21 nt siRNA-like molecules. The siRNA-like molecules can, in turn, bring about gene-specific silencing in the transfected mammalian cells.

The length of the stem and loop of shRNAs can be varied. In some embodiments, stem lengths could range anywhere from 25 to 29 nucleotides and loop size could range between 4 to 23 nucleotides without affecting silencing activity. Moreover, presence of G-U mismatches between the two strands of the shRNA stem does not necessarily lead to a decrease in potency.

In some embodiments, the nucleic acids can be modified. In some embodiments, the nucleic acids can be modified to include a phosphorothioate (PS) backbone. The modification to the backbone can be throughout the molecule or at one or more defined sites. In some embodiments, the nucleic acids can be modified to encompass peptide nucleic acids (PNA). In some embodiments, the nucleic acids can be modified to encompass phosphorodiamidate morpholino oligomers (PMO).

In some embodiments, the nucleic acid molecules can include derivatives such as S-oligonucleotides (phosphorothioate derivatives or S-oligos). S-oligos (nucleoside phosphorothioates) are isoelectronic analogs of an oligonucleotide (0-oligo) in which a nonbridging oxygen atom of the phosphate group is replaced by a sulfur atom. The S-oligos of the present invention may be prepared by treatment of the corresponding 0-oligos with 3H-1,2-benzodithiol-3-one-1,1-dioxide which is a sulfur transfer reagent. See Iyer et al., J. Org. Chem. 55:4693-4698 (1990); and Iyer et al., J. Am. Chem. Soc. 112:1253-1254 (1990), the disclosures of which are fully incorporated by reference herein.

In some embodiments of the invention, a dsRNA has one or more non-natural nucleotides, such as a modified residue or a derivative or analog of a natural nucleotide. Any modified residue, derivative or analog may be used to the extent that it does not eliminate or substantially reduce (by at least 50%) RNAi activity of the dsRNA.

A person of ordinary skill in the art is well aware of achieving hybridization of complementary regions or molecules. Such methods typically involve heat and slow cooling of temperature during incubation, for example.

In some embodiments, the nucleic acid molecules are encoded by expression vectors. The expression vectors may be obtained and introduced into a cell. Once introduced into the cell the expression vector is transcribed to produce various nucleic acids. Expression vectors include nucleic acids that provide for the transcription of a particular nucleic acid. Expression vectors include plasmid DNA, linear expression elements, circular expression elements, viral expression constructs (including adenoviral, adeno-associated viral, retroviral, lentiviral, and so forth), and the like, all of which are contemplated as being used in the compositions and methods of the present disclosure. In some embodiments one or at least 2, 3, 4, 5, 6, 7, 8, 9, 10 or more nucleic acid molecules binding to DNMT1, DNMT3a, or DNMT3b RNA are encoded by a single expression construct. Expression of the nucleic acid molecules binding to DNMT1, DNMT3a, or DNMT3b RNA may be independently controlled by at least 1, 2, 3, 4, 5, 6, 7, 8, 9, 10 or more regulatory elements. In certain embodiments, at least 1, 2, 3, 4, 5, 6, 7, 8, 9, 10 or more expression constructs can be introduced into a cell. Each expression construct can encode 1, 2, 3, 4, 5, 6, 7, 8, 9, 10 or more nucleic acid molecules binding to DNMT1, DNMT3a, or DNMT3b RNA. In some embodiments, nucleic acid molecules binding to DNMT1, DNMT3a, or DNMT3b RNA may be encoded as expression domains. Expression domains include a transcription control element, which may or may not be independent of other control or promoter elements; a nucleic acid; and optionally a transcriptional termination element.

In some embodiments, the one or more other components necessary to achieve recombinant AAV biosynthesis are provided to the eukaryotic cells by transient transfection. In some embodiments, the one or more other components necessary to achieve recombinant AAV biosynthesis are stably transfected into the eukaryotic cells. In some embodiments, the one or more components comprise AAV Rep, AAV Cap, helper virus genes Adenovirus E2, E4, and VA RNA. Protocols for the generation of adenoviral vectors have been described in U.S. Pat. Nos. 5,998,205; 6,228,646; 6,093,699; and 6,100,242; and. International Patent Application Nos. WO 94/17810 and WO 94/23744, which are incorporated herein by reference in their entirety.

In some embodiments, the helper virus genes are provided on a plasmid that further comprises Herpes virus genes known to support recombinant AAV generation in cell culture. A “plasmid” is a form of nucleic acid or polynucleotide that typically has additional elements for expression (e.g., transcription, replication, etc.) or propagation (replication) of the plasmid. A plasmid as used herein also can be used to reference nucleic acid and polynucleotide sequences. Accordingly, in all aspects the invention compositions and methods are applicable to plasmids, nucleic acids and polynucleotides, e.g., for introducing plasmids, nucleic acid or polynucleotide into cells, for transducing (transfecting) cells with plasmid, nucleic acid or polynucleotide, for producing transduced (transfected) cells that have a plasmid, nucleic acid or polynucleotide, to produce cells that produce viral (e.g., AAV) vectors, to produce viral (e.g., AAV) vectors, to produce cell culture medium that has viral (e.g., AAV) vectors, etc.

In another embodiment, the invention provides a method of making a plasmid DNA in bacterial cells, wherein the plasmid DNA comprises nucleic acid comprising a sequence of interest that is flanked by AAV inverted terminal repeats, wherein the nucleic acid comprises CpG dinucleotide sites,

comprising transforming the bacterial cells with the nucleic acid, wherein the bacterial cells are modified to express a polypeptide capable of methylating CpG dinucleotide sites, whereby the bacterial cells produce plasmid DNA comprising the nucleic acid, wherein at least a portion of the CpG dinucleotide sites in the nucleic acid are methylated.

In some embodiments, the plasmid DNA having CpG methylation is transiently transfected into eukaryotic cells to produce recombinant AAV vectors.

In another embodiment, the invention provides a method of making CpG methylated nucleic acid in vitro, wherein the nucleic acid comprises a sequence of interest that is flanked by AAV inverted terminal repeats,

comprising contacting the nucleic acid with a polypeptide capable of methylating CpG dinucleotide sites, whereby at least a portion of the CpG dinucleotide sites in the nucleic acid are methylated.

In some embodiments, the nucleic acid comprising a sequence of interest that is flanked by AAV inverted terminal repeats is made synthetically in vitro.

In some embodiments, the nucleic acid is made in vitro using rolling-circle amplification to produce quantities of concatameric DNA that is then processed to create closed linear double-stranded DNA by enzymatic digestion (DOGGYBONE DNA).

In some embodiments, the nucleic acid having CpG methylation is transiently transfected into eukaryotic cells to produce recombinant AAV vectors.

In some embodiments, the nucleic acid produced comprising a sequence of interest that is flanked by AAV inverted terminal repeats is methylated in at least 20% of CpG dinucleotide sites, is methylated in at least 50% of CpG dinucleotide sites, or is methylated in at least from 60%-90% of CpG dinucleotide sites.

In some embodiments, the recombinant AAV produced by the eukaryotic cells is methylated in at least about 20% of CpG dinucleotide sites, is methylated in at least 25%, is methylated in at least about 30% of CpG dinucleotide sites, is methylated in at least about 35% of CpG dinucleotide sites, is methylated in at least about 40% of CpG dinucleotide sites, is methylated in at least about 45% of CpG dinucleotide sites, is methylated in at least about 50% of CpG dinucleotide sites, is methylated in at least about 55% of CpG dinucleotide sites, is methylated in at least about 60% of CpG dinucleotide sites, is methylated in at least about 65% of CpG dinucleotide sites, is methylated in at least about 70% of CpG dinucleotide sites, is methylated in at least about 75% of CpG dinucleotide sites, is methylated in at least about 80% of CpG dinucleotide sites, is methylated in at least about 85% of CpG dinucleotide sites, is methylated in at least about 90% of CpG dinucleotide sites, is methylated in at least about 91%, 92%, 93%, 94%, 95%, 96%, 97%, 98%, 99% or 100% of CpG dinucleotide sites. In some embodiments, the recombinant AAV produced by the eukaryotic cells is methylated in at least from 60%-90% of CpG dinucleotide sites.

In some embodiments, the recombinant AAV produced by the eukaryotic cells is methylated at less than about 20% of the CpG dinucleotide sites, is methylated at less than about 10% of the CpG dinucleotide sites, is methylated at less than about 5% of the CpG dinucleotide sites, or is methylated at less than about 1% of the CpG dinucleotide sites.

### Therapeutic Methods

In another embodiment, the invention provides a method of treating a disease or condition in a subject, comprising administering to the subject an effective amount of the recombinant AAV vector according to the invention.

In some embodiments, the recombinant AAV vector is administered according to a route selected from the group consisting of intravenous, systemic, intramuscular, intracranial, intraparenchymal and combinations thereof.

The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of blood clotting diseases such as HemA and others known to those of skill in the art.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented.

The particular dosage depends upon the age, weight, sex and medical condition of the subject to be treated, as well as on the method of administration. Suitable doses can be readily determined by those of skill in the art.

In some embodiments, recombinant AAV vector encodes a therapeutic protein. Non-limiting examples include a blood clotting factor (e.g., Factor XIII, Factor IX, Factor X, Factor VIII, Factor VIIa, or protein C), apoE2, TPP1, arginino succinate synthase, copper transporting ATPase 2, acid alpha-glucosidase, fibrinogen A, fibrinogen B, (3-Glucocerebrosidase, α-galactosidase, C1 inhibitor serine protease inhibitor, CFTR (cystic fibrosis transmembrane regulator protein), an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor (e.g., insulin-like growth factors 1 and 2, platelet derived growth factor, epidermal growth factor, nerve growth factor, neurotrophic factor −3 and −4, brain-derived neurotrophic factor, glial derived growth factor, transforming growth factor a and (3, etc.), a cytokine (e.g., α-interferon, β-interferon, interferon-γ, interleukin-2, interleukin-4, interleukin 12, granulocyte-macrophage colony stimulating factor, lymphotoxin, etc.), a suicide gene product (e.g., herpes simplex virus thymidine kinase, cytosine deaminase, diphtheria toxin, cytochrome P450, deoxycytidine kinase, tumor necrosis factor, etc.), a drug resistance protein (e.g., that provides resistance to a drug used in cancer therapy), a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitopes, or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB 1 (Connexin 26 deafness), ACHM 2, 3 and 4 (Achromatopsia), PKD-1 or PKD-2 (Polycystic kidney disease), TPP1, CLN2, gene deficiencies causative of lysosomal storage diseases (e.g., sulfatases, N-acetylglucosamine-1-phosphate transferase, cathepsin A, GM2-AP, NPC1, VPC 2, Sphingolipid activator proteins, etc.), beta-2-microglobulin, zinc-alpha-2-glycoprotein, alpha-2-HS-glycoprotein (fetuin), serum amyloid protein A, haptoglobin, profilin, desmocollin, thymosin beta-4 and -beta-10, apolipoprotein C-III, uteroglobin, ubiquitin, gelsolin, collagen, fibrin, as well as fragments of these and other proteins, one or more zinc finger nucleases for genome editing, or donor sequences used as repair templates for genome editing.

A “therapeutic” peptide or protein is a peptide or protein that may alleviate or reduce symptoms that result from an absence or defect in a protein in a cell or subject. Alternatively, a “therapeutic” peptide or protein is one that otherwise confers a benefit to a subject, e.g., anti-cancer effects. Therapeutic peptides and proteins can also include, but are not limited to, CFTR (cystic fibrosis transmembrane regulator protein), dystrophin (including the protein product of dystrophin mini-genes, see, e.g, Vincent et al., (1993) Nature Genetics 5:130), utrophin (Tinsley et al., (1996) Nature 384:349), clotting factors (Factor XIII, Factor IX, Factor X, etc.), monoclonal antibodies (Lewis et al., 2002), erythropoietin, the LDL receptor, lipoprotein lipase, ornithine transcarbamylase, β-globin, α-globin, spectrin, α-antitrypsin, adenosine deaminase, hypoxanthine guanine phosphoribosyl transferase, .beta.-glu cocerebro sidase, sphingomyelinase, lysosomal hexosaminidase, branched-chain keto acid dehydrogenase, hormones, growth factors (e.g., insulin-like growth factors 1 and 2, platelet derived growth factor, epidermal growth factor, nerve growth factor, neurotrophic factor −3 and −4, brain-derived neurotrophic factor, glial derived growth factor, transforming growth factor .alpha. and .beta., and the like), cytokines (e.g., α-interferon, β-interferon, interferon-γ, interleukin-2, interleukin-4, interleukin 12, granulocyte-macrophage colony stimulating factor, lymphotoxin), suicide gene products (e.g., herpes simplex virus thymidine kinase, cytosine deaminase, diphtheria toxin, cytochrome P450, deoxycytidine kinase, and tumor necrosis factor), proteins conferring resistance to a drug used in cancer therapy, tumor suppressor gene products (e.g., p53, Rb, Wt-1, NF1, VHL, APC, and the like), and any other peptide or protein that has a therapeutic effect in a subject in need thereof.

Further exemplary therapeutic peptides or proteins include those that may used in the treatment of a disease condition including, but not limited to, cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Gaucher's disease, Hurler's disease, adenosine deaminase deficiency, glycogen storage diseases and other metabolic defects, retinal degenerative diseases (and other diseases of the eye), and diseases of solid organs (e.g., brain, liver, kidney, heart).

In some embodiments, the sequence of interest encodes an antigen that provides a target for immune recognition by a subject's immune response when the recombinant AAV is administered to the subject.

As used herein, “antigen” includes any antigen including patient-specific neoantigens. An antigen includes any substance that can induce an immune response.

As used herein, an “immune response” is the physiological response of the subject's immune system to an immunizing composition. An immune response may include an innate immune response, an adaptive immune response, or both. In one embodiment of the present invention, the immune response is a protective immune response. A protective immune response confers immunological cellular memory upon the subject, with the effect that a secondary exposure to the same or a similar antigen is characterized by one or more of the following characteristics: shorter lag phase than the lag phase resulting from exposure to the selected antigen in the absence of prior exposure to the immunizing composition; production of antibody which continues for a longer period than production of antibody resulting from exposure to the selected antigen in the absence of prior exposure to the immunizing composition; a change in the type and quality of antibody produced in comparison to the type and quality of antibody produced upon exposure to the selected antigen in the absence of prior exposure to the immunizing composition; a shift in class response, with IgG antibodies appearing in higher concentrations and with greater persistence than IgM, than occurs in response to exposure to the selected antigen in the absence of prior exposure to the immunizing composition; an increased average affinity (binding constant) of the antibodies for the antigen in comparison with the average affinity of antibodies for the antigen resulting from exposure to the selected antigen in the absence of prior exposure to the immunizing composition; and/or other characteristics known in the art to characterize a secondary immune response.

In such embodiments, it can be desirable that the recombinant AAV is made according to the methods of the present invention to have enhance immunogenicity. In some embodiments, the antigen is a non-human antigen. In some embodiments, the antigen is presented at the surface of a transduced cell when the recombinant AAV is administered to a subject, thereby providing a recognition target for effector functions of the subject's immune response.

In some embodiments, the recombinant AAV vector encodes an antigen from a pathogen, such as a virus, wherein the vector confers protective immunity for the pathogen when administered to a subject.

In some embodiments, the pathogen is a bacterial or viral pathogen. In some embodiments, the pathogen or its associated disease is selected from the group consisting of Streptococcus pneumonia, Neisseria meningitidis, Haemophilus influenza, Klebsiella spp., Pseudomonas spp., Salmonella spp., Shigella spp., and Group B streptococci, Bacillus anthracis adenoviruses; Bordetella pertussus; Botulism; bovine rhinotracheitis; Brucella spp.; Branhamella catarrhalis; canine hepatitis; canine distemper; Chlamydiae; Cholera; coccidiomycosis; cowpox; tularemia; filoviruses; arenaviruses; bunyaviruses; cytomegalovirus; cytomegalovirus; Dengue fever; dengue toxoplasmosis; Diphtheria; encephalitis; Enterotoxigenic Escherichia coli; Epstein Barr virus; equine encephalitis; equine infectious anemia; equine influenza; equine pneumonia; equine rhinovirus; feline leukemia; flavivirus; Burkholderia mallei; Globulin; Haemophilus influenza type b; Haemophilus influenzae; Haemophilus pertussis; Helicobacter pylori; Hemophilus spp.; hepatitis; hepatitis A; hepatitis B; Hepatitis C; herpes viruses; HIV; HIV-1 viruses; HIV-2 viruses; HTLV; Influenza; Japanese encephalitis; Klebsiellae spp. Legionella pneumophila; leishmania; leprosy; lyme disease; malaria immunogen; measles; meningitis; meningococcal; Meningococcal Polysaccharide Group A, Meningococcal Polysaccharide Group C; mumps; Mumps Virus; mycobacteria; Mycobacterium tuberculosis; Neisseria spp; Neisseria gonorrhoeae; ovine blue tongue; ovine encephalitis; papilloma; SARS, MERS and associated coronaviruses, including SARS-CoV-2; parainfluenza; paramyxovirus; paramyxoviruses; Pertussis; Plague; Coxiella burnetti; Pneumococcus spp.; Pneumocystis carinii; Pneumonia; Poliovirus; Proteus species; Pseudomonas aeruginosa; rabies; respiratory syncytial virus; rotavirus; Rubella; Salmonellae; schistosomiasis; Shigellae; simian immunodeficiency virus; Smallpox; Staphylococcus aureus; Staphylococcus spp.; Streptococcus pyogenes; Streptococcus spp.; swine influenza; tetanus; Treponema pallidum; Typhoid; Vaccinia; varicella-zoster virus; and Vibrio cholera and combinations thereof.

In some embodiments, the recombinant AAV vector is targeted to cancer cells when the vector is administered to a subject, wherein the cancer cells are specifically rendered targets for destruction by effector functions of the subject's immune response. In some embodiments, the cancer antigen is a patient specific neoantigen.

In some embodiments, the immunizing composition is administered in combination with one or more additional cancer therapies as provided herein, including, for example, radiation, chemotherapy, surgery, immunotherapy, immune checkpoint inhibitors, and the like.

In some embodiments, the cancer comprises a tumor that is a solid tumor. In some embodiments, the tumor is a primary tumor. In some embodiments, the tumor is a secondary tumor comprising cells that have metastasized. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor.

In some embodiments, the cancer is a hematologic cancer. In some embodiments, the hematologic cancer is a leukemia. In other embodiments, the hematologic cancer is a lymphoma. In some embodiment, the cancer is selected from the group consisting of: acute myelogenous leukemia (AML), Hodgkin lymphoma, multiple myeloma. T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

In some embodiment, the cancer antigen, fragment or variant thereof comprises one or more of CD20, CD22, CD19, CD33, CD40, CD52, CCR4, WT-1, HER2, CD137, OX40, EGFR, VEGF, EPCAM, alphafetoprotein (AFP), CEA, CA-125, Muc 1, epithelial tumor antigen (ETA), tyrosinase (for a more extensive list, see Polanski and Anderson (2006) Biomarker Insights 2:1-48); PD1 and CTLA4 (Suresh et al (2014) J Hematol Oncol 7:58), cancer/testis (CT) antigens (e.g., MAGE-A-A4, MAGE-C1, SSX2, SSX4, NY-ESO-1, SCP1, CT7. NH-SAR-35, OY-TES-1, SLCO6A1, PASD1, CAGE-1, KK-LC-1); cytokines (IL-2, IL-8, IL-6R, IL-12, IL-23, IL-17, IL-22, IL-26, RANKL), Jak kinase inhibitors, TGF-β, α4β7 integrin, α4β1 integrin, TNFα, CD52, CD25, CD20, annexin A2, proteins involved in the classical complement pathway including C1q, growth factors, and/or proteins found in the brain and other tissues such as α-synuclein, amyloidβ (Aβ), NGF, TrkA, CGRP and/or NGF. See, e.g., Tanida et al (2015) World J Gastro 21(29):8776-86; Neurath (2014) Nature 7(1):6; Rice et al (2015) J Clin Invest 125(7):2795; Palmer (2013) Br J Clin Pharm 78(1):33-43); Turner et al (2015) Semin Cell Dev Biol. October 8. pii: S1084-9521(15) 00188-3. doi: 10.1016/j.semcdb.2015.10.003); Liu et al (2015) J Neuroinflam 12:153); Hirose et al (2015), Pain Pract doi:10.1111; Bigal et al (2015) Lancet Neurology 14(11):1091, Gow et al (2015) Arthritis Res Ther doi:10.1186); Cabellero and Chen (2009) Cancer Sci 100(11):2014-2021.

In some embodiments, the therapeutic protein that is encoded by the recombinant AAV vector is an antibody that recognizes a cancer antigen, such as one of the cancer antigens described above, or a patient specific, neoantigen.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments, dual affinity retargeting antibodies (DART)), single chain Fv (scFv) mutants, multispecific antibodies such as bispecific and trispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

In some embodiments, the recombinant AAV vector comprises a nucleic acid that produces a transcript when transcribed that has a therapeutic effect. Such transcripts can be RNA, such as inhibitory RNA (RNAi, e.g., small or short hairpin (sh)RNA, microRNA (miRNA), small or short interfering (si)RNA, trans-splicing RNA, or antisense RNA). Non-limiting examples include inhibitory nucleic acids that inhibit expression of: huntingtin (HTT) gene, a gene associated with dentatorubropallidolusyan atropy (e.g., atrophin 1, ATN1); androgen receptor on the X chromosome in spinobulbar muscular atrophy, human Ataxin-1, -2, -3, and -7, Cav2.1 P/Q voltage-dependent calcium channel is encoded by the (CACNA1A), TATA-binding protein, Ataxin 8 opposite strand, also known as ATXN80S, Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercoloesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Reddl also known as DAN damage-inducible transcript 4 protein, in diabetic macular oedma (DME) or age-related macular degeneration; vascular endothelial growth factor receptor I (VEGFR1) in age-related macular degeneration or choroidal neovascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS (including SARS-CoV-2) infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; mutant rhodopsin gene (RHO) in autosomal dominantly inherited retinitis pigmentosa (adRP); or the inhibitory nucleic acid binds to a transcript of any of the foregoing genes or sequences.

In some embodiments, the recombinant AAV vector comprising a sequence of interest is administered to cells ex vivo, and then the transduced cells are administered to the subject to treat a disease or condition in the subject.

The present invention further provides a method of delivering the recombinant AAV vectors to a cell. For in vitro methods, the virus may be administered to the cell by standard viral transduction methods, as are known in the art. Preferably, the virus particles are added to the cells at the appropriate multiplicity of infection according to standard transduction methods appropriate for the particular target cells. Titers of virus to administer can vary, depending upon the target cell type and the particular virus vector, and may be determined by those of skill in the art without undue experimentation. Alternatively, administration of recombinant AAV vectors of the present invention can be accomplished by any other means known in the art.

Recombinant virus vectors are administered to the cell or subjects in effective amounts. An amount of the virus vector is an amount that is sufficient to result in infection (or transduction) and expression of the heterologous nucleic acid sequence in the cell. If the virus is administered to a cell in vivo (e.g., the virus is administered to a subject as described below), an amount of the virus vector is an amount that is sufficient to result in transduction and expression of the heterologous nucleic acid sequence in a target cell.

The cell to be administered or targeted for the recombinant AAV vectors may be of any type, including but not limited to neural cells (including cells of the peripheral and central nervous systems, in particular, brain cells), lung cells, retinal cells, epithelial cells (e.g., gut and respiratory epithelial cells), muscle cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stem cell (e.g., neural stem cell, liver stem cell). Moreover, the cells can be from any species of origin, as indicated above.

In particular embodiments of the invention, cells are removed from a subject, the recombinant AAV vector is introduced therein, and the cells are then replaced back into the subject. Methods of removing cells from subject for treatment ex vivo, followed by introduction back into the subject are known in the art. Alternatively, the rAAV vector is introduced into cells from another subject, into cultured cells, or into cells from any other suitable source, and the cells are administered to a subject in need thereof.

Suitable cells for ex vivo gene therapy include, but are not limited to, liver cells, neural cells (including cells of the central and peripheral nervous systems, in particular, brain cells), pancreas cells, spleen cells, fibroblasts (e.g., skin fibroblasts), keratinocytes, endothelial cells, epithelial cells, myoblasts, hematopoietic cells, bone marrow stromal cells, progenitor cells, and stem cells.

Dosages of the cells to administer to a subject will vary upon the age, condition and species of the subject, the type of cell, the nucleic acid being expressed by the cell, the mode of administration, and the like. Typically, at least about 10² to about 10⁸, preferably about 10³ to about 10⁶ cells, will be administered per dose. Preferably, the cells will be administered in a “therapeutically-effective amount.”

A “therapeutically-effective” amount as used herein is an amount of that is sufficient to alleviate (e.g., mitigate, decrease, reduce) at least one of the symptoms associated with a disease state. Alternatively stated, a “therapeutically-effective” amount is an amount that is sufficient to provide some improvement in the condition of the subject. A further aspect of the invention is a method of treating subjects in vivo with the recombinant AAV vectors. Administration of the recombinant AAV vectors of the present invention to a human subject or an animal in need thereof can be by any means known in the art for administering virus vectors.

Exemplary modes of administration include oral, rectal, transmucosal, topical, transdermal, inhalation, parenteral (e.g., intravenous, subcutaneous, intradermal, intramuscular, and intraarticular) administration, and the like, as well as direct tissue or organ injection including eye, ear, kidney, lung, heart, lung salivary glands, lymph nodes, alternatively, intrathecal, direct intracranial, direct intramuscular, intraventricular, intravenous, intraperitoneal, intranasal, or intraocular injections. Injectables can be prepared in conventional forms, either as liquid solutions or suspensions, solid forms suitable for solution or suspenions in liquid prior to injection, or as emulsions. Alternatively, one may administer the virus in a local rather than systemic manner, for example in a depot or sustained-release formation.

In one embodiment of the invention, the nucleotide sequence of interest is delivered to the liver of the subject. Administration to the liver may be achieved by any method known in art, including, but not limited to intravenous administration, intraportal administration, intrabilary administration, intra-arterial administration, and direct injection into the liver paraenchyma.

Preferably, the cells (e.g., liver cells) are infected by a recombinant AAV vector encoding a peptide or protein, the cells express the encoded peptide or protein and secrete it into the circulatory system in a therapeutically-effective amount (as defined above). Alternatively, the vector is delivered to and expressed by another cell or tissue, including but not limited to, brain, pancreas, spleen or muscle.

In other preferred embodiments, the recombinant AAV vectors are administered intramuscularly, more preferably by intramuscular injection or by local administration (as defined above). In other preferred embodiments, the recombinant AAV vectors of the present invention are administered to the lungs.

The recombinant AAV vectors disclosed herein may be administered to the lungs of a subject by any suitable means, but are preferably administered by administering an aerosol suspension of respirable particles comprised of the recombinant AAV vectors, which the subject inhales. The respirable particles may be liquid or solid. Aerosols of liquid particles comprising the recombinant AAV vectors may be produced by any suitable means, such as with a pressure-driven aerosol nebulizer or an ultrasonic nebulizer, as is known to those of skill in art. See, e.g. U.S. Pat. No. 4,501,729. Aerosols of solid particles comprising the inventive virus vectors may likewise be produced with any solid particulate medicament aerosol generator, by techniques known in the pharmaceutical art.

Dosages of the recombinant AAV vectors will depend upon the mode of administration, the disease or condition to be treated, the individual subject's condition, the particular virus vector, and the gene to be delivered and can be determined in a routine manner. Exemplary doses for achieving therapeutic effects are virus titers of at least about 10⁵, 10⁶, 10⁷, 10⁸, 10⁹, 10¹⁰, 10¹¹, 10¹², 10¹³, 10¹⁴, 10¹⁵, 10¹⁶ transducting units or more, preferably about 10⁸ to 10¹³ transducting units, yet more preferably 10¹² transducing units.

In particular embodiments of the invention, more than one administration (e.g., two, three, four, or more administrations) may be employed to achieve therapeutic levels of gene expression. According to this embodiment and as described above the recombinant AAV vectors of the present invention are administered to reduce the occurrence of neutralizing antibodies in the subject to be treated or to prevent the development of an immune response in the subject. The subject may be presented with seemingly new virus vectors by packaging the rAAV genome within an array of hybrid or chimeric capsids.

The recombinant AAV vectors, reagents, and methods of the present invention can be used to direct a nucleic acid to either dividing or non-dividing cells, and to stably express the heterologous nucleic acid therein. Using this vector system, it is now possible to introduce into cells, in vitro or in vivo, genes that encode proteins that affect cell physiology. The vectors of the present invention can thus be useful in gene therapy for disease states or for experimental modification of cell physiology

In some embodiments, the sequence of interest comprises a nucleic acid sequence capable of silencing expression of a gene in the subject's cells.

In some embodiments, the sequence of interest when expressed in the cell encodes a small interfering RNA.

In some embodiments, the sequence of interest encodes a therapeutic gene product.

In some embodiments, the therapeutic gene product is selected from the group consisting of an antibody (e.g., a heavy and light chain sequence, a single chain variable fragment (scFV), etc.) coagulation factor IX, coagulation factor VIII, dystrophin, microdystrophin, and alpha1 antitrypsin.

In some embodiments, the sequence of interest when expressed following administration to a subject provides a therapeutic benefit for a genetic disease or condition. In some embodiments, the genetic disease or condition is selected from the group consisting of hemophilia B, hemophilia A, Duchenne's Muscular Dystrophy, and alpha1 antitrypsin deficiency.

In some embodiments, the sequence of interest when expressed following administration to a subject provides a therapeutic benefit for a disease or condition without a clear genetic etiology.

In some embodiments, the disease or condition is selected from the group consisting of cancer, Parkinson's Disease, Alzheimer's Disease, macular degeneration, and diabetes.

### Combination Therapy

In some embodiments, the method further comprises one or more additional treatments for the disease or condition of the subject, such as cancer. Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). In some embodiments, combination therapy comprises a therapeutic agent that affects the immune response (e.g., enhances or activates the response) and a therapeutic agent that affects (e.g., inhibits or kills) the tumor/cancer cells.

In some embodiments, the combination of an agent described herein and at least one additional therapeutic agent results in additive or synergistic results. In some embodiments, the combination therapy results in an increase in the therapeutic index of the agent. In some embodiments, the combination therapy results in an increase in the therapeutic index of the additional therapeutic agent(s). In some embodiments, the combination therapy results in a decrease in the toxicity and/or side effects of the agent. In some embodiments, the combination therapy results in a decrease in the toxicity and/or side effects of the additional therapeutic agent(s).

In certain embodiments, in addition to administering a recombinant AAV vector described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

Therapeutic agents that may be administered in combination with the agents described herein include chemotherapeutic agents. Thus, in some embodiments, the method or treatment involves the administration of an agent of the present invention in combination with a chemotherapeutic agent or in combination with a cocktail of chemotherapeutic agents. Treatment with an agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book. 4^{th} Edition, 2008, M. C. Perry, Editor, Lippincott, Williams & Wilkins, Philadelphia, Pa.

Useful classes of therapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomy sins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; be strabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; g acyto sine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARES TON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin. In certain embodiments, a combination of cisplatin and paclitaxel is administered in combination with the recombinant AAV vector described herein.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel. In certain embodiments, the additional therapeutic agent is albumin-bound paclitaxel (ABRAXANE).

In some embodiments, an additional therapeutic agent comprises an agent such as a small molecule. For example, treatment can involve the combined administration of an agent of the present invention with a small molecule that acts as an inhibitor against tumor-associated antigens including, but not limited to, EGFR, HER2 (ErbB2), and/or VEGF. In some embodiments, an agent of the present invention is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In certain embodiments, the additional therapeutic agent is an agent that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the RSPO/LGR pathway. In some embodiments, the additional therapeutic agent is an inhibitor of the mTOR/AKR pathway.

In some embodiments, an additional therapeutic agent comprises a biological molecule, such as an antibody. For example, treatment can involve the combined administration of an agent of the present invention with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits β-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

In certain embodiments, the recombinant AAV vector described herein is administered in combination with at least one immunotherapeutic agent. In some embodiments, the immunotherapeutic agent is an immune response stimulating agent. In some embodiments, the immunotherapeutic agent (e.g., immune response stimulating agent) includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, I1-12, IL-15, IL-18), an antibody that blocks immunosuppressive functions (e.g., an anti-CTLA4 antibody, anti-CD28 antibody, anti-CD3 antibody, anti-PD-1 antibody, anti-PD-L1 antibody), an antibody that enhances immune cell functions (e.g., an anti-GITR antibody or an anti-OX-40 antibody), a toll-like receptor (e.g., TLR4, TLR7, TLR9), a soluble ligand (e.g., GITRL or OX-40L), or a member of the B7 family (e.g., CD80, CD86). An immunotherapeutic agent (e.g., an immune response stimulating agent) can be administered prior to, concurrently with, and/or subsequently to, administration of the recombinant AAV vector described herein. Pharmaceutical compositions comprising the recombinant AAV vector described herein and an immunotherapeutic agent (e.g., an immune response stimulating agent(s)) are also provided. In some embodiments, the immunotherapeutic agent comprises 1, 2, 3, or more immunotherapeutic agents. In some embodiments, the immune response stimulating agent comprises 1, 2, 3, or more immune response stimulating agents.

In some embodiments, the additional therapeutic agent is an antibody that is an immune checkpoint inhibitor. In some embodiments, the immune checkpoint inhibitor is an anti-PD-1 antibody, an anti-PD-L1 antibody, an anti-CTLA4 antibody, an anti-CD28 antibody, an anti-LAG3 antibody, an anti-TIM3 antibody, an anti-GITR antibody, or an anti-OX-40 antibody. In some embodiments, the immune checkpoint inhibitor is an anti-4-1BB antibody. In some embodiments, the additional therapeutic agent is an anti-PD-1 antibody selected from the groups consisting of: nivolumab (OPDIVO), pembrolizumab (KEYTRUDA), or pidilzumab. In some embodiments, the additional therapeutic agent is an anti-PD-1 antibody selected from the groups consisting of: MEDI00680, REGN2810, BGB-A317, and PDR001. In some embodiments, the additional therapeutic agent is an anti-PD-L1 antibody selected from the group consisting of: BMS935559 (MDX-1105), atexolizumab (MPDL3280A), durvalumab (MEDI4736), or avelumab (MSB0010718C). In some embodiments, the additional therapeutic agent is an anti-CTLA-4 antibody selected from the group consisting of: ipilimumab (YERVOY) or tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MED16469, MEDI0562, and MOXR0916. In some embodiments, the additional therapeutic agent is an anti-4-1BB antibody selected from the group consisting of: PF-05082566.

Furthermore, treatment with a recombinant AAV vector described herein described herein can include combination treatment with biologic molecules, such as one or more cytokines (e.g., lymphokines, interleukins, interferons, tumor necrosis factors, and/or growth factors).

In some embodiments, the recombinant AAV vector can be administered in combination with a biologic molecule selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-β. TNF-α, VEGF, P1GF, gamma-IFN, IL-1, IL-2, IL-3, IL-4, IL-5, IL-6, IL-7, IL-12, IL-15, and IL-18. In some embodiments, the recombinant AAV vector can be administered in combination with a biologic molecule selected from the group consisting of: macrophage colony stimulating factor (M-CSF) and stem cell factor (SCF).

In some embodiments, treatment with recombinant AAV vector described herein can be accompanied by surgical removal of tumors, removal of cancer cells, or any other surgical therapy deemed necessary by a treating physician.

In certain embodiments, treatment involves the administration of the recombinant AAV vector of the present invention in combination with radiation therapy. Treatment with an agent can occur prior to, concurrently with, or subsequent to administration of radiation therapy. Dosing schedules for such radiation therapy can be determined by the skilled medical practitioner.

In certain embodiments, treatment involves the administration of a recombinant AAV vector of the present invention in combination with anti-viral therapy. Treatment with an agent can occur prior to, concurrently with, or subsequent to administration of antiviral therapy. The anti-viral drug used in combination therapy will depend upon the virus the subject is infected with.

Combined administration can include co-administration, either in a single pharmaceutical formulation or using separate formulations, or consecutive administration in either order but generally within a time period such that all active agents can exert their biological activities simultaneously.

It will be appreciated that the combination of recombinant AAV vector described herein and at least one additional therapeutic agent may be administered in any order or concurrently. In some embodiments, the agent will be administered to patients that have previously undergone treatment with a second therapeutic agent. In certain other embodiments, the recombinant AAV vector and a second therapeutic agent will be administered substantially simultaneously or concurrently. For example, a subject may be given an agent while undergoing a course of treatment with a second therapeutic agent (e.g., chemotherapy). In certain embodiments, a recombinant AAV vector will be administered within 1 year of the treatment with a second therapeutic agent. In certain alternative embodiments, a recombinant AAV vector will be administered within 10, 8, 6, 4, or 2 months of any treatment with a second therapeutic agent. In certain other embodiments, a recombinant AAV vector will be administered within 4, 3, 2, or 1 weeks of any treatment with a second therapeutic agent. In some embodiments, a recombinant AAV vector will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

### Compositions

Nucleic acids (plasmids), vectors, recombinant vectors (e.g., rAAV), and recombinant virus particles can be incorporated into compositions such as pharmaceutical compositions. Such pharmaceutical compositions are useful for, among other things, administration and delivery to a subject in vivo or ex vivo. In particular embodiments, pharmaceutical compositions contain a pharmaceutically acceptable carrier or excipient. In some embodiments, such excipients include any pharmaceutical agent that does not itself induce an immune response harmful to the individual receiving the composition, and which may be administered without undue toxicity.

In another embodiment, the invention provides a recombinant AAV vector generated according to the invention.

In another embodiment, the invention provides an isolated eukaryotic cell useful for making a recombinant AAV according to the invention. The term “isolated” may refer to a compound or complex that has been sufficiently separated from other compounds with which it would naturally be associated. “Isolated” is not meant to exclude artificial or synthetic mixtures with other compounds or materials, or the presence of impurities that do not interfere with fundamental activity or ensuing assays, and that may be present, for example, due to incomplete purification, or the addition of stabilizers.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a nucleic acid, vector, viral particle or protein to a subject.

Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobrornides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery. Thus, pharmaceutical compositions include carriers, diluents, or excipients suitable for administration by various routes.

Compositions and methods may be sterile. The compositions may be made and methods may be performed in containers suitable for such processes. Such containers include dishes, flasks, roller bottles, bags, bioreactors, vessels, tubes, vials, etc. Containers may be made of materials that include but are not limited to glass, plastic and polymers, such as polystyrene, polybutylene, polypropylene, etc.

The compositions and method steps may be performed in a designated order, or rearranged order. The method steps can be performed in stages or at intervals with intervening time periods. In other words, a method step can be performed, and then an interval of time between the next step can occur, such intervals ranging, for example, from about 1 second to about 60 seconds; from about 1 minute to about 60 minutes; from about 1 hour to about 24 hours; from about 1 day to about 7 days; or from about 1 week to about 48 weeks.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Recombinant vector (e.g., rAAV) sequences, recombinant virus particles, and pharmaceutical compositions thereof can be packaged in single or multiple unit dosage form for ease of administration and uniformity of dosage.

The invention provides kits with packaging material and one or more components therein. A kit typically includes a label or packaging insert including a description of the components or instructions for use of the components therein. A kit can contain a collection of such components, e.g., a nucleic acid (plasmid), PEI, enhancing agent, cells.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, Labels or inserts can include information identifying manufacturer, lot numbers, manufacture location and date, expiration dates. Labels or inserts can include information identifying manufacturer information, lot numbers, manufacturer location and date. Labels or inserts can include instructions for using one or more of the kit components in a method, use, or manufacturing protocol. Instructions can include instructions for producing the compositions or practicing any of the methods described herein.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

All patents, patent applications, publications, and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features are an example of a genus of equivalent or similar features.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94%, etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-850, includes ranges of 1-20, 1-30, 1-40, 1-50, 1-60, 10-30, 10-40, 10-50, 10-60, 10-70, 10-80, 20-40, 20-50, 20-60, 20-70, 20-80, 20-90, 50-75, 50-100, 50-150, 50-200, 50-250, 100-200, 100-250, 100-300, 100-350, 100-400, 100-500, 150-250, 150-300, 150-350, 150-400, 150-450, 150-500, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions.

### Sample Embodiments

This section describes exemplary compositions and methods of the invention, presented without limitation, as a series of paragraphs, some or all of which may be alphanumerically designated for clarity and efficiency. Each of these paragraphs can be combined with one or more other paragraphs, and/or with disclosure from elsewhere in this application, including the materials incorporated by reference, in any suitable manner. Some of the paragraphs below expressly refer to and further limit other paragraphs, providing without limitation examples of some of the suitable combinations.

1. A method to generate recombinant AAV vectors using a mammalian cell line stably transfected with a therapeutic expression cassette composed of a sequence corresponding to a gene of interest, a promoter, a-polyadenylation sequence, flanked by AAV inverted terminal repeats, which is expanded and then transiently transfected using DNA with other components required to achieve rAAV biosynthesis/generation, namely plasmid or synthetic DNA expressing AAV Rep and Cap, and a plasmid DNA expressing helper virus genes Adenovirus E2, E4, and VA RNA, said method providing the benefit of prevention of packaging of unmethylated CpG sequences that are immune-stimulatory and contribute to detrimental immune responses.

2. The method of paragraph 1, wherein the plasmid DNA expressing helper virus genes expresses Herpes virus genes known to support rAAV generation in cell culture.

3. The method of paragraph 1, wherein the stably transfected cell line is HEK293 or HEK293T.

4. The method of paragraph 1, wherein the stably transfected cell line is BHK, CHO, HeLa, Vero, or any other mammalian cell lines know to support rAAV generation in cell culture.

5. The method of paragraph 1, wherein the therapeutic expression cassette contains one of the following genes of interest, coagulation factor IX, coagulation factor VIII, dystrophin, microdystrophin, alpha1 antitrypsin, and any other gene known to be missing a contributes to genetic disease.

6. The method of paragraph 1, wherein the therapeutic expression cassette that when expressed following administration to human subjects provide a therapeutic benefit for genetic diseases including, hemophilia B, hemophilia A, Duchenne's Muscular Dystrophy, alpha1 antitrypsin deficiency, and any other genetic disease.

7. The method of paragraph 1, wherein the therapeutic expression cassette corresponds to any gene that may have a therapeutic benefit for any genetic disease.

8. The method of paragraph 1, wherein the therapeutic expression cassette corresponds to any gene or any combination of more than one gene that when delivered to patients may have a therapeutic benefit for diseases without clear genetic etiology, including cancer, Parkinson's Disease, Alzheimer's Disease, macular degeneration, and diabetes.

9. The method of paragraph 1, wherein the resulting vector, upon suitable purification and testing, is administered to human subjects through the following routes of administration; intravenous, systemic, intramuscular, intracranial, intraparenchymal, etc.

10. A method to generate recombinant AAV vectors using a mammalian cell line transiently transfected with therapeutic expression cassette composed of a gene of interest enriched in CpG motifs, a promoter, a-polyadenylation sequence, flanked by AAV inverted terminal repeats, which is expanded and then transiently transfected with other components required to achieve rAAV biosynthesis/generation, namely plasmid DNA expressing AAV Rep and Cap, and a plasmid DNA expressing helper virus genes Adenovirus E2, E4, and VA RNA, said method expressly intended to increase the packaging of unmethylated CpG sequences that are immune-stimulatory and contribute to immune responses for elimination of vector transduced cells.

11. The method of paragraph 10, wherein the plasmid DNA expressing helper virus genes expresses Herpes virus genes known to support rAAV generation in cell culture.

12. The method of paragraph 10, wherein the stably transfected cell line is HEK293, HEK293T.

13. The method of paragraph 10, wherein the stably transfected cell line is BHK, CHO, HeLa, Vero, or any other mammalian cell lines known to support rAAV generation in cell culture.

14. The method of paragraph 10, wherein the therapeutic expression cassette contains any non-human gene that provides a target for immune recognition by the human immune response.

15. The method of paragraph 10, wherein the therapeutic expression cassette contains a non-human gene that encodes a protein that is presented at the surface of the transduced cell, thereby providing a recognition target for effector functions of the human immune responses.

16. The method of paragraph 10, wherein the resulting rAAV vector is targeted to cancer cells in a human subject, such that said cancer cells are specifically rendered targets for destruction by effector functions of the human immune response.

17. The methods of paragraph 10, wherein the resulting rAAV vector encodes a viral gene, such that said vector is designed to cause protective immunity for the corresponding virus.

18. A method to generate recombinant rAAV vectors in a mammalian cell line that has been modified to express increased levels of DNA methyl transferase specific for methylation of CpG dinucleotides such that said rAAV vectors have increased CpG methylation.

19. A method to generated recombinant rAAV vectors in a mammalian cell line that has been modified to express decreased levels of DNA methyl transferase such that said rAAV vectors have decreased CpG methylation.

20. A method to generate recombinant rAAV vectors in an insect cell line that has been modified to express increased levels of DNA methyl transferase such that said rAAV vectors have increased CpG methylation.

21. A method to generate recombinant rAAV vectors in an insect cell line that has been modified to express decreased levels of DNA methyl transferase such that said rAAV vectors have decreased CpG methylation.

22. A method to generated plasmid DNA corresponding to an AAV expression cassette in a bacterial cell line that has been modified to express DNA methyl transferase such that said plasmid DNA (vector plasmid) has CpG methylation.

23. The method of paragraph 22, wherein said plasmid DNA having CpG methylation is used to produce AAV vectors by transient transfection in human or other mammalian cell lines.

24. A method to generate synthetic DNA such as Doggybone DNA corresponding to an AAV expression cassette including a DNA methylation step using DNA methyl transferase such that said synthetic DNA has CpG methylation.

25. The method of paragraph 24, wherein said synthetic DNA having CpG methylation is used to produce AAV vectors by transient transfection in human or other mammalian cell lines.

26. A method to decrease CpG motifs in the genome of rAAV vectors combined with methods to increase methyl transferase activity during vector production and generation to decrease the quantity of unmethylated CpG motifs and thereby avoid immune responses and achieve long term therapeutic transgene expression.

27. A method to increase CpG motifs in the genome of rAAV vectors combined with methods to reduce or eliminate methyl transferase activity during vector production and generation to increase the quantity of unmethylated CpG motifs and thereby achieve specific and targeted immune responses for vaccines and cancer immunotherapeutics.

28. A method to generate recombinant AAV vectors using a mammalian cell line by transient transfection using a plasmid or synthetic DNA molecule containing a therapeutic expression cassette composed of a sequence corresponding to a gene of interest, a promoter, a-polyadenylation sequence, flanked by AAV inverted terminal repeats, which is prepared and expanded in a manner such that CpG dinucleotides are methylated, and using DNA with other components required to achieve rAAV biosynthesis/generation, namely plasmid or synthetic DNA expressing AAV Rep and Cap, and a plasmid DNA expressing helper virus genes Adenovirus E2, E4, and VA RNA, said method providing the benefit of prevention of packaging of unmethylated CpG sequences that are immune-stimulatory and contribute to detrimental immune responses.

29. The method of paragraph 28, wherein the DNA molecule containing a therapeutic expression cassette contains methylation of at least 20% of CpG dinucleotides, or better at least 50% of CpG dinucleotides, or best in the range 60%-90% of CpG dinucleotides.

30. The method of paragraph 28, wherein the plasmid DNA expressing helper virus genes expresses Herpes virus genes known to support rAAV generation in cell culture.

31. The method of paragraph 28, wherein the stably transfected cell line is HEK293 or HEK293T.

32. The method of paragraph 28, wherein the stably transfected cell line is BHK, CHO, HeLa, Vero, or any other mammalian cell lines know to support rAAV generation in cell culture.

33. The method of paragraph 28, wherein the therapeutic expression cassette contains one of the following genes of interest, coagulation factor IX, coagulation factor VIII, dystrophin, microdystrophin, alpha1 antitrypsin, and any other gene known to be missing a contributes to genetic disease.

33. The method of paragraph 1, wherein the therapeutic expression cassette that when expressed following administration to human subjects provide a therapeutic benefit for genetic diseases including, hemophilia B, hemophilia A, Duchenne's Muscular Dystrophy, alpha1 antitrypsin deficiency, and any other genetic disease.

34. The method of paragraph 28, wherein the therapeutic expression cassette corresponds to any monoclonal antibody.

35. The method of paragraph 28, wherein the therapeutic expression cassette corresponds to any gene that may have a therapeutic benefit for any genetic disease.

36. The method of paragraph 28, wherein the therapeutic expression cassette corresponds to any gene or any combination of more than one gene that when delivered to patients may have a therapeutic benefit for diseases without clear genetic etiology, including cancer, Parkinson's Disease, Alzheimer's Disease, macular degeneration, and diabetes.

37. The method of paragraph 28, wherein the resulting vector, upon suitable purification and testing, is administered to human subjects through the following routes of administration; intravenous, systemic, intramuscular, intracranial, intraparenchymal, etc.

Application of the teachings of the present invention to a specific problem is within the capabilities of one having ordinary skill in the art in light of the teaching contained herein. Examples of the compositions and methods of the invention appear in the following non-limiting Examples. Accordingly, the following examples are intended to illustrate but not limit the scope of the invention claimed in any way.

### EXAMPLES

### Example 1. Transfection of CpG methylated nucleic acids in a packaging cell line

The upstream steps of one improved process for which the vector plasmid used in transient transfection-based production of a recombinant AAV vector in HEK293 cells has been modified to contain methylated CpG dinucleotides so that, when packaged into AAV vector particles, provides vector genomes containing methylated CpG dinucleotides, is set out. Step 1 requires obtaining a commercially available strain of E. coli that is used for plasmid generation. Such strains are readily available in the commercial marketplace from suppliers such as Stratagene, Promega, Novagen, Invitrogen, and New England Biolabs. Further, many useful strains are available through the American Type Culture Collection (www.atcc.org) and the E. coli Genetic Stock Center at Yale (cgsc.biology.yale.edu). Examples of useful strains include DH10B, DH5alpha, JMI09, MC1061, TOP10, XL1 Blue, and XL10 Gold. A listing of various available strain genotypes is reported in Brown, A. (ed.) (1998) Molecular Biology LabFax I: Recombinant DNA. BIOS, Oxford.

Step 2 requires synthesizing the gene sequence for the desired methyltransferase. As explained above, DNA methyltransferases are well known in the art and their sequences are available in the literature. The methyl transferase chosen must provide methylation on CpG dinucleotides in a manner that corresponds to that found in human DNA. Standard techniques in molecular biology are used to synthesize the methyltransferase gene sequence.

Step 3 requires introduction of the sequence for the methyltransferase generated in Step 2 into the E. coli of Step 1. The sequence can be introduced either into the genome of the E. coli or as plasmids within the E. coli. In either case, the introduction of the sequence from Step 2 into the E. coli is performed using standard techniques that are well known in molecular biology. The result of Step 3 is an E. coli that expresses a methyltransferase protein. If the sequence is introduced into the genome, the E. coli will have a genome that expresses a methyltransferase protein. If introduced as plasmids, the E. coli will express methyltransferase proteins based on the plasmids and thereby methylate CpG motifs in the resulting plasmid DNA. The resulting E. coli then is a platform recombinant strain that, upon further recombinant DNA steps to introduce an AAV expression cassette containing a human gene of interest, can be used to produce a vector plasmid DNA of interest that can be used to generate AAV vectors by transient transfection, as described below.

Step 4 requires synthesis of a gene of interest, such as the gene for Factor VIII or Factor IX. It is known that there are approximately 30,000 genes in the human genome and a defect or deficiency in many of these genes may be create a condition in which gene therapy can be useful. Examples of such genetic disorders include hemophilia, cystic fibrosis, alpha- and beta-thalassemias, sickle cell anemia, Marfan syndrome, fragile X syndrome, Huntington's disease, and hemochromatosis. The sequences of the genes are known and can be synthesized using standard techniques from molecular biology.

Step 5 requires creation of a plasmid DNA that includes the gene of interest including the features required for its expression from a rAAV vector, including but not limited to a promoter, with the expression cassette flanked by AAV inverted terminal repeats, from Step 4, using standard molecular biology techniques.

Step 6 requires the introduction of the new plasmid DNA that includes the gene of interest from Step 5 into the platform E. coli of Step 3 that has been engineered to express a methyl transferase as a plasmid or into the genome of the platform E. coli. The resulting E. coli will thereby include both the gene of interest and the gene for the methyltransferase. The gene of interest is in the form of an expression cassette that includes the gene of interest and a promoter element flanked by AAV inverted terminal repeats (ITRs) on both ends. The platform E. coli thereby will replicate and in so doing the expression cassette of the gene of interest will be methylated by the methyltransferase created by the plasmid DNA.

Step 7 requires the E. coli of Step 6 that includes the expression cassette and methyltransferase DNA to be cultured and the quantity increased using techniques well understood in the art. Then using techniques well known in the art, the E. coli is lysed and the plasmid DNA containing the AAV expression cassette is purified and separated. The purified plasmid DNA containing the rAAV expression cassette will have CpG motifs that are methylated because of the methyltransferases expressed during the expansion of the E. coli. The result of Step 7 is the purified recombinant plasmid DNA that is substantially methylated at CpG dinucleotides, and contains the AAV expression cassette for the gene of interest.

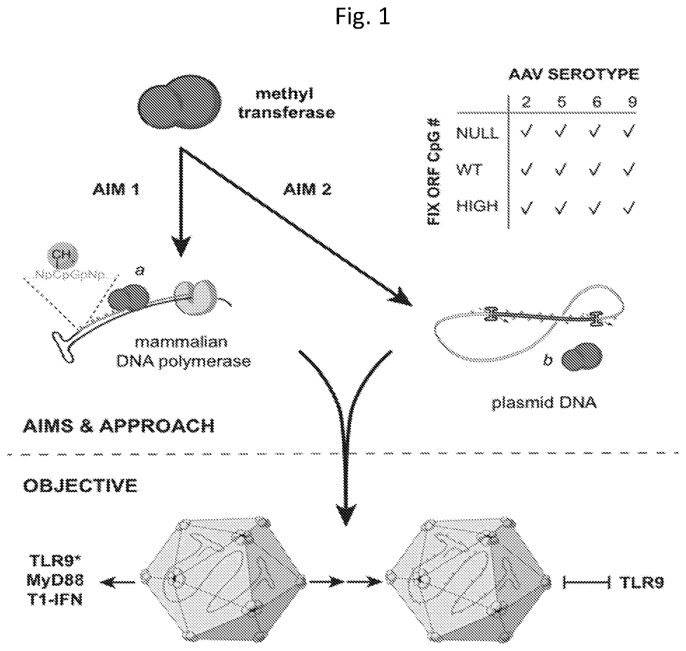

Step 8 is the subsequent use of the plasmid DNA containing the purified, CpG-methylated expression cassette to be packaged within AAV capsids, resulting in the desired rAAV vectors, as illustrated in FIG. 1. The recombinant bacterial plasmid DNA containing the AAV expression cassette is transfected as recombinant bacterial plasmid DNA into a production cell line such as HEK293, along with the other plasmids required to provide the requisite packaging and helper functions for AAV vector production using well known transient transfection techniques. The difference between the rAAV vectors resulting from the example transfection procedure described in the current invention and that of the prior art is that the bacterial plasmid DNA that is directly packaged in rAAV vectors has the CpG motifs methylated. In this manner, when the resulting rAAV vectors are administered as a gene therapy to a human subject, the subject's immune response will not be stimulated based on unmethylated CpG motifs because the CpG motifs are methylated. While in this example the final purified rAAV vector population will still have two sources of DNA, one resulting from DNA synthesis from the bacterial plasmid DNA template and other resulting from directly packaged bacterial DNA. In contrast, using the current standard practices for transient transfection-based production, the directly packaged bacterial DNA is unmethylated, and is therefore a PAMP that stimulates immune responses that are deleterious to durable gene expression. Ensuring adequate methylation of CpG motifs in all DNA present in rAAV vectors prepared for use in human gene therapy will provide key advantages in avoiding inflammation and achieving long-term therapeutic gene expression, the objective in this example.

### Example 2. Preparation of CpG Methylated Nucleic Acids In Vitro

Step 1. Methylation of CpG dinucleotides in an expression cassette containing a gene of interest (GOI) cloned in Doggybone DNA by incorporation of a methyl transferase (MT) during the synthesis of the Doggybone DNA.
Step 2. Use of the synthetic Doggybone DNA containing methylated CpG dinucleotides for transient transfection-based generation of AAV vectors in HEK293 cells.
Step 3. Purification of said AAV vectors expressing a useful GOI under GMP conditions, and administration to human subjects to achieve long-term expression of the therapeutic transgene.

### Example 3. Preparation of CpG Methylated Nucleic Acids in Cells that Overexpress a Methyltransferase

Step 1. Introduction by a stable transfection step of a methyl transferase gene into HEK293 cells using methods known in the field to obtain ‘HEK293-MT’ cell line.
Step 2. Introduction by a second stable transfection step of an AAV expression cassette containing a GOI into the HEK293X1 cells using methods known in the field to obtain ‘HEK293-MT-GOI’ cell line.
Step 3. Introduction of additional genes (helper and packaging genes) for generation of an AAV by transient transfection of HEK293-MT-GOI cells.
Step 4. Purification of said AAV vectors expressing GOI under GMP conditions, and administration to human subjects to achieve long-term expression of the therapeutic transgene.

### Example 4. Preparation of Recombinant AAV with Altered Immunogenicity

Gene transfer vectors based on adeno-associated virus (AAV) have demonstrated safety and transformative therapeutic effects for treatment of genetic diseases including RPE65^{-/-} retinopathy (FDA-approved 2017), spinal muscular atrophy (FDA-approved, 2019), and hemophilia A and B (pivotal trials ongoing), validating their enormous potential. However, host immunity remains one of the most challenging barriers to AAV-based product development, especially for those indications requiring high vector doses administered via immunologically responsive routes. AAV capsid-specific cytotoxic T lymphocytes (CTLs) are often observed following systemic AAV vector administration in clinical studies, sometimes causing partial or complete loss of therapeutic transgene expression by destruction of the transduced cells. This is a serious concern because patients are seroconverted to high titer AAV antibody status and precluded from future benefit using improved AAV products for many years, possibly for life. Thus, to realize the full potential of AAV therapies, the factors that contribute to efficacy-limiting immunotoxicity must be better understood and more effectively addressed.

It is proposed herein that activation of the TLR9-MyD88 innate immune pathway by Me^{neg}CpGs in AAV vector genomes is the trigger for capsid-specific CTL-mediated destruction of transduced cells and loss of transgene expression. Modifying AAV vector production to increase CpG methylation will remove this danger signal.

The overall objective of this example is to develop and test methods that increase CpG methylation [Me^{pos}CpG/total CpG] in AAV vector genomes to a level comparable to that in human DNA, thereby eliminating (Me_{neg}CpG)-associated PAMPs. The approach, based on the two possible pathways of AAV vector genome rescue, replication and packaging corresponding to prokaryote or eukaryote-origin genomes, is to provide targeted methyl transferase (MT) activity during these steps. AAV vectors expressing human coagulation factor IX (AAV-FIX) will be used as a model transgene to enable correlation of results with the extensive clinical data available for hemophilia B, with results obtained predicted to be broadly applicable. AAV vectors will be generated by standard transient 3-plasmid transfection of HEK293 cells (control), modified as described in the specific aims below (experimental), and purified by a standard process using affinity chromatography and CsCl gradient ultracentrifugation to provide highly purified, empty capsid-free vectors.

The following specific aims will evaluate the immunological effect of increasing Me^{pos}CpG in AAV achieved by directing methyltransferase activity to vector genome DNA before it is packaged into AAV particles:

Aim 1. Evaluate the immunological effect of MT provided during eukaryotic DNA replication and packaging on vector genome CpG methylation and TLR9 pathway activation. Using a plasmid expressing MT (pMT), AAV vectors will be generated by HEK293 transfection (pFIX, pAAVPK, pAdHLP, pMT), purified and characterized.

Aim 2. Evaluate the immunological effect of MT treatment of the vector plasmid DNA from which AAV vector genomes are rescued and packaged directly, on vector genome CpG methylation and TLR9 pathway activation. Vector plasmid DNA will be treated with methyltransferase, and AAV vectors will be generated by HEK293 transfection (pFIX-Me^{pos}, pAAVPK, pAdHLP), purified and characterized.

For both aims, experimental and control vectors including, three FIX ORF sequence variants (CpG-WT, 19 CpGs; CpG-null, 0 CpGs; CpG-high, 94 CpG) and four AAV serotypes (2, 5, 6, 9) reflective of clinical experience will prepared, to facilitate correlation with clinical data and provide a relevant and robust range of data. Comparative analysis of experimental and control batches will include: i) quality control testing to confirm purity and functional activity; ii) quantification of vector genome CpG methylation by bisulfite PCR sequencing; and iii) quantification of TLR9 innate pathway activation in human dendritic cells exposed to vector.

The primary outcomes of these studies will be; i) characterization of the immunological effect of modulation of AAV vector genome CpG methylation on human TLR9 pathway activation in vitro; and ii) the feasibility of novel methods to increase CpG methylation during vector production. The results will aim to define a new critical quality attribute for AAV vectors and inform future in vivo pre-clinical and clinical trials using improved AAV vectors that avoid triggering efficacy-limiting immune responses human gene therapy.

Table 1 shown below reports parameters for 8 AAV based gene therapy trials for Hemophilia B reporting long term follow-up.

TABLE 1
AAV gene therapy clinical trial for Hemophilia B

| | Se [symbol] otype/ | #CpG | | Dose (×10¹²): | | Immunology | | Outcomes: | | |
|---|---|---|---|---|---|---|---|---|---|---|
| Sponsor | config [symbol] | in OR [symbol] | Prod'n | ( [symbol] g/kg) | ( [symbol] cp/kg) | [symbol] S [symbol] | CTL [symbol] | Peak FIX | Duration | |
| 1. CHOP, | AAV2 [symbol] FIX/s [symbol] | 19 [symbol] (wt) | HEK | 2 | 2 | [symbol] | ++ | 12% (n [symbol] ) | <3 | mon |
| Stanford, Avigen | | | | | | | | | | |
| 2. UC [symbol] , St Jude | AAV8 [symbol] /sc | 0 [symbol] | HEK | 0.2 [symbol] 2 | 1 [symbol] 10 | + | + | 2 [symbol] 11% (10) | >1 | yr |
| 3. Shire | AAV8 [symbol] FIX Padua/sc | 99 [symbol] | HEK | 0.2 [symbol] 3 | NA | ++ | ++ | 4 [symbol] 45% ( [symbol] ) | <3 | mon |
| ( [symbol] AX335) | | | | | | | | | | |
| 4. CHOP | AAV8 [symbol] FIX29/ss | 94 [symbol] | NA | 1 [symbol] 2 | NA | ++ | ++ [symbol] | NA | NA | |
| 5. Pfizer | AAV [symbol] FIX Padua/ss | 0 [symbol] | HEK | 0.5 | 1.5 [symbol] 2.5 | + | + | [symbol] % (10) (15) | >1 | yr |
| (SPK-9002) | | | | | | | | | | |
| 6. Uniqure | AAV [symbol] /sc | 0 [symbol] | [symbol] | 20 | 40 | + | + | 7% (5) | >1 | yr |
| (AMT060) | | | | | | | | | | |
| 7. Dimension | AAV [symbol] FIX/ss | 96 [symbol] | HEK | 1. [symbol] 5 | NA | ++ | ++ | [symbol] 8% (6) | <3 | mon |
| (DTX101) | | | | | | | | | | |
| 8. Uniqure | AAV [symbol] FIX Padu [symbol] /sc | 0 [symbol] | [symbol] | 20 | 40 | [symbol] | + | 47% ( [symbol] ) | >1 | yr |
| (AM [symbol] 061) | | | | | | | | | | |

[symbol]

genome configuration [symbol] , single stranded genome [symbol] sc, self-complementary genome [symbol]
immune suppression, [symbol] not used [symbol] minority of subjects [symbol] majority of subjects [symbol]
C [symbol] -specific [symbol] minority of subjects [symbol] majority of subjects [symbol]
N [symbol] American Society for Hematology Annual Meeting [symbol] H [symbol] m Waterman lecture [symbol]
H [symbol] and Ang [symbol] (201 [symbol] ) [symbol] [symbol]
indicates data missing or illegible when filed

The strategies developed to reduce efficacy-limiting CTL responses; immune suppression until capsid peptides have been cleared from transduced cells, and codon modification of the transgene open reading frame (ORF) to remove CpGs, are only partially effective, and orthogonal approaches are needed. A novel strategy is proposed herein that aims to evaluate the immunological effect of increasing CpG methylation in the AAV vector genome.

It is proposed that activation of the TLR9-MyD88 innate immune pathway by Me^{neg}CpGs in AAV vector genomes is the trigger for capsid-specific CTL-mediated destruction of transduced cells and loss of transgene expression. Modifying AAV vector production to increase CpG methylation will remove this danger signal.

The aims, experimental approach and objective of this proposal are summarized in FIG. 1.

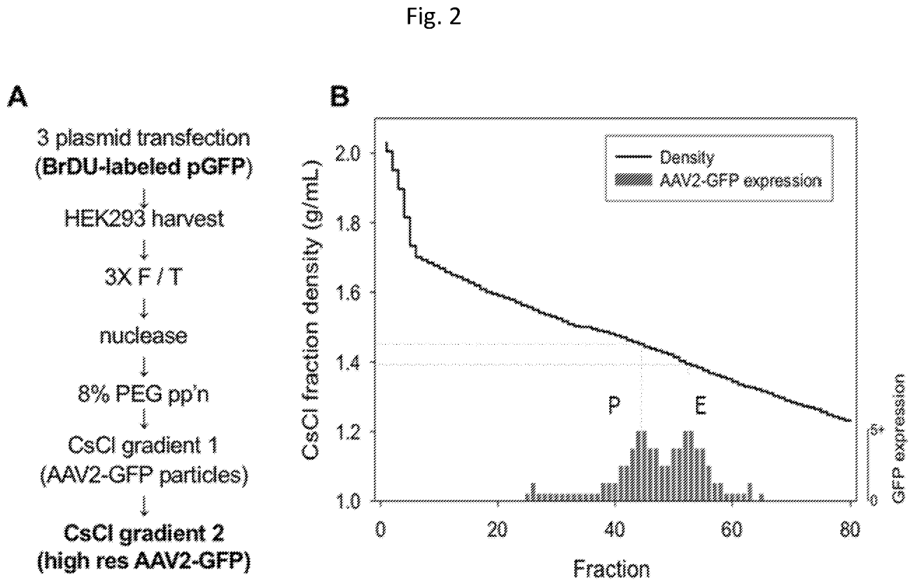

AAV packaging pathways: evidence for two sources (eukaryotic and prokaryotic) of packaged vector genomes. Knowledge of the provenance of AAV genomes during their synthesis and packaging is needed to effectively target CpG methylation during vector generation. The canonical view is that DNA packaged into AAV particles correspond to genome copies generated by eukaryotic replication after ‘rescue’ of the expression cassette from vector plasmid in HEK293 cells (Ward P, Elia P, Linden R M (2003). Rescue of the adeno-associated virus genome from a plasmid vector: evidence for rescue by replication. J Virol 77:11480-11490). However, calculation shows that the typical ‘input’ vector plasmid copy number (˜10⁵/cell) is approximately the same as the ‘output’ copies of packaged AAV genomes generated in high yield production, supporting feasibility of an alter-native pathway that doesn't require replication i.e. de novo synthesis of AAV genomes. We have performed an experiment to examine the provenance of DNA in AAV genomes generated by transient transfection (Hauck B, Mingozzi F, Arruda V, et al. (2006). Investigation of biochemical factors that may influence immunogenicity of AAV2 vectors. Mol Ther 13:S45). FIG. 2 shows the results using BrDU (higher density)—labeled vector plasmid that supports two packaging pathways; i) the packaging of AAV genomes generated by replication (normal density, right peak ‘E’), and ii) vector plasmid AAV genome rescue (excision) and direct packaging (higher density, left peak T′). Strategies to increase CpG methylation must consider both pathways.

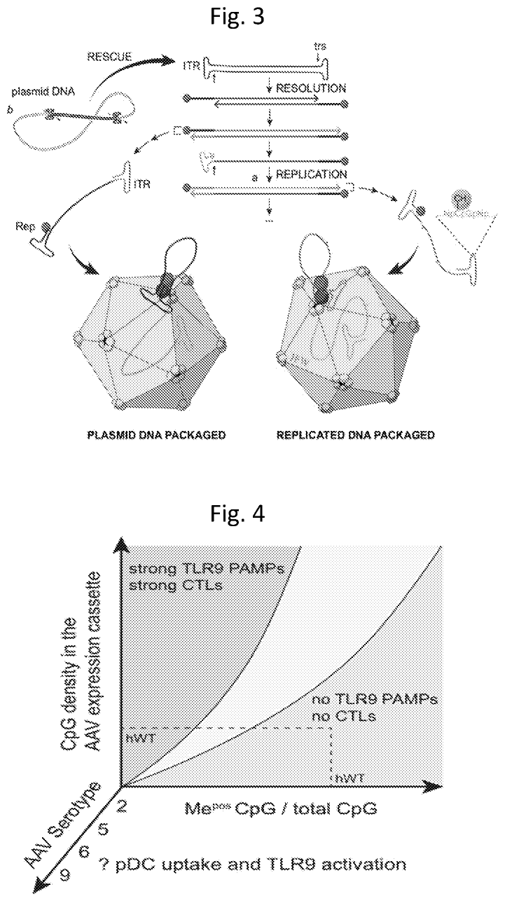

Model of AAV vector genome rescue, replication and packaging. Two pathways for AAV expression cassette packaging by transient transfection-based production in HEK293 cells are shown in FIG. 3. Both are consistent with mechanisms proposed by others, as well as our preliminary data supporting that both pathways are significant (Samulski R J, Srivastava A, Berns, K I, Muzyczka N (1983). Rescue of adeno-associated virus from recombinant plasmids: gene correction within the terminal repeats of AAV. Cell 33:135-143; Ling C, Wang Y, Lu Y, et al. (2015). The adeno-associated virus genome packaging puzzle. J Mol Genet Med 9: doi: 10.4172/1747-0862.1000175). Taken together these considerations predict that the AAV in the production harvest (and final purified vector) will contain significant fractions of particles derived from both pathways. The prokaryotic origin AAV genomes are predicted to be unmethylated at CpG dinucleotides, while those generated by replication in HEK293 cells may have some CpG methylation, but less than expected for mammalian DNA synthesis due to the rapid kinetics of viral genome replication and packaging (Toth R, Meszaros I, Huser D, et al (2019). Methylation status of the adeno-associated virus type 2 (AA2). Viruses 11, 38; doi:10:3390). Based on this model, increasing CpG methylation requires provision of methyl transferase activity at two stages to prevent a residual population of hypo-methylated AAVs; i) during the vector genome replication process (a), and ii) directed to the input vector plasmid prior to its use in transfection-based production (b), reflected in the specific aims of this proposal.

The research strategy is to increase CpG methylation in the vector genome during its production. Higher CpG methylation of extant CpGs in AAV vectors would allow the use of wild-type ORFs, mitigating the risk introduced by codon modification (Mauro V P, Chappell S A (2014). A critical analysis of codon-optimization in human therapeutics. Trends Mol Med 20:604-613; Alexaki A, Hettiarachchi G K, Athey J C, et al. (2019). Effects of codon optimization on coagulation factor IX translation and structure: implications for protein and gene therapies. Sci Rep 9:15449). It would also reduce TLR9 pathway activation by expression cassette elements such as the ITRs that cannot be readily modified to remove CpGs. The specific strategy is to direct methyl transferase M.SssI activity to vector genomes prior to their packaging during production in HEK293 cells to eliminate Me^{neg}CpG-associated PAMPs. Ideally a CpG methylation efficiency [Me^{pos}CpG/total CpG] comparable to that in human DNA would be achieved. Both pathways shown in FIG. 3 will be addressed because their relative contri-butions are unknown AAV vectors expressing human coagulation factor IX (AAV-FIX) will be used as a model transgene to enable correlation of results with the extensive clinical data available for hemophilia B, with results obtained predicted to be broadly applicable. This strategy recognizes the importance of using a human protein model for quantification of immune responses in human cells and leverages the abundant pre-clinical and clinical data available.

### Common Procedures Applicable to Both Specific Aims:

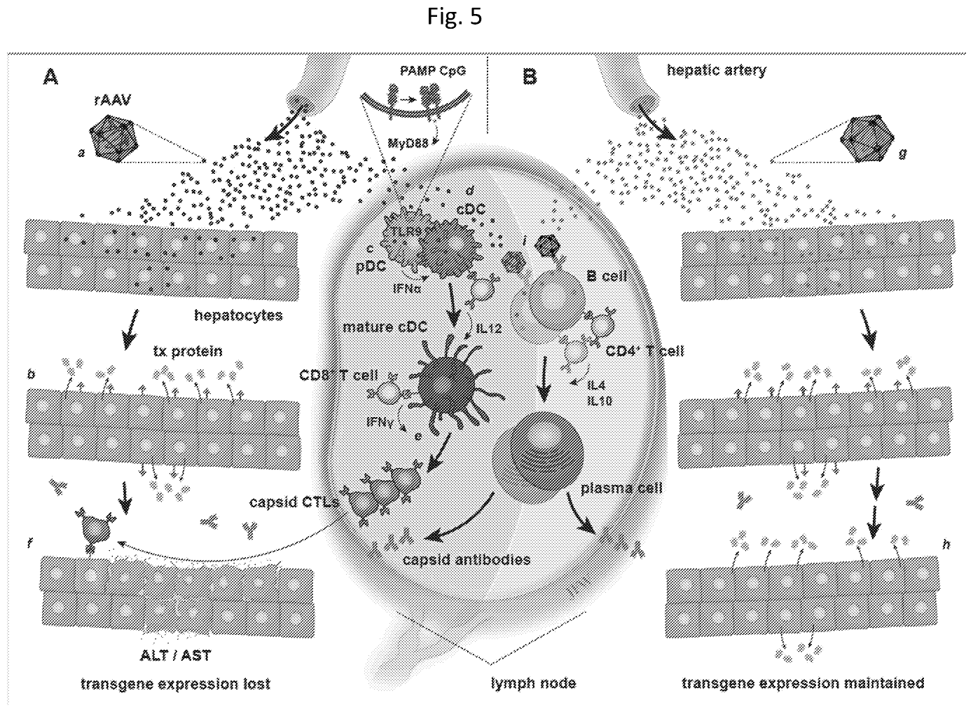

An AAV-hFIX expression cassette containing three CpG densities and packaged into four AAV serotypes. To increase the breadth of data provided by the experiments that focus on increasing CpG methylation by modifications to the conditions of vector generation, two vector construct design variables will be incorporated in the research strategy; i) three levels of CpG density (content) within the transgene ORF by differing ‘codon optimization’ approaches relevant to clinical experience; and ii) four serotypes that are prominent in clinical development. This multi-parameter strategy is represented in FIG. 4. For control and experimental vectors, the ‘hFIX16’ vector construct described by Manno et al with the wild type hFIX ORF (19 CpGs) in under the control of a hAAT promoter will be used (Manno C S, Arruda V R, Pierce G F, et al. (2016). Successful transduction of liver in hemophilia by AAV-factor IX and limitations imposed by the host immune response. Nature Med. 12:342-347). Two FIX ORF variants CpG-null ‘FIX39’ (0 CpGs) and CpG high ‘FIX19’ (94 CpGs) (High K A, Anguela X (2016) Modified factor IX, and compositions, methods and uses for gene transfer to cells, organs, and tissues. US Patent Application 20160375110). These FIX constructs are efficiently packaged to generate AAV vectors and express functionally active FIX protein. Each of the CpG content variant will be packaged into four AAV serotypes; 2, 5, 6, and 9 using published capsid sequences and packaging plasmids.

AAV Vector production and purification. Batches of AAV vectors will be generated by standard transient 3-plasmid transfection of HEK293 cells (control), modified as described in the specific aims below (experimental), and purified by a standardized method affinity chromatography and CsCl gradient ultracentrifugation to provide consistently pure, empty capsid-free vectors. This strategy will minimize vector production related variables and is based on methods developed by the investigators (Wright J F (2009). Transient transfection methods for clinical AAV vector production. Hum Gene Ther 20:698-706; McDonald C L, Benson J, Cornetta K G, et al. (2013). Advancing translational research through the NHLBI Gene Therapy Resource Program (GTRP). Hum Gene Ther Clin Dev 24:5-10). Briefly: the standard AAV-FIX ‘batch’ will be produced by transfection of adherent HEK293 cells by transient transfection using 100 ug per plasmid per RB. Following a medium exchange, the total harvest (cells and media) will be harvested at 4d post transfection, lysed by microfluidization, and clarified by 0.45 micron cartridge filtration. Vectors will then be purified using affinity chromatography (Poros AAVX resin) and isopynic CsCl gradient ultracentrifugation, and diafiltered into final formulation buffer: 180 mM NaCl, 10 mM NaPhos, 0.001% poloxamer F68, pH 7.3. Batches of the 12 unique ‘AAV-hFIX’ vectors (3 CpG densities X 4 serotypes) will be prepared by the standard protocol and by the two modifications described in the specific aims i.e. 12 for each of control (C), AIM 1 (A1), and AIM 2 (A2) arms, resulting in a total of 36 rAAV-hFIX vector batches that will be subjected to comparative analyses.

AAV vector characterization. Comparative analysis of the 24 experimental and 12 control batches include:

Standardized quality control assays to measure titer and confirm purity and functional activity (Wright J F, Zelenaia O (2011). Vector characterization methods for quality control testing of recombinant adeno-associated viruses. Meth Mol Biol: Viral Vectors for Gene Therapy, Methods and Protocols. Mohamed Al-Rubeai, Otto-Wilhelm Merten (eds.). Humana Press, New York, N.Y., 737 (Chapter 11): 247-278). Briefly, each AAV vector batch will be assayed by the following quality control tests against pre-established specifications: vector genome titer by qPCR; AAV particle titer and purity by optical density measurement²⁶; purity by SDS PAGE with silver staining; purity by endotoxin measurement; absence of microbial contamination by bioburden assay, and functional activity by transduction of HepG2 cells and quantification of FIX expression (Sommer J M, Smith P H, Parthasarathy S, et al. (2003). Quantification of adeno-associated virus particles and empty capsids by optical density measurement. Mol Ther 7:122-128). Batches that fail to meet the standard quality criteria established by the specifications will be replaced.

Quantification of vector genome CpG methylation. Bisulfate PCR sequencing (BPS) of packaged viral genomes will be performed according to the method reported by Zoltan and colleagues (Toth R, Meszaros I, Huser D, et al (2019). Methylation status of the adeno-associated virus type 2 (AA2). Viruses 11, 38; doi:10:3390). Briefly, bisulfite treatment of AAV vectors will be performed using the EpiTect Bisulfite Kit (Qiagen), and conversion of unmmethylated cytosines will be quantified by Sanger sequencing.

Quantification of the TLR9 innate pathway/danger signal activation in human dendritic cells. AAV vectors will be added to cultured human cells (dendritic cells or PBMCs), over a multiplicity of infection range from 10² — 10⁵ AAV per cell, and production of type 1 interferon will be quantified by intracellular staining and flow cytometry, or by ELISA (Zhu J, Huang X, and Yang Y (2009). The TLR9-MyD88 pathway is critical for adaptive immune responses to adeno-associated virus gene therapy vectors in mice. J Clin Invest 119: 2388-2398; Shirley J L, Keeler G D, Sherman A, et al (2019). Type 1 IFN sensing by cDCs and CD4+ T cell help are both requisite for cross-priming of AAV capsid-specific CD8+ cells. Mol Ther. https://doi.org/10.1016/j.ymte.2019.11.011; Kuranda K, Jean-Alphonse P, Leborgne C, et al. (2018). Exposure to wild-type AAV drives distinct capsid immunity profiles in humans. J Clin Invest 128:5267). Drs. Herzog and Roncarolo, who have both established DC interferon secretion assay established in their laboratories will provide advice and direction for this procedure (letters provided).

Aim 1. Evaluate the immunological effect of MT provided during eukaryotic DNA replication and packaging on vector genome CpG methylation and TLR9 pathway activation. Using a plasmid expressing MT (pMT), AAV vectors will be generated by HEK293 transfection (pFIX, pAAVPK, pAdHLP, pMT), purified and characterized.

Significance and approach. This aim will target the canonical source of vector genomes during AAV vector production by providing a supplemental plasmid pMT encoding methyltransferase M.SssI under the control of a CMV promoter with three standard production plasmids required for vector production in HEK293. Plasmid pMT will be constructed by standard molecular biology techniques and grown and purified by Aldevron at a scale sufficient to obtain 10 mg of purified research grade plasmid. Three input levels of pMT will be used; 50, 100 and 200 ug per roller bottle. The provision of MT activity during vector genome replication is predicted to increase CpG methylation over the level (<5%) reported using standard transient transfection without supplemental MT. 12 ‘A/’ constructs will be made and characterized as described in ‘Common Procedures’.

It is known that CpG methylation efficiency during packaging of small DNA viruses is inefficient, and it is thought that rapid DNA replication and packaging kinetics play a role. In the case that no increase in CpG methylation is observed, a modified approach will be developed a fusion MT protein targeted to the site of AAV genome packaging, as has been reported using Cas9 (Lei Y, Zhang X, Su J, et al. (2017). Targeted DNA methylation in vivo using an engineered dCas9-MQ1 fusion protein. Nature Comm 8:16026). A flexible linker will be used to link MT to a moiety known to target the AAV genome. Two specific candidate targeting moieties in are; i) the N-terminal domain of AAV Rep that is known to bind to the AAV ITR; and ii) the domain of Rep known to bind the 5-fold symmetry of axis pore structure on AAV particles (King J A, Dubielzig R, Grimm D, Kleinschmidt J A (2001). DNA helicase-mediated packaging of adeno-associated virus type 2 genomes in preformed capsids. EMBO J 20:3282-3291). When bound to M.SssI, such fusion proteins are predicted to focus the methyltransferase activity to the site of AAV vector genome replication and packaging during vector production in HEK293 cells. Another concern is that supplemental MT added may silence protomoters during production, or during subsequent vector transduction. If evidence of silencing is observed, a strategy using promoters engineered to be resistant to CpG methylation induced silencing (Moritz B, Becker P B, Gopfert U (2015). CMV promoter mutants with a reduced propensity to productivity loss in CHO cells. Sci Rep 5:16952).

Aim 2. Evaluate the immunological effect of MT treatment of the vector plasmid DNA from which AAV vector genomes are rescued and packaged directly, on vector genome CpG methylation and TLR9 pathway activation. Vector plasmid DNA will be treated with methyltransferase, and AAV vectors will be generated by HEK293 transfection (pFIX-Me^{pos}, pAAVPK, pAdHLP), purified and characterized.

Significance and Approach. This aim will target the alternative provenance of vector genomes during AAV vector production by transfection by methylating the vector plasmid in vitro using methyltransferase M.SssI. This CpG methylated vector plasmid (pFIX-Me^{pos}) will be used for 3 plasmid transfection vector production in HEK293. In vitro CpG methylation of AAV plasmid DNA using M.SssI has been reported by Zoltan and colleagues, and shown to not adversely affect AAV production by transfection (Toth R, Meszaros I, Huser D, et al (2019). Methylation status of the adeno-associated virus type 2 (AA2). Viruses 11, 38; doi:10:3390). To the extent that packaged vector genomes are the result of plasmid vector genome rescue and direct packaging, the use of CpG methylated vector plasmid DNA is predicted to increase CpG methylation in packaged AAV genomes. Twelve ‘A2’ vector constructs will be prepared and characterized as described in ‘Common Procedures’.

In the case that AAV vectors prepared using CpG methylated vector plasmid do not have increased CpG methylation, this would indicate that packaging of vector genomes by the prokaryotic pathway in FIG. 3 is not significant. If increased CpG methylation is observed but associated with reduced transduction efficiency, an alternative strategy would be use of a methylation resistant promoter (Moritz B, Becker P B, Gopfert U (2015). CMV promoter mutants with a reduced propensity to productivity loss in CHO cells. Sci Rep 5:16952).

The primary outcomes of these studies will be; i) characterization of the immunological effect of modulation of AAV vector genome CpG methylation, density and AAV capsid serotype on human TLR9 pathway activation in vitro; and ii) the feasibility of novel methods to increase CpG methylation during vector production. The results aim to define a new critical quality attribute for AAV vectors and inform future in vivo pre-clinical and clinical trials using improved AAV vectors that avoid triggering efficacy-limiting immune responses human gene therapy. If achieved, CpG methylated AAV vectors would represent novel prospective investigational products, and require further characterization for consideration for clinical development, including rigorous studies to assess potential genotoxicity and transcriptional silencing (Jones P A, Rideout W M, Shen J-C, et al. (1992) Methylation, mutations and cancer. BioEssays 14:33-36; Medvedeva Y A, Khamis A M, Kulakovsky I V, et al. (2014). Effects of cytosine methylation on transcription factor binding sites. BMC Genom 15:119).

### Example 5. Quantification of CpG Motifs in rAAV Genomes

Gene therapy using recombinant AAV (rAAV) vectors has demonstrated definitive benefits for genetic diseases and has enormous future potential, representing an important part of the next paradigm of human therapeutics. However, many clinical trials with rAAV have reported varying degrees of immunotoxicity. Analogous to the identification and removal of immunogenic features of early era monoclonal antibodies thereby ‘humanizing’ those products, while recognizing the immutable viral nature of the vector capsid, a similar strategy of humanizing addressable features of AAV vectors during their design is an opportunity to accelerate successful clinical product development. The synergistic nature of the multiple pathways that comprise human innate and adaptive immune responses combined with the consequences of failure to adequately control them after AAV-mediated gene delivery, including potential loss of transgene expression and AAV antibody seroconversion preventing re-administration, support the need to identify and remove ‘microbial legacy’ immunostimulatory features such as pathogen-associated molecular patterns (PAMPs) (Barton, G. M., and Kagan, J. C. (2009). A cell biological view of Toll-like receptor function: regulation through compartmentalization. Nat. Rev. Immunol. 9, 535-542). This example focuses on one such PAMP, the unmethylated CpG motifs (PAMP CpG) commonly found in AAV vectors due to hypomethylation of vector genomes during production and presence of expression cassette elements of microbial origin that are rich in CpGs (Toth, R., Meszaros, I., Huser, D., Forro, B., Marton, S., Olasz, F., Banyai, K., Heilbronn, R., and Zadori, Z. (2019). Methylation status of the adeno-associated virus type 2 (AA2). Viruses 11, 38). Herein are described approaches to quantify the TLR9 innate immune pathway activation risk for DNA sequences of interest e.g. AAV expression cassettes under consideration as investigational products, based on CpG/motif content and methylation, providing a tool to assess and guide reduction of TLR9-associated immunogenicity.

Hepato-immunotoxicity after systemic administration of AAV vectors— TLR9 as prime suspect. PAMP CpG binds and dimerizes Toll-like receptor 9 (TLR9) molecules expressed in plasmacytoid dendritic cells (pDCs), leading via MyD88 to cellular immune responses (Ohto, U., Shibata, T., Tanji, H., Ishida, H., Krayukhina, E., Uchiyama S, Miyake, K., and Shimizu, T. (2015). Structural basis of CpG and inhibitory DNA recognition by Toll-like receptor 9. Nature 520, 702-705; Hartmann, G., Weiner, G. J., and Krieg, A. M. (1999). CpG DNA: a potent signal for growth, activation, and maturation of human dendritic cells. Proc. Natl. Acad. Sci. U.S.A. 96, 9305-9310). FIG. 5 illustrates how high PAMP CpG levels in the expression cassette of an AAV vector lead to CTLs that eliminate transduced hepatocytes (A), while a vector genome with a sub-threshold PAMP CpG level does not activate the TLR9-MyD88 pathway and spares transduced cells, leading to durable transgene expression (B). Other factors not shown including dose and vector serotype contribute to these pathways. Transient immune suppression is frequently used and partially effective in managing CTL responses in rAAV clinical studies but adds complexity and risk (Samelson-Jones et al. (2020) Mol Ther. Meth. Clin. Dev. 17, 1129-1138). Avoidance of TLR9 activation by reducing PAMP CpG in AAV vector genomes during investigational product design is a promising approach to directly address the root cause.

Quantitative approaches to measure TLR9 activation potential. With recognition of the innate immunogenic risk of PAMP CpG in recombinant AAV, vector design strategies including codon modification of open reading frames and sequence changes in non-coding elements to reduce CpG dinucleotides are becoming best practices. Complete CpG removal from an expression cassette is possible but has potential to cause transprotein misfolding due to non-wildtype translational kinetics and adversely affect the performance of expression cassette elements such as ITRs. Understanding the PAMP CpG threshold for human TLR9 activation, coupled with a method to quantify the TLR9 activation potential (‘K_{TLR9}’) in candidate expression cassettes, would be helpful to guide clinical vector design. Three risk factor (RF) equations were developed and used to estimate K_{TLR9} in 15 relevant DNA test sequences with results shown in Table 2. The equations progressively incorporate three attributes of DNA sequences known to activate the TLR9-MyD88 pathway. Risk Factor 1 (RF₁) considers only the frequency of CpG dinucleotides, ranging from 0.965% in the human genome (suppressed compared to 6.25% for random nucleotide utilization) to 9.42% in the bacterium K. peumoniae genome for the 15 sequences analyzed. Risk Factor 2 (RF₂) multiplies RF₁ by the fraction of CpG dinucleotides that are unmethylated (CpGMe^{neg}/CpG_{T}) in each type of DNA test sequence; — 0.25 for human DNA (RF₂=0.25RF₁), 1.0 for bacterial DNA (RF₂=RF₁), an estimated 0.95 for the viruses and recombinant AAV vectors listed in Table 2 (RF_{2=0.95} RF₁) (Toth et al. (2019) Viruses 11, 38). Risk Factor 3 (RF₃) modifies RF₂ to incorporate known tetranucleotide immune-stimulatory (S4) and -inhibitory (14) CpG motifs reported by vaccine research aiming to enhance cellular immune responses using oligonucleotide adjuvants (Ohto et al. (2015) Nature 520, 702-705; Bode et al. (2011) Expert Rev. Vaccines 10, 499-511; Pohar, et al. (2017) J. Immunol. 198, 2093-2104). The S4 and 14 CpG motifs were enumerated and summed for each test DNA sequence. The motif sequences selected and their TLR9 activation ‘weights’ used for the RF₃ equation in Table 2 are preliminary and directional. A broader motif selection and more accurate, data-based, motif weighting factors would improve the predictive potential. Modification of RF₃ by incorporation of immune-stimulatory (S6) and -inhibitory (16) hexanucleotides CpG motifs resulted in a similar range of values as shown for RF₃ in Table 2 and preserved the relative ranking of the test sequences (not shown). A normalized value for RF₃ (NRF₃) was calculated by dividing the RF₃ value calculated for each DNA test article by that for the human genome (0.191) i.e. the sequence assumed to represent the lowest risk of TLR9 pathway activation. The NRF₃ for the complete human genome is by definition unity, with values ranging from 0.92 to 2.68 for selected genes and a CpG-rich portion of chromosome 1, providing an indication of intragenomic variation. In contrast, an average NRF₃ value of 20.7 was measured for three bacterial genomes known to be strongly TLR9 activating (Dalpke et al. Infection and Immunity 74, 940-946). Together the human and bacterial genome data define a NFR₃ range from 1 to ˜20 corresponding from negligible (−) to high (+++) values for K_{TLR9}. NRF₃ values for the genomes of helper viruses used in rAAV production ranged from 13.2 to 28.1, demonstrating the PAMP CpG risk represented by residual helper virus DNA impurities in purified AAV preparations. While it is challenging to obtain complete expression cassette DNA sequences for clinical vectors, the availability of sequences, clinical immunotoxicity and therapeutic outcomes for the four AAV-FIX vectors listed in Table 2 provide an opportunity to further qualify the NRF₃ equation (Wright, J. F. (2020). Mol. Ther. 28, 701-703). The better clinical performance of AAVSPK-FIX Padua/ss and AAV8-FIX/sc, including long term transgene expression and lower incidences of CTLs and immunotoxicity, correspond to vector expression cassette NRF₃ values of 3.09 and 6.80, respectively, lower than the values of 7.80 and 12.7 for AAV2-FIX/ss and AAV8-FIX19/ss, respectively, clinical vectors that gave higher immunotoxicity without durable transgene expression (Wright, J. F. (2020). Mol. Ther. 28, 701-703). These data support that AAV vectors with lower NRF₃ scores approaching a ‘humanized’ value have lower immunotoxicity and better long-term clinical benefit, while those with scores above a threshold value of ˜7 are associated with deleterious immune responses not well-controlled by immune suppression leading to loss of transgene expression. Use of such quantitative tools to evaluate TLR9 activation potential after further refinement and validation with information for other clinical vectors represents an approach to improve AAV vectors by reducing their potential to cause immunotoxicity.

TABLE 2
TLR9 Activation Risk Factors for Selected DNA Sequences

| | | | | | |
|---|---|---|---|---|---|
| DNA test article | RF₁ | RF₂ | RF₃ | NRF₃ | K_{TLR9} |

Human

| | | | | | |
|---|---|---|---|---|---|
| complete genome¹ | 0.965 | 0.241 | 0.191 | 1.00 | − |
| F8 gene² | 0.921 | 0.230 | 0.179 | 0.94 | − |
| F9 gene³ | 0.749 | 0.187 | 0.176 | 0.92 | − |
| Dystrophin gene⁴ | 0.797 | 0.199 | 0.200 | 1.05 | − |
| Chr1 CpG-rich fragment⁵ | 3.704 | 0.926 | 0.511 | 2.68 | − |

Clinical rAAV

| | | | | | |
|---|---|---|---|---|---|
| AAVSPK-FIX Padua/ss⁶ | 1.027 | 0.976 | 0.590 | 3.09 | − |
| AAV8-FIX/sc⁶ | 1.757 | 1.669 | 1.298 | 6.80 | + |
| AAV2-FIX/ss⁶ | 2.037 | 1.936 | 1.490 | 7.80 | ++ |
| AAV8-FIX19/ss⁶ | 3.530 | 3.354 | 2.418 | 12.7 | +++ |

Bacterial

| | | | | | |
|---|---|---|---|---|---|
| Escherichia coli⁷ | 7.471 | 7.471 | 4.683 | 24.5 | +++ |
| Klebsiella pneumoniae⁸ | 9.421 | 9.421 | 4.421 | 23.1 | +++ |
| Staphylococcus aureus⁹ | 2.548 | 2.548 | 2.750 | 14.4 | +++ |

Helper viruses

| | | | | | |
|---|---|---|---|---|---|
| AAV2¹⁰ | 5.847 | 5.555 | 4.968 | 26.0 | +++ |
| Adenovirus5¹¹ | 6.717 | 6.381 | 2.522 | 13.2 | +++ |
| Autographa californica¹² | 6.183 | 5.873 | 5.363 | 28.1 | +++ |

¹NCBI Homo sapiens GRCh38;
²NCBI NG_011403;
³NC_000023;
⁴NG_012232;
⁵NC_00001 (1000000-2000000);
⁶Wright (2020);
⁷CP_009685;
⁸FO_834906;
⁹NC_007795;
¹⁰NC-040671;
¹¹AC_000008;
¹²NC_001623

| Motif: | Weight: | Sequences included: |
|---|---|---|
| CpG_{S4} | +2 | Σ ACGT, TCGT, CCGT |
| *CpG_{Ex} | +1 | CpG_{T} − CpG_{S4} − CpG_{I4} |
| CpG_{I4} | −1 | Σ GCGG, CCGC, GCGC |

$${RF}_{1} = f [ {CpG}_{T} / nt ] \times 100 \%$$

$${RF}_{2} = f [ {CpG}_{T} / nt ] \times f [ {CpGMe}^{neg} / {CpG}_{T} ] \times 100 \%$$

$$\begin{matrix}{RF}_{2} = f [ {CpG}_{Ex} + 1 {CpG}_{S 4} - {CpG}_{I 4} ] \times [ {CpGMe}^{neg} / {CpG}_{T} ] \times 100 \% \\ = f [ {CpG}_{T} + {CpG}_{S 4} - 2 {CpG}_{I 4} ] \times f [ {CpGMe}^{neg} / {CpG}_{T} ] \times 100 \%\end{matrix}$$

$${NRF}_{3} = {RF}_{3} ( test article ) / {RF}_{3} ( human genome )$$

1. A method of generating a recombinant AAV vector with reduced immunogenicity, comprising:
providing eukaryotic cells with a nucleic acid comprising a sequence of interest that is flanked by AAV inverted terminal repeats,
wherein the nucleic acid comprises CpG dinucleotide sites, wherein at least a portion of the CpG dinucleotide sites are methylated, wherein the eukaryotic cell expresses one or more other components necessary to achieve recombinant AAV biosynthesis,
whereby the recombinant AAV vector is generated by the eukaryotic cell, wherein the generated recombinant AAV vector comprises nucleic acid wherein at least a portion of the CpG dinucleotide sites are methylated.

2-3. (canceled)

4. The method of claim 1, wherein the nucleic acid is methylated in bacterial cells modified to express a methyltransferase protein capable of methylating CpG dinucleotide sites.

5. The method of claim 1, wherein the nucleic acid is made in vitro using rolling-circle amplification to produce quantities of concatameric DNA that is then processed to create closed linear double-stranded DNA by enzymatic digestion (DOGGYBONE DNA).

6-25. (canceled)

26. The method of claim 1, wherein the eukaryotic cell has been modified to express a polypeptide capable of methylating CpG dinucleotide sites.

27-73. (canceled)

74. A method of generating a recombinant AAV vector with increased immunogenicity, comprising:
providing eukaryotic cells with a nucleic acid comprising a sequence of interest that is flanked by AAV inverted terminal repeats,
wherein the nucleic acid has been engineered to be enriched in immunogenic CpG containing motifs,
wherein the eukaryotic cell expresses one or more other components necessary to achieve recombinant AAV biosynthesis,
wherein the eukaryotic cell optionally has a reduced capability of methylating CpG dinucleotide sites,
whereby the recombinant AAV vector is generated by the eukaryotic cell, wherein the generated recombinant AAV vector comprises nucleic acid wherein at least a portion of the CpG dinucleotide sites are unmethylated.

75-78. (canceled)

79. The method of claim 1, wherein the nucleic acid is provided to the eukaryotic cells to achieve recombinant AAV biosynthesis by transient transfection or by stable integration into the genome of the eukaryotic cells or by infection of the eukaryotic cells with a recombinant virus.

80-82. (canceled)

83. The method of claim 74, wherein the one or more other components necessary to achieve recombinant AAV biosynthesis are provided to the eukaryotic cells by transient transfection, or by stable integration into the genome of the eukaryotic cells, or by infection with a recombinant virus.

84-85. (canceled)

86. The method of claim 74, wherein the nucleic acid encodes a therapeutic gene product that further comprises a promoter, and a polyadenylation sequence flanked by AAV inverted terminal repeats.

87. The method of claim 1, wherein the nucleic acid has been engineered to decrease the frequency of immunogenic CpG dinucleotide sites.

88. The method of claim 74, wherein the nucleic acid has been engineered to increase the frequency of immunogenic CpG dinucleotide sites.

89. The method of claim 74, wherein the recombinant AAV vector comprising a sequence of interest when delivered to a human subject is capable of treating a disease or condition in the subject.

90. The method of claim 74, wherein the sequence of interest encodes an antigen that provides a target for immune recognition by a subject's immune response when the recombinant AAV is administered to the subject.

91-92. (canceled)

93. The method of claim 74, wherein the recombinant AAV vector is targeted to cancer cells when the vector is administered to a subject, wherein the cancer cells are specifically rendered targets for destruction by effector functions of the subject's immune response.

94-126. (canceled)

127. A recombinant AAV vector generated according to the method of claim 1.

128. A method of treating a disease or condition in a subject, comprising administering to the subject an effective amount of the recombinant AAV vector according to claim 127.

129. The method of claim 128, wherein the recombinant AAV vector is administered according to a route selected from the group consisting of intravenous, systemic, intramuscular, intracranial, intraparenchymal and combinations thereof.

130. (canceled)


SEQUENCE LISTING (4 sequences)

SEQ ID NO: 1 - DNA, 104941 bp, Homo sapiens
ccaggagagactgcaaattgctgagccagcccaggcatatgagggcggtcacttcctccccagccttcaggtccccacacaagactgagatttccgctctgatagctctcagacgaggagaaaacctaattttcttcgtgcttcctcctctgcggattaaacacctggtttctagagtgtccaggcaacacttctcacttggtagaagaggcagtgaggttttgccaaggtccaagtgcgccccaagatctggcccagaacccagcagaccctgaaatcgagctcaggaaaaggggcaaagtgtagaattgactagtcactttctgactttctttgtatccccccttcaaagccccatcaccctgtgttagaattgtctggtttcggccgggcacggtggctcacgcctataatcccagcactttgggaggctgaggcaggtggatcgcctgaagtcaggagttcaagaccagcctggccaacatggtgaaaccccatctctactgaaaatacaaaaattagccgggtgtggtggtgtgcgcctgtaatcccagatacttgggaggctgaggcaggagaattgcttgaacctgggaggtggaggttgcagcgagccgagatcttgccagtgcactccagccttggcgacagagcgagactctgtctcaaaataaataaataaataaataaataaataaaaataattgtctggtttcttctctgtctcctccatcagactgtcagctttggatggcagcaaccaattaggcatcaccaggcttagcacagagcattacatgttgtgttacatacacacatatattgcatgcctatatgtagcattaacgtgttgattttttttttttttttgagatgggctcttactatattgcctgggctggtctcagattcctggcctcaagcaatcctcccgcctctgcctcccaaagtgctaggattacagttgtgagttatcgtgcttggcctgttgattttttttttttccagacagtgtcttgctctgtcaactaggctggagtgcagcagtgtgatttcagctaacttcaacctccacctctcaggctcaagtgatcctcccaccccagcctcatgagtagctggaactacaggcacacacaaccatgcttggctaatgtttaattttttgttttttcttttgtagagacgaagtctcactatattgcgcaggctgatctcaagctcctgggcacaactggtcttcccaccttggcctctcaaagtgctgggattacagacatgagccactatccatgaccaattttttaaaatttttaatatattttttgagatggagtttcactctgtcgcctaggctggagtgcagtggcacgatcttggctcactgcaacctccacctcccgggttcaagcagttctcctgccccagcctccagagtggctgtgactacaggcacctgctaccacgcctggctagttttttgtgtttttttttgtttgtttttttgtttttttgagatgcagtctcactctgttgcccaggctggaatgcagtggcgggatctcggctcagtgcaacctccgcctcctgggttcaagtgattctactgcctcagcctcccaagtagttgggactacaggtgtgcaccactgtgcccagctaatttttgtatttttaatagacaccgggtttcaccatgttggccagactggtcttgaactcctgacctcgtgatctgcctgcctcagcctcccaataattttggtatttttaatagagatggggtttcaccatattggtcaggctagtctcgaactgctgaattcaggtgatccccttgcctcgacctctttgtacatttttttgaattggtaatagaagctaaagatttcaaacagcaggctgggcgcagtggcttatgcctgtaatcccagcactttgggaggctgaggcaggcggatcacctgaggtcaggaattcgagaccagcctggccaatatggagatacctcgtctcaaaaaaaaaaaaaaaaaaaaaaaaaagatttcaaacagccctccagggtataccatgaaaagctgtttcttcttacctcgtttccctgccccctggttcctctccctaagagtaactgttgtgactagggtctggtgccttctcccagagacagactgtgtgtcacaggcaaatgtgtgtgtatttatatagttcttataaatacacccagaaatgcaatgcatactggtctacactgtactttttcaacttaatatgttgaagaccatttcccatcagtacacacaagactactccatttatttatttagttttatttatttatttatttaaaatggagtattgctgtgtcacccaggctggagtgcaatggcatgatctcggctcactggaacctctacctccagggttcaagcaattctcctgccttagcctcccgagtagctgggactccaggcacacactaccattcccggctaatttttgtacttttagtacagatggggttcgccatgttggccagcctggtcttgaactcctgacctcaggtgatccacctgcctttgcctcccaaagtgctggggttacaggtgtgagccattgcacccagcctctttatttttattttattttttgagacggagtcttgctctgttgcccaggctgaagtccaatagcaccatgttggctcactgcaacgcccgcctccccgggttcaagcgattctcctaactcagcctcccgagtagctgggattacaggcgcccgccaccacgcccggctaattttttgtatttttagtaaagaaagggttttgccatattggccagactggtctcgaactcctgacctcaagtgatccacccaccttggagtcctagagtgctgggattacaggcgtgagccaccatgcccagccaatttttatattttttgtagagatggagttacaccatgttgcccaggctaatctagaactcctagggtcaagcaatccacccaccttggcttcccaaactgttgggattacagtcgtgagccaccgtgcctggccagattatgtttataacatataatgtctacattttcacaaatggtaccctcataaaaagccatatttcccacctgaacataagatccactgggttggccaggcacggttgttcatgcctgtaatcccagcactttgggaggccaaggcgggcagatcatgaggtcaggagatcgaaaccatcctggccaacatggtgaaacctccatctctactaaaactacaaaaattagctgggcatggtggcgcatgcctgtaatcccagctactcaggaggctgaggcagaagaatcgcttgaacccgggaggcagaggttgcagtgagccgagatagtgccactacattccagcctggcgacagagctagactctgtctcaaaaaaaaaaaaaaatccatctggtcaaggagtttttcctttttgttgctgttgttgttgttcactgctgtgcccagtaggtgcataaaaaatatttgttgccaggtgctgtggtttacatttgtaatcccagcacttggggaggccgaggcaggcggatcacttgaggtcacgagttcgagaccagcctggccaacatggtgaaaccctgtctttactaaaaatacaaaaaaatcagccaggcgtggtggcacgcgcctgtatttccagctacttgtgaagctgaggcacaggaatagcttgaacctgggaggcagaggttgcagtgagctgagatggcaccactgcaccccagcctgggagataaaggaagactctgtctcaaacaaaaaaattttgttgctggaatgaggggctttcctatcctgttgtcgaactgctggttacacacacacacacacacacacacacacacacacacacacacatcattcgatgaaggtgtagtgtccatcatttccgcaggcaggcacacagaaatgcccaggaattagtgaaaattggaaatctggatagaagactgggcccgacgctggaaggcaggcacaaactgggccccgccagaggggacagtgatctacagaggcaggaaagagtgtggagtgtccccagtcccccaagggaaacagggcagcacctgaattctggaggtcctatcaagggagagactggtcaggcacagtggctcacgcctgtcattccagcactttgggaggctgaggcagtaggattgcttgaatgcagcagttcaagaccagcctgggcaacacagcgagaccccatctctacaaaaaactgcaaaaaattagccaggcatgatggcgcacctgtagtcccagctactctggaggctgaggcaggaggctgaggctcccgtgagctatgattgagccaccacactctggcctggatgacagagcaagaccatgtctcaaaaaaagaaaaaaaaaaagagatcaattttgtctttgggacaggacatcgcacttagtaggaatttatttgttgggtggacacacagtaggcacttaatgcctatttgctgcgttgacacctagtaggtacttccaaggctctttgttgagtcaataaatgtgacctcgtgttttcttccaagtccagggtcccaggggtaagcccagaggtaacctggggttgaagacagttgtttcaggttgaggccacctggccaggagtactgtgacgtcaccaggctgcgtgacgtttcgcgccggccggttctcacgggtgggggctgggaggggcagagtgagagatcgggtggggggggcggacaaagggccctccgtgcagctcctgtcggcccccgcgtgcgccccggccagccggccggtcacatctggaattcattgcagccgccgccggaaaggggaggagaggagggcggggcgagcggggggcagaggcggcggctctccgcccccccctctgtccctcgtcccagccccgggacttagccgagcccggctggcaaccccagccccagccgcggccgccctggggacgcagacgccaaggcccctccggccagggccgggagccgggccggcctagccaggtgagcgcgcgtggcgacaaggaggcagaaaaggcgggcctggtcccctgaggtctcgggatccgagctgaggggccccaatcggggggctggggcgcgcacgcgccgcctgggttcctgctgcacggagcgggtggtcacagtgcgccctctccaagaatccgggcggcccccactgcctcggccgcgaacccgcagactgtgctccccgcctttctcagcggcaggaagttggcgccggggagaaggctgaatccgagcgctttcgactggagtctttgggaggcaggacgccgtgctcggggaccggggtcttggggagtgtgtttgggggggggcaacacccctcctggctgactccggctgagcgagggcaaaaggatgtgcgcgccgtggagcctccaagccaaggggcggcacccacggggagggcggtcggacgtcgggctttctgcctcggtttccccaagttgtttgcctcctccggtcccccggcggggggcgtggctttgttttgtaaggaaagagaaagtgactgcgtggctcagacgcctcctctttcaactccggctcagggatgggtcgtgggtgaaggcgcgcgtggcgcggacctcgcgtggccccgagccgggcggagcgtcggaactcgcccccttctcaggcacgggttgactcacccggggatttccaccccctcctctgtctctgtccttgtcagatgctccgggccccacgcgctgagcccacagtccccctcgtggggcgacacggccaggcgcggaactcgcggtgcagctgctgctgcgatctggtcccccacccccagtcttcccagaatctcgaggcgggacccctggcgggacggagaggaatccttccagtgccgcttggagacgtgggggccccgcgcgtgtggcggtctcgggggcgggggggagaggaggcgtggctcccgcgcgagcgagcgtggataccgcgcattcctaaaccagataaggaggccgagagtgcgggccagcacggggtcgggtcctgctgccatctccatggagacacgctgggctttgcctggttgatggtgggcgatgggggcgctgcccgcggaccagaggtgtctccaggctcctttgttgctgtttgggtctgggctgagcttaggagtttccacccttgggaacccactagggtctcctttgttcattggttcattcattcattcattcatttgcccattccacacgttttctgagtgttttatttgtgccaggcatccctgtaggggctgataactgacttgctccctggccttaggcattgactgtctaggtggaagccagacagaaaaggaaagatatcatgatagtttcccatagtggcaagcgctgtgcagataataaaacgagattctgtgattagaaaaggcttggagatggctgctattgtagactgggggtcaggggagtcccctctgaggaagtgacctttaaatagaaaggtcactttctataaggtagggtggctaccacgggctccatgggtgaccagggggaactgtaaccacagggcctgcctttctgaaagatggggagacagtcattcaaagatttgaggaaaggccaggcgtggtggctcacgcctgtaatcccagcactttcctaggcttaggcaggtgaatcacctgaagtcaggagttccagaccagcctggccaacatggtgaaaccccatctctactaaaaatacaaaaattagccaggtgtggtggtaggcacctataatcccagctactcaggaggctgaggcaggagaatcgcttgaactcaggaggcagaggttgtggtgagccgagatcacaccactgcactctagcctgggcaacagagtgagactctgtctcaaaaaaaaaaaattttttttttgagaaaaagagtgtgccccaagaagaacagcaggggtaaaggcttggaggtaggggtaggggtagcatggaggaggtttagtgtggttggagtggaatggccctgggggaagagtgggaggaggcttcatcggcaggggccatcctacttgggctatagggagccatggaagaatttggagttggggagtgacatggagttacaagtttaaaagctccccctggctgcttggtagagaatggcagggtagggggtcctgacaaccacagagaaaccaaaaggtagggtggctaccacgggctccatgggtgaccagggggaactgtaaccacagggcctgcctttctggaactgggagtgggtggagaggaccttggaagccactgaggcttcctccatccttcccctctcctaatctccagacaaacagtcgtctgagtctatctctcttcccttccttccaaaccccatcacttgtccctcctgttccatttcaggcctggacattctatgtgagcccagtggcctctttctgagcttctgagagggagtcagggcagacttggtgaaggtgaccgggagacaggctctgtgtccttctcaaaacctggtaaccttacacagggaggggacatgggctaggccacgccatccacctagggggtgactcttctgccagctccaacctcagtgccctgcttcctctttctgggaggcgtcaaccgcccaagtgcttgcctcagtaaagtcctagtggttaagatgagtcttggatagaatgtgggcctcagtttccccttctctaggagagctgcttctccctctgtgaatgccttagcctccccgggctgcagaaggtgagagtgcagcacccccagtcttcctctcagggaaaaagacagactgcttagttagtccctggggtgcaagggctgaacacatttcattttgttttgttttgttttgttttgttttgagacagtctcgctctgtcacccaggctggagtgcaatggctgcgatcttggctcactgcaacctctgcctcccgggttcaagcgattctcctgcttcagtctcccaagtagctgggatcacaggcgcacaccaccatgcccggctaattttttttttttttttttttttttgagtagaggggggttttaccatgttggccaggctggtcttgaactcatgatctcaggtgataggcccgcctcggcttcccagagtgctgggattacaggcatgagccactgcgcccggcctaaacacatttcttatttatccaggaagccacagcaaatctcttggtgagcagcagagggggcggctgcactgcacagcgtgctccccaggcctggaaagggaccttgattctcacttatgaaccccaggaggggcccagcaggccagggcagcctgggggtgtctggtcggggtgacccagaggtcaggctgcggttcaaactccccctagggacgtccacatagcattaactctatcctcagaaagcctgagctcccgtcgcctataaaggaccccatcggtagggctggccgtagagcgcccccaactggaccaatcagaggcagaagatggaaagcccgggccaataggcaaattgctcagtgcccttggattgaaaatgagccaatcagagcatgggctgtttccggcccatcagctgtgtctgatccagccgcagcctgggcccaagcctgcctgggactggctgcagcgtgcagagggcatcaagatgcccggggctgcgtccagcaagggaacaaggggcctctgtgtggctatctgggaggggcagcagtccccggggacgtgcacaagcccttgtctgttcccaaactaggacggatgtccctccgccctgggggagcagctgcctctatccagccaggggccaggatgccccttggagggggcggccagggactgagatggctgggtgactccggaaacctccatcactcctgggctcaggagaactggcctaaccagacttttctggaacggtgcacacagtccttgccagacgtgtttctagggacctgaggaggactccccctactctcccccctccctcccatttagctaaggctggaacttcctctcccttcatgaaaggcatacactgcactggcgcctctgacaacccctaaccctacccccaagctgggggaaggccatggtgtctgcctctcatcctctctgccgaggagcgcctctccccgccccctcccaccacggctcagttttccttggtgggtggcgatggggggggatgggagggggtagagggagggatggaagagggtggaggggagggatgggagggggttgggggggtgactcaggttttcatggaacaaatgccttaccccctcccggcggggctgcacaccccttcacggctcacatgacttcagcaaagctgccccatcctcccagccaggacctcccagatggggtctctcccatcctgactccccctacatcccgccttcctcacaccggatgtgactgcagagtgaggactccaccctgtttgttccaggggcctcacatctggatttcatcctggctggggagggggtgccagcgccctgcccatcccggcaggaccttctgtcccagggcagagctagatggtgctgatgagaacaggctgttggagatctcgtctggttggcttacatcccttcccttcccaaaggactggaggaaagaggggagctaataacatttggcagtatttgttgagtgcttactatgagctaaacgtgcctcatgcgtcatttaaaaggattttcccatcacccccatgaagtgggctttcttctctctattgcacagataaggaggttcaggctgagacagcgtctagggacttgcccagtgtttcagaggtggaatgggagcaaattctggattgccttcaaggtctgtgtccttgaagatggtgtgacctctgggatgtttgaatctcagggccgccctggtgtctccccaagaccttatcttgggagcacccgtggcctcaaggcttagacccagcctgaaatccagcgttagtgggtggtaatgaatgagcagggcacggagctatcagtggtgagactctccctgacccaggtcagcatgagggcgcaggcgcctgtgtgttccagaaaagagacaaaccctccaggacctgcctagggcttgctctctgggtaggtgggcaaggtttctggaagtccacattatatttttcatgccatttgggggagcttctttccctttgaaagcgtgtagctactcaggagactgaggcaggaggatcacttgagcccaggagtagagacaactgtgagctatgatcatgccactgcactccagcctgggtgacagagcaagaccctatctctagaaaataataatgaaaattaaaagaggcaagatgtcatggttcatacctgtaatctcattgctttgagaggccgaggagaattgcttgaagccaggagttcaggaccagcctggacaacacagtgagacccaatctctaaaaaacaaaaaaactagccaggcggctgggcccggtggctcacactggtaatcccagcgctttgagaggccgaggcaggcggatcatgaggtcagaagttcgagaccatgctggccaacatggtgaaaccctgtctctactaaaaataaaaaaaaaattagctgggcctggtggcaggtgcccgtaatcccagctactcaggaagctgaggcaggagaatcacttgaacccaggaggaggaggttgcagtgagccaagaccgtgccgcagcactccagccttggcaacagagggagactccatctcaaaaaaaaaaaaaaaaaaaaaaaattagccaggcgtggtggcatgtgcttgtagctgaagctgaggtgggaggctgaggtgggaggatcacttgagcccaggagttcaaggctgcagggagctatgattgcgccactgcactccagcctgaatgacagagcaagaccttgtctttaaaatatagataaataaaataaataagaaagagcgtagcctagggcttatagaggccgcagagccagtctctctggccctgccgctggttaaatttgagactttaggaaagtgaattaatctctctgggtttcccgtctgtaagtggggatgatgatgattctgaattcatgtggcccttcctcgaagagtaacagagcaggaaattgaatctcagcccatccgaggccagagcccaggtagcagccacagctctgacctattgtctgtctgtctgtctgtcttctctgcagttctgaaagccccatggccccagcaggcctctgagccccaccatgggcagcttgtactcggagtacctgaaccccaacaaggtccaggaacactataattataccaaggagacgctggaaacgcaggagacgacctcccgccaggtggcctcggccttcatcgtcatcctctgttgcgccattgtggtggaaaaccttctggtgctcattgcggtggcccgaaacagcaagttccactcggcaatgtacctgtttctgggcaacctggccgcctccgatctactggcaggcgtggccttcgtagccaataccttgctctctggctctgtcacgctgaggctgacgcctgtgcagtggtttgcccgggagggctctgccttcatcacgctctcggcctctgtcttcagcctcctggccatcgccattgagcgccacgtggccattgccaaggtcaagctgtatggcagcgacaagagctgccgcatgcttctgctcatcggggcctcgtggctcatctcgctggtcctcggtggcctgcccatccttggctggaactgcctgggccacctcgaggcctgctccactgtcctgcctctctacgccaagcattatgtgctgtgcgtggtgaccatcttctccatcatcctgttggccatcgtggccctgtacgtgcgcatctactgcgtggtccgctcaagccacgctgacatggccgccccgcagacgctagccctgctcaagacggtcaccatcgtgctaggcgtctttatcgtctgctggctgcccgccttcagcatcctccttctggactatgcctgtcccgtccactcctgcccgatcctctacaaagcccactactttttcgccgtctccaccctgaattccctgctcaaccccgtcatctacacgtggcgcagccgggacctgcggcgggaggtgcttcggccgctgcagtgctggaggccgggggtgggggtgcaaggacggaggcggggcgggaccccgggccaccacctcctgccactccgcagctccagctccctggagaggggcatgcacatgcccacgtcacccacgtttctggagggcaacacggtggtctgagggtgggggtggaccaacaaccaggccagggcagaggggttcatggagaggccactgggtgaccccagatagagacttggggctactgagccagatgcccccgccccacagacctgggtgatgttgcaaatatttcacacctggaaaggccagataaggcactgactagtcacatagcagtgttgcagtgcggtcctgagggccagtccagtggctagtgtgacccctttagaactggatcctggggaggccagggcaggggacctgtgaagagccagggtgagggcaggcagcatttaaggggagctcagggcaggagcactttaccacctggtacaaaggatttttttttttttttgagacggaatcttgcactgctgcccaggctggagtgcagtggcgtgatctcggctcaccgcaagctccgcctcctgggttcatgtcgttctcctgcctcagcctcccaagtagctgggactataggcgcctgccaccacacctggctaattttttgtacctttagtagagatggggtttcaccgtgttagccaggatggtcttgatctcctgacctcgtgatccgcccgcctcggcctcccaaagtgctgggattacaggcgtgagccaccgtgcccggctttttttttttttttttttttttttttttttttttttgagatgaagtctcgctctgttgcccaggctggagagtgcagtggtacggtctcagctcactgcaacctccacctcccaggttcaagcgattctccagcctgagcctcctgagtagctgggattacaggtgcctaccaccacgcccaggtaattttttttttttttgtatttttagtagagacggggtttcaccatgttggccaggctggtctcgaactcctgacctcatgatccgcccgtgttggcctcccaaagtgtgggattacaggcgtaagccacctcacctggcggtacaaagaatttctgcattttcttccctggcccctagtcctgcaccgatttctccttttcgaatgtattcctcctgccaccttctctgggcaacttcgtgcgactacagaaccactgtcctgaggagctagaggcctcctctctgaccatccagagcccaaatccacagcttccccaaatttcatcagctgccacttgacgacttctccccgtctctctgaggcccggaaaccacggctggaggtggggaggggatggcggctgaggtccattcctcattctcagacctcattgctcagttgcactatttggggcacagaataatcaccaaaagtgagaaaaacgagtttgggtggctggggaggactttgggactcttgatgcaaggcgcaacttgagaaaattctgggtgtgatatttgcacagacaccctcctttcaaaaacagccaccccccaagctattctcagctccacacctgcagccccagctaaggtaccaggtctcctgagcaaggcagagagaagccttgagccttctctgtgtcttctttcaagaaccccgctgtgtcttctttcaagattttttttttgagacagtttcaagatttttgttttgtttttgagatggagtctcactgtgtcacccaggctgaggtggcagtggttcaatctccgttcactgccacctccacctcccgggttcaagcgattctcctgcttcagcctctcgagtagctgggactacaggcacctgccaccatgtctggctaatttttgtatttttagtagagacagggtttcactacgttggccaggctggtctcaaactcctgacctcaagtgatccgcccgcctcggcctccccaattgctgggattacaggcgtgagccactgtgcccggccttcttctttcaagttatatagaatggagcatgggggtggcagtggctagggacatttcctggggacactctcccctaaccccccagaaggacttcacaaaaacctgtggataatggaagggatgttacggtacaaacgtatatttatgtgtgtgtgtgtgtatgtgtgtgcgcgcgcgcgtgtgcacataggcgtgatgtctgtgaccctcctctcctcgtcacatttcccccagaatgaatgctgtcctgtctgctcatgtttgtgttgaagctgccaaagtcggggagctctggtcctgcccagacccctttggaattgctggcccatcctcccactggagagctggggtgcagctcaccttggggaaggaaacctcatgcctcagagtaatttcttgtgaatgcaaagcctgggggagcgggtctttggggggcaaggagccagtcaggggcttgtttcccctcatagagctccccagacgtgcctccgcaatgcctgaaacccagacctaggctaataaacggttcaatttctgtttcctgtggttggtgtgtcttttcggggagcaatttcccccaggagcgccccatggctgggctggatggaacaggaggggggctgctgcctgagtaactcatcctttgccaaccttcccattctgtgctcctgacacccttaacccggcctgagccaggagaaaacagaggtcaaccgggatctttcccaggggcaactcacctgcacctcccagggattaactcggtctgggatctccctgctttctctgaaggagacagcctggcaggtggtgggaggctcctacatcctcccaattgtatgtgcctcccggggagctggagagatggtttgagggggctacaaagaacccagatacccacccttagacacggtgttcccaaaagtaatggctgatcttcaccgagcactttccaggcataaagacttgactgctcaggaggtggaagttactataaactcttatggaggtgtgagttcagagagatcaagcaaactgctcagagtcacacagcgctagtcggggtagcgcctgtctggatttgaacccaagcaggctgactccagaccatactggatccaccactgggctagagaaaggccactcagctgagtgcagtggctcactcctataattccagcactttgggaggctgaggtgggtgggtcacttgagatcaggagttcgagaccagcctgccaacatagggaaaccccgtctctgcaaaaaatacaaaaattagctgggtgtggtggcgggtgcctgtagtcccagctactcaggaagctgaggcaagagaattgcttgaatccgggaggtggagaatgcagtgagccaagatcacggcactgcactccagcctgcgcgacagagcagagcaaaaaaaaagacagagagaggccacttagctccctctttgccatggaaggaggagccacgaagttcatcaagaaccaggagaagccaagataggatgcagcctcgggtgggtccctcggtgggttccctggtgatactcttctcttttgtctgcaggcagccggctcagtggctcctttgaagaaaagtctagagtttttttgtttgtttgttttttaagacagggtcttgctctgttgcctaggctgtagtgcagttggtgccatcatggttcacttcagcctcccaagtagctgggaccacagacatgcaccaccatgcctggctaattttttttttttttttttttgtagagatggggggtctccctatgttgcccaggctggtctcgaactcctgggctcaagcgatcctcctgcctccaccttggcctcccaaagtgctgggatttcaggcatgaaccctttgatgggggaccctaggcacccaagctggagccccacacagcaacaccagctcaaagactaggacagccagtgggagagaaggcagaccgcagaggctgagggcctgcaacccccatcggaattccccttctctagcagttgagacaacaacctctgtttggaaacctgaaccctagatgggatggggggtggggtgggggaaaaaggcacaggccaggctggagtcaatagattttccccaagtacccacagggttctctatctcccccttcaggactctgctcaaatgccacctcctcagaaaggagtcacttgcttattacctggcagtatatattattaagttggtgcaaaagtcattgcggtttttgccattacctttaatagcaaaaattgcaatgacttttgctatttcttttaatggcaaaaaccgcaatgacttttttttttttttgagatggagtctcgctctgtcacccaggctagagtgtatggcccgatctcggctcactgcaaactccgcctcctgggttcacgccattctcctgcctcagcctcctgagtagctgggactactagtgcccgccaccataccaggctaattttttgtattttttttttttagtagagacagggtttcaccgtgttagccaggatggtctcaatctcctgaccttgtgatccacccacctcggccccccaaagtgctgggattacaggcgtgagctaccgtgcccagcctacaatgacttttaatggcaaaaaccgcaatgattttagcaccaacctaatatgtatcaatttatctgtctctacccccattaaaatacaagcactgtgaatataggactgcacctgccttatttactaatgtatctccagtgttgtttttttgtttttttttgagacaaggtcttactctgttacccaggctcaagtgcagtggtgcaatcacagctcactacagcctcaacctcctgggctcatgggctcaggtgatacttccacctcaggcttcctagtaactgggattataagcctgctaattttctttttttgtagagacatgattcttccatgttgcccaggctggtcttgaactcctaggcttaagagatctgcctgcctcaacctcccaaagtgctgagattacaggtgtgagccaacttgcctggctatttatggctctctaactttttttttttttttgatacagggtctgactctgtcacccaggctggagtgcagtgacgtaatcacagctcaccgcagccttgacctcctgggctcaagccatcctcccacctcaatctcccaagtaggtggaactacaggtatgtgccaacacactggcctgcttttttgtattgtttgtagagacagggtttcaccatgttgcccaggctgctctcaaactcctgggctcagtgctcctcccacctcagcctcccaaagtgctgggattacaggcatgagccaccatgcctggctatctctaacatcttaaacactacttggcatgttcccaccccagggcctttgcacctgctgttccctctgcctggaatgcctacacacagtaggtcctcagtaaatacttgttgaataaataaaacaaaagcaaatgatggtcatttcagtgagtgttaagtgctaggaagaaaataaaatagggccgggcacggtggctcatgcctgtaatcccaccactttgggaggccgaggtgggcagatcacctgaggtcaggagtttttttgttttgttttgttttgtttttgagacggagtcttattctgatgcccaggctggagtgcaatgatgtgatctcgactcactacaacctccacctcctgagttcaagtgattctcctgcctcagcctcccgagtagctggcattacaggcatgtgccaccaagcctggctaatttttgtatttatagtagagacgggggtttcatcatgttggtcaggctggtgtcgaactcctggcctcaagtgatctgcttgcctaggcctcccaaagtgctgggattacaggcgtgagccactgtgcccagccaaggtcaagagtttgaaaccagcctggccagcatggtgaaaccctatctctactaaaaatacaaaattaggccatgtgcctaattttgggattacacctgggatcatgcctgcaatcccagcactttgggaggccaagacgggtggatcatgaagtcaggagttcaagaccagcctggccaacatggtgaaaccctgtctctactaaaaatacaaaagctagctgggcgtggtggcacgcacctgtagtcgcagctactagggaggctgaggcaggagaattcttgaacccgggggatggaggttgcagtgagccgagattgcgccactgcactccagcctgggcaacagagtgagactctgtctcaaaaaaaaaaattagctgggcgtggtggcgagcgcctgtaatcatagctaccggggaggctgaggctggagaattgcttgaacccgggaggcagaggttgcagtgagccaagatcgcgcctttgcactccagcctgggcaacaggagcgaaactgcatctcaaaaaaaaaaaaaagggggggcggcatttgtccctgcgacagaactgctgccgccattgccgtctctaatatctaatataataatatctaatatctaatataataaggtctcaccacagccaccaggccttggggaagccacagtgtggccgactggccagtcagacccccaccggtgctgcctcccagcccctacagatggggctctgcacctacaagatcaggcagttcctggactgttcgaccactgagaatgacctgtccctgtgtgagggcttcaatgaggccccaaagcagtgcaagtacaaccatggtctgagctccctgccttgaagacgttggtgcagactcgagggccagctctgcacccacctgtaccccttgcccacagccagaccacaacaccagattgtacctggatagctgggattggaagtgaggaataggtttctcaccccacagataacccaagacacaaatgtgcaattaaaaaatttattttagaccaccaaaacaaacaaaacaaaacagaaacaaataaaaaaaagaaaagaaaatagtgccaggtgtgggagctcactcctgtaatcccagcactttggaatgccgaggcgagtggatcacctgaggtcaggagtttgaaaccagcctggccagcatggtgaaaccccatctctactaaaaatacaaaattaagcaggcgtggtggtgggcacctgtaatcccagctacttgggaggctgaggcaggagaatggcgtgaacctgggaggcggagcttgcagtgagccgagatcgcgccaccgcactccagcctgggtgatggagcaagactctgtctcaaaaaaaaaaaaaaaaaggaaagaaaagaaaatagggccaggcatggtggctcatgcctgtaatcccagccctttgggagaccaatgcgggaggatcacttggggccaggagtttgagatgagcctgggcaacatagtgagacctcatctgtaaaagtaagtaaataaatacatatataccctggtgtggtggaacgtgtctatagtcctggccactctggaggctgaggtgggaggattctttgagcctaggtgaccgaggctgcaatgaactgtggtcatgccactgcattccagcctgggcaacagagtgagaccctgtctcaaaaaataaaaagtctaatgtgtgctcgtgtgctgggcactgtgagaatcatttgacatctgcaaacttctggaatcctcacaataatcctatagagtaggtgctattattggctttttgacagataaagaaaccgaggcacagagaggttaagtaattcacccaaggtcaagtagcatgggagtcaaggggccaggattggaacccagtgagtcactgaagaccacacttatatctttttaaaatgtttttcagctgggcgcggtggctcacgtctgtaatcccagcactttgggaggccgaggcgggcggatcacctgaggttgggagttcaagaccatcctgaccaacatagagaaatcctgtctttactaaaaatacaaaattagccgggtgtggtggcacatgcctgtaatcccagctactcgggaggctgaggcaggagaattgcctgaacccaggaggcagaggttgtggtaagccggagatcgcaccattgcactccagcctgggcaagaagagcgaaaccccgtctcaaaaaaatgattatttatttatttatttttggagatagagtctcactctatcgcccaggctggagtgcagtggcacagtctcggctcactgcaacctccacctcccaggttcgagagtttcttccgcctcagcttctctagtagcggtgattacaggcacgcaccaccacgcctagctaatttttttttttttgagacagagtttctctcttgtcacccaggctggagtgcaatggcgcagtcttggctcactgcaacctctgcctcccaggttcaagcgcttctcccgcctcagccttccaagtagctgggattacaggcgtgcaccatcatacccagctaatttttttgagatggagtcttgcactgtcgcccaggctgtagtgcaatggtgcgatctcggctcactgcaacctccgcctcccaggttcaagagattctccttgcctcagcctctcaagtagctgggatgacaggtgcccgccaccatgcctggctaatttttttgtatttttagtagagacagggtttcaccatgttagccaggctggtcttgaactcctgacctcatgatccacccaccttggcctcccaaagtgctgggattacaggcatgagccactgcgccgggcccacaatcatatctgatcctcctcaatatctggatctgaagatcccatttgttgatagctctcaaatcttggagcccaaattaaagtggtccttccagcaggaaacacaagatgactttcacctcctttttcccctagggaggctgttgcctaatagggaccaacaaaggaatgacctggaagaacctgcttaaatgcaaattctcaagctctatggtcagagattctgactaaacaggtctggaatgaacctagaaatgtgtgtttcttaccaggaacccctaataatttaaaaatatgtagtccttggccggggaacagtggctcacgcctgtaatcccagcactttgggaagctgaggcgggtggaccatctgaggtcaggagtttgagaccagcttgatcaacatggagaaaccctgtctctactaaaaatacagaattagccgggcatggtggcgcatgcctgtaatcccagctacttgggaggctgaggcaggagaatcgcttgaaccctggaggcagaggttgcggtgagtcaaggctgcaccattgcactccagcctgggcaacaagagcgaaactctgtctcaaaaaaaaaaaaaaaatatatatatatatatatatatatatatagtctttgcactatattctgttttgttgttgttttggagacagggtctcactctgtcacccaggctggagtgcagtggtgccaattcggctcaccgcagcctcaacctcccaggctcaagtgattctcttgcctcagcctcccgagaagctgggactgtaggcgcttgccaccaagcctggctactttttaaattttttatttgtagagatgggcttttgccatgttccctaagctggcctcaaactcctgggctcaagtgatcctcccatctcggcttccgaaagtgctgggattacaggtgtgagccactgtgcctggcccttgcaccatattctaagaagcattctcggctccataattcagagcagcactgtcatgagcattacaaatacgactaatgtgaggcaggcaggggctgaattttacatattattgaattttaattaacaaatcattagctacaatattgggcagtgcaattctagatttcatatttctagtttctgcagcctctgagtaactgagctcttttgattactatctcaacaatttttcttttctttcttttttttttttttttttgagacagagtctcacactgtcgcctgggctggagtgcaatggcggctctcggctcactgcaacctctgcctcccaggttcaagtgattctcctgcctcagcctcccgagtagctgggattacaggtgcccaccatgcctggctaatttttttttttttgtatttttttgtagagacagggtttcactatgttggccagactggtctcaaactcctgaacttgtgatccgcccgtctcggcctcccaaagtgctgggattacaggcatgagccaccgtgcccggcccatctcaacaattttttttttttttttttgagacagcgttttgctcttgttgcccaggctggagtgcaatggtgcagccttggctcactgcagcctccacctcccaggttcaagtgattctcctgcctcagcctcccaagtagctgggattataggcgcatgccaccacgcccggctaatttttgtatttctagtagagacagggtttcaccatgttggctagactggtctcaaactcctgacctcaggtgatccgcctgcctcagcctcccaaagtgctagaattacaggcgtgagccaccgcacctggccagaacaaggcgttctctaacataacctcagtaaaatgatcaaaagcagaaaatctgcaaacctaattagatttcacccattgtcacatcactgacctttatagcaaaagaaaatcccagatcagttgttgtttccagttgtctttttagtttctttcacggagatcaatttctgagcctttttgtgttttgcgaccttgacgttttaacaaaatgtaggttagttattaggcagaatgtctttcaacttgggtttgtctgctgtttccttatgattagattcaggttgtgcctttttggcaggaagaccccagcgtgtctctgctataaagttactgctttccggtttgtaattattaagtttcttgtggggaggtatgtggagactctataaatatcctattattcatcagactttcactctttcagcattcattcttgcctgaatcaattattattcaaaggatttttttttttgagacagtgtctcactctgttgcccaggctggagtgcaatggtgtgatcacaactcactacagccttgacctcccaggcccaagcgatcctcctgcctcagcctctcaagtagctggggctacaggtgcaagccaccatgtctggctaatttttgtaaagaagggtttcgctgtgttgcctaggctggtctccaactcttgggctcaagcaatccacccaccttggcctccccaaattatgggattacaggtgtcagccaccacacccagccagcctcactggatattttgaagcaaatctgaggcctcaaataatttcatttgtaaatactccatttttttatctctaaaaggtagcaacacttaaaaaaatgctacaataccttttttgggcagggtgtggtggctgactcctgtaatcccagcactttgggtggctgaggtgggaaaatagcttgagcacagaggttggaggccatcctgggcaacatggtgaaactctgtgtctaccaaaacaaaaaaaaaaaaaaaaaagctgggcatggtggcatgcacctgtagtcccagctactcaggaggctgagcagggaggattgcttgagcccaggaggcagaggctgcagtgagcagagactgcccacctgcgctccagcctgggtgacagagtgagaccccccatctcaaaagcaaaacaaaacaaaaaccgaaaaacctttctgtagaaccaaggtctcactatgttgcccaggctagtctggaactcctgggctcaagtgatccttctgcctcagcctcccaaagtgctaggattacagtcatgagccactgtgcctggcctttctttccacttttcactgataactaagaatgatccagggattcggatgccatccgcctgaccatgcctttgtaaattccccacttggcgtttccctaaaggtttgatcatccatggataaccattgcctggaactgttgtttcattagcagtcttcaaagggtttctggatgggcgtggtgtggcccatgtctgtaattccagtgctctaggaggctcaggcgggaagattgcttgaggccaggaattcgagactagcctgggcaacacagtgagacacccccagctctacaaaaaatcttaaaaattagttgggcatggtagcaggaacctatagtcccagctattcaggagggcaaagcaggaggatcacttgaacccaggagttggaggatgcagtgagctatgatggcaccactgcactccagcctgggcagtagagtgagactgcctctctaaaaacaaacaaacaaacaaacaaacaaaaaacctacaaacggtttctaaaccgtctctccatcctcatatctagccattttttcccatttgtctacaaagctgacatacttgcccaaaattcagacacccaataccaccaccttccccctttttttggacagatttaactattctcggataagaaattcaaagaaagcaataccaccacccttccttgaatgtcttatccaagaatagacaaatctgtccaaaaaaagggaaatgaagtgggtcggatgtgattcattgttgtcggcaattgtggtggcataactgttaaaagtgcatactatcaagttaggaaaagcagatttgaatctcacatcccacactcaacaacccaagtagcttttggggatggcacatgacaactgtcagctggcctcatctataaaatgagtatgttactttattttttttgagatggagtctcactctgtctcccaggctggagtgcagtggcacgatcttggctcactgcaacctccgcctcccaggttcaagtgattctcctgcctcagcctccccaggagctgcgattacaggtgcccaccaccacgtccagctaatttttgtatctttagtagagacggggtttcatcatgtagccaggctggtctcaaactcctggcttcaagtgatctacctgcctcgtgatccacccgccttggcctcccaaagtgctgagattacaggcgtgagccactgcactcagctcattacttttaatagcaaagatcgcaattacctttgcaccaactaatgatccccaacttgcagattgcttttggagaatcagctgtggttaattggtggtcttttccaggaaggagtctcctcacgtgacatccctctggcttaatttaaggccagggcaggcctagcttttattcctgctcaccgcagtgtagacagggaaactttctttgcagttcaggagcagacgctgtgtattttcacgtcattctccaggaggtgggcaagtctctggcctgtaactggcattttttctctagggcttgtggctttcatggacaatggccatgtcatggaccctggcaggcagaaggaggctgggaagagttgagggaggaaggcagggtgtaagtggcctggaaggaaaaaaggaagcctaagtttgaagtgggtgagaaaaatgaagataagtgccttggtaatttctataatcagaattacacagtatgtaccctcctatgactggtttctttcatacagcataaggtttttgaaattcactttttttgtttgtctgttttttctaagatagggtcttgctctgttgcccaggctggagtacagtggcataatcatagctcactgcagcctttatctccagggctcaagcgatcctcccacctcagcttcccgagtagctgagaccacaggcttgtgccaccatgcccagctaacttaaattttgattttttttattttttgctgggcgcagtggctcatgtctgtaatctcaactctttgggattacaaaaaaagaaaaaaattagctgggtgtggtggtgcatgcctgtagtctcagctactacttgggagcctgaggcagggggattgcttgagcctgggaggttgaggctgcagtgagctatgatcacatcactacacaccagcctgggtgacacagcaagaccctgcctctaaaaaagaaagtttatgacccttgtgtataatattccatgtgaataagtaactatttatctgttctattattgttcatgtagtcttcagcttttggctattgtgagtaaactattatgaacattccttgttcatgtcaattggtagatatatacactcattccaggcatgggattggtgggtcctgggcagcagtagacttggctttagcaaatgttgccaaatcattttccaaagcggctataccatttgagacttcctccagcagcatagaagagttctggttttgccaatacttgaaaaaaatttttttttcatttgtgtgtgtgcgtgtgagggtgatattgtattgcagattgagtttgcatttccctgatgactaatgaagttgagcagcttcccacgttagccttggccatctgggcatcctcttttgtgaagtgcctatttagctcttttagccacttggtaaaaaagtgattgtcattaagttgaaggagttctatatgtattataggtatgagtcttttgtcagacgtatgtctgtggcttttatttttttgtttgagatggagtcttgctctctcacccaggctggagtgcagtggcgcgatctcggctcactgcaacctccgcctcctgggttaagcaattctgcctcagcctcccaagtatctgggattactggtgcccactactgcgcctggctaacttttgtatttttagtagaggcagggtttcaccatgttggccaggatggtctcaagctcttgacatcatgatccacccgcctcgacctcccaaagtgctgtgattacaggcgtgagccaccgtgcccggctgtctgtggcttcctttttaatggcatcttttgatgaaccaaaactcattttcagctgggcatggtggctcacacttgttatcccagcactttggaaggccaaggtggaagggtcacttgaggccaggaggtcaagacctgcctgggcaacatagtaagaccctgtgtcgaccaaaaaaaaaaaaaaattagccaggtgtggtggtgcatacctatagccccagctactagctactctggaggctgaggtgagaggatcacttgagcccaggagtccaacattgcagtaagctgtgattgcaccactacactccaacctgggagacagaccctgtctcaaaaaaaaaaaaaaaaacccaaaaactcattttcatagagtccagcatattttcttttattaatgtgctttgtgtcctgccgaagaaacacttgcttgtccacaagtcacatagatgttctgtgttttatttcaaaagctttcctgactttgctttcgtatttagatatgtgatgtgtctgagttgtctttttttttttgagacagggtcttactctgtcacccaggtgggagcacagtggtgtgatcatggctcattgcagcctcaacctctttgggctcaggtgatccttccacctcagcctccagagtagctgggactacaggtgcctgccatcatgtccagctaatttttttttttgagagggagttttgcttttgttgcccaggctggagtgcaatggcgtgatctcagctcactgcaacctctgcctcccaggttcaagcgattctcttacctcagcctcccaagtagctgggattacaggcatgtgccaccatgcctggctgattttgtatttttagtagagaaggggtttcaccatgttggtcaggctggtcttgaactcctgacctcaggcgatccacccagcctaatttttgtatttttttttgcagagatgagtcttgtcatgttgctcaggctggccttgaattcctatactcaagcaaatccacctgcctcggcctcccagattgctgggattacaggtgtgagccactgcccagcctgttggtaattttgatggatatttgtaaactgagcatttaatgtgggaagaacaggcttcgtaccctctctcatctgtgtagggctccataacactttattgggtgctaactacatcctgggtgctttgctacacactgggcatcggtgggggtatggctgatgtaaatctcaaaatatatgatcaacagctggggtaaaggctctgaaagaaagggcctgacttctaaaagaccatggaacaaagacctgaaagggctaggggaggtcaggaaaggctccttgaaggaggtcatggttgagccacaatagggagaataattgtagattatccaagcaggagaactgggcgtgtagaataggactttccaagctaaggaaacagcacatgcagaagccttgcaatacatatctatggctgcattaacaacaagcctcaaaccccagtggctcagaacaacacccatgcattatatcctgtggtttctgtgggctgggaatttgggagcagctctgttaggcgtctctcatgaggttacagtcacatatcatctgtggttccacccatctgcaggtctgatgggggctggagtgcagctacttccaatatggctcactcacctggctgataaattggtttctcttcctatgggcctccctgcagggctgtttgagcatccttacaatatggcggctggtttcctggggaacacaaggtggtgctgcaatgccttttatgatctagccttggaagtcactcaccttctcttggccacataaactggccctgactatggaagaaggcttggcagggcatgaataccaagaggcaagagacattgagggccttcttggaggctggccaccacatgtcttaggcagaaagaggctggaactgtccatgatgtggctcaagtggagtgggttaataggtgggcagcaggagctgaaaggacaagacagggctgggcactgaggccttgtgggctgcagcggagaaggtagggttgggtttaccctaagagccctgagaagccattcaggggattcggtgaggtagggaggagctaacatgatcagatttgggttctgatgaacttcttccgtggtgaatggatggtgagggggccaagtagcagcccaggaatgaggctgggcaggcatccagcagaaaggtgagggtgacctgggccaggggcccaggaggggcagatcagctgactcaagggctctttgaaggaaaaacccacagggcttggcagcaagcctcttgtgatccccaacagagagctgtctgcctgctcaaaggccccacgaggagtaggcggctctacccagcacacgctatgggacttgggtttctgcagatctgattcctagcttaggcaagtcgagcctggagagagtgcttctgaggcccttccagggtaagggtggtaaagtgtccccattgttactgagtgctgctgttcctgcccagggcctctgatgggcctggcactgagcacggtgtctgggatgactcaccttcagccttctgactgaccgtagctcaggacacgtggccagctctccaccagccttcgtgctctgggctgcctggacctcggttagactggtctctgggcctctcctggcctccctggaatgtggcctgggaagcaaggagctatggcctggcagatctttccttttcatttacattttttctttttggtggggacagagtcttgttctgtcacccagggtggagtgcagtggcatgatcttggctcactgcaacctccacctcccaggttcaagtgattcttgtgcctcagcctcccgagtagctggaattacaggcgcctgctaccatgcccagctaatttttgtatttttagtagagatgaggtttcgccatggtggccagtctcggacacctgacctcaagtgattcacttgccttggcctcccagtgtgctgggattacaggcatgagccaccgtgcccggcctaaatttattttttgttttttagagatgtggacttactctgttgcccaggctagagtgcagtagcatgattatagctcactgcagcctcaaattcctgtccttaagcaatcctgccccagcttcccaaagcgctgggatcacaggcatgaaccaccatgcctggcctggcaagtctgtcttgtggcatattaggaaatgtgactttggaggctgagaggtggaggcagagtgaatgcccatgaaatctgtacctgttttcttttagaggctccacttccttctctcaggtaaggcatgagccactgtactcgacctttgtttggtctttcttttctcctttcattttttttttttttgagacagagtctcacactttcacccaggctggagtgcaatggtgcaatctcggctcactgcaagctccgcctcccgggttcacgccattctcccgcctcagcctcccgagtagctgggattacaggtgctcaccaccacgcccagctattttttttgtatttttagtagagacggggtttcactgtgttagccaggatagtctcgatctcctgacctcgtgatccgcccgccttggcctcccaaagtgctgggattacaggcgtgagccaccgcgccaggccttctcctttcttttcttttctctttcctttctctctctctttctctctctctcttcctctctttctctctcttcctctttttctctctttctctctctctttctttctctctctttttctctttctctctttctctctctgtctctttctctctttctctctctttttctctttctctctttctctctctttctctctctctctttctctctctctctttctctctctctgtctctctctctctttctcttgctttctctctctctgtgtgtctttctctctctctctttcttgatggagtctcactctgttgcccaggctggagtgcagtggcacaatctcagctcactacaacctcaaactcccgggttcaagcgattctcctgcctcagcctcccaagtagctgggattataggtgtgagccactgcacccagctaattttcatatttttagcatagatgaggtttaaccattttggccaggctggtctcaaattcttgacctcaagcgatccacctgcctcggcctcccaaagtgctgggattacaggtgtgagtcactgtgcctggccctttgtttgagatttctagtggtcaagtgaacaaagcaaatgacccaggaactggagacaaaactaggtttgtaacctgatctggtttgggtttctgtttgtgaaatggaaatgacactgttgttgggggaaaaacaactttacttttatttattttgagacagggtctcgctctgtcgcccaggttggagtgcagtgttgtgatcttggctcactgcaacctccgcctcccgggttcaagcaattctcctgtctcagcctcctgagtagctggaactacaggtgcatgacaccacacacggctaatttttgagggttttttttgagacagagtcttgttctgttgcccaggcttgagtgcaatggcacgatcttggctcactgcaacctccgcctcctgggttcaagcaattctcctgcctcagccttctgggtagccgggactacaggtgcacgccaccatgcccagctaatttttgtatttttagtagagacagggtttcaccatgttggccaggctggtctcgactcctgacctcaggtgatctgcctgccttggcctcccaaagtgctgggattacaggtgtgaaccatcgtgtctggcctaatttttgtgtttttagtagagatggagtttcacaatgttagccaggctggtctcgaactcctgacctcaggtgatctacctgacttggcctcgcaaagtgctgggattacaggtgtgagccaccatgcctggcctcatgtcctatatgttaattcaacctatatttgcattcctctgaagcttgtagtctgtggaggagtcagcacactaaggcctgaaggccaaatctggctggccatctgtttttataaataaagttttactggaacacagtcatacctactcatttacatattggtgatggctatttctgcactttgatgatggcaggtttgagtagttggggcatagaatgtgtggctcatgaaacccgaaatatttatctggttctttagagaaaatgtgtgccaatccctgctttgttcttgtgggacagacagattacgatagatagtaaataagaaaatctcaggctgggtgcagtggcttacacctgtaatcctagcactttgggaggcggaggcaggcagatcacctgagttcaggagttcgagaccagcctggccaacatggtgaaacccccgtctctactaaaaaaatacaaaaaattagccaggtggtgggtgcctggaatcccagccactcaggaagctgaggcaggataattgcctgaacctggaagatagaggttgcagtgagttgagattgcaccactgcactccagcctgggtgacagagcaagactctaagaaagaaaaaaaaaaaaaaaaagaatctttgacagtggtaaatgctacgaaaaaacagtaatagtaaagcatgactaggtgggtaaagggttgcttcccagtggtctggggaagcctctgttttgaggcaacgttgagctgtgtgaatcttccagaaggttgatttgggcagaagtggcagcaaaaacaaaggctttggggcacttaagaacttgagaggccaggggcagtggctcatacttgtaatcccagtactttgggaggctgaggcaggagaatcgcttgagcccaggagttcaagaccagcctgggcaacatagcaagaccctgtctctaccaaaacaaaacaaaactagtcagacgtggtgatgtgtacctgtagtcccagctacttgggaggctgaggcagggtgattgcttgagcccaggagttggaggctgcagtgagctgtgattgcaccacgacactccagcctgggcgacagagtgagactctgtctcaaaaacacaaaatgtaaataaagctaggccgggtgcagtggctcatgcctgtaatcctagtactttgggaggctgaggtgggcagatcacttgaggtcaggagttcgagactagcctggccaacatggtgaaactctgtctccactaaaaatacaaaaattaaccgggcgtggtggtgcgtgcctataatcccagctactcgggaggctgaggcaggagaatcactggaacccaggaggcagaggctgcagtgagctgagatgacaccactgcattccagcttggggaacaagagtgaaactccgtctcaaaaaaaaaaaaaaaaacaccccaaaactggtgctcagtgaagggaggaataagactcacccaaacaggaccctgtaaaaatagtaaaatgcttagattttatttcaagaacaatgggaagttctggaaggttttagtggtgaagaaggggatggcatgttccaatttatttttgtaaacatttctcagggccaggtgcagtggctcgtgcctgtaatcccagtgctttgggaggctaaggtaggaggattgcttgagcccaggagtttgaggctgtagtgagctatgatcatgtcactgtactccagcctgtggtgacagtgagaccctagctgggttgctgttgttgttgtttaaaaaaaggccggacacgtagctcacacctataatcccagtactttgggaggctgaggcgagtggatcacttgaggtcaggtgtttgagaccagcctggccaacatggtgaaaccccatctctactaaaaatacaaaaaattagctggacatggtggctcgttcctgtagtcccagctacttggaaggctaaggcaagagaattgcttgaacctgggagatggaggtagcagtgagccaagatcacaccattgcactccagcctgggcatcagagtgagactctatctcaaaaaaaaaaaaaaaaaaaaaaaaaaaggcctggtgcggtggcttacacctgtgatcccagcactttgagaggccgaggtgggtgtatcacaggtcaggagttggcctggctaacatggtgaagccttgtatttactaaaaatacaaaaattagccgggcatggtggcaagtgcctgtaattccagctacttgggaggctgaggcaggtgaactgcttgaatccgggaggcagaggttgcagtgggctgagattgtgccactgcactccagcctgggtgacagagtaagactctgtctctaaataaataaataaataaataaataaataaaataaagctttctagccgaagcgtttggaagggactctccagggcagctgggaggtcagggaggaggcttctagaatgaagtccaggtgagtgatgatggcagtttgggctggggtagcaggagcgtggataagtagaaagacttggcttatattttagaggaagaaccagcagagcttgctcatgttttgcatgtgaggcaaggagaggcatgaaaggagatgcctggattttttgcccagacaacttggaagcaaattccaacttgcagggtacccttaaggcaaatcgtttccatatctgagttttttccccatatctggaagatgggtcaagctatctcttcctgacaaggttgacaatgctgttacaaaggaaatctagcttagaggaagtctccaataaatgcagctggtctccttgcgttgaagtccctgagtaggaggtctgttcatctgtcccctcttcttatttcctcccctggctctaaggtgggttaggggccctgatcatgggttctgacaggtttatgggcctaaggttcactctggtgagactgtcttctgtcattccctttctgttcctaccctagtccagcttccttcaagtgctatccctttggtctcccatggcctggacaactggccatccctggggccactgttttgccctcaggttctggatgaaagacagctggattcatggtccgtactgctgagtgagtctatgatcttgggtaagtggcctctgagagtcctatgtgatgaatatttcaggccatggcaaacagggtcctgctacctaccttttatggttaaggattaaattaaataattcccaggctgggcaacatgggacaccctgtctctacaaaaatatataaaaaattagtcaagcctggtggtgtgtgcctgtgggtcccagctacttgggaggctgagatgggaggattgcttgagcttgggaggctgaggttacagtgaatggtgatcgtgccactgcattccagcctgggtgacagagtgagaccctgtctcaaaatagaagaaggctgggcacagtagctcaagcctgtaattatagcactttgggaggctgaggcaggaggatcacttgagttcaggagtttgagaccagcctgggcaacacggcaaaaccttgtctctacaaaaattaaccaggcgtgctggcgcatgcctgtattcctagccactcgggaggccgaggcaagataattgcttgagcctggaaggtggaggttgcagtgagccgagattgcacaccattccattccagcctgagagacgggagtgaaaccctgtctcaaaaaacaaaacaaaacaaaacagccaggcgcggtggctcacgcctgtaatcccagcaatttgggaggccgaggcaggcagatcacgaggtcaggagttagacgccagtctggccaacatagtgaaacctcgtctctactaaaaataaacaaaaaattagctgggcttggtggcgggcacctgtaatcccagctactcaggagactgaggcaggagaattgcgtgaacccgggaggcggagcttgcagtcagccgagatcgcaccactgcactccaacctgggctacagagcgagactcagtctcaaaaacaaaaaacaaacaacaaaaacaaaaagccccaggcacagtggctcatgcctgtaattgcttgagctcaagagttcaagaccagcctgggcaacatagtaagaccctgtctctattaaaaaaaaaaaattgaaaaagaaaaagttatctaaagagcctggtagactacgtaagtgctgattcaatattaatcgctgggaattttagtcttattattcagtcttcctgggtcctgccaagtgtgagcaattcatagcaaatggagggtctgtgtgaaggttcacttctggccaggatgacccttccctaccagctgttgggggaactgtgtgtgcatgtatatgtacatgcatataaacacattcacttgtgaattttataatacaaatattttgcaatacaaaaatgtaaaagtatgccctaattttaaactcctttctggtgcttcttgtggttgaaggtaaagtactttttagggctaggtgccgtggctcatgcctgtaatcccagccctttgggaggccgaggtgggcggatcacttgaggtcaggagttcaagacaagcctggccaacatggcaaaacactgtctctgctaaaaatacaaaaattagctgggcgtagtggcgcgtgcctgtaatcccagctactcgggaggctgagggaggagaattgctggaacccaggaggcagaggttacagtgaaccgagatcgtgccactgcactccagcttgggcaacagagtgagactctgtcaaaaaaaaaaaaaaaagaaaaaaaaaaagaaaaaaacttagccccatttactaatagtattttatttaccatcttatcaatttttcagtgtacagttcagtatatacacattgttgtgcaacagatctctggaatcattttgtaaaactgaaactcttgaacaataactccttttcctccttcccccccggccttggtaaccactattctactttctgtttctaagattttgattactttagatacttcataagtggaattgtatggtatttgtctttgtgtgattaggttctctcactgtgcgtaatgtcctcaaggttcatccatgttgtagcatataataagatattttttcttttcttttgttttttggagacggagtttcactcttgtcacccaggctggagtgcaatggtgtgatctcagctcactgcaacctccacctctcgggttcaagcagtactcttgccttaacctcctaagtagctgggattacaggtgcatgccaccatacccagctaattttgtatttttacttttatattatttatttatttatttatttatttattttgagatggagtttccctcttgttgcccaggctggagtacaatggtgtaatctcagcttacctcaacctccacctccagggttcaagcaattctcctgcctcagcctccggagtagctggggttataggcatgagccactatgtccagctaattttgtatttttagtagagatggggtttctccatgttggccaggctggtctcgaactcctgacctcaggtgatccacccgccttggcttcccaaagtgttgggattacaggtgtgagccactgcacctggccattttttcttttttaaagactgaattaggctgggtgcagtggcttacacctgtaatcccagcgctttgggaggccaaggccagcagatcgcttgagctcaggagtttgagaccagcctgggcaacatggagaaatcctgtctctacaaaaaatataaaaagttagcccattgtggtggcacatacctgtagtcccagctacttaagaagctgaggtaggagtatgccttgagcccaggagtttgacacctgcctgggcaacacagcaagaccccatctcttagcagcaatatgatctttttttttttttttcctgtctcttggctgttgtgactaatgctgcaatgaagacgggtatgcaaatatctctttaagatcctcttttcaattcttttgaatttatatccagaaatgggattgctggatcatatgataattctatttttaatttttttgaggaacttccatgctgtttctcatagcagctccaccattttattcgactctcaaccgtgcacaagggttccagtttctccacatcttcaccaacacttattatttgacagtggccattctaaggggtcttagggatatttcattgtggttttgatttgcatttccctgatgaccagtgatgttgagcatcttttcatatgcctgttttctatttgcatatctcctttgggaaagtatgtattcaaatcatttgctctttttgtttgtttttgagacggagtctcgctctgttgcccaggctggatggagtgcagtggtgcaatctcggcttgctgcaagctccacctcctgggttcacaccattctcctgcctcagcctcctgattagctgagactacaggcgactgccaccacgcctggctaattttttgtatttttagtagagatggggtttcaccacgttagccaggatggtctcgatctcctgaccgcgtgatccgcctgccgcctgggcctcccaaagtgctgggatcacaggtgtgagccaccgcacccagcccatttgctcatttgtaaatcgagtttttgttgttgcattgtagaaatttattattttttaaatttttatttgagacagagtttctctcttgttgcgcaggctggagcgcaatggtgcaatctcggctcactgcaacctccgcatcccaggttcaagtgattctcctgcctcagccttccgagtagctgggattacaggcacaccaccatgcccagcaaatttttatattttgaggagagacagggtttcaccatgttggccaggctggtctcaaactcttgacctcaggtgatccacccgccttggcctctcaaagtgctgggattacaggcgtgagccaccaagcctgacgggtagtaatttattttttatataatttttttcttctttttttggtagagacgaggttttgccatgttgctcaggctggtctaaaactaccgggctcaagtgatctgcctacccgggcctcccaaagtgcagggatcacaagcatgaaccaccatgcctggctgtaatttttcatatattccggatattaaccctttaccagataatacagtttgcaaactttttttttccccattctgtaggttgccttttcactgttggtcatttcttttgctgtatagctatccttaagtttgttgcaaacccatttgtctatcttttgtcttgttgcctgtgttttgctgtcatatccaagaaatcattgccaaatccattgtcatgaagcttttccccatgttttcttctaggagcactatagtttcaggtcttatgtttaatctttaataagttttgtgtttttgtatatggtgtaaggtaagggtccaacttcattcttttgtatgtggttatacagttttctcagcaccatttgttaaagacacaatctttcccccatgttctggtgctttaaaaaaaaaaaaaattctggctggttacggtggcttaggcctataatcccagcactttgggaggctgaggcaagtggactgcttgaggctaggagtcccagactagcctggccaacatggtgaaaccctgtctctaccaccgaagatacaaaaattagccaggcgtggtggagtacgcctgtaatcccagcctactagggaggctgaggcatgagaatcgcttgaacctgggaggcagaggttgcagtgagccaagatctcaccactgcacgccagccggggtgacagagtgaggcagggtcttaccctgtcgcccaggcaggagtccagtggcccaatcatggctcattgcagcctacactgccagggttcaagccatcctcccacctcagcctcccaagtagctaggattacaggtgtgtgtcaccatgcccagcaaatctttgtattttttgtagagatggggtatccctatgttgctcaggctggtcttgaactcctaacctcaagcgatcctcccacctgggcctctcaaagcactgggattacaggcgtgagccactgcgcctgacatggtgcttcttaatttattcttactttttatttttatttttttgagacaaggtcttgctctgtctcccaggctggaatgtagtggtacaatcatggctcactgcaacctctgcctctccggttcaagtgatcttcctgcctcaacctctggagtagtttggactatgggcacatgccacaacgactagctaatttttgtttttctttttttctttctttctttctttctttttttttttttttgagatgcagtttctctatgttacctaggctggtctaaaactcctgggctcaagcgatcctcccaccctggcctcccaaagtgctgggatgacaggcgtgagccacgtggtgcttaaaaaaggcaacaaaaaaccccccacacactgggtatagaagtggcatgggcctctatacactgtgagattcttggtactagctacaaattctgtgtatactcaagattttctagagtaggtgcaattaccccgttttacagatgaggacacagaggctgagccgtagtgacccacctaaggtcgtatagccagcaaatagatggaggttggattggaactgaggactttactcaagggctctcacaaacccttgggggcttctcgctgctttatccccatcacacctgaaagaatgaatgaatgaatgcctcgggcaccgtgcccacctcccagcaaaccgtggagcttggacgagcccactgctccgcgtggggggggtgtgtgcccgccttgcgcatgcgtgttccctgggcatggccggctccgttccatccttctgcacagggtatcgcctctctccgtttggtacatcccctcctcccccacgcccggactggggtggtagacgccgcctccgctcatcgcccctccccatcggtttccgcgcgaaaagccggggcgcctgcgctgccgccgccgcgtctgctgaagcctccgagatgccggcgcgtaccgccccagcccgggtgcccacactggccgtcccggccatctcgctgcccgacgatgtccgcaggcggtaggtaccatgggggggaacacggactcagggggacaggcagggcgctgggtggggggtcgcttcccctcggggtggccggtggccgctgctgacagacgggcgcgcatgcgtggggtggtgcggcgcgcagcggcagttggcgcgggcagggtggcacttccggtcgcgcgtgcccgggctgtttggcgccaaaatggaccgtggattcccccgtagctccctggtggctagaaactaggcggggtgggggttctcttttgatccccaaatacagcaagctttgggttcgtttccggggtccccttcttcaagcagcggtgggccgggctcttgactccagcttggtcgcacaggaagtgggcagcccgcggccaacggacacccctcgggcaccggcacttcggcctccacttccggtgtccggcccggtccccgggggcgcttctgtggtggggggtccctcagtgcctttcccccaaagctgtgcatctcgatggcggcctccagagacggatgtcacccgtcggggcctgggctgggctggctcatgttcgagagcccagccttccgtggggtactcattctgtcagttgggactgtgtggttcttgctccttgggaaagtccatgttttggcaggaactttaaccgtgcattgcagcgcagggcccgggaagcgtgaaggctgcatggttggtcttttcctgtaaatctgtgcctgtgagccgtgggcatcagggaagtcttgctaatgagaactggctacactttcgtgtgtgtttgtgtgtgttcatgggtgtgttctttagtgtgtgtctaatgttggaggccctggctttagcagttcatgcctctgtccaacttccgtgcacaaggatcagtgggagcactggtcccagccccttgttcatgaacgccatcagttcattcctgaaaacttgttcaagtgaggtgtgcgtgagtcagaggaaccctcaccaggcgtggacacttccagctggatttgtgaaagcactctttgccatttttctatttccttaggcttgttaactgacctggttgggccagtccggttctgatttgggtctttggggagggatggatatttggttcaacccccgctttttttcttttctttttttttttttttttgaaggctgggtgctgtggcttatgcctgtaatcccagcaatttgggaggcagtggcggacgaatctcttgagccaggagtttgagaccagcctgggcaacaaggcaaaaccccatgtctactaaaaatacaaaaattagcctggagtagtgccgtgcgcctgtggtcccaactactcaacagactaaggtgggagtatcacttgagccccagaggttgaggctgtgtgtagcgagctgagatcgcgctattgcactccagcctgggtgacagagtgagaccctgtctcaaaaaaagaaacactttttatttttagagatgggtctcactctgttgtccaggctggagtgccctggcacaattataactcactgtagccttgaactcctgggcttaagggatcctcagcctcctgagtagctgggaccacaggcgcccaccaccatgcccagatgactttcttagtttttgtttctgtatacgtgaggtttcaccatattgcccaggctggtcttaaacccctgccctcaagcagtcctcctgcctttgtgtcccaaaggttaggattacaagtatgagccatcatgccttaactactacttttttttgtttgtttgtttgagatggagtctcattctttcacccaggctggaatgcggtggcgtgatctcggctcactgcaacctccactttcagggttcaagtgattctcctgcctcagcctccgaagtagctgggattacaggtgcacaccaccacgcctggctaatttttgtgttttcagtggagtcggggtttctccatgttggtcaggctggtctcgaactcctgacctcgtgatccacctgcctcagcctcccaaagtgctgggattatagtcaaaccacttcttaaccagttagtgggtatatatcttcttgaatcatctgtgccagtattctcccaaaataaaacttgatttaaaatggaatggaagtgaaattatgaaagtgcctggcttcatgatgttacacccaattcagagaagtttgggcccggccttccttttttattccttttttttattttgtagagatggggtcttgccatgttgctcaggctggtcttcgattcctggcctcaagcgatcctcctgcctcagcttcccaaagtgttgggattacgggtgtgagccaccatgcccagcctgcccagccttcttgaaaacatgacctccactggctcaatgaccttaaacaagcgtatgccacctctatatccatagtctaggaataatgacatgggttttcagctggtattgggttaaatgagaatatgcatggcacataaaggttcacatgctagtggctaacttagcttgtttttttcagtttttttttaagagacaggatctcactgtgtcacccaggctggaatgcagtggcacagtcatggcttactgcagccttgaacccctgagctcaagcaatcctcctgccttagcctcctgagtggctggcacatgccaccatgcccaagttaatccttaaaaaattttttagaaggctgggcgcggtggctcacgtctgtaatcccagcactttgggaggctgagatgggcagatcacctgaggtcaggagtttgagaccagcctggccaacgtggcgaaaccccatctctactaaaaatacaaaaaaattagccaggtatggtggcaggtgcctgtaatcgcagctactcgggaggctgaggcaggataatcgcttgaacccaggagccgagattgcaccgttgcactccagcctgggcaacaggagcgaaactctgtctcagaaaaaaaaaaaatttttttttttagagatgagggtcttgctttgttgcccaggctggtcttgaactcctggactgaagcaatcctctgcgttggcctcccaaagggctgagattacaggcgtgagccacagggcccggccattattcctcaagagttcttaaaagtggatgctgcaggagttaactgccaggaagcaggaattcctgctaaggtctgctgcttgccaaagcctttcccttcaacaggggttgccagtaggccactggctcaggggctctgttggccctgtgcaaatattttgtttggctgaattggaataccattaggtgagccttacaatccaccactgaccctgggttccacatttctttacactcagattgcttcactgtaggcattggagttttggaggactgcttttgaccatgtggtagacattgaaattgtagtttatatgactcactccacagacttttttttttttttttacattttgaaaatatttcattaaatgaaaaataaacatggtatccaacagtacttgaaacttagatttttttttttttttaattgaggcagggtcttgctctgtggcccaggctagagtgcaatggtgtgatctcagctcactgcagcctccgcttcccgggtttaagcaattctcctgtctcagcttcccaagtagctgggattacaggtgtgcaccaccacgcccagctaatttttctttttccccctcgaggctgagccttgctctgtcgcccaggctggagtgcagtggccgtgatctcggctcactgcaacctccacctcccgagttcaagcaattctcctacttcagtgtcccaagtagctgggattacaggcgcccgccaccaagcccaggtaatttttgtatttttattagagactgggttttgccatgttgaccaggctggtttcaaactcttgatctcaagtgatccacttatcttagcctcccaaaatgctgggattacaggtatgatggtgtccagccggttcattgtttttattttattttattttattttattttattttattttattttattttattttatttttgagacagagtcttgtgctgtcgcccaggctggagtgcagtggtgcgaacttggctcactgcaacctccccctcccgggttcaagtgattctcctgccttagcctcctgagtagctgggattacaggcatgtgccaccacgcccggctaactttttcttatttttagtagaaaccggggtttaccgtgttggccaggctggtgttgaactcctggactcaagtgattcgcctgcctcagccccgcaaagtgttgggattacaggcatgagccaccgtgcctggccttttaattcattcttaagcctgctctcagacagctaggcttgcttctccacaataaagagtgttgaattttgtcggtgtatcccgcatggtgccattataacactgaaattagtgattatttcagggtctgttaccagagcgtgtctgccgtattcactgtatctgcagcttggagcacagtagactcaaatgcttgcatgccagctcttcccctgtacagcctggattaacactgatgctagcagactcctttgcacaactgattagctttcgtaactaccacagctttcttactgctagtcattctgtgatgggattaagctaggtcttgaactccttaactccagttccttggctactactgagtacatactgaataaggcaaaattgacttacctcttttgttttgctcccctgtggtagaattctaggggtacagactgctgggtttcaaatgccgccctacttgccagccgtatggctaagttattgaacttcttggtgccttggtgtttcatctataaaaagggggtggggctgcctgctcgacaggggtgttaggaagactgagcttatcagtacagatatgttgagagcagcacctgtccaaagcccttcatcactggttattacgtggtactgtgtgtacagtggggatgctgtgggtttggttccagaccactgcagtaaagtgaatctagcaagaaagtgagtcacacacacattttggtttcctagtatgtgtaaaagttattagactaggccgtgtgcagtggctcatgcctgtaatcccagcagtttgggaggccaaggcaggaggatcgcttgagccaaggagttcaagactagtctgggcaacataggaagaccccccccatctcaaaaaaaaaaataaagccaaaagtggtggcatgcacctggggtcccagctactcgggaggcagaggtgggaggatcacttgaacccatgaggtcgaggctgcagtttgccatgattgtgccactgcactccagcctgggtgaaggagtgagactctgtctcaaaaaaaaaaaaaaatcagctgggcgtggtggctcacgcctgtaatcccagcactttgggaggccaaggtgggcagatcacgaggtcaggagatcgagaccatcctggctaacacggtgaaaccccgtctctactaaaaatacaaaaaaaattagccaggcatggtggcacacgcctatagtcccagctccatgggaggctgaggcaggaaaattgcttgaacttgggaggcggaggttgcagtgagccgagatcatgccactgaactccagcctgggcaacagagcgagactccgtctcaaaaaaaaaaaaaaaatcatctgagtcttcagcgattcataatctttttgcaggtggagggtcttgcctcaaacttgccggcttaaaggacatacatttattaccttatgcaggtcagaaatctgatgtgggtttcaccggatgaaaatcaaggtgtaagcaggctgcattcctttctgggggctccaggggagaatctgtttcctattttggggtgttggcagaaggtggtagttgtaagactcaggtcccattttcttcactggttctaaactgagggtcgttgcccgcttctggaggcggtctcatccctcggctcatgcccccttcctctacctttaaagtcagcatttgcgggttgcatctctctctgacccgtcctccaacgttgcccgctctctgcagctagaaaggttctgtgttcttaagggctgaagtgattagattgggcccaccccagataatccaggataatctccccatctcaaggtctgtattcttagttacatctgcaaaatcgctttggccacgtaaagtaatatattcacaggttccagggattagggcatgaacatttttcggtgggtccactctttttgagatggagtctcgctctgtcacccaggctggagtgcagtggcatgatctcggctcactgcaacttctgcttcctgggttcaaatgattcttgtgcctcagcctcttgagtagctgggattacaggcgtgagccaccaagcccggttaatttttgtatttttagtagagatggggttttgccatgtttcccaggctggtcttgaactcctgagctcaggtgatctacccgcttcagcctcccaaagtgctgggattacaggcatgagccactgtgcccagtgggtctactcttttgcctactgcggatgccttcttgggcagccctgctccagctgccatgcagccggcctgaagaagctggtttgacagagacttgtaacttgtcttgcatggtccactatattttatttttagagacaaggtcttgctgtgtcacccaggctggaatgcagcggcacaattatagctcactgggctctagtgatcctcctgcctcagtctcctgagtagctaggaccacaggtgcatgctgccacaccaagctaattactattttttgtttaggcagggtctctgctgtgttgcccaggctggtcttgaattcctgagctctattgatcctcccgctttggcttcccgagtagctaggcctataggcacatgctagcacacccagctaattactaattactattttttgtagagacagggtctcgctgtgttgctcaggctggtcttgaattcctgggctctagtgatcctcccgcctcagcctcctgagtagctaagactataggtgcatgccaccacacccagctaattactattttttgtagagacaggttctcgctgtgttgcccaggctggtctcaaattcctgggctctagtgatccttccgcctcggcctcctgagcagctaggagtacaggtgcgtgctgtcatacccagctaattactgttttctcgtagagacagggtctcactgtattgcccaggctggtcacaaattcctgagctctagtgatcctcccgcctctgcctcccgagtagctaagactataggtgcatgctaccacagccagctaattattctttcttgtagagacagggtctcgctctgttgcccaggctagtctcgaattcctgggctcaagtgatcttcctgtcctggcttcccgagtgtagagattacaggcatgagccactgcgcccagccatggtctgctgtatttcagacgagcatttctggcgtagaggtgttagagtaatctaacattttataatttcttcctatccaatctatactagatacctctgctgggttagttttcttaacacggctctgatgtcattggttctacttccccttgactgctttggtgtttgttaactatggaacaagatggataagtgtgatgcttttgccctccctccgactgtccccagccacctctctggaggtgcctttccctccctgtatgtggccagtctcacgtttgtttcttgtgtgtttccagaaccagccacttctcaatggtgcccctgtttgagcagcttcctgtgcccggttcacacagagctgtcaacttcttcctgaggctgttataccctttctcttgggaatcccgcaggcgtgagctgtatttgccttattcctctgtggttgtttatatatatctttttttttttttttttttttgagacggagtttcactcttgttgtccaggctggagtgcaatgacgtgaccttggctcaccgcagcctccacctcccgggttcaagcgattctcctgcctcagcctcctgagtagctgggattataggcatgtgccaccatgcctggctgattctggatttttagtaaagacgaagtttcttcatgttggtcaggctggtctcgaactcctgacctcaggtgatccgcccgcctcggcctcccaaagtgctgggattacaggcatgagccaccgtgcctggccagctgtttatatatatcttatctcccctctgcagagggttcatctctgagggaagcactaggaaggaatcatagggtctcaggctgggcactaagccggcattctgagtcctctgctccgtccttcacggctgtctgcgaccctcctggcttcagcaggtgtgatcggtgtcatcacagttttaccagggaagggatgtcgctaaagttaccctcccgccagctcctcactgcctgcctgggatcccacctgccactctggggccaggcaggttaaatccagtttctactggatgaaacattctaggggttttgaaggtttctctattctctttgccaggctaaatatcccagttcatatgaggggttgaaagaaagcagacccattctgcaaggacaccttggtctccgcaatcagttgaaccttgtccttcacaccccaggcaacctcgggatcaggggactggctggcttggtcgaggtagaagccatgtccttccagttttctcagcggcgtgactgagaagtcaagtgtaacgctattaccacagcgtgtgtgcctctggctggaagtaccagtttctgtagccctcctgacttaaggctctccttggaccccagttgctacaagggtagaatgacttttgcctggaagaggttccttgtgctcctggaaatcctgatgtcaaagcatctttttttttttttttttttcttttgagacagggtcttgttctgttgcccaggctgaacaggctggattgcagtggtaccatcataactcactgcagcctcaacctcccaggctcaagcgatcctcccacctcagcctcccaagtagctggtaccacaggtgcacactaaccacacccagctaatttttacattttttttgtagagatgggatctctcagtgtagcccaggctggtctcaaactcctgacctcaagtgatcctcctgccttggcctcccaaggtgctgggattacaggcatgagccactgcgtcttgcctggccttgtagcaccttgagtccaagtgatacagtatatataatatacagtctatattaccactgtgtttgtagggtgctaagtgtctcttctacagagcagtcttttttttttttttttgagacagagttttgctctgttgcccaggctggagtgcagtggcatgatcttggctcactgcaagcttcacctcccgggttcacgccattcttctgcctcagcctcctgagtagctgggactacaggcgcccaccaccacgcctggctaattttttgtatttttagtagaggcggggtttcactgtgttagccaggatggtctccatctcctgacctcatgatccgcccacctcagccttccaaagtgctgggattacaggcgtgagccaccgtgcccggccctacagaatggtctttcctaaccacacagcaagtcaggaagtccctgcctagtgacacctacatctaatcacttagctctttgcctggagcagggcctggcattcagcaggtgctcatatgtgagcggcgctggtggggaatcagggcccgcatgacccagagcagctgttgtaagacatgggtccagcaaagcattcctcatgctgctgcctgcacttaccctctaggaaggccagcctggaggagccacttctgcccttcaggcaaatgataactgagcacctgtctgtgcgggtgctaggtggaaaggtccagagatggcagtggcctggtccctgcctggggcaccttctctccaccaacagacacttacccagctggttaggcctgttctcaccccgcagtctgtagttagcacccaggctgggaacaacagcctgtgttcctctccctttccttggtaccttctcttcttgttcctttatttttttctgaagagataggatcgccctatgttgtccaggctggactcgaactcctgcccacaagccatcctcccaccacagcctcctgagtagctggggttacaggcacgcagcaccgcggcactgcaccggctattgttcttttatttttttccctctttgtccctgaaagagtcaagctactaattgtcagtaatcaaatcagaccacgatttcccaggcaaactcctggcagttctacatttaggaatgactagctagagacatcctgaagaatgagttattcggggaggcgccagccacctcctctaacttcacctctatctgccctctgtgtgggtaccccttgcttccctggatgcttgactcccccatttcatcctcaaaatgccaccaccccccaccaggcctttaggaacatcagctggctgttccccacagtgtcctgtggccctgggctactcattctgacactggccatactgtggcacaccttgttatgggctgttgtcagacccaactggagaaagaccagctgtaggtcatttcccttacgggagtgccccaactatatgacctgccccctctttcctggtatctttttgagtcagggtctcactctgtctcctagattggagtgcagtgatgcaatcacggctcactgtggcctcgacctcccaggctcaggtgatcttccttcctcagcctcccaagtaactgggaccacaagcacatgccaccaaacccagttatttttattttattttattttattttattttgagacagagtttcactcttgttgcccaggctagagtgcaatggtgtgaccagctcactgcaacctctgcctcccgggttcaagtgattctcctgcctcagcctcccaagttgctgggattacagccacccaccaccacgcctggctaatttttgtatttttagtagagatggggtttcgccatgttggccaggctggtctcaaacccttgacctcaggtaatccacccaccttggccctcaggtaatccacccaactgctgctgtatgttgggattccaggcatcagccaccacgcccagccactaatttttgtatttttgtagagatggagtttcgccatgtttcccaggctggtctgaacgcctgggctcaagtgatccgctcgccttggcctcccaaagtgctgggattataagcgtgagccaccatgcctggtctctggtaccttttaaaatatacaggctgggcatgatggctcatgcctgtaatcccagcactttgggaggctgaggcaggtggatcgcctgaggtcgggagttcgaaaccagcctgaccaacacggagaaaccctgtctctgctaaaaatataaaattagctgggtgtggtggtgcatgcctgtaatcccagctactcgggaggctgagccaggagaatcgcttgaacctgggagtcggaggttgagctgagatcacaccattgcactccagcctgggcaacaagagcaaaaccctatctcaaaaaaaaaaaatatatatatatatacacacgtatatatacgtgtatatatatatacacacacatatgtatacatatatacgtatgtatacacatatatacgtatatatacacatatatatgtatatatacacacatatacgtgtatatatatacgtgtatatatatatgcatgccagacatggtgactcatgcctgtaatcctagcacttcaggagactgaggcaggcggatcacttgaggtcaggaatctaagaccagcttaaccaacatggtgaaaccctgtctctactcaaaatacaaaaaattaacgaggctggtggcacctataatcccagctacttgggaggctgaggtgagagaatcacttgaacccagaaggtgagggttgcagtgagctgagatcgcaccactgcactccagcctgggcaacagagcgagactccatgtctgtctgtctgtctatctatctgtataatgtatatgtatgtatgtatatatgtgtgtgtatatatatacacatatatacatacatatatacacacatactctgttacagagctgctgtgtgtgtgtgtatatatatatacacatatgtatatatacacatatacacatatatatgtatatatatacacacatatatatacacatatatatgtatatatatacacacatatatatacacatatatatgtatatatatacacacatatatatacacatatatatgtatatatatacacatatatatgtatatatatacacacacacacacatacacataattgtgttacagagctgctatgtaatctcacaatcatcagaaaaatgacccccaaaaggggaaccttgttcagatcagatgacttcttagcattaggcattccagtaggacactctagactcttgcggggagacaaaagccagcttagttttttctaacactcatatgttaaacttgtttgtgtccaaaacttctttagaactgtgatattcttacaggcaaatgaagttgcttaacaagtgtttgtattttctcccctatttcttcctcccaggctcaaagatttggaaagagacagcttaacagaaaaggtaatctccaccttaaaatttttcttattaccaaatctgactgacacactttgtggctcataaaaagaaatttgttttctttaaatggattttgcattttttcccatggagtttcaaagataatttggatattcttgttaaatgtcagcactaatttgctgctaatagttgggtggtggtggtgtttttttttgttgttgtttttgttttttgagacagagtctcactctgtcacccaggctagagtgcaatggcatgatctcggctcactgtgacctctgcctcccggattcaagctgttctcctgcctcagcctcccaagtagctgggactacaggcacgcaccaccatgcccagctaatttttatattattagtagagatggggtttcaccatgttggccagactggtcttgaacgcctgctcgtgatctgcccaccttggcctcccaaagtgctggaattacaggcgtgagccaccatgcctggcccaggttttttttttttttaaccaatctcagttcctaaacaatctactctggattgtaacttgtcctggtaacactgttttattgtgtttttgttattgttttgagatagggtctcattctgtagcccaggctggagtgcagtggcacaattttggctcactgcaaccttcgcctcccaggctcaagtgattttcccactcagcctcctgagtagctctaactacaggctcaagccaccatgcccagctaatttttaaatattttttgtaaagatgggattttgtcatgttgccccaggctggtcttgaactctggggctcaaagcaatccacttgcctcggcctcccaaagtgctgggattataggtgtgagccactgtgcctggccgacactttacagaagcacagtattattcttataaaccatgatatgtctccatctcacctccagctttcccatttttcaccactttggagacaggagtgaagtgatcctaatggaaattccctgaacacatttcatgactgtttagtgttttgactgagacagcattgcctgccattcactcattgtgatgtgatcaggcagctcaataatttgtgtattagtccactagtgaatagcttgggaatgtgggtactgctaaacctatatccttcccttaggaatgtgtgaaggagaaattgaatctcttgcacgaatttctgcaaacagaaataaagaatcagttatgtgacttggaaaccaaattacgtaaagaagaattatccgaggtaagtcagttctcagcatcctagcctctagaaaaatgtctcctcctagtaacttgtctgtgaccagggaggcagcaagatccccagctgtcctcattgcctgatgatgatgatgatgatgatgatgatgaaggaaacacatgtgtttccttgttctctgacacttgtttacattcactgctacttaaattatcctttcctgctgtaggagggctacctggctaaagtcaaatcccttttaaataaagatttgtccttggagaacggtgctcatgcttacaaccgggaagtgaatggacgtctagaaaacgggaaccaagcaagaagtgaagcccgtagagtgggaatggcagatgccaacagcccccccaaacccctttccaaacctcgcacgcccaggaggagcaagtccgatggagaggctaagcgtaagagcagatgattccttttatttttaattgtttttgagatggagtctcactgtgttgcccagtctggagcacagtggtgtaacctcggctcactgtaacctctgcctcccaggttcaagagaccctcctgcctcagcctcccaagtaactgggattagaggtatgtgccaccacacccagctaatttttgtatttagtagagatggggtcttaccatgttggccaggctggtccagaactcctgacctcaagtaatccacctgccttggcctcccaaagtgctgggattacaggcataagccaccacgcctggccctgcttaactctctttctttctttcttttttttttttttttaatgacagagtcttgctctatcgcccaggatagagtgcaatggcgcgatctcggctcactgcaacctccgcctcccagtttcaagcgattctcctgcctcagcctcccaagtagctagtattacaggcgggcaccaccatgcccagctaatctttgtatttttagtagagatggggtttcaccatgttggccagactggtctggaactcctgacctcaagtggtcctcccgccttagcttcccaaagtgctgggattacaggtgtgagccaccgtgcccggccccacttggctcattttcaacctagaagcaggctttggtagcatataagggggcagttaaacttggaccccaggtcagtgtgtccaggttcctttcttttgtacgcatcctcactagtaattactggacacttcctaactagcttagatgtgtacgtctttaaattttgtcttagctgggggtgtggtggctcatgcctgtaatccctgcactttgggtggccaaggtggctggatcacctgaggtcaggagttcgagaccaccctgcccaacatggcaaaaccctgtctctactgaaaatacaaaaaatttgccagacgtggtggcgggtgtctgtaattccagcctgggcaacaagagtaagacactgtctcaaaaaaaaaaaaatgttgttttcgaccaagttgcagcattagatcccaaaacatggctatgagtgtgacaggttgcagatgacaagtgctacaaatacttactccttctaagaccttatatgcctggttaaacatttattctcagactcttgcccagttttaggcaaaggaactctaatggaaattttttttttttttttttttttttgagatggagtcttgttgtctcccaggctgaagtgcagtggcgcaatctcggctcactgcaagctctgcctcccaggttcatgccattcttttgcctcagcctcccgagtagctgggactacaggcgcccgccaccatgcccggctaattttttttgtattttttagtagagacgggatttcaccatattagccaggatgatttcgatctcctgacctcatgatccgcccgcctcggcctcccaaagtgctgggattacaggcgtgagccaccgcgcccagcctcttctggaattttttaaaaaacaaacaataggatagtacttgagctcaggtctggttctcagctacttcatttttattttgttttattttattattatttttgagagagaatctcgctctgttgcccagtggcacgatctcggctcactgcaagctccacctcccaggttcatgccattctcctgcctcagcctcccaagtagctgggactacaggcgcacaccacgcctggataattttttgtatttttagtaggatggggtttcactgtgttagccaggatggtctcaatatcccgacctcgtgatccacctgcctcggcctcccaaagtgttgggattacaggcgtgagccaccatgcccagccttattttattttcttttaatttttattttattttttgagatggaggcatgcgccaccacgcccagttaatttttgtatttttagtagagacagggtttcaccatgttggccaaggtggtctcgaactcctgacctcaggtgatccacctgccttggcctcccaaagtgctgagattacaggtgtaagccacctcacctggccctttattttatttttttttgagatggagtcttactctgttgccaggctggagtgcagtggcacaatctcggctcaccacaacctccacctcctgggttcaagtgatttttttgcctcagcctcctgaggagctgggactacaggcacgtgccaccacgcccagctaatttttgtatttttaatagagacggggtttcaccatgttggccaggatggtctcgatctcttgaccttgtgatctgcccgtcttgcctcccaaagtgctgggattacaggcatgagccactgcacctggcctatttcttttttttctttctttcttttcttttttttttttttttaagagacagggtcttgctctgtcgccaaggctgtgtacagtggcgcgatcttggctcacggcaacccccatctcctgggttcaagtgattcttgtgcctcagcctcttgaatagctgggattataggtgtgtaccaccacgcccagctaatttttttgtatttttagtagagatgggggtttgccatgttgtccaggctgttctcgaatacctgagcttggattattttcccacctcagcctctcaaagttttggtattatagatgtgagccactgtgcctggcccacttcatcttttgatattttaacaagaggctctgtttcctggccttctttagcatttggcccctcaaataactgacatgatatggctcaacaaactggcaggtgctaactgccaacattagcaagctggttgttgactagaataaaaatgcaaagatgcttagtccttagaacctggcttcctgcaatagcttagtaatgttgaactgcattattgctgtgggctttctattgatagtggcttttttttttctttttaatgctttttcttctttaaacagctgaaccttcacctagccccaggattacaaggaaaagcaccaggcaaaccaccatcacatctcattttgcaaaggggtcagtatacgataaattggcggctgccttttttaggggccggctgttttgggatggaattggtagggcgtcacgtggcaattctgtcttccgtgttgtataggttttcatcttgcggttctgttctctagacatttatgaatagcatttctgaaaacgttacctgtttttactcatataggaaagttcaaatgtacacaaaatcatgagttttgtataatgaaccctgaaatacccatcatctagtgccaacagttatcaagttacagccgttgtttcatctatacccccatattgtgtgtccctgccccacctggattactttgtccatatatagtttagtatatttctagacaatgagatctcttttgacaactttttaaaaaattactcatattttacaataattgctaacatttagcagtcttttttttttgagacagcatctggctgtcacccaggctggagtgcagtggcacgatctcggctcactgcaacctctgcctcccgggttcaagtgattctcctgcctcagcctcctgagtatctgggattacaggcgcgcgccgccacgcctggctaatttttgtatttttagtagaggtggggtttcactatgttggccagactggtctcaaactcctgacctcaggtgatctgcccacttcaacctcccaaagtgctggggttacaggcgtgagccaccgtgcccagccaaaacagtctttttaacaaaccaaagtataatactgttctgatgtctttataaaaatgtaagaattccctgctatcagatatatagttggtatttacgtttttccctttgtggtgaacaagccgtgtggcaaacagacagcgtttggttgacttgttccttaaagttatagattccttccctcctattttacttaaaattcctcacagattttaaattccttcatctcttatcttcttgccatttacttgaagaaattcatttgtccaatagttcctagattgggccgattgcatccttgtggtgttccacaggtccccttgccttctgttccctataaactgataggattacaggactgatcagattccagctttttattttttgagactgacttttttttttttttgatacggagtcttgctctgtagcccaggctggagtgcagtggcatgatttcggctcactgcaacctccacctcccaggttcaagcgattctcctgcctcagcctcctgagtagctgggattacaagtgcccgccacaacccctggctaatgtttgtatttttggtagagacagggttttaccacgttggccaggctggtctgaaactcctgaactcaggtgatcctcatgccttggactcccaaagtgctgggattataggcgtgagccactgcacctggtctgaggctgactttatggatggtgctgtgtccctccatcagaaaggtgtgtgaggtctcttctctgacactagcagctgttgatcggtgtttagacccgtgatttcttaggacttacaagatggcaagacaacattctaaacccgtcattcagagaaacattaaacttgaagcctctttcaacatcctggtgaatgagggtccacttcaggccagctggaggcctagggtcttgttccactaatggttggcctcactgtgtgtgacagccctgccaaacggaaacctcaggaagagtctgaaagagccaaatcggatgagtccatcaaggaagaagacaaagaccaggtagggccagtgctttcatttcctgactctaccttaacttggtgtatttgatgattgtgacttcatatgtgttctgtccaagtaaataaaaaccctgtctagggctctatttagggctctccagagagacaggaccaatagaatgtatatgtgtgtatacagctataggtacatatatacatatgcacgtgtatgtatatatatataattcgtttgatttttttcttgtttttatgggtacatagttggaatatatatttatggggagagactttattttgagaaattggctcacaagattgtgaaagccagcaaatttgaaatctgcagggcaggctggaggtccaggggagaaatgatgtcatagctcaagtggcaaggcagtcttacatacatacatgttatatatatgtgtgtgtgtgtgtgtgtgtgtgtgtgtgtgtgtgtgtgtgtgtatgtatgtatgtatttagacttctttttttgagacttttttttttttttgagacagggttttgctcttgtggcacaatcttggttcattgcaacctcagcctcccgggttcaagtgattctcctgcttcagcctcccgagtagctgggactacaggcgcgtgccaccacgcccggctaatttttgtattttagtagggatggggtttcaccatgttgcccaggctggtctcccatgttgcccaggctggtctcgaactccttgagcttaggcaatctgcccgcctcagcctcccaaagtgccaggattataggtgtgagccaccgtgcccagccaatttgttttttttgttgttgttgttgttgtttttaattttttttagatggagtctcactctttcactcaggatggagtgcagtggcacgatctcagctcactgcaacctccgcctcctgggttcaagtgactctccatcctcagcttcctgagtagctgagactacaaggcatgtgccatcatacccagctaatttttatatttttagtagagatgggatttcactgtgttggccaggctggtcttgaactcctgacctcaagtgatccacccgcttcagcctcccaaagtgttgggattacagttctgtcttttttcttttcttttcttttctttgagacaggtctcattctgtcaccaggctggggtgcagtggcgcagtcatggctcactgcagcctcaacctcctgggcttaagcgatcctcccacctcaccctcccaagcagctgggactacaggcgcatgccaatgagctaagttttgtattttttgtagagatgaggtttcccatgttgcccagactggtctctaacaaataaattaaacaaattcctgacacaagatggagaatctttgattcttttacatagtatcaccaggcgatctctagtgatgggcagtgtgcctgcctacttctcggacctgcttttgttttggggttggtggggattaataccagagtaagagtttctcagatcttctccccaaaacccaggccccttcttttcccactcttgctctaaccatgtcaaatgtgttaatatttcaactcacacttttggtgttgaccttcccttgaaaccagtattctaatcttttttgttcttccttccctccacacaggatgagaagagacgtagagttacatccagagaacggtaagaatagttactatacctttctttttgttctacgagttgtgtaatcttgatcacaaaactttttcagaaagtttaaggccgggcacggtgactcatgcctgtaatctcagcactttgggaggccaagatgggcgaatcacttgaggtcaggagttcaagaccagcctgaccaacatggcgaaaccccgtctctactaaaaattcaaaacttagctgggcgtggtggaatgtgcctttaatcccagctactcaggaggctgagtcagaaggattgcttgaacccaggaggtagaggctgcagtgatctgagatcattccactgcactccagcctgagtgacagagcaagactccatctcaaaaaaaaaaaaaaaagtttaaaaagaagaactttatcgtgtgtttccatggcaaccatatcgttatgaagtatctgaatttggggtcagatcattagcgtttagattcaagtagaattctcctgttttcttggaaggaatgaatcgagggcagctggcattcgaaccaggtgtaaaagtcatatcaaatactaaatgttaaatgagtatgtgtctgtcagggctcctgagataatgaagattgcttttgggggaaagaggagctttatgaaaactgcttctttggggaagctcctggcactcacacttggggtctgtgttattttgcttgacagagttgctagaccgcttcctgcagaagaacctgaaagagcaaaatcaggaacgcgcactgaaaaggaagaagaaagagatgaaaaagtaaagctctatcacctctaagtttgttaattctcccttaatcctatatggtcccaatttccttccaggacgtggggacagggggtggggaacgtggaagaacaagatgggttttgctagtgatcgaaatgcctttgactttggctaaacacctttcaaacttgttttctcgtggtctctacatgaaaggtgggttatgagattgtgatagtgaggacccttgctacttatttgtgatattttattttattttatttatttatttatttttgagacagtctcccactctgtcacccaggctggaatgagtacagaggtgcgatctcggctcgttgcaacctctgcctcccaggttcaagtgattctcatgtcccagcctcttgagtcactgggactacaggcgcccgccgccatactcggctaattttagtggagatggggtttcactgtgttggccaggctgatcttgaactcctgacctcgagtgatccacctgcctcagccaccaaagtgctaggattacaggcatgagccaccacaccggccaacctatgccacttaatcaactagtcacagagcttcagtttgtcacctcgccttcttgcttctctggctgatgagatctttgtgaagatctaattttcagacgtacgaggtgctaaaactttttttttttttttttttgagatggagtcttgctctgttgcccaggctggaatgcagtggtgtgatcttgtctcaccgcaacctctgcctcccgggttcttgtgcctcagcctcctgagtagctgggattacaggtgcctgccaccacagccagataattttgtatttttagtaaagatggggtttctccatgttgatcaggctggtcttgaactcccaatctcaggtgatccacctgccttggcctcccaaagtgctgggattataggcgtgagccaccacacccggcctaaaatacttttcaaacttatttttttgtagttctctacatggaaggtgggttatgaggttgtcatagcgaggatctttgctacttatttgtggtatttatttaatttaatttaattttttttttttgagacagtctcccgctctatcacccaggctggagtgcagaggtgcgatcttggctcattgcaacctccgcctcccaggttcaagtaattctcatgtcccagcctcccaactagctggggctacaggcgcctgccaccacacctggctaatttttgtgtttttatttatttatttatttatttatttatttatttatttatttgagacggagtctcgctctgtcgcccaggctgaagtgcagtggcttgatctccgctcactgcaagctccacctcctgggttcacaccattctcctgcctcagcctcccgagtagctgggaccacaggcgcctgccaccatgcccggctaattttttgtatttttagtagagaaggggtttcaccacgttagccaggatggtcttgatctcctgacctcgtgatcctcccatctcggcctcccaaagtgctgggattacaggcgtgaaccaccgcgcctagccaatttttgtgtttttagtagagacggggtttcaccatgttggccaggctggtctcaaactgctgacctcgaacggtccacctgcctcagccatcaaagtgctgggattacaggcacgagccaccacaccggccaacctatgctacttaaatcaactagtcagtttcagtctgtcacctcagccaccttctctggctgacgagatcttggtgaagatctaattttcagatgtaggaggtgctaaaattctttagcatcatccagacataaagttgtattctcctctctgaagtcagaccagctctttccccagtccctcatcttctttgtaaaatctgagtctaaggttctatgcaatacagatgctgggtcctgtttttttttctgtttgttttgttttttaataagatgactgagggccgggtgtggtggctcatgcctgtaattctagcattttgggaggccgaggtgggtggatcgcttgagctcaggagttcagggccagcctgggcaatgtagtgagaccccgtctctgcaaaaaatacaaaaatcagctggatgtggtggagcttacaacaggcagtgtgcctgtagttccagcttctcaggaggctgaggcaggaagatcacttgagctcaggaggctgaggatacagtgagccatgattgtcccactgcaatccagcctggatgataaagtgagactggtcttaaaacaaacaaacaaacaaacaaaacaaacaaacaaaaaaaaaccaagatgactagaagtttcttaccaacttaggtaggtgacgagtgccttacttgggagcttagtaaaggatggctgtccattggggaatcaactaaaatgtacagtaattttttttgttttttgagacggagttttgttctacttgcccaggctgcagtgcagtggtgtgatctcggctcactgcaacctctgtctcctgcgttcaaacaaattctcctgccttagcctcccgagtagctgggattacaggcttccgctaccacgcccagctaattaactttttgtacttttagtagagacaggggttcaccatgttggctaggctggtctcgaactcctgaccccaggtgatccacccgcctcggcctcccaaagtgctgggattacaggcgtgagccactgcatccggccgctgtacaataatttttgaaccattcagtacttgaggttggtctttcttgctttttttctttttttttaattgagatggaattttgctctttcgctcaggctggagtgaagtggcgcaattttagctcactacaacctccaccccctgggttccagcgattctcctgcctcagcctctccagtagctgggattataggcgcccgccaccccgtccgcctaatttttgtatttttagtagagacagggttttgccgtgttggccaggttggtctggaactcctgacctcaggtgatccacccacctcagcctcccaaagtccaggattacaggggtgaaacaccacgcccggccgaggctggtctttcattgatcacctttcctaccctgatcctagttaaattagacagcagttgcagtcagtgcatacctcgaatttgtttcagaacaagcagtcctaacgggctagttcaaccaccaccttccaaagaaggaacctggggcaagatcgaaatccctcacctgtaagcatactctcaggatttcttcttgtagtaggaagatttgctaagcgttttttttatttttatttgagacggagtctcactctgtcgcccaggctggagtgcagtggtgcgatctccgctcacttcaagctccgcttcccaggttcacgccattctcctgcctcagcctcccgagtaactgggactacaggcacctgctaccacgccaggctaatttttttttgtatttttagtagagacggggttatttatttatttacttatttattttttgagacggaatctcgctcttgtcgcccaggctggagtgcagtggcacgatctcggttcactgcaagctctgcctcccaagttcacgccattctcctgcctccacctccccagtagctgggactgcaggcgcccgccaacacgcccggctaatttttttttttttttttttttgtatttttagtagagacggggtttcactgtgttagccaggatggtctcgatctcctgacctcgtgatctgcccatctcggcctcccaaagtgctgggattacagtagtgagccactgtgcccagctgcaaagtgtttcttaatattcctgacctttgcgatctcttcacagactaccacatgtggagtttctgagggttggatttgacttgcctgattctctggcaagatgtgtctggttctttaaatgcagcctcaggctgggcatggtggctcacgcctgtaatgccagcactttgggagaccaaggcagacggatcacttgaattcaagaccagtctggccaacgtgatgaaaccctgtctctactaaaaatacaaaaattagctggtcatggtggtgtgtgcctgtaaactcagctacttgggaggctgaagcaggagaattgtttgaacctgggagttggaggttacagtgagccaagagtacaccactgcactccagcctgggtaacagagtgagactccgtctcataaataaataaatgaataaatgcagtctcagaggatgtgttgttgtgtctcagttggagctacttttcagattgggccatcctatttcattcctttaatatcggaagtagagatttataataaagttactttctgtgaggctatcaacacttaactgatgacaagtagtagaggaattttgaagtaaaaatactgaaactgcagtgttgtagtatctcctttctaagagtttcatttcctgtctacttaccccactgaagaagaaaagaaaattgctgtcttgaacacctggcacaagtgatcctctcacctcagccttccaaagttctgagattacaggcatgagctactgtgcccactggtagacagtctttactcccaccagtggactctagaatcagttcaggtgttttatttccataggacactttaatagaaagatccaaaccaaatggaaaaaattaacttgtcttttttccctgcaacttaggaagaaaagagactccgaagtcaaaccaaagaaccgtaagtgcagcgaacctgcctttgtgctttgttgtgaaactgaattgctaacataagtatcttggtaaaataacgggttggtgtggaacagtgggcactaatcatatgtctcttatgtgggcaagttctgcttgtgaaaggtgagaccaccctgaagtgaaggctgaagttaacttttttaactttaatttaatttaatttaattttgagacggagtcttgctgtgtcacccaggctggagtacagtggtgcaatcctggctcactgcaacccccacctcccgggctcaagcaattctcctgtctcagcctcccgagtagctgggagtatgggcctgtaccaccatgcccaactaattttcgtatttttagtagagacagggtttcaccatattggtcaggctggtcttgaactcctgacctccgtgacccacctgcctcgcctcccaaagtgctgggattacggggtgaagttagcttcgatttctcctcccaaccccagggcccttctcagggttggtcacacagctgtgtgtcatgtggtagtgaaaggccatcaggtgagcagctgtgtcacttacctttcactgtgaaaatgctgtgtgacaaatggctccaaggccagaggcctagaacagtgagcatttttcaagctcacttgtctacagcttggtggctgtcggctgatcttgaggtggcttagtggggatggctgggctgacccagctctgcctccgcaatttggccaggctgggtacacgcttgtggctgtgccagaccacaagggcagaaatgaaaagtagcactttttcaaacctctgttgaaaaaagttgggccaggtgcagtagctcgcgcctataatctcagcactttgagaggctgaggcgggtggatcacttgaggtcaggagttcaagatcagcctggccaacatggtgaaaccccgtctctaataaaaatacaaaaaattagctgggtgtggtggtgcatgcctgtggtcccagctacttgggagactgaggcaggagaatcacttgaacccgggaggcggtggttgcagtgagctgagatcacatcactgcactccagggtaggtgatagagcgagactctgcctcaagaaaaaaaaaaaggaaaaaagaaaaagaaaacaattagctgaagcaaattttgagactgagcctgaatccagaggtgggcagaataccccacctgagcggcagggccctggcagagtgggaggtgaagactcggacccctgtgtgcagtcagctgtccacggtcactgactgacgctgatcgcacatcagcccagctctcaacctaggcaggccagacatgggtcttcctccaccgcaggctggattcccatcaggccacggaaatcatgacatccctttgtctgctttttgtattaaaccactggcatctctgaccaacattcagatggggtattgatggtgttcctgaagaccttgagtcctggagtagtaaatgccctctgggcttcctgcagtgaagacaggagaggcagcctgtcctctgaacctggggaggagcttgtgtcagcccttaggagctgttggccccggtgcagggccccccccgagctgaccagcctgtgtgtgtgttgtcttctgtgacagaacacccaaacagaaactgaaggaggagccggacagagaagccagggcaggcgtgcaggctgacgaggacgaagatggagacgagaaagtaaaggcgggttatttgtttctgaactccccctcattcttctgctcctgtttgcttgggcgggagtgcgcagggtggggctctgggttcaagtccagccccatccacccaggctccatggggccatccagactctgggaaggtcaccttttcctccttttgggggccgggatttgtggctggatgcctgtgctggatgcctagtgaacactcagttgtcctctgtctttcacactagagagggtctgtgtgagcctgggctgagatgtgcatgaagcatcctctcctgaggcctcactgctgagaagccttgacacacagtcagctctgacgcacatgtcagccacacctttgattctttcccagcctcctacgttctgagtctagctctccccctgccagcctccccagtctagtttagccaagtgcatgcgtttaaaccctaaagcatcatgtggaacactgctgggcctctgtcaaagtacaggatggccaggttgcctgcaggccctcctgcatgaactctagggaagggagctaggtcatcggtggggggcggcgggtccctatctaagagatgccgaagaccccaagagggtctctgggtttctgcctgttacaatctagagagtggagcccaaggtcacgggcttgctgggtgacaagcaccctcacttctgacccggtcgccagtgggcttccatccccaccccatagcaggggagcctttggtattgcctccagcctcccatccctcacctcttctgtccaaatgttggtcaccttactgtcagcactccttcagactggagtagtcacattactccctgccctctattggaaactggcaggggcctgagctcacactgccagcacttttgcctcatggtagaaagggatgtgagggctgggcatggtggctcactactgtaatcccagcactttgggaggctgaggtgggtggatcacttgaggtcaggagtctgagaccagccaggccaacatggtgaaatccggtgtcacctaaaaatataaaaattagccgggcatggtggcatgtgcctctaatcccagctacttgggaggctgaggtaggagaatcacttgaacccaggaggcagaggttgcagtgagccaagattatgccgctgcactccagcctgggcaacagagcgagactctgtctcaaaaaaataatatggtaataataataaaagcagtgcccagagaacaaggattgttggctgttccaccccagggggccccttgcacaggaggtgccatctctgcctcccaaagctctaagagccactgtcccccatcccaagagacagggtcttgctttgtcacccaggctggaatgcagtggtgcaatcacggttcactgtagcctcgacctcccaggctcaggtgatcttcccgcctcagcctcccaagtagctgggactataggcaggcaccaccatgtccagctaatttttttttttaattttttgtagagatagggttttgccatgttgcccaggctggtctcagtcaaactcctagactcgagttatccacctatctcagtcccccaaagtggcggagtggtaggattacaggcatgaaccgttgcattcagtctgtttggtttgttcttttttttttttttttttttttttttttttttttttttttttttaagatagtctctctctgtcatgcaatggtgtgatctcgagtcactgcaacctccacctcccaggttcaagtgattctcctgtctcagcctcctgagtagctgggattacaggcgtgtgctactatgcctagctaatttttatatttttagtagagacagggattcaccatgttggccaggctggtctctaactcctgatctcaaacaatccacctgcctcagcctcccaaggtgctgggattatgtgtgtgaggcaccacgcccggcgtgtttgtttgtttgtttgtttgtttgtttttgagacaggatcttgctctgtttcccaggctagagtgcaggagtgcagtggtgctatcatagctcactgcagtctcctgggttcaagtgatcctcccacctcagcctcccaagtagctggcactacaggcatgcaccaccatggatgtctactttttaaacttattttttgtaagccaggcgcggtgtctcacgcctataatcccagcattttgggacactgaggtgggcggatcacctgaagtcaggagtttgaaatcagcctgaccaacatggtgaaaccccagctctactaaaaatacagaattagctgggtgtggtggcgaatgcctgtaatcccagctactagagaggccgaggcaggagaatcgtttgaacctgagaggcagaggtgagccaagatcataccattgcactccagcctgggcaacaagagtgaatcaatctctctctctctctccatatatatgtatatattaaccatatatatatgctgctttttgtattaaaccactggcatctctgaccaacattcagatggggtattgatggtgttcctgaagaccttgagtcctggagtagtaaatgccctctgggcttcctgcagtgaagacaggagaggcagcctgtcctctgaacctggggaggagcttgtgtcagcccttaggagctgttggccccggtgcagggccccccgagctgaccagcctgtgtcatatatatatatagtatattttgcagagatggggtctcagcatgttgcccaggctggcctcgaactcctggcatcaagcaatcctcctgccttagcttcccaaagtgctgggtttacaggcacatgccactgcacacagcctgtaaataaagttttattggaacagagccacactttctcatttacattttgcctgtgggtgcttttgtgctaccatagcagagatgatcatcctgacacagaccacgtggtcagcaaagctgaaaatactgatcctctggctcttacaaaaacagtttgccaagccctggtctaggaggttctttagtgccctcttgttagtttcaggtggggaattaagtgagggaaagtgaagcctcgtgtctacccggtagatctgcccaggcctgcctgtcttccagctccacagtgctgggatccctaacctgtaacttcagtgtttgcattttatctcactctggctcagaaactaattttttcccttctttatctctctacctcccccttatttttctgtcaggatgagaagaagcacagaagtcaacccaaagatctgtaagtgtttaaaatgcttgtgcttttgtgtcatctggatcagtagaaagcctgttctaggccaggtgtggtggcttgcacctgtaatcccagcactttgggaggctgaggtgggtgaatcacctgaggtcaggagttcgagaccagcctagcctggccaacatggtggaaccctgtctgtactaaaaatacaaaaattagctgggtgtggtggcgggtgcctataatcccagctactcggaaggctgaggcagaactgctagaagccaggaggtggaggttgcagtgagccaagattgcgccactgcactccagcctggacaagagtgaaactctgtctcaaaaaaaaaaaaaaaaaaaagcctgttctagaaacctgtggtgatgtagatgtgtggggaccctggggcctctgaaggcctcgtctcgggatgacaagctggcagtgtgcctatagagctcccatgggtatcttgctttcccatggggaggcattagtttgtcactttccgtgcgagttggcgatgtggttagtgtttctaagctgctacttgctgtgtatctgttcaccctgcagagctgccaaacggaggcccgaagaaaaagaacctgaaaaagtaaatccacagatttctgatgaaaaagacgaggatgaaaaggtaaaggtctcacttttctttctttcttttttttttttttttttttccccaagacggggtttcgccatgttgcccaggctggtcttaggaaaaagaagactgggcgtggtggcgcacgcctgtaatcccagcactttgagaggctgaagcgggtgaatcacatgaggtcaggagttcaagaccagcctgaccaatatggtgaaaccctgtctctactaaaaatacaaaaattatctgggcatggtggtgcacgcccgtagtcccagctactcgagaggctgagataggagaatcgcttgagcctgggaggtggaggttgcagtgagccgagatcgcactccaggctggggacacagcaaaattagctgggcgtggtggcatatgcctgtagtcccagctgcttggaagcctgaggcatgagaattgcttgaacccaggaggcggagattgctgtgagctgagatcgtgccactgtactcctgcctggacaacagagcaagactctgtctcaaaaaaaaaaaaaaatgctgggcacagcggctcacgcctgtcttttcagcactttgggaggccaaggcaggcgcatcacctgaggtcaggagtttgaaaccagcctggacaacatggtgaaaccctgtctctactaaaaatacaaaaattagccagttgtggtgataggggcctgtaatcccagctgcttgggaagctgaggcaggagagtcacttgaacccaggaggtagaggttgcagtgagccgagatcacaccattgcactccaggctggggacacagcaagactctgtctcaaaaccaacaacaaaaaatacaaggacattggtcttattggatggggccccacctttattacctcatttaaccataattgtctcttaaagactctacatccaaatacagtcacatagcaggttagtcttaacatatgaattttgcaggggacatgactcggtctgtataccccctcatcaattgtttgcttacctttacattttagtatttatttttgagacagggtctcactcttgtcgaggctggagtgcagtggtacagtcatagctaccatagccgcaaactcctgggctgaagcaatcctcccacctgggccttcaaggtagctgggactacaggcagatggcactacacccagttaactttctatttttgtagagacagggttttactgtgttgcccaggctggtcttgagctcctgtgctcaagcagtcctcccacctgggcttcttcaagtgctggaattacaggcatgggccactgtgcctggccagttatttatttatttatttatttatttatttatttatttatttacttatgtatttgttttttgagacagagtctcgctctgccgccaggctggagtacagtggtgcgacatcggctcactgcaacctctgtctccctggttcaagctattctcctgcctcagcctcctgagtagctgggactacaggcatgcgccaccgcgcccaactaatttttgtatttttagtagagacagggtttcaccatcttggcgaggatggtctcaatctcttgatctcgtgatccatccgcctcggtctcccaaagtgctgggattacaggcgtgagccatcgcgcccggccctgtgcctggccaattatttgtttaccttgaggtgtccacttacacaggaaagctaaggttttaaaaaattggcttttcctctaaatcttttcaggaagtctttggttatgagatttatttattcatttattttgagatggagtctcactcactctgtcgcccaggctggaatgcagtggcatgatctctgcttactgcaacctctacgtctcgggttcaagcaattcttctgcctcagccttctgagtatctgagattacaagtgcccgcctggctaatctttgtatttttggtagagacgtgatttcgccatgttggccaggctggtctggaactcctgacctccagtcatctgtgtgccttggcctcccaaagtgctgggattacaggcatgagccactgtgctgggcctgactataagattttgtattctatgactttagatggtagagtgagtcagagctcacctgctggccctctcactgcctccctccccttctctctgttttatgataatcacttatacaaagttcttaacaccgaagcactatctgggtgtaaaacactctcttagcctttaatcctcttttgttttccctgtgtaggaggagaagagacgcaaaacgacccccaaagaaccgtaagaatttattcttgacattatcgaaagcagatggtaatgttaaaatgatggttctagaacaaaagggaaggggtttcagtaattgccagagcaaccttaggctcattttggatcgggttggtgaacataggaccaaggtgcgtcaggcaggatgcccctgcggtgaagagccactgtgaccacgcgcctgggcctcactgccacagccggtgcctccctgtgccacaggcacctcaggcagaaacctcacaggggcgattttgatattttctgaaacaattaaacaaacgatcttgtgatttttttttttcccccagaacggagaaaaaaatggctcgcgccaaaacagtcatgaactccaaggtaaacatctgccgggaataaagccggtggcggcgctcacgaggcggctgggagctgctctctgagtgccatcatctgtgttcctgctcccacagacccaccctcccaagtgcattcagtgcgggcagtacctggacgaccctgacctcaaatatgggcagcacccaccagacgcggtacgtacagctctcttcccagccttcctctgcctgtcccttgtcccactgctcaccagccccgtgtccttcaggtggatgagccacagatgctgacaaatgagaagctgtccatctttgatgccaacgagtctggctttgagagttatgaggcgcttccccagcacaaactgacctgcttcaggtaagtgcactttcgtgtgcatgtttgcttcgtggaaggaggcacatccccagaggtgcctgcgggctcacgattctggtgatgttttttagtgcctgacctgcaacatgtcttaagctaacacttcgtggataagagccaccgtggagggctgggcgcggtggctcatgcctgtaatcctagcactttgggaggccgaggtgagcagatcacttcaggtcaggagttcgagaccagcctggccaacatggcgaaacctaccgaaaatacaaaaagtagccaggtgtggtggtacgcgactgtaatcccaactactcgagaggcttaggcaggagaatttcttgaactctggaggcggaggctgcagtgagccgaggtggtgccactgcattccagcctgggcgacagagcaagactccgtctcaaagagccacgtggaaagctgatgaagggacaatagagctttgctgatttttggtttggtttttgggtttttacactacacatattacccctccattctgcccacatcctgagggtcacacgctcagatgaaggcctactctgtgctaggtaccagtgatctaggtgtcgtggatgtagctgctcaggtcctgccttcacggagccagaaatgggtgatacctgtcgggtagtgacagccacgacagattttaaaagcctggtgacatgcagagagaataagtgggaggaggaagttctgtgaagacgggagaggtaatgattttcagtaaagaccttgatggcctgtgagagaagaggccgaggggatggccggggtggggcaccctaggtagaaggggacaccattaccagggctcggaggggcctgtgccagctgtggaaggtgaatggtgagtctggtgtggtggttgctgggttggggctgggccacacagggccttgcaggcctcagcactgactctcctgtcctctgagggaaggtgcctggcgtccggcccgtaccttcaccgaggctcacgtcagctcttgagtgggcagaggccctgtgctctcaggcatccccagccccattttgctccctctccctgcgggtgcagttaccctccagggcttcaccttgttcttcgtgggaaatgcctcagctccaggaacggccctgcctccttttaggcctgggtggtattttccctctctgtgaagaggagggggaagtgccttggggacccctgttggaagcacccccaggcagcggttcctgtacccactgggagggcacccacacgttccccgtgcttggctgatggttgttcccctccctctccttctatgggatccttctatctagatcaagatgctgttgcgtccaccagcaaccgctagccccccgaggcctggcccaaagcagggccacacacctctgcctgctggtgtgagtgaggtgtgctgtggtgggcagagctggcaaccatgcccgagggcctcactctggccttcaccagggacaaaggcttgggggagcctggggctggggctgccagagtcctgagtgtctcagcttcctccctaagttgagatctatcatagattcgttcctggagggtggatggtggcctggaggacactttccagaagtcacatgccttctgtccactggagctggactgcctgagttatttaggggctctccatactgctgtacttggcttgaaactggcgatagaagccagtcatgggtggggtctggcctaccaaccagtgggtcctgcccctgcctggggcttagagcttctctcccatgtctcccctatggagagagcctccagcccacctgtgcagcggggagcgggaccccctcttctgtaggccctcccttgatccacgctggcttcatctctctgtctcttcttctgggccttacatcctgaggcagggctgcaacctattctgtctgccatgcctggtacgcagtaggggagctccgcaggaggtatgcagtgttttgtttgagcctttctggcttccaaggcactaagctgggtgagtgtggtggagggtgctctccagagacctgtgcagacttcatatgggggctctgaggatagcccagcatctttggccgggaaagggtggtccttgtctagatgagcaggtttgtaagcagaacacccagttcatgtctttttccttgagtttgacatcttgcctctctgtcttcactaaagaacgtgctcccgaatgtcaaggggcatctggacagtggggccgcaggtgtttgagatttatgcccaaaaggaggcagaagtccttccttcccacatcccttttcacactgttctataacctgctttattttctaaattgaggtctaactcgtataatataaaattaaccatatgaggtatcttgaataggtgaattcataggtatagaaagcagattggtggttgccgggggctgggggctgagggccggttgggaggagactggagagtgactgctacttgatgggaatgaggctttattaacatttgagtgacagaaatgttctgcagctgaatagagctagtggctgcactgcatagtagaaggtgttctagaaaccggtatttcccgcactgtaagtctgactgatcttttggtgttgctgttgcagacacacatacacttgatgcttaggtgggagaataaggtagaaactctgggtgatagaacgctgtcttaatccagtgttcccgcaaccaaaaaatgagtgtcggggccaggcatggtggttcagcctgtaatcccagcactttgggaggctgaggtgggtagatcactggagataaagagtttgagaccagcctgctacacatagtgaaaccccgtccctactaaaaatacaacaattagccgggcatggtggttcaggcctgtaatcccagctactcgggaggctgaggcaggagaattacttgaacccgggaggtggaggctgcagtgagccaagattatgccatcgcgctccagcctgagggatagagcaagactctgtctcaaaaacaaacaaaaaaagagtgtcagacttgtacattctctcattgcctcgtgcctgatctgaagtctgcacgaagacccgccttcacggcttagctggtaagcatgtgctttgtttcctgtctagtgtgtactgtaagcacggtcacctgtgtcccatcgacaccggcctcatcgagaagaatatcgaactcttcttttctggttcagcaaaaccaatctatgatgatgacccatctcttgaaggtaaggaatagtccgggattatgtttggggcacactttaaaaacagccaggcagggtggctcacatctgtaatcctagcactttgggggtctgaggccagaggatcacttgagcccgggagtttgagacgagcctggacaacatagcaagagcctgcctctattaaaaaaaaatatatagcatatatatatagtatacatatatacttatatatatgtataaataaaaaacatacttatgaagcaatagtcagctggagttgctgctgcctcttaaagttagggtgtgagactttgtgccaggagcagctgtaggccaagtcattgtgatgactgaggagcacccgacctggctgatgggatgtctgctgagtgggcgcttagagcgcctcctgggtttgggatgtgagtcagagcctttagtccatttcctttttctgctctaggtggtgttaatggcaaaaatcttggccccataaatgaatggtggatcactggctttgatggaggtgaaaaggccctcatcggcttcagcacctgtaagtgtgtggcccatcataggctggccggggtctgaaaggggccttcatgttctccttcctgggggctgacggggctctggtgggaattctcagcaggcttgcagaaggccatgtgactgggaaccttagcaggttcagttggggtagcctcttgtgttagttatgactggctctggaactgactctccagttgtaagagcaggtcttggccggatatggtggctcacacctgtaatcccagcactttgggaggccgagttggggggatcatttgaggccaggaattcgagaccagcctagcctggccaacatggtgaaaccccctctctaccaaaaatacaaaaattagccacgcatggtggtgcatacctgtaatcccagctacttgggaggctatggcaggagaatcacgtgaacccagcagacagaggttgcagtgagccgagatcacgccactgccctccagcctgggtgacggagcaagactctgtcctcccctgaaaaaaaaaaaagagcaggtcttattcttgcaacccaaacccagactgaattccaaacaccagagatgagctcaatgaaggcctactcagctagtcttcgctctctggctggctcagacaggcttcttcagaacaagccagctatgatgtgttgtgccctatgtttctgacatttgggtacgggatgacttttagactgttgggtgagtttggtagactcctccatgccctgtggccactgtaggcgccatcagattccagccccttttccacacctcctctgttcaccccagcatttgccgaatacattctgatggatcccagtcccgagtatgcgcccatatttgggctgatgcaggagaagatctacatcagcaagattgtggtggagttcctgcagagcaattccgactcgacctatgaggacctgatcaacaagatcgaggtaagagatcgagggtcctcagcatccgggattcccactggaaacttgccttcagaaccagcagacactgttcttcagttggatttaggccagtttggcttaagcatgagagaaacctgttctctttcaagaccacggttcctccttctggcctcaacttgaaccgcttcacagaggactccctcctgcgacacgcgcagtttgtggtggagcaggtggagagttatgacgaggccggggacagtgatgagcagcccatcttcctgacaccctgcatgcgggacctgatcaagctggctggggtcacgctgggacagaggtaaggatgcggcagggaccagagtgaagactggagaccggggagggtagagcatggcccacatcctctgtcccagtcctctgagatgctggaacctctcccgtaggcgagcccaggcgaggcggcagaccatcaggcattctaccagggagaaggacaggggacccacgaaagccaccaccaccaagctggtctaccagatcttcgatactttcttcgcagagcaaattgaaaaggatgacagagaagacaaggagaacgcctttaagcgccggcgatgtggcgtctgtgaggtaacctcacctgtgggtgctcccgctcccctaaggtggcccagcctctggcctgatctgaggactgctccatctttctctctgtggcttgagactctggctgctcaaatgtgacccctgagacagaaattagttagtggtctgggaggaccctgcatacttcattgaacgtcaataggacagatgtccacagaggcatgcttgggtgctttgctgataatgccactgtccttaattgaagagcctggcttctgggtggctctgctgtgcctgcatctcacagtctgaaggcccctgttggactgctaaaggggtattcacatctaagtgggcactttgtccagcttagatttgaggccctcaggaggcgtccctttctgtgagtttttaaaatatatgtttacttcactcaggaggaactaacttcctagtcctatgacccaaagaccaggactattttgatacctattacctgactttcttttggataaagactggcttcagggctttgcttttgatccccgctggcatctcttaacaggagcctgcatgtcctcaccattgttattttggttttattttatttttcttggttaacacacgtgtgtccaatgttttataacttctaaaatcttttttttttttttttaatttttttgagacagtctctctctgtcacccaggctagagtgcagtggcaccatctcagctcaccgtaacctccgcctcccgggttcaatcagttctcccacctcagcctcccgagtggctgggattataggcacccgccaccatacccggctaattttttttgtatttttagtagagatggggtttcaccatgttggccaggctggtcttaaactcctgaccttaagtgatctacctacctcggcctcccaaagtgctgggattacaggtgtgagccactgtgcccagccctaaaatcatttgtttttatttgtgtaactgttacgtgttgatactggtagatctagttatttcattttaaatattctgtagtattttgttaaatgaatatcctaacttactcctcaatgataggcacctcttcagtattgtaaatatgtggcagcatccttgagcaaagcttctcgcgagtgtatctgcttagaagtagcgttgctgggttgtgggtgtgtattcttaactttgatctagaagattgctctccaaaatggttctaccaacttaccatttgagcagtgtgcgttcttctttcctcacaccacctatctggcttcttaacccatctggagaatgggaagggaattttatttctctcacttaccatatttctaatgaggttgaatattgctttttttttttttttttgtagagacagggtcctgctgtgttgccttgcctggtctcaaactcctggcttcaagtggtcctcctgtcttggcctcccaaactgccgggattagaggcatgaggcactgcacccagccaaataggcacatttttaaaagtcttggttttgttgttgttgttgttgttgttgttgttgagatgggtctcgttctgtcacccagtctggaatgcagtggcatgattgtagctcactgcattctcaacctactgggctcaagtgatcttcccaccttagcctcccaagtagctgggaccacaggtgcacgccaccatgcccagctaattaaaaaaaaacattttgtagatgggattttgccatgttgcctaggctggtcttgaatgcctgaactcaagcagtcctcccgccttagcatcctgagttgccgggactgcaggaatttgtcacacgcctggctaattttaattttttgtagagttgggggtcttaatatgttgcccaggctggtcttgaactcttggactcaagtgatccatctacctctgcttcagcctcccaatgtactggttcaggtatgagccaccatgcccagcctaaaagtcttaattcaccgtttgtttcattgttgctaagggctgtgtgactcactttttttttttttttttaattttttgagacctaaaagtcttaatgcatcatttgtctcattgttagtaagggctgtgtgatttgccctttttttttcctttttttttttttttttttttttttttttgagatggagttttactcttgtcgtccaggctggaatatggtggtgtgatcttggctaactgtaatctccacctcccaggttcaggcaattctcctgcctcagcctcccaagtagctgggattacaggcacaccctaccatgcctggctaatctttgtatttttagtagagatggggtttcaccacgttggccaggctggtctgcaattcctgaccttaagtgatctgcctgcctcggccttctgaaatactgggattacaggcatgaaccactgtgcccagccctgatttgcctttttaatgccattttttaggtgtgtcagcagcctgagtgtgggaaatgtaaagcctgcaaggacatggttaaatttggtggcagtggacggagcaagcaggcttgccaagagcggaggtaggtcaggccgagtcttcctcctgtggcagaggacttgccagctggtggcagatgcactgtggagaagggcctgcatgtgtgggacagcaccaggattccttcgttaggcattgtctcaggacctgtccctgttatgaagaaaacagccccggttggtcttacttagaaaaggggccttaggtataaccagtgacattgcaggtgtcccaatatggccatgaaggaggcagatgacgatgaggaagtcgatgataacatcccagagatgccgtcacccaaaaaaatgcaccaggggaagaagaagaaacagaacaagaatcgcatctcttgggtcggagaagccgtcaaggtaacccttggagccccttggtcagctcactgccatgttcccaataagcaggttgccctctagtatctcgccaacctgacctctcaggcagcctgagccccggcactaagaaacctgtctgtttgcccctgagaagagcgggactggccaagagaccatgttccatggctcagtaccagcaaggcatttttgtgtggccacaaggctcagttctcctaatgtcccaaaatgggcatttgacacagttctaggggcatcaggcaggtgagccctcatagcttgtttttctgttttgttttgtttaagatggaatcttgctctgtcacccaggctggagtgcagtggcacgatctcagctcgctgcaacctctgcctcccaggttcaagcgattctcctgccccaccctcctgaatagctgagattacaggcgtgcaccaccatgccaggctaactttttttgtatttttagtagagacggggtttcatcatgttgcccaggccattctcgaactccggcctcaagtgatccacccacctcagcctcccaaagtgctgggattacaggcaggagccactgtgcctggcccctcatagcttgtttccaagagagctgtcacagccccctctggtgtggggtgggtctttttggggcaccaacagatggcctcccatccgttctgtggtcattggcttcactgaaggtcagttcctttaccacagtgtgagagagaggagtgagtatcctctgtgttgcttctctgacgtccataaattaaagatgcaatcagttcacttaattatgaggcttgccacctgctggctgcctgggccgtggctgagcccctgcaggaggacagacatgggtactgctgcatggcatgagtgcctctgcagggtagggggctgggaggaggctggggccacgaggccttgcagaggtgacacctgggctgagatggagaaggtcaggtaggacctagccacacgacctgggggagagctgcactgaggtggcagtcagcttactttgttggagaaaaggaaggaggcccctgcagctggagtggcttgagccaggggatgatgggcaggacaggggtcagggttcaggctgtggccttacagaccatggtgaggtattgtgggctgtgctgttaaaatcaaatcctgggacccgagtggcagagcagaagtgggagggacatggatgcccttggccacagcatctggccaaggaaggtggatgttcttcttttttcttattattgaagcccttgtagaacaggatgttctctttagcagcaactcttcattgacttcttaatgaaatcgagtcatgtcttactttaagtaacagaggaccatgcaagtcaaggccagcaaagaccctcagaatgatcctccatgaacttatgctctcattttcagactgatgggaagaagagttactataagaaggtgtgcattgatgcggaaaccctggaagtgggggactgtgtctctgttattccagatgattcctcaaaaccgctgtatctagcaaggtttgcatctttctttttgcttgacttctgcatgcactttctcatcaagtaggagatgcctgttttgccaaaatagcaccagcagctttttgccctagatgaaaactgaaaaggacgagtgctcctgcatagacttggtcacatatctgctggccaagtcagaactttctccccatgcccgtcttctgattccagggtcacggcgctgtgggaggacagcagcaacgggcagatgtttcacgcccactggttctgcgctgggacagacacagtcctcggggccacgtcggaccctctggagctgttcttggtggatgaatgtgaggacatgcagctttcatatatccacagcaaagtgaaagtcatctacaaagccccctccgaaaactgggccatggaggtgagtgcctggtgtccctcgtgagcccctgccttatcgtggagggtcactgctgacgtgcgttctgttttgttttgatttgtatttttttttaatttggttttttttttgtttttttttttgttttgagactgagtttcgctcttgttgcccaggctggagtgcaatggcatgatctcagctcactgcaacctccacctcctgggttcaagtgattctcctgcctcagcctcccgagtagttgggattacaggcacccaccatcacgtccagctaattttttgtatttttagtagagacggggtttcaccatgttggccaggctggtctcgaactcctgaccttaggtgatccacccgcctcggcctcccatagtgctgggaatacaggcatgagccactgcgcctggcctgacacgcattcttatctcccaagggaggcatggatcccgagtccctgctggagggggacgacgggaagacctacttctaccagctgtggtatgatcaagactacgcgagattcgagtcccctccaaaaacccagccaacagaggacaacaagttcaagtgagcactggggctggactcggggtcagcaggcactttcagcccacatcactcccttttcccgtgtgcttccgtctggttggcttccgccaacggccccccagtttgtgtctcttgctcgtcaaaggccagcctgactatgaatgatgctcaagtgtgtagatggcctggagttgctttaagtgtgtggatgacctggcgttgtctttgtaagagggttccctacccaggaacagggttcgtcttgccatttcaagacttcccgtgtgttcttcagtgtgtgcccagctcttgcatgtttttggctgtttcttagatattttctcctgtaaatgagacctcttatttgattcttttctttcctttttttttttctttttttttttttgagacggagtctcgctctgttgcccaggctggagtgcagtggtgcgatcttggcttactgcaaactcagcttcctgggttcatgccattctcctgcctcagcctcctgagttgctgggactacaggcgcctgccaccacacccggctagttttttgtatttttagtagagacgaggtttcactgtgttagccaggatggtctcgatctcctgaccttgtgatctgcccgccttggcctcccaaagtgctgggattacaggcgtgagccaccacacctgtcctcgattcttttgtaactgggtgactgctttgagggaagcttttattttttattttattttattattatttttttaattttttttttttttttttttttgagacagagtttcgcttttgttgcccaggctggagtgcagtggctcaatcttggcccaccgcaacctctacttcctgggttcaagcgattctcctgcctcagcctcctgagtagctgggattacaagcatgggccaccatgcccggctactttttttgtatttttagtagagatggggtttctccatgttggtcaggctggtctcgaactcccgacctcaggtgatctacccgcttcggcttcccaaagtgctgggattacaggtgtgagccaccgcgcccggcgagcttttattttttaatgtccaatgccccatgccttctttttaacctccgtacctcacttgtttcaactaaaagcagcagccagcacccccagaggagcaggtggcaaccagggaaacccaggaggccaagagggctccccgggggcatctgtgcttctcccataagtctcgtgctagctttggcttgatctctattttcattttattttttacttttttgtgatggagtttcactcttgtcacccaggctggagtgcagtggcacgatctcggctcactgcaacctccacctcctgggttcaagtgattctcctgcctcagcttcccaagtagctgggactacaggcgcctgccaccatgcccggctaatttttgtatttttagtagagacagggcttcaccgtgttggccaggctggtctggaactcctgaccttgggtcatctgctcccttcaggctcctaaagtgctgggattacaggcatgaactactgcacccaacctcatttacttatttatttatcacccaacctcttttatttatttatttatttatttatttatttatctgagacagggtctccctctgtcacccaggctggaatgcagtggtgtggtcacagctcactgtagcctcgacctgctgggctcaagtgatcttcctgcctcagcctcctgagtagctgggactgcaggcatgcaccaccctgcctggttaatgttttaaaaattttttgtagagacggggtctcactgtgttggccaggctagtctcaaacttctgggctcaagcgaccctcctgccttggcctcccaaagggctgggattacagacatgagccaccatgctgggccttgatctctatttttaaggccatcttcaagtctcctttaagggcttactagtcaagaataatgttgaatttcctcaaatacctgttgagcctttatggagagaacagtaggagtctctgctggattgagttcctcactgagccactcgggactgttgctcgtggtgtgttcttaaccatattctagaccctgttcattgatatcttagggtttttgtctcaatgactcaaaagttgcctgattttcttgctatgttctctctgggctttggtctcaaataagtgccttcaaaatgtcaacacaaagcacaagaaagccacctcttccttctagcatctgcaaatgttttttcctcttggttaccagagtctctggattaagctggcagtagctgctgcggccactgccggccacctcagggccttatgtttctgtccctttgtttccttcagattctgtgtgagctgtgcccgtctggctgagatgaggcaaaaagaaatccccagggtcctggagcagctcgaggacctggatagccgggtcctctactactcagccaccaagaacggcatcctgtaccgagttggtgatggtgtgtacctgccccctgaggccttcacgttcaagtaagtgccccctcggagcagccggggccagggcgctccaggctgagccaggctggctttgtggtgtccccctccttacagacattgccaatccctccgcaccagctgttgtcctagccgtcacgttggcataggtggcaaacagacaacataaatcgtgtagactgaccatgatgctattgcagagcaaagctgagcgtaaaagtcaactcgctggtttggctgggtgtggtttctcacgcctgtaatcccagcgctttgggaggccgaggcagtggggaatcatctgaggtcaggagtctgagaccagcctggccaacatggtgaaacctcgtctctactaaaaatacaaaaattagccgggtgtggtggtgcgcgcctgtagtcccagctactcgagaggcagagacaggagaatcacttgaacctgggaggcagaggttgcagtgagccaagatcatgccactgcactccatcctgggcaacagagtgtgactccgtctcaaaataaaaaaagaaaaaaaaagaacttactggataaagcgcgagggacagctgggggcccacccccaagttctgcctctgcccaaggacacctcctctccgtcccctactgtgaagtgggagagtgggatcttgcctgctggggctcgaagggttgagtgagttgaaagtgctccatacagtacctggcttaaagtaaaggtttaacggacatgagttacttcttggttagagggaatcacatcagatgacttctctgtgtcagaggatcgaggctcttccaggaaaaacctaaagccacagatcgtttccacactgatccattctgtgttttttctaagaatcagcgtgtggttggaattacagccaacgttttccagatgccttcccggctcacaattccatttccttagagccccagctcccttgccttagttcagttactgtgaattatgagaactggagaagcaggttccctccagccttggcacagttggggcgcgttaccccctctggttgtccttggtgggaaggggacacctccccgtagtgttttctgccagttgctcctggcaaagtgggagagtcagcacagagacgcctttggggcccgtcagactcctggggggcctagtggggagaacgtgacagctgacttccctaacggcaggccttcttcaaagaacagcttttccacactagtcagaccacactcccattgtcccaggccctgtggcgccttacattggcctccagtgtcgcttctagatcagagccttagtcagggtagcgtaggttgtatcatagcaacaaccccaaatcacagtgacttaaaaccacagaagctggactgggtgcggtggctcatgcctgtcaggagttcaagaccaagaccagcctggccgacatacctgtctctactaaaaataaattagccagggttagctgggtgtggtggtgggcacctgtaatcccagctacttgggaggccgaggcacgagaatcgcttgaacctgggaggaggagttgcagtgagccgagattgtgccactgcactctaacctgggtgacagagtgaaactctctcaacaaaaaccacggtagccgctgtctcgctcatacgaggtgcccatcacaggtcagcagaggactccacctgttgtgtcccttggatatctgggccgctggagtcagaagttgccagtgtttgagcccaagggaaagcacacaggacattccccaggcagtgacatcctctttggtcacttctgcccacgtcacatgccaggaaacacaagatgccaagtagtcgggaggcaggagcctggggtatttgagcagtgtcagccaccaccgtagccagtaatgcgcagagggtgaatgcgtgcttcaaactgtgagactaaatctgcagctaaatcttttttttttttttctgtgagatggagtcttgctctgtcacacaggctggagtgcagtggcacaatctcagctcactgcaacctctgcctcctgggttcaagcaattctcctgcctcagcctcccaagtagctggaattataggcatgtaccaccacacccggctaatttttgtatttttagtagagacagggttttaccgtgttggccagggtggtctcgaacttctgaccttgtggtccacctgccttggtctcccaaagtgctgggattacaggcgtgagccacaatgcccagcctaaatcatttcttagggtacacacctaccttaattcatcaggtgcttgactttaaatggttattttcactggtcagtcatgcctgactgaccactgcaaggtggaaggttcattgatgtcaagtgggtgcttctctgcagcatcaagctgtccagtcccgtgaaacgcccacggaaggagcccgtggatgaggacctgtacccagagcactaccggaaatactccgactacatcaaaggcagcaacctggatgcccctgagccctaccgaattggccggatcaaagagatcttctgtcccaagaagagcaacggcaggcccaatgagactgacatcaaaatccgggtcaacaagttctacaggtcagcagaggcctctgttcttccacgaggccacagactcttctagaaggctctgcctgaaacaaggttgtggcatagcacccacagttgttctaagagttaggttgaagtagctgtttttccaagtgtttctgattatcggtgggcactgataatgactagaattctagttcccagccctcacttttaggtccttattttaatgttgcagagccctttttccaaaagaaatccttaagctggctgggcactgtagctcacgcctctaatcctagcactttgggaggctgaggcaggaagatcacttgagtctaggagttcaagaccagcttgaggaaacgtagtgagaccccggccctaccaaaaaaaaaaaaaaaaaaagactagctgggcgtggtggcacacgcctgtaatcccagctacttgggaggctgaggctgaagggatcactgaaacccaggaatttgaggctgtagtgagccatgatcccaccactgcactccggcctgggtgacagccagaccccgtctcaaaagaaaaaacgggtagagtgcctgggcacccccagagaacctcccaagctccatctatgactgtccaggttctgtcccgctgtccccacctcttcccaggtcctcctttctgtctctgggattttttctctttcctaagcttttggcctattacgttttctttttctaagacccgagtaacttgcggtgattaggcattaacctgaaactagcctgtacaggaagctatacacttgcctcacctcttagctaacatagatcaaagatttcctgaagcctttgtaataacaggaaataaaacaagggctgtgagtggtacaggtgagagaaacacactcgagaggctgctaccggcaggcggacacagcagggcagggccaggcgtgcagagcaagcagccaagctgacctaatggttggggggtctctaccctgtggttccctaattgggggcgaatctaccttgcagttccctcatttggggaggggttatctttacctgcggttccctaagtaggggggtctttaccctatgactccctaactggggaggtctttaccctgcggttccataactggggggtctttaccctgcggttcccaactgggtcgggggtcattacctggcggttccctaattgtgggggaatctaccttgcagttccctaatgtgaggaggggatatctttaccctgtgattcccttactgggaggggtctttaccctgcgattccctaacttgggggatctttaccctgtggttccctaactagggggggtctttaccctgcagttccctaaatgggggggagtctaccttgcagttccccagtgtggggaggagatatctttaccctgcagttccctaactgagggggtctttaccctgcggttctcgaactagggggtctttaccctgcagttccctgagcaccccaggaacatggtactgtttaattcagcataagctcaccacaaaggagagctcctaacgaggcctctcccgctcgcaggcctgagaacacccacaagtccactccagcgagctaccacgcagacatcaacctgctctactggagcgacgaggaggccgtggtggacttcaaggctgtgcagggccgctgcaccgtggagtatggggaggacctgcccgagtgcgtccaggtgtactccatgggcggccccaaccgcttctacttcctcgaggtggtgcccctgcttgctagagggaaggcttcggggtcaaagttggccagaaggagtctgatgtcgggttatacacaaggcggcttggctgcagggtttcagcttttgtaagaagtgggtggttggctgacgtgaagctgttctgcaggagctttacgggggtctgagcagggttctccagagcacaagttgtatgagattcaagtggtcccggagtttaacactcctggtttttaaaaagtacgtcattagagaacctaacgttttagtgtcttggtgcaactgaacgttgaggaccatgaggcagcttccaccagcctgactggtgtcttagtatgagttggagaaggtgcccctgctcctgggtcccttgggccaggcgcctacgtccaggcccagcccacatgcctgtgcgggtggccctcgttatggagagggcccacgagtcagctgatcaagctcatgacttgtaaattgttggggcttggcctttccgtgttgttaccttaaaacgatacatccacgttctcccaggcatggatgaggtagcccaattggacagcagattctgcatagcactttttcctgttctagaaggtactctcaagtgcttgtaataggcatttcagtgtcagccagatcaaatgttagtattaataaaatggattgttttaagtttctattgtagcaaagtcaaactactcttattggtggctaattggtcatggccccactgagggagaaattaagtgactatcaaattgccttcttactagtctgcgttagagaggggacagtgcgtttctctcccaaacgattgcagttctctccttttcaggcctataatgcaaagagcaaaagctttgaagatcctcccaaccatgcccgtagccctggaaacaaagggaagggcaagggaaaaggtacgtcattgtatgagtttcttttcaagttattcttctgtaacttggaggctgcctgtgaatccctcagtgtaaaaccacctctggtgttactgactctgggacagcgaggccgcctgagttaacaaggcgcttgagagcaaggtggacttggactctgaggatcgggtttagcctctggcctctctcccccagggaagggcaagcccaagtcccaagcctgtgagccgagcgagccagagatagagatcaagctgcccaagctgcggaccctggatgtgttttctggctgcggggggttgtcggagggattccaccaagcaggtgagcgcccgtaggctccatctctgaatacctggtgagcccagaccgggcaggtgctacctgaaacgacttccaaccccggtcaccttcctgactcaagaatctccttccgaggccaggcacggtggcgcacgcctgtaatcccagcactttgggaggccgaggtgggtggatcatgaggtcaggagtttgagaccatcctggccaacatggtgaaaccccgtctctaataagaatacaaaaattagctgggcgtggtggtgtgtgcctgtagtcccagctactcaggaggctgaggtgggagaatcgcttgaacctgggaggcagagcttgcagtgagccaagatcacaccactgcactccagcctgggtgacagagcgagactccatctcaaaaaaaaaaaaaaatcttctggagagttgaaagcatggcttcgtgcttgatctgccaggcatctctgacacgctgtgggccatcgagatgtgggaccctgcggcccaggcgttccggctgaacaaccccggctccacagtgttcacagaggactgcaacatcctgctgaagctggtcatggctggggagaccaccaactcccgcggccagcggctgccccagaagggagacgtggagatgctgtgcggcgggccgccctgccagggcttcagcggcatgaaccgcttcaattcgcgcacctactccaagttcaaaaactctctggtggtttccttcctcaggtaaacgggtagaagccccccagtgttgccagacggcccggggctgtgccgcatgtcagcagtggtcattttcgccctgcactgaggcctctgccccccggaagctcacagctcagctctcaccagggagagactttgataacattcgtgaggggcttccggcacagtgggtcgtttcttccctctgtctgtggaggtgactcctgcagtctctcctgccccctacagctactgcgactactaccggccccggttcttcctcctggagaatgtcaggaactttgtctccttcaagcgctccatggtcctgaagctcaccctccgctgcctggtccgcatgggctatcagtgcaccttcggcgtgctgcaggtgggccctggggctggggcgggcagacagatgtggccagcacgtgacccggccagcagccagccatcccttactgaaggcagggttcaatggcataggcctgccatccaggcagcagaggctggcatggtgctctgtcccactggcggagtcaggggagatgcgtgctcagccggcccccagcaagtccaccgctagtgtctcttgagctgtgttctcgaacatgcctgggagggctggctacaatgggcttctcgccatgcagctcctggctcctttccctcgatggggaagggtgagcagcccaccatggagcactgtggaatggctgagtctgcacagagcgggaggggaagtgaggtgcgcagagccatgtgtaggcggcagctgttcgattggtttaaaagacagttttgctctgggtaactgcgcacacaaacccccacagatggccgagggttgcactccaggtggctgtgtctgcagggacagggatggacatagtctgtggggcccaggccttatctgaaaagcatcccccatcctctctgtatcagctcagcaattgagggcataattaaaaatgaaacagctatccagatgctagcacaggatgtctgcctaagtgggcaagcacttggctacccggtcccctagctcagcctcagggcgactcaggctcaccagacaagacttgttgcagccacgttctctgtccttgtgaccacagccatcagggaagcgacagggctcctggtctccagtgttctccccgagtgctaagaccttggggcccttgcagtgctttcagagctggccggacttccccgcagggtttgctcccaccttccccagtggctgtcccctgggctcagctgcttttgttctttatctgctccaaactcctctgaccaggagcacgcctgctgaggtaactggggatttctgcaagtgcactccgcattggccagccactcccgactcaggctgctgaccctgggcctgggtctggccagtccagttgggagtgtcccactgacggtggggttgtccgtccttctcccccacaggccggtcagtacggcgtggcccagactaggaggcgggccatcatcctggccgcggcccctggagagaagctccctctgttcccggagccactgcacgtgtttgctccccgggcctgccagctgagcgtggtggtggatgacaagaagtttgtgagcaacataaccaggtaggtggcccccgtcgctcctccacacactgccgagcaggcctcagtagctcatggggcccgtgagtactccctggtgaggcttggggaggagaggatggtacagcatctgccctgccacacggtgctctgccacatggcactctgccacctgtggtctccagttctgaccctgcatcatctgcttccctagtagatagcatgtgcagagcggccagccgcatgcttggctgtggtcatccacaaagcccacgccatagccccatccccccttccagatggcatccaggcacactgccacccatgtgacctcgggcagtgctgtgatctcgggagaaggccatctgagcaggcagggggtggcacctgtgatgaggggacagctgctgcgtgcatctccagaggtgttgacctcctcctctgttgcaggttgagctcgggtcctttccggaccatcacggtgcgagacacgatgtccgacctgccggaggtgcggaatggagcctcggcactggagatctcctacaacggggagcctcagtcctggttccagaggcagctccggggcgcacagtaccagcccatcctcagggaccacatctgtaaggtaatggcaccctgacagagcggctcctcctcgaggcccagcccagcagcctcgtgggaacagtcagcctgcccaagactcaggggagacatggaatctgatcccaggctcctcctcccgagtcctcagcctttgtgtgacccctggtctctcttgcatggtcatgggaagtgtcagagctcccgcagtcctggacttggtctggaatggtgtcaagccactgcccacccaccactgccttcccccttgctgcctctccccgcatgttttcccaggatgccaagaccagccctttcaccatcccctggcaccactccccttcccccaccctaaaggctcagcttagattcccacgctctcctacacctgcgctgcctcaggtccctgccgtggcctcgagccaagtgcgatatccctgactgtctctttcccgatgggcgtgagccccagggggcttcctggtcaccatggtgttcccagctcccagcccaccatggatgctcggatgctgctcacatgaggtgacagctgggccccccacactctttcaggacatgagtgcattggtggctgcccgcatgcggcacatccccttggccccagggtcagactggcgcgatctgcccaacatcgaggtgcggctctcagacggcaccatggccaggaagctgcggtatacccaccatgacaggaagaacggccgcagcagctctggggccctccgtggggtctgctcctgcgtggaaggtgggtcctgtaagttgtggttcccggtgggctgaggggaaggaaggcagagccctgggcctttggcctgtgaacctggaggccagggcagggcacagacacaccaagcctgggtgtcccagagaaggtagctgctgaccatgttgagtagtaaaagcaggctgggttcatggcccacacctataatcccagcactttgggaaaccaaggcgggaggatcacttgagcctgggaggttgaggaggctacagtgagctgtgattgtgccattgcactccagcctgggagacagagtgagaccctgtctctaaaaagaaaaaaaaaaaacaataaataaataataaaagcaggtcaggcacgatggctcacctctgtaatcccagcactttgggaggccaaggcaggcagatcacttgagatcaggagctcgagatcagcctgcccaacatggtgaaaccccatctctattaaaactacaaaaattagccgggcgtggtgtcgcacgcctataatcccagctatttgggaggctgaggcaggagaatcgcttgaaccccataggcagaggttgcagtgagccaagatcatgccattgcactccagcctgggcaacagagcgagacatataatactaataataaaaacagtgcccagagaacgaggattgttggctgctccaccccagggggccccttgcacaggaggtgccatctctgcctcccaaagctctaagagccactgtcccaagcctataccccatcccacaactgcagcctcatcactgtcctgtcttccagccggcaaagcctgcgaccccgcagccaggcagttcaacaccctcatcccctggtgcctgccccacaccgggaaccggcacaaccactgggctggcctctatggaaggctcgagtgggacggcttcttcagcacaaccgtcaccaaccccgagcccatgggcaagcaggtaggtggggagggggcatccgagggcctgggtcaggccctgtacttggcggcctaactaggtggaagtgtgggtttagccaagtgggggacagcaccccaggaccccaggcacctggcctctgcctcctgcccctccacgtccacgggacaagctcataggccaaggccatggccgtatgctgtcacagtgccatttccctccctgtccccgacggtgacccggcctgggtgctactgccctcgcccaccgcgcctctttcccccagggccgcgtgctccacccagagcagcaccgtgtggtgagcgtgcgggagtgtgcccgctcccagggcttccctgacacctaccggctcttcggcaacatcctggacaagcaccggcaggtcagtggggcggcgcccgctgggtctggacaggaaggaggcttctgtgcctgtcaccaggtggggctggggccagcgcagtcacttccgggaggacgcctgctggctgccgggtagccccatcactctcagtgggaagacggcccacaggagggacagtgctgagacatccgagtcaggctgcgtccctatgctggttccttactgtttgtttttgaggcagagattgagccagaaggacagtccgtcgcgctaaaggagcaggctaaaacccatggtggcgacagctcactcgctgtaggttcaaatgaactagcaccttaattcctacctgcttctcaggtgtcgggttggcttaacaagttagtctatgagttagtctcagttaagtgtattttcatcatagggcacgcgcattgcttaaaaaccagtgtccacggttcttgtgaaaaccacatttcacccctccccccgaatctggttctcgggaagcagccactctctcttttttttttttttttttgagatggagctttgttctttcgcccaggctggagtgaagtggcgtgatctcagctcaccgcaacttccacccccctggttttaagtgattctcctgcctcagcctcttgagtagctgggattacagtcatgtgctaccacacccggctaatttttgtatttttagtagagatggggtttcaccatgttggccaggctggtcccgaactcttgacctcaggtgatccgcctgcctcggcctcccaaagtgctgggattacaggcatgagccactgcgccggccagggaagcagccactcttaactgggagcgaaacgtgtctaggtggcccatttgtcttttttttttttttttctgagacagggtctcactctgtcccctaggctggagtgcagtgacatgatctctgcccactgcaacctccacctccagggttcaagtgattctcctgcctcagcctcctgaggtgctgggattacaggcatgtgccaccatgcccagctaattagtttttgtacttttagtagagacggggtttcaccatgttggcgaggctgctctcgaactcctggcctcaactaatccacctgccttggcctcccaaagtgctgggattacaggcgtgagccactgctcccagccccacgtgtctttgtctcaagtctttctgaagctcttcaaaggcccagtgacttgtggctgtggggcgggatgatgggccagttggagggtccaaggatcttgtgctggaagggttttgggcccatgtgagcaggaccagaacccttccccaaggggtgcaatgcccaggttgtcctccatctgagcaggggctggcagtacacctgcccccgggccttgggcctgggtgtccacatcaggcattgcccttctcccctcctgcaggtgggcaatgccgtgccaccgcccctggccaaagccattggcttggagatcaagctttgtatgttggccaaagcccgagagagtgcctcaggtatggtggggtgggccaggcttcctctggggcctgactgccctctgggggtacatgtgggggcagttgctggccaccgttttgggctctgggactcaggcgggtcacctacccacgttcgtggccccatctttctcaaggggctgctgtgaggattgagtgagttgcacgtgtcaagtgcttagagcaggcgtgctgcacacagcaggcctttggtcaggttggctgctgggctggccctggggccgtttccctcactcctgctcggtgaatttggctcagcaggcacctgcctcagctgctcacttgagcctctgggtctagaaccctctggggaccgtttgaggagtgttcagtctccgtgaacgttcccttagcactctgccacttattgggtcagctgttaacatcagtacgttaatgtttcctgatggtccatgtctgttactcgcctgtcaagtggcgtgacaccgggcgtgttccccagagtgacttttccttttatttcccttcagctaaaataaaggaggaggaagctgctaaggactagttctgccctcccgtcacccctgtttctggcaccaggaatccccaacatgcactgatgttgtgtttttaacatgtcaatctgtccgttcacatgtgtggtacatggtgtttgtggccttggctgacatgaagctgttgtgtgaggttcgcttatcaactaatgatttagtgatcaaattgtgcagtactttgtgcattctggattttaaaagttttttattatgcattatatcaaatctaccactgtatgagtggaaattaagactttatgtagtttttatatgttgtaatatttcttcaaataaatctctcctataaaccaccctgggtgggtattcgtgatttgcacgggacttctggctgaggtcagacaggaacaagctcttccatgctgaaggcgacccacgtggagtttgaagctagcacgtttgaagctagacactccgtgtgtctgtctgtgtttgttcagaactgagtcacgtgagttgatccccatgacatgcaaatggaacttgctagtgtagcttccaggaaagccttttaaaggaagtcttggctggccttccccgtggctggtaggccttctttggtctgaagtgggttgtgatgcctggcagtggagcagccatcctgtgaccatgaggcagagctctgtgctgcaactggtggaataaggagagagaagtcaccactgagctgcccttggagccccggccaccccatccccaagcaaaacctctttagttgggttttttttctttccaaagaatttttttttttttttgagacagtcttgctctgttgctcaggctgtagtgcagtggggcagtctccgttcaatgcagcctctgcctcctgggttcaagaaattctgcctcagcctcccgagtagctgggattgtaggcgcccatcaccacgcccagctaatttttgtatttttagtagagacgatgtttcaccatgttggccaggctggtcttgaaatcctgacatgatccgcccacctggacctcccaaagtgctgggttacaggtgtgatccactgcacccagcctcccattagcttttttttttttttttttgagacagtttcgttcttgttgcccaggctggagtgcggtggcacgatctctgctcactgcaacctccgcctcccaggttcaagtgattttcctgcctcagcctcccaagtagctgggattacaggcatgcgccatcacacccaactaattttgtatttttagtagagacggggtttctccatgttggtcagggtggtctcaaactcccgacctcaggttatccgcctgcctcggcctcccaaagtgctgggattataggtgtgagccaccatgcctggccagcattttttaatagtgacataacacttctgtgtgtcctttggccactggatgtaccgtttctctgtgatggccacatgactgtctttgctgggagctccctgggcatttgggatgactgcccctgctcagtcatcctggcttatcatgtctgtgccatacagagacctcccatttttaggaagtcaatttatatttgtcactcttagttttttgtagcctgtttatttttgagacggattctcactctgtcgcccaggctggagtgcagtggtgtgatctctgctcaccacaacctccgcctcccgggtttaagcaattctcctgcctcagcctcccaagtagcagggattgagtagctgggattacaggcgcctgccagtacatctggccaatttttgttaatttagtagagacagggttttgccatgttggccaggatggtcttgaactcctgacctcaagtgatcctcccaaagtgctggcattacaagcatgagtcaccgtgcccggcctttgtagcctatttagaaacgcctttcctccccccagcaagagatcttaaacatgtatcttcttccacatttctgacattcccattttaacacatgaatcacttttgtaaggactaaaaatggggatctcagcttattttttgaataagactgatagcaaatgcacacagcaaagtacaacagctcagcttgacatatggtaaagttcccccatccgagatttttttttaaatatactttctgctgctattttaagctaaaaaattcattttaaagtatataatgagtataatgagttttctttttctggattccaaggcaccttgatcaagacaactgaatctttgtactaatgcatagtgtgaccttgtgagaaaacaatcacccatgtacctattatctttttttattttctatttttttgagacagggtctcagtctgtca

SEQ ID NO: 2 - DNA, 114717 bp, Homo sapiens
gcagtgggctctggcggaggtcgggagaactgcagggcgaaggccgccgggggctccgcgggctgcggggggaggcacttgacaccggcccggggagaggaggggccgctgtccctgcggccagtgctggatgcggggacccagcgcagaagcagcgccaggtagcggggcgcccagcgtggggagggctgcggcgaggcccggagctcggctacgactcgaggggctctgagtggccgggagcgcggccgaggcggggcgcccggggcccggcccccggtctgggcggtgcggggtccagacctccgccaagcggcgtgcggggaccccggcggggccgcagaggccgggaagtcgcgccgagggcgctaggaccggccgcgcgcccaggccgctcccgccgcggcgccccggggccccccagtcggcgctgcctcccgggccggctccgcagcgccggggccggggccagcgcggccgcggggggcgagcggcgggcggaggcctgggcccgcgggcggccggagggagggaaggaggaaggcggagagaaggggtgggagggggggacccggcggaggaggcggagagagggagcgcgacagcgagcggagggagggagcgagcgagcgaggcaggcaggcgggccgggagggagggagggcgcgcgggcggcggcggcggcgagagcagaggacgagccgggacgcggcgccgcggcaccagggcgcgcagccgggccggcccgaccccaccggccatacggtaatgagcgccgctgctggcggcccggaaggccggcccggaggcggcggggagccggggagccgggcggggggcgccgcggctggcagtgcccggctcggggcacgctcccggacccctgcaactcggcgccgggcgcccgtggggccgggactgcaggcggggacggcgggccgggctggccggggcggcgcgggggatttgagggtttgtttatgtccccgcgggctcggcaggcgccccctcccgtcccgcccgctcccggcgcggacccccctccccgcccccgcccggctccgggctcaagttcaagtctatacaatgccgcggggggcccctccaccgccggcctgcgccgcggtgcccggctcgccgctgccgcaggggccccggccgggccgcccccgccttccgggctctgccccagccccgcggccccgcctggagcccgcggccagggcgccggagggcgcggaccccgacccagaaggatcggcccccgcggcggcccgggggagcgggggagggggcgggggccgggccccgcgcaggctccgcggcggcggcagaaaatggcagcctggcacgacccggccccccgcccctccccccggcgcccggccctcctcggccccgcggggcgggggacccgggaggcccccgggggcgggccgaggggtgatggcggggccgggccggggagcgccgcgccgcgatcctcctccggccacctgtggctgggggtgcggggcgccggcggggccgggaggggcggcggggccggggagagcgcccgccttgggggcacaccttcccccggagtgccggcggcacggagaggggtggtcgtgcccggctcctccctgtccccaccgcgggccgtgggtgcccgggcggccgcggtggcggcgccgcaatgtgggggcggggcgggggcgggcgaggatcccgggcccgggcccgggccgcgggggccggaaaacgcgggggtggaagtagcggggccgggaccgggagcggcgcagggggggtacgcccggggtcgcccctccagcacccccgcgcacactcccacgcacgcggacacactcacgcacacgcaccccgccgggccggtgcagacgtgtccttgaaagcttcgcatctgcacagtgaaggacgaggcctcgccgcggctgcggagcggggtcttcggccccacgtccccgcaccccgggagacctcgagcttcggagccgcaggcggcggctgtccgtgtcccgccctcaccccagttcccctcccccaggccgccctgccttccctccccttcggcgcggaccgggctggcgcgggtccagggcccgcccctggcctcccccagcccctcatcgctggggcccgcggccctggggctgccaaactgcttttcgctttcctgaagcctcccttcttgggggcaccctctcccaccccagcgcccctctgccatttctccccctctcctcatcccctccgtcctggcaccctctctccgctgcacggccagttgaggaggtgttggcgccctgggcggggagcagtgccctctggttcccaggggctgcctcccccggctcctgaggggagccccctgcggctgcaccttgacccaacatggcgtctggcacccagcagggcagctctggcctgcggcaggcgggagggtgggcagggccgagccccagggccgcgcagctaccccccctccaaaccagcggggacagaagccttggggagaagcgctgtccgccctgtgacccaacttgcccctcactgccgtgcccagagactgccatcagcatccctgagggcctgctcagtcttcatgtttcaccctaggctgccagcggctgaacccgcctcctgggcgggcggctacgaagggatcctgggatcaggtgctgctccagtgtcgcctcctccctcgcgggcagatgtgacagctgcagccaggggagctattcgccttgctattgcctgggtgttaggactggcctgtgggatttggggggccaggcatcccggggaccctctttggttggctagcaggggccatctccaggtagttttctgagctgcctgcctttggagagttgggttgtagggagttcctgctgggtttctggggcaaaatggtccccctctgcctccaggttctaccgaggccctgcttgggctgtgacaggtggctctgtcaaggtgggagtctgtgtgtgcacatgtgctggctccaggcgagggaccaggctggcctgtgcagcagctctggcacctgcctgcctccctggtgggtccccctcctctgtgcagctcctccccagataagcagctgcggattcaggccgaccaaacttccctcgttaccccacccaggcaccagtgccttccataggagactgaccctaagaaaggtgaattccttctagactcaaggagtggactggtccacagggctgccctctgagtccttcctggagaagcctaggaaaggacgccagcagccatctgtcagggtgtctgcggacaggattcctctgtggggtgggggttggactagagccggaaggtccattccagctttgacattctatggctctgattctagtggtggaatttcaggcctagcaatggggacagggtggtctgtttttcggcacatggcagtaataggggaaggcttccaaggagaaggagatatgagagagatcagggcgcccagcgagttgggccttgggaaggagaggcagtgtgaggagggtccctgcaagagacgggttggtcacgtccgactctgcagagggtgtaaaaagtacagcctcctttaagcctcggcacgtgtgtatcctgtaggtttgtgtataaatccctgtgtgtggataatttctgaggtttccttttcaagtccaattcacagtaagctcctcagtcccttatggatgttttactatcgctttcacacccacctctgccagtggttctcaaaaagctaggaagacaggacaaacggtaaaatccttgtccagggtcaccggcaggaagaagtggagccaggcctgcactcgggggaagatgccccatgctatacctcttacctgggctgctgcatgtgtctgtccccgctctcagtttgtgatctccaggaaacaggggccttgcctccctgggtccccattggacaagcggtgaggcctccgagggtgtctgctgagcgaggggagcgcactgccctccagtcctctccccgaggcagcgaggcaggttagtgctgggctcccttccccctgctgctgtgcgccgctgcccagcggccactgcctacttacaggccagctcctcagggtcacacctccctgaaagacccaagttctttcccagcaagagccagactcccccaaggcttctctttttgggatccaggtagaaagaggactctaatgggtgagttaaaacagaccaccctctccccatctttgggcccgaaatgtcagcattagaacccaaaccttagaattgcagtctgaaagaggagtttaagctctgagagctcttagtgctgggtacgggactgtttgtgggaggtggtgcccagttcttcccagggttgtccatgctacccagagcctgaggcctggagggccattcacgtgccagtcacatgcctctcccagggtacttcctgctgcctccaacttgctggcctggggtcctggacttggtgctcgtatcctccccaccccgtgccccactgggagttgagtgtgtctctggcctccagcaggtagggccagagcatctaccccaatgccattccccatgctgcggccctcggggaatagcttctacttttgtttgaatattagtatgtgagcagagagcccgtcagaagcccacctggcctgaggctgggtttctgcagggactttgctgttcctgagcagggctttaatggtctactaaaggtgccagatccctttccactttaattttgagggctcattttcaatcatttctctctgacgatttttccctaagaacatgaccaaatctgagatccatgaagtgtcagcctgagatttatgtctgtatccgagcctgcctccacttggctaagtgtgtgggcaagacgaggttcagtgcacatgtgtctgtgcatgatgtgtgtgtacgtggtgtgccgccacctgtggatgggtaggacttgcttccacatgagccagtgtcaaggcagcttgaggccctcgagtcacagggagctgcactctgacacctcccagcggcaggaacttgggcaacaactcgattccctgggcctcagtttcctcacctgtaacatgggagtgtgcgtggattctgcttacaggtggttgtgaggatggagtgagatcatgtgcgttacagccttagaggagcgggagccctcctgttagctgtgtctgttttcatcatttgggtggctcacgtggactagcactgcaggaagtgctctaagaagcctcgtccaccttcctgtgtcttttgcagaatcttggtcagatgaggaaagtgccgattctgattaaaaatcctgggaggtgggaggcttctcagcgacacttagcaatctgttgagttgaacaaatctcgtgtcaacaggttgttttgcttccacctgaaatccctcccgctgtagtttcagctcgttattctcatcctgtcttgagtgcagcagcactttcccaccgtattcgcttccattttccagagatgtgctgagcatttcctgtaagccatgccctgtgctggctttgaggggacacggtgtgtttaggatgcgcccagctaacgaagaactgcagagtgggactggggtcactggagagggagaaataaagtgctcaaaggagagtgaggtcacaaccaactaaggtcatcagggaaggcttcttgaaggaggtggcatctcagaagcgccccagagcacagaagtggggcttcaggggcaggacatcattgtgggtggctgtagtgcccgggaggtggactggggcctgggttgtgctggaggccaggctgaggccctgccttggtttggggaggagatccctgcactccggaactcctctgtggcccacggaggatcgctctgaactgcctcagcgtggcggccagtgggggtaggggtggagagaggacagcagggagggaggggaaggggccaaatgagctcatttggtgtcggatcctgattctttctattccatagaccagagcttaagagctcagacttggagagcaacagaacagggtttaaattacactgaccccagggggttgtgacgattaagggggaggccagggtcaggtctggtctgcgagcgagtgatagcagtcatggttgaggatgtggcaggtggagaagggtcccttttctggcctcccagaggtctccatgtccccccttgcccactccatcaggctgaggcgccccagcatccagaacattcctgcaaggggctgccagtgccagggaaatgggggtgaagacgtcaggttcctgtctctgcagcctgaccctgtcccccaacacacacgctgggcagaaccgagccaccaacccagaggggaggtgccctgcagacggcccaggcccccaggcatgagtgcaggtgcctggcagccccctgcaagaacagctggtgggcagggccttctgggccaaatctcccttgctgatcacatcacaggccaccaacctgtctggaggacagcaagggtgcttggtgggcagtggaggtgggggtaagggaggtgcagacgcctggggctttcctagctaaatcccagtatggagtggtttgtttttttttttcagcagcagctctatgttgcctactcctgtcctactgtggtcctctctgacccttaaaggctgcccagccttatttagatatatgaccaggttcctattttctctttcctgtggtcacggccctgctcaccaccatccctattgatctttcctctctgcttctgtttcaaatgtcaaggtcatttttaattctgagccagttattagctgccccgtctgagtgcacagaggggacggaggaccatctcttatagccagtaaccggagagctggaacttgggtctcctgactcctagtctaatgctcttcttactataatgaaaggggaggggggctcagtgcagatgttcgagatctgatagatcagctgggtcatgggttccagtgatccttccttgaaaaggtaaaaagatgcttagagattatggggtgtgggttagatttgcatctcactaagctgttgagtgtgggctgtgtcctggggaatacttgggaacggccatggtctgggggcaggaggccttcggagtgtccctcccccgccactccctggaggaccccgggagacacaggcgagttcgcccctgcccctgccaagccagtgggattccccagcaccccagtgagcagaacctgtttggaaagaggcttctggaaggcctgcacgattacttgtataggttcccgaagtgataaataaatgcatgggtaaaggggaatccacttagattccctaaaagcctttgaaacaaggtggtaggcgagtctgttctgatgtgttagatccccatgcagtagtgttgtggttaaaaaaaaaaagttcatagtgtaatctcagttttgtgaaattctttaaaatcgatacaaatatacacatgtttaggtttgcacatgcatggaaaagttgttctggaaagatacacacaaacctgtcaacaagtttttgctttttaactttgtacactttcgttttctttgaaaaaatgtaaatgaacttgagttactttcttaatgacaacaacaaaaataaaactacactttttaaaagcctgtggatgagaaagtagaaaaaagaattaagtcacctaaggctgagggatggactcttttttataatggataagaaactggtttagagactgacagcaaggggtaagcttactcaggtacgtgtcttttccggaaaggttgcagggagaaatatcgcagatattaaggcctctgagcaaaggcctaggctggtagggattaggtgagagcagaaaataggccgctgactgcatgcaccctggcccacgccagggacaggggacaaagttagctttataggatggcgacttgaaactcatttctgacccaaagtggagaacaaatcactgaggatcttcccccaaaagcatcagtctgatgggctgttgtggcccaaatggttcctggggaactggaggtataactggggcccggcccgggtatggagctcaggtgacccagccctggcatagtgagagcctcttgggaggccacacccaagggagacaaagtggggcttcaggaggccccacgaaggggtagaaggggtgtctaggaacttgcttggcacgtgggttcaactctgatctaggggtccctgagcaggtggtgatcacccagatgatggggtggagaggtgaggtgggggcaaatacaagttcctctgtctgcagtcgtcttgctaccccaggaagctcagggctggttgcttagcaacagcacttaccaccctcttccaccttaccctcccgcaccccacccatgggtgcctcttggggcttcccttcccccgaggctgcccctgttcaataggacatgggaagggtctgtgcacttttttgaccctgcccctggaataaggcaggaacagacataaaaggacctatgacttggaggcaagcacagaactggcccctctacaaaccagctgtttaatccaaggctggtaggggacagatgtgcagggcaaaaaaggcgttcgggagtcaggccgacctggaggactgtctcagccccatttgtgctcagttgctgctggccccaaaccacctggagctgcaggtttttcatctgtaaatcggggataagaatagcagtctcatcttacaggggttgcctgaatgacgtgcaatattttgtgaaacagctagcactatgcctggcacagagaaggtgctcaggaggcatgagctgcttttctttacgtgggggttgttccagggctgcctggttagggtgacccccacaaagccctgctgctctgtaaggccctgctgctctgtaagaccctgcaagtggccaggtgcagtggctcacgcctgtaatcccagcgctttgggaggccgaggcgggtggatcatctgaggtgaggagttcgagaccagcctggccaacatggtgaaaccacctctctactaaaaatacaaaaatttagctgggtgtgatggtgcacgcctgtaatcccagctactccggaggctgaggcaggagaatcacttgaacccgggaggtggaggttgcagtgagccgagatcgcgccactgcactccagcctgggcgacagagtgagacttcgtctcaaaagaaaaaaaaaaaaaagacctgcaagtgtggcctgtcctccccatggcccctcacggtgaggggaagagataatcaagttgagaatgacccccaagcccatggtggactctgggctgggagtcaggggccacaggggagtggcagggatagagaaagatgtctctgtcctccttagcttccgttatctgacccaggtccctttttccagctgccaataagttgtccctttttctcttagcctttctgcacctccacttcttcctgtgaagccaggatttgcctgtaccttttttgatctcttcagagtcccctgtccatgtggggacccagtagcctgctcaaggcctgggccctctttccctccctgcccactggtctctttggagctaagcggagagtggcctttgctgggttctggctctgccccacggcctggaggcaggggagggtctagaccacagcccggcctggcagctggcacgcagcaagggctcctaatggttttattaccagcaacaatgtggtgcttgttgccatccacgcacaggctggtggttcccagcccaactggtggtggcctgagggccctgcctggtactggaagctcagctgggaagcccagtggggctgggcagtcagtcacctctgcgttttcttctgtcaacaccctgccagcagcctggaatcagagggagccagggcacggcatggcgagatgctggcccccgagttggcaggcctggcagcacttgagttttctttccagaggtccctgcctcagatccacaaatgatgttcatccattcatctaagagacctactgtgtgccagacacaggctgcatagatgaagggggtcacactcatctcaatgaccctgcagcttaggtggggtctggcagggcggaaggacccctggcacaccatgtccagggattaccagggctcagagaaggaagtcggattcagggggagagaagatctggaagggctttctaaaggaggtgacgcttgagctgagtctcgaagcggagggagcagttagctaggtggacgacggcactttctggccccatgtggctgcagtgtagggaacgatgggatacgaagccaggggcctgggccgggggacatggaaggcgggatgttatgaacggacgttgggcttgggatttatatttgagaaggatcattctagggcatcgaggacgagagctcgggcaatagggtttaggggcagggagcccgggtggaaggctggcacagaagtccggggcagcaatgtgggcctggacagggtgtgcgtggtgtgacatttaggaagtgcagtcagtaggaacaggcgggataaaagcaatgaggggagttggggtggctgtctggtttctagctcagccaactgtgtgggtggggatgcccttcatggagggggcatgggttggggggagtcggagagagcaggaggtgcctcccacatggagctgggttgtagctggggcttgagggcgggtgggatttccccagggggaggctgagccaggccaggcttgcagaaggaggctggagcctgagtcataaggcctggaggcggggtgccttgtgcgatggatgagaaaggcaggtgcatttcagtctgtaactggtacgacccctgcagatgtttgaaggaagattttgaggaaggtgctcgcacccttgcattgggaatgctgatgagttagcgcctctacttcacagtgatttccccaaaagatgagctgcaaggcctagaccctggccctccacccaggacctgcggcgagcatcatcaccgtctgagaatctttgggtgggctggaagttgatgtcaagctgtccagcctttccgtcactctccattcagaggcaggtctgcggagagagataggagggacaagctggggacagcatttttggaaagcaaacagaaaagaagcaaaaactgttcctctaaaatagaaaggaggcttatgtgtgttatgtgaaggataatgtggaaggctatttccaaagaatttctcaaaaaatgacgtgagccatgtcagcatcattgaatttagatagaggccaggagcagtgtggcatcgtgctggggccaccaggctggggactggacagatgatggtttcacctcacccctgccacctccttgctgcgtgtcattggaagatcactcaacctttcttaggctcagttttaccatctgtaaaatggacataacttcacaggctggtgttgtttgttttgagcattgcttcttggactaaacctggatattttttccttccctccctcgcttctgcacttcctctggttctttgcctcccaagaagcctgtttcctcctgttccctccaaaactccaagttttgagttcccccttcctgggaaggtccattcgtcatgttctattgactctcttgttcccaattattccctgcaaacctggttcacccagtgtatttgaagagcatctactgagctaaatgggagttattgggggtcagttcctgttttctttttaataaggggagggagggtctgtgcagagctgtccgggagagcagagggcatgggggacaggggggatgttgcaggacccagtgggaccacagctgggaccttgccctgtagctggctcagtgtgccaggggataggggcacagagccctggtgggtctgggagatgagcttgggaaggacttggcctggacatgggctaggctgccctgctttccgagccagacctcaggagatggcagagagggccgcttcctctgtgagggccgtacatgatggcccttgggctgtatggtggccttgtgacatgcagccacacccctggggagacctttgaagcatctctgcattctggaggatctgctgggaagatggcttcgcagcggggctcttgttgatgctgccaagaaggccgggtgggagtcgggctgtctctggctgggggtggggtgggggctgcggtccatccgagtgtggaaatccacgtttctctggtttgtgcagacctgtgcaagcagccatgctgcaggactggccccctggaagtggggcatacatgaactcagggtgtggcgttgtaatcaaatgaaatgtacagcataattaatctggcatacccttaatgatatgcataattgtgctgcatggtaattaggtgtaaatggtaagcagttcacataaatgtttttgtaattggtattaattttgcagaggcaaaccaagtttatgtggtttttctcttcttgctctgttagttttctagtacaggcatttccaagcttttggaggaattctattgcaaatgtttgtttatagggcaatgttttgtgttttctctcagaacaatgttatgaatggttatgaggttcccaagctggtttccaaatgcctctttggcccatacagtaggtgaaatgtgcaacgcgtgtgcctgtgtcggaaggcccgcccagcatcctctggctccaggtgccccagcgtgtgcagcatgaggggggctgtgtgcgtgtgtgaacctgtgcgcctgggtcatctctgcatttgtctaggtatatctctgtgtgtgcatctatgtgtgtttgtatctgtgtgtctgtgtgtgtgtgtgtgtgtgtgtgtgtgtgtgtgtgtgtggggtctgcatgactgtgagagactcctgaggccacagtggcaggtagacattctctggtaaatacattccccattctagtggcacggggcctgggctggagacttaggggcatcagctgggagggggagcctggcacagcagccgttcctaggagtgagcacaagccactctgaagtgtggtctgaagccagagtctaggtttcctaagaacatatggcttgcctttctttctgatcaagcacataataatttttaaatgatcatttattgagtacctgccacgtgccaggccctatgttaaacacttgactcagattccctcctatcttaaaatagacttatgaaatagggactctttctcaccccgctttacaacttgggaaactgaggcccgaatctctcagtgacagagccagggtttgaggcagggactgctggaggcagggcccatggttctgagatgtgctgctcagaaccctgggctcttctccctccctcggccccctccacaaatggtttgtttttgttgggacagacgatgaggagaaaggcggccctgtgggaaatctgagagcagcgagctcccttcctgctgaattggttggggtggtgggggacgagctccaacagccgctcccaagccctgaagaccctttgggggacctcctggttttgacccaagagggagctggtttaggaagtggggttgggggactgtggccatggacggtgtggggagagggtggtcacaggccagacaaggactcagcccccctgattcctgtggttcactgatagcctaggccctcaccaaccccttcaccttggggatctggggcaggtgagcctgcagacccacagcaggtgtatgttctgccctcggagctcctgcaaacagcctttccactgacgcagtgccttgggggctctgccaagcgacccctagaatggggattgtggggggtcgctctaggcaccgcagcactgtgctggggatgttgcagctgcctgggagtgacttcacacagtcctctctgcctccagggtcacccggaacggggttgcgggggacaagctgtgggcgggggggacgggcagcaagatacaacgcatgaactctgacctcgtctctttgagaaatcagacacatctatatacgcatggaacactcaatgacccctgggtgggtggtcgaggagaaggggacggtgttttgggggaggagagatgttgggttgaaggactgggagagaggagctgagtgggccctgtgggagagggtgggagccaggggtgtcattccgggacagcagccgggcctggaatgatgcagctgggagaggtgggggcacggccagagcagtgggcggaggctgcgttgtagccagccctggtgagaccaggccagagcgtatattttaccacgtgtgcaaatctttggcatcaccggatttttaaaaataatagctttaagatataattcacattccatacagttcacccatataagtgtacagttcgatggttttagtacattcacaatcaactttttaaaattgtcatgaggtgtcataggttttcatagagggagccatgtgtgggcaatccttgctgatagggtggggtgggaggtagagatgccagaggcaggtggacccgctccagggcatctaccaggttattgtgccacagacacaagaggtacaaagggcctggagggaatagggggacgagagaagcccaaggaccaggtacctgctggatgtgaggacccaggggaaaggtgggacctgagttttctccagggtttgggacttaggcagctgaagggaggggaatgttacttccacatagagggaaaaaccagaaaatagagccggctctgaaagaagcatgttctggaagagtaggattttagggactgcaagtcatccagttgggatgaaaataaatagagctggagcccctggcagtaggaggcgtatgttaacttatgcccaggtgtcttagaggaagagcatttagagagaagctgaaggtagaacatgacctttgaggagcacccaccgtgcagaagtgggatcaagactcccggacagccccagagacaaagctgcagaatctgaggctggagacccagaagcaaggagccagatggagtcatggctgggagagaacacacagaaggcagttgctggtgagctccagaagcaggggtctcctttcactccagggcccggctccacaatggcactcaagaaatggctgttgagtgagtgcttgaagcagggagtgattagctggccaggatgttgaattgagctcaagggccatgaggatggaaaaaagccctttggggccgcgggtgactgtatcagtcagggtccattcagaagacagaaaccacatggtgattggaacaaggaaagtttcacataacgaattattaactctaattggattggaataatgaggaattggctagtaagaagtaaaaataactccaaagaatacaaaaataatagatatgagaacagcccctacctctagggttgccacagagtgcccaaggaagaggccccgcagggttgagatccacaccttgatggagagggtgtggcgatggctccctggatggcaacgtctctgtaatgccatgttggcagaacttgctggaaatccaccctctggaatttactggaaatccatctttttgggtcccaaggaaagttgtttgtgggaggtatcttgctggaagcacaccgctgctacgttttcccaggggagtgctgggagaagctcctggctgttgggtactcctgactgacgtgttgctggagccaggtgctggagaagctgtctgcattgcaacagcctggcactcctgcaggagctgggcactggaaagctgcatgctggagaagctgcagcagcacaggagctgggtgctgggaagcttcatatctggcaagaccctgatgctgggaagcgcccatgcagcaagatctggctgctagagaagttgtatacaatgcaggaacccaacaagagcacactgggtctagaaaggaaacccacctcctccttcaatgcctcttcagtgccctccacagttgtgccagctgacaaaggaaaagtagttaaagggcccagctccatcttcacagcacaggcaatgatgggcgaataaactgataactggcacagtttacccctttggctgcacagcttccatttgcacctttctacaacatctgaactcccatacaaaaacaaacaactggatgtctacctatcaagatagttatccttcttatgaaattctcacccttttgcccagagagccccaaccatcattctaccaatccctggctacaataacttctcaaagtcagtcacagtctcactgaatattctgttacctaaagactacattataaagctaactgtcaacaacttgtatagaaaagaatgggaaggagaaaaaaagaagaaaatggttaccatattgcaaacacaccacacacacacacacacacacacacacacacacacacacacacatatatatcaagttaatagaaaatgtgcaaaaccactagaggcctggtttctgcatttgttcacaaggctacactttacacttgctacctggcttcattgtcttcttatcccattctgtgtttcctccacactcagccagagccttagcaggtcatgcttcttttacttggtggagtaacccaaaccctcattcccaaaggacctgaatccttagttatcccgcttttgttgttgttaccataactttccattcagccttcctttgggcatggacgttcttaagaggtgcctgagagaattcgctgggttccagacacagtcttgcttggctctgtggctgatcagcaatccagttttctccttggtaactaggatcaatcactccagacagtggcataactccttttttccactgtcagttcagtggcgtaaggagttcgagaaagccaagtgggtgtgtgtgtcttctctcccagggaaagcattataatatgtccctggtgggagcactctcccccgaggacgaggtcagcatccccagaaggttttagagttgtggggtggggaaacagggccacgcgcaggggactaagtgatagaggctctggagctggcagctgtggaccatgctgtagagaaggttggaaaagtagagaaacatgcagatgaccgtgggccgcttccgctgtggatgataaaatcagacccatgctggtcactgccagaccccatccaggaccatgtgtctgattctcacatggactctgtgccaccctgtgcccctgactcccaggccgtgggaagcccatctgacccacagcacgtgtgacccctagacagggcttctcctggcttgcagcacttggaaatcggtgctgccttacggaagtctgtgttggctcattttggctcctgagaacaacttcagctccttaacagcatctattttaggtataaatcccccccccgctcacttttggggctgtgtcatggcaaccattagtgtgagcaaagcagtctgactcagactcccatgtggccgccgtagtggcagcaggtttggtgtgatcataagtgctcctgctggtggtgccagtgctggtggtgccagtgctggtggtgccatggcctcgagagtggtgtgagagccacaagccgtcttattgtgactctagagccagataaatcagctttgcagagggcaggactgctggcctgtcaccagtggcttttggtattctatggacagctggtagtttgggtgcaagtaatgtttctgaaagtcagggcaatggaatggtgatggccatctcactggaaaatggcaccaagacccccctccctctccctcctccttatccatccccagtcttatgaccaagagaggttgtgacaggagggggaagagttgacagatagacatctccgtctaggaggtctcctccactttctcctggcccctagctgacccctaccctagcagatgattgggcagggggtgggggtggggcacaagatactctgctgccttctgtcacccacatttgcacctcctcctaagctcaaataggaaatttagccatgggactgttatctgtgggccagggtcaccccagaggcacaaacatcgatcatggcgctggctctgagatgtgtgtttagcatgctcattcatgaccaagaactgccacatttgcgaagggttctgagttacacggacctggatttataatcctgccctggggctttctagctgtgctaccctagagaggtattaaacctctttaagcctccatgtcttctataaaatggggataacaataggacctgcttcataggattgttttgaggattgaggatagtgcatgttccttagcacagtgcttggcacatggtaggtgctcaggaaatagctgcatgaatgaatgagtgaatgaccactgcattgggcagggcattgaatactgtgattagcatgctccagtgccctccttccccgctcccactgcagacaagcctgaccaggagagagagactgactggaaactttgttttctggccagtccgtgcccaaagttaatatagatctgcagcagtcacatgctgattgtgagagagggagtggagagctgtgagcttctgagtggggtggacctggactgtgcctggctccaccccttacccactgtgtgaccttgggcagttttattaatctctccatacctcattattctcatctggaaaatggggataaaaatagtacccacttcaaagggttggggtgaggtgtgaggattaatgagatgttgacattgtaaagctctgctcacggggcctggcatgtcggaggagcttaagtacaagctcctgcctgagcgaaggccacttagaggaactccagggatttcaagaaaggagagaaatctgcgggctggttgaatctgagagtaatcatcgttaagatttattgagtactttctctgtgccaggcgctcttggaagcatttttagggcattccattttaattttcacagcagcgctgtgaggtggaaagttctttctcccactcgacagctgagaaaactgaggcacagtttcgcctcaggccacacagctggtaagaaataaggccagtgttggaaaccccgcagttggcctcccagggagccctggaggggactgagattagcccttaggtcaggagggtgtcaaccacaagtcatagactcataccaaagtagtgaaagctcacagtccccggaagttcagagacgtccctgcctccaggaacagcaagaccaggggcccagacagtgactacaacgctttatccctctgtcttcatctctgagctcttcgttctcaggcaggctcacccacatggcgggaagacagcagctggcagcccctgactcacatcgaccatgttagcatccttccatcgcatcgataaaaaggttccagggaggcctctgattggctctgcctggtcacgtgctcaccacttgcctggggtgagggttgaggtcagcacaggcacaacgaaaaccttttgacaagcgtttcccaccgggaaggaggggaaggcatctaggtagagcagaacaaccagagtccactagaggaaaaggagaatgcaagcagagggagggagaaggacctgctacagaaatggtcagatatttttcagccattcagacagagaacttgagacggagaagaaggggcccttagggtgagaatattgggatcgtgtttggtggagtgaggatttaggtttgtgggccgtgacagttttggttttggaagaggataattatgtgcaagcccttgttgaggagagactgtggggtggcaggggggccagcagtcctcaatctgaagatggagaggaggcttgggtggtcatctcgggggtgctgatgaggctggggttggtgaggaagtgggggcggcagtggggacagtgtggtgcaggggagcagtcctgaggcttcaggagatggtggagatgtagctctttggtctgagaaggggctgagtgagcgggggttggggttacggatgcaggcagcactgacttgcgctgcccatgggctggacgcagcgggaatgatgtgtttggaggattaaacatttcgtgagggtggggggtggagagggaggtaccagtgttgtagctggtgatggaactctgtgtgggtgtgaggagctgatgatggttattctgtgggacgtgatgatttcatagaacaacagaggtggaaaggcctcagctacactcttatttatagagaaggaaatggaggccaaacgcaaccgatggctgcagttgggacgtaagccctttctcccatccccactctctctgtttaaaaattcatttttatttactcacagtagtacatgcatataatttaagaagtcgaatagtactaaaacctcctaacaaaatgcagtgattccttgcctactcctacccttatcctcttctcagaggcaaccgctttcgattctcttggctgtttcttccagcaattttcatctcatatttctgagtaaagtgcatatttttgattcatctatttagacattttccattgatttcctatttagtagatcctcttacatgtccaacctttctgctcttcagccatcctccttaaatattgacagtactcttcttatattttttcaaaaaattgtttgttgttgattgttttgagacaggatctcgatctttgcccaggctggagtgtggtggcacaatcacagctccctgcagcctcgacctcctgggctcgagcgatcctcccacatcagcctctcgagtagctgggactacaggcatgtgccaccacactcggctaattttgtaaatttttgtagagacaggatctccctgtgtcatcagactggtcccaagtgatcctcctgtcttggcctctcagagtgctgggattacaggcatgagccattgtgtccagcctcaaaaatgttttgtgcatttggtagccagaataatggcccccctaagatggccaggtcctaatcccctggacctgtgaatatgtcaccttacatggcaaaagggactttgcagatgtgattaaattaatgacctagagatgaggggattatcctggattatctgggtgggcccagtgtaatcagaagggtcattagaagtgaaagatggaagcagaagagtagtcagagcaggagatgttgatgaggcagagagtactgtgatgggaaaaagactggccatcgctggctttggagttggaaggggaccacgtggcaaggaatgagggcagcctctagatgttgaaaatgtcaaggaaatggattctccttagagtttccagaaaagaatgcagccctgccaacatcttgagtttagcctagtaaggagaatctttttttttttttactactgacttgcagaattgcaagataatttttttttttgagacggagtttcgcttttgttgcccagggtggagtacaatggtgccatctccgctcaccgcaacctccgcctcccaggttcaagtgattctcctgcctcagccttcccgagtagctgggattacaggaatgcaccatcatgcctggctaatttttgtatttttagtagagacggagtttctccatgttggtcaggctggtcttgaacttccgacctcaggtgatccaccctcctcggcctcccaaagtgctgggattacaggtgtgagccaccgcgcccggccgataatgtgttgttttaaggcattaattttgtggtacatacaaaagtaatttgtgtgtacatacatgcatatatacatatatatgtacacacacatatatttgttaaacctacacacagtgtttattttattatttcgataaaactattattcacagctgagccttgtggtgcactgtaatagtttcttttcgtttttttttccctgaagttaataattaccttgtttctcatctgccttatttttttgtacctgttgctaattcctaccacaacttctaatataattttatttttctttaacaatactgttaccttaggacttctttttctttttttctttttaactgacacataataattgtgcatctctatggggtacaatgtgatgtttcaatacatgcatacattgtgtaatgatcaaaacaggatagttggtatatccatcacctccagcatttatcacttctttgtggtgggcctaccacactttccaatagccatcagtattgctttccacacgggcagtcacagcaggtaaaagatgtttcagttccattttttaagttatctatcctctttcactctgggctagttgctttctaggcacattgtagctgatgctctgtgacttccttttgtggtcatctttggaattcccattactttccggttggaatctctgttttctggctttcgtgtcctcctcatgcttggctcactcccctcattagagcacatcttccaggacacacatctgtccatatcaaaatccattcatggctgggcacagtggctcacgcctctaatcccaggactttgggaggccgaggcgggtggatcacctgaggttaggagtttgagaccagcctggccaacatggcaaaatcccatctctactaaaaatgcaaaaattagccgggcatggcggcgggtgcctgtaatcccagctactcgtgaggctgaggcaagagaattgcttgaacccacaaggcagaggttgtggtgagccgagaatgcgccactgcactccagcctgggctagagagcaagggtccatctcaaaaaaaaaaaaaagaaagaaagaaaaaatctattcagttcctgggtttagatagcagagtatggccacgagcgatacctgtgtagttttgtcaggtaaaaagacagttcgacagcagagggcatggtgatgacggcactcgtgccccagtctcagtgggaaggctggccctctctggctcatgccaggtgactggcatgcggcctcttttccccttcatcctggagcctcccttcccatctctcctgtactggatcttatgcgtattagaggacatcttcctgtccttcctgggaaaggagcgtgaggcctcatatgtctgaagatgtctttactagggcccagcacggtggcttacgcctgtaatcccagcactttgggatgctgaggtgggaggatcacttgagcccaggagttcaagatcagcctgggcaacatagtgagaccccatctctatttaagaaaaaaaaaagaccccgggcgtggtggctcatgcctgtaatcccagcactttgggaggctaaggcgggcggatcaccaggtcaggagctcgagaccatcctggctaacatggtgaaaccccgtctctactagaaatacaaaaaattagccgggtgtggtggtgggcacctgtagtaccagctacttgggaggctgaggcaggagaatggcatgaacccgggaggtggaggttgcagtgagccgagatcccaccactgtactccagcctgggcgacagagcaagactccgtctcaaaaaaaaaaaaaaagaaagaaagaaaagaaaagggccaggcgtggtggctcacacctgtaatcctaacactttgggaggccgaggcaggcagatcacgaggtcaggagattgagaccatcctggctaacacggtgaaaccctgtctctactaaaaatagaaaaaaaattagtcaggcatggtggtgggcgcctgtagtcccggctacttgggaggctgaggctggagaatggcatgaacccctgaggcagagcttgcagtgagccgagattgcaccactgcactctagcctgggcgacagagcgagactccatctcaaaaaaaaaaaaaaaaagaaaagaaaaaagaaaatgtctttattttactctcacacttaaatgataatttggctgaaaataattctaattggaagattgtttaagatttggtttattctttaagaattttggctgggcacggtggctcacacctgtaattccagcactttgggaggccaaggtgggtggatcacttgaggtcaggagttcaagaccagcctggccaacatggcgaaagctgtctctactaaaaatacaagaattagccgggagtggtgacgggcgcctgtggtcccagctactcgggaggctgagaaatgagaatcacttgaacccaggaggcggaggttgcagtgagtggagatcatgccacttcactccagcctgggcaacagagaaagactccgtctcagaaaacaaaaacaaacaaaaaaacccaactgatcggaagctctgagaatgatagtggacttgttggttttcaagtctacataggctgacctgaataagtcattttgaccccattttggtactgatgttggtattggcatccttaaacctttcctcttagactcattcgattctccagagaaggaaatggttgtctggctgccagtattcttagagcccaatcggggaatagcctgagagactggacatcagcatttaaaataaccttcccggccggcgcggtggctcatgcctgtaatcccagcactttgggaggctgacgtgggcagatcacctgagattgggagttcgagaccagcctgaccaacatggagaaaccccatctctactaaaagtacaaaattagccaggcttggtggtacatgcctgtaatcccagctactcgggaggctgaggcaggagaatcgcttgaacccgcgaggcggaggttgcggtgagccgagatcgcggcattgcactccagcctgggcaacaggagtgaaactccatctcagaaaataaataaataaataaccttcctctccttggtttgatatatcatctttgtcttcagctgtgccaagtgactctgtgcaaagaccccctattttatgccctccagtgactacagataaaccttcaaacttgtagaagagggtctaggaattcactactctcactcagcctctcgtttctcacacaagcatcctcttcagccctagtgccagagtacctggtgctgtcatataagcatctgttgaagactgtgttatatatcagcgttcacacgcctgtctgaaatttggctgtcacaagtctgctttccagattccacaattttgttggtggtattccttctcttgttcagcctaatcttgtgattttatgtcttaaaaaatcttttggccaggtgtggtggctcatgcttggaatcccaacactttgggaggctgaggtgggaggatcctttgaagccatgtgttcaagaccagcctgggcaacatagggagaccctgtttctacaaaaaaaaaaaaaaaaagccaggtgtggtggtacatggctgtagtcccacctactcaggaggatgaggtgggaggatcacttgcacttgagcccaggagtttgaggctgcattgagctatgatactaccactgcatccagcctgggtgacagagcaagaacctgctctaaacaaacaaacaaaaaatgtttctattgcattgagtttagaaggtagtgagccttggtatgcatattcaaagctttcagttctttttccacaacagttttatagttgacgcagtggagatggtgatcatatcagctgtgatacgaagatgttttcatggtgggattccttgaaattttttaagtggctgttaacaaacattattcatggtcactgtggactctaaaaggaggaaataaaagctgcttttcatggtggtagggttcttggcaagggttttttggtggcagtggtggtggctgtgcagcccttggcccaggtgggaagaacgctttggtatcggaagcagccagggctgtgcggtggaaggaccgatgtggtggacacccgctgctgtgttgggactgtgggcccaggagagcccactccccatggctgggctgcctgaaggaagaatgaagcgtctacgggtaggtgggcaatgctatgtcaaggtctgggaccttggccaccaactggggccccctagggttgtgtagtacatggcccgcccagacacatactggaggattttaggtgatggacacgattttgagggactgctactgacagggctggtccattgagcaacacgaaagggttgacccaggtcagctgcattttctgtgaggacagtttctgcagctcagatgtcagagtcttgggtggaggaaggggaggaggaagagaatcttcctttcctttcctgtctggggtgtctgcccttggggccaatgccctgtgaagttctgcttttccctccagcttccttcctctccagagcttctctgaacctcgccacacccgtggcggcacagatgccctctgggtgtccctaagcctagggcagcatcaagacaaccctcccgccccgcttcctttgtgcctgggtctcccccgactccatttggggtgccctgtccttgtcagccccttgtgtgtccactgtgcctgtggggccgcaacagcttccttggccggtcagagctattaatagaaggggtcctgctctcccatttcattggtggggaaactgagggccggggaagcctccgtcaggagtagatcccacccctgcggactttgggctcaacgttctttccaatcagactctgcccctcttagcttctgaccctttcgaaggtttttgttttagtgtggctggagatgtctgtggtaacaaatggggagtgaggcttgacagggaatcactggctgctaagggctggcagccgagtgcttctgggttggaccagttctcttttgggatcttcagagttgcttctgtggtgtgtgttgggggacgtgaacccaacccctcagaactttagtgtcagagagatctgggttcatgcctctgatgggtcctggccttgttgatcttaccgtcacctgtaaactgggcacggcacagcagctgcccagatgcagagcacgagccaggccctgagtggcatgtggccgatggagctgctctccgctgccgtccaccctctcgggacccgggcagcaaaccttccttaagcccagcactgcgctgtgcctaggggtatggggaatggacgcagtgcacctgggctgcctcagggctcagctgtggggcaggtccgcttcaagcctttctgggttgacctgatggagctcttcctgtcctggccctaagaagctcctcctgccagctccttcctcccactaccccctggggcacagccagggctggggcacccactcccaggcaagaatgcacccctggagaaaggggctaggcctgtaggagagccaagcctgtgctttctcccggtcctggttttccagggagccctgactggagggggatgggaggagaagcccaggagggagggggagctggggaagcagaaccaggaactggaaggggaagggtgtgggtgggcccagatccagcctgcctcccgctctcccttgcctgccaatcccctcctgggccctggcacactcaaggcaggggcttggcggccgcctcctccgggggtggggagcaggggagctcttgtgtgtctctgagtgagggggctgagacagcaacacccccaccccagctgtggcctgagctgcaggctttgttatttctctggcttgaggcctacacctggagcagagggaagaaaagcccctaaactcagccgtgtgggttcagaggacccctcaagcccaccaactctgcaggcaaatgaggctggttcatggggtgggggagagagcagcctcactcctgcctcagtgtggccgtggcctgtggccttggccgtggccgtggcctctccctcctaggccagagccaaccaggcctgcagcctgagctcagacctcagctttctctgggcccaggcaccggtcctgaagtgaggtgacattcatttcactacatcctgtgactgcactcacttcctccacgtggcgggaggtgtgggggtgggggtgaggacagattagaaggaaaacgtagctgggctaggggcctctgggggaggctgggatttcccaggggctggttggggtgggagggaggatgcgggaagctttcaaaggagagagaggctgtgaatgaagagaagggaggaggcggaggcagctggaggtcccagaagtggcacccattggccgcagcgacacggaggggagactgctgccactccaaggctctcagaggctgctggggtaggaggtggccctgaagggagcctgcctcggtttctctgctctctggggtgctggtgaggccagaaggaggagcaacaccccaggcctcacaggccaggtgtggccctgggctgagggcctgacccagggagggtggggtgctccgccccactgatcctcttctctcccccacaggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggtgagcagcgggctctgctggggaccctctggcctgggagagggagagccctgtccctatgtgatgacagccggctcctcatactgagtgcatgaccgcatcactcagtgctcacgacagggtgtgggaccatcacatccctgtttgggtgatgaggaaacggaggcttgcagaggttcaattctggtgatggagcaggcatttggttccaggacccaggcttttaaccaccatggcattcgcacagtggctcaaagcccaggtctgtgccattgtgagggttcggcctcacagagagtcccagaacctggattccttctcggacccgcctcttactagccgtgggccttcacgtgaatcactttctctctcagctgcctctgtttccttaaggtgttcagtagcttctaggggaccaaagacaagttctggggtggggcacggcagacacgaggccgcgggggcccaccctcccacacagacggaggcacaggctgggctcagtgtggcgccagcacactttggagatggtgatttcctgtccccgccccccaccccagactccaggtgaatgtcgctgggttatctttggtatggcttttggccacggagctggtgacctccatgttttcagaatctagttgggggtggcctctccacccttctgctgaactccaggttgtgcctgaatcagtcagaggagattacatcagactgatgggagtccctgctgggtctccacaggctgtcttgggaccttaccaagctgtgtccccatgataggaaaagctgccccttaacccccagcactggactgtgttgccctctgtcagtggaatggtgaactcactgccaccctccaggggatcttatgatggccataggccttcctggcatgtctgtctgctgtgcctctgtccacagttccccctgcctgctccccttcccctgagtagcgctgggggttgtgctgcttggtggtgtctgggagtagctgggcatgctcatgggggcaccagttggttcttttggaacaaaggtggacacctggcaccagctttgggggccctgggcactctggggctagaggcagccagcaccctacctgaagggtgtcagctctgagacctccctcttgcgtgctcagctccttcctgggtttaccattcttcagagtgtgtttctgtttcctagttttccatcaatggttctatttggtccttggggacaactcttgaaacaaatggggcagggacagatggccccagtttacagatgaggaagctgagccccgctgggcagtggcaggtccagatgacagcctgttctcctgtctcccaggtccgagcccttcccacagccctacctgcataggtcctgctagggacacatgtacacatcccaggagtgcacatgacgcccagggacctgccttcccagcccagcaccctgtagagcaccgtgggggtgtgtgcacccagtcagccagagcaggtcagaaagccagcatgcgctgcggcccactgggcaggacgcccggggcgaccgatcaggctcctcccttctgaatccatgggaaaagaactgatagtcctggatacctgcgggcccctaggctgcccccgctcaggactcaagcagcatgaagacccaagccctttggccgtagagttggggacacctgccgctgtctgtgctctggagtggcaactgtggccatggctgactccctccccttcttgggcctggcttgctttctctgtcggataaggtgaccccgagtgtcgccccactctgggtccgatgtcctgacagttgctgacgcagctccccatcaatgcactggcagtcactggtcagctgctccccacaggtggggagggtggggacagctgggacaaactcctcctgttactggactgtactcccacgccgctgctgctgctctgggctgcggagccccaggccaccaccaatggacgggagcaggtcaggatctgcattataatagctcaggactgagaagcaacgctggaccaggagtcagaaatcccaggtcctagtcctgagtatgccacttactggagatgtgacttcaggccatctgggcttgtgtttgctcagctggaaaatggggatagtgactccagccttgcctgttttaaagggtggttgtggaagtcagataaggtcatgggcatggaagtgacctgcagacagcaaagtctggtccaattatgagggattacgattgctattattatctacccatagtaactgctctggctcaagcggcaacaaacacacacccagccctagatcccggtgctgatagagtgcacactcacaccccagcaggaggggtcatgccaaaagatggcagtgatctggatagataagggctgggccatagccaggcctcctccccagccgcccacacctacaccacgcatgctggctcacctggcacacttccccggcacctctgggctggctccagcacctccagggagccgcccagggagggtggggagagacactgacagccaccacagcagcccatgaaagccaggcctgcggcaggggtgctggtagctgcagaggggaagacacaggcttggtctgcagcccagggagcctggcacaggtgccaggggcactgtgcacaaaggaactgtttggcaagccagatggaaggggtgtggccaaccaatggcccttccactgcctggccccatgtggccctctagcctctgcccaccgccgccccccctccccgcccccgcccccagccccgcctccatgcacctttcactccagccccactcccatacttcctggagtctccaggtctttgtttatgctgctccctctgccgcatgggccctttccctaatctcttctcctccctggtcaaagcctcaaagcccaggctgtagttggccttgtattatagtttgcaggggaccagtggttctcaaagtgggatccccaaatgagcaacttcagcatcacctgggaaggcaaagctcaggccccattccagacctgctgagccagaactcgggggatgggacccagccgggcacctttaaccatccctccaggggatactgttgcgcacagatgttgagaactattggtgtagataacttctgcccactgctagcctggagcgcagggtcacggcccttttcttccatgtgaatcccccggacctggaatagcacagagtgggtgcttgggaaatattttacggatagaacggaaagaaaaatgagagtagccaagaccctcccacaggagaataatcccggagcccaggatcagggaggcggagtgcagagctgtgggtgctgggctgaggcagccatgctgtggctgggagtgtacagagtggcctgtgtgtgtccccctggcacctgccgcctgccctacccgccatccatatggtgggcagagcagggctgcttggggagatggggaggaggcaggccgagctctggggctgtggtcagggcgagtcagctggtaaatgttgccgcagcagagggagggggctggcctcatggagggtgggtggggaggcaagcagtggggaggctgagagcttagaggggagtatggagcagagggtggggggcagagggagcaggacaggggaccaagaacagtccaagggccacctcagatccccaaatatgaatcagaggggagtccacagccccaggccaggcccctgttctcagttctgtcccctttcctctggcctcagaaacacaatggctaagatgcattgaacgccgactgccgggtgctatgtgcatgcattatcttattccattctttttttgtttgagacggagtcttgctctgttgcccaggctggagtgcagtggcgtgatcttgtttcactgcaagctctgcctcccgggttcacgccattctcctgcctcagcctcctgagtagctgggactacaggcgcccgccatgcccagctaattttttgtatttttttttttagtagagacggggtttcaccatgttagccagcatggtctcgatctcctgacctcatgatccacccgcctcggcctctcaaagtgctgggattacaggcgtgagccaccacgccgggcctatcttatttaattcttacaattgccgcataaggtagacactattattttcatcattctatcttattaagggctaaactgaagctcagagaggttaagtcacttgcctagagtcacacagcttgttaagtgctagtgctgaggctggaatgaggctggcttctgcgctccttaggctgtctcctgcgtgaagggggatggagtgaacagtgccccccggtggtgatgtgatgagaattcgtagtcctcccctggacttttgctgcaggagaaagcctttcactgcataatggcgctcttgcccttggggagaagttgggggcacttcatctccagcaagaactgcacacaccacctgcgtgtacaggcacacacaccagatggaggcactgctgcagacatgcagcttcttaccatttcttcctgcctcctgccctccttccttccctgcctctccctccctggacatacagctcctcttttcttctcttcctccctccctttcttcagacataggtacctttccaggtagttagcatgtagcagggctccgcaaggctgttggatgaactgagtgaccgtgcttggagggcctagtagaggggtaggaagacatcgaggtgggtaagccatgcctgccccccaggggtgggtgaggcctgctgggcagctcctctgatcaagccttctcctgcctgtagtagaagctgcagctggggtgcatctttgtgctggggaaacacagcacttatgtgtttgcacatgtgcgtgcgtgtgtgtgtgtgtgtgtgtgtgtgtgtgtgtatgcatctgtgggcagcattggggctgcggttcctcctttcccctccccatcaaagtctggcccagaggcctggcagccactgatttctcgtgggctctctccactctggtctgcaagtacaagggcctccatccttgggtggggtccctgccagttccagtggcagtcattcttgctgcccgcggtctgtcacctgctcctgggcatgttctgcctttcttgcactcagatgcccccacatggtcacttagggctggctctttgcttgacagtggttgcggtaaggtcatttcaaagagcatccttcttagtatgtttaaatagctatagaaaagccgctgcaccctcacctcttcagagacccagccagcgataatggcgcgctcactctgatctcgggggagttaggagagggttgggcgtgcttagcaggtttgtgatgtaaacagtgcccatccctgagctgagcaggacctacattttgggcttcggtttctctaaaattctcttaaattggacattattacatgtcaaatgtagttaatcttggtcagtacagtcttggatgggggttgtttggcccctgcagaagggcctgtgttgaagctggactctgtcacgtgacttggaaacatggctgtgagcaggaaaacagctcctagttggcttgagaggttcagacgagatggtgtgtgcaagtggttaacaccaggacatagcatatgttaagttagctttataatttattttggatatctacctacccatccacccacccacctacctgcctacctacctacctacctacacctttgtggtttacacccactgccgccccatgaattgcaggggcgtgggagaagagtaggaaaccatgggtggggacagagtgtgggcttagagtccactgagagcctagcacgtgaagggaatgttgtctctctttgggtcatcagtcactaaaggcctgagttccctttatcccttgaacaaaagtaaaacaaggcaagtggtgatgagacctaactcctgatggcaggggctgtagatcccttcaggggcctaggtgaggggccacagtcacttgggtccctcctgctcttaatggccgggttagggatggagcccagccctgctgataaggtgagtcgcccagggccagggcaggaaaggccaggctcagctgtgcagaatagaagaatgggccaggcgcagtggctcacacctgaatcccagcactgtgggaggccaaggcagatggatcacttgaggtcaggagttggagaccagcctggccaacatggcaaaaccccgtctctactaaaaatacaaaaattagcccagcgtggtggtgcgtgcctgtaatcacagctacttgggaagctgaggtgggagaattacttgaacctggtaggtggaggttgcagtgagccaagatctcgccattgcactccagcctgggtggcaagagcaaaactccatctcaaaaaaaaaaaaaaaaaaaaaaagctgcggtgtgtggtggctcatgcctgtaatcctagcccttcgggaggctgaggtgggcaggtcacctgaggtcaggagttcaaggccaacatggtgaaaccctggctgggaaaaaaaaaagaaaaagaatagagctgagtggtggagcagtgtcccaggataattgggaaatgagcctttcctctctggtacctgggcttggcacaggagtcctggtcataccctgcagctgcctctgcagagagccacagggcattagggatattccttgtaatcatcccacgtaatgtttgtgctgcatggaagggcatgtaaaggagtgttctctgcacggtaattgactcattctgtcatcatgattagttagggggcgcccagagcaggatgatggtcagaggttcctcatccccattttaccgatgaggaactggggcttagagaggccacacggctacaaagtagttttatctggatcttcccctctattaagcaagggctggcagagctgggtgtgctactgaaggagcagacatgtgtgggcttggtgccatggaagtggccacaggcagagcagtagcacactccagtctgggctcagcatggcatcaggtacttgtttacatgttgttgtctccctctgaaccgagtttcctatttgtttatctttgtatccagtacacagtaggtgctcaatagatttttttagtgcacccctgtgtctggccttgtgacatagagacctcagaggtggacagggctgaggtgtgctgctcgtgggtgtgaggtctggcccaccctgcacctccagaagacaatgaggccccagccaggtggacaggctgtggacaaggggctgcagccctggcacgagggcctgggaaccagatgggcaccaccgttgtcagctatttagcatttctggaacacctctgtgtgcaagtacccaggagctgtgggggactcagaggcgccaatgagacacggcccctcccctccaggagagtggcctttcaggaggatggaaggctttcctcggtaattatgacacgacctaagcagtcaatggttacttgtcggatattgttgaataattgaaataaagggctgatgagccttgggaggtgactgagatcagaaaaagtagggggctttggagggctctgggcccatgcgcatctacagtgattcccagcccactgcctgacccctcacgctgggatcagcctctgcaggttggcctgggcacgggtgtgtgtgcaagcatatgtgagtgtctgtgtggcattggggtacgcaagcgtgtggctgtagctctgcagagcctggtgctcaggcaacccaggacagggctctgcctcttgcagaagccaattcgtttcactgaattccatgaccacagacagtccctgggaggccagccagccagctggcaaaggctgggcatgcttcttgtcccagggggcccgggcaagtagctggctagatccggcaagttcatcccctcgactcaagcaccaactcgagacatctgctcaaccgatgctgggtcagtagcatccggtttaagtactgaagggaatacacgtatgtcaggctggctgtctctaggtgagggcctcaagagacataaatctcttgtggggaggggtcctgtgtctgggcacagccagcccagagtgtctgtgcctgtgaatgccagtgtgccttgaccttggattgcctgtatctttatggtgtcactgtgtgtgtgcctgttgacatggtagggtatagtggcatgttgcagtagaaagaccgatggactggtaaacaaaagatgtaaattttggttctaaaggcctcctttcctggctctctgatccctgcaagtcagataatctctctgagcttcaggtgtttcatctgtgtaacagaaaaaaataataccagctctgctttctgtaaagtgtaagaatacaatgaaataacatatgtgaagatgctttgttaattaccacgttcagtgcaaatgtgagagattattatcgtcatagcccagcgcatcagaatgagacgttgtatatggcttaaaataatccatccagtgagcagtgttgaaatgtaggtcagatcgtagcctcagatgtttagagagaggaaccacctaatctcacaggccaccaaatgtgaaggtgctttgaaagctgcaaagaatgacataaatgcaaagtgacaattatgtttaagtctagaagggacgttttagtcctcttgtttcctaatctggaatcgtaaagatccgagaggcactggaacctcgtacacagttgtatttgtacgggtttgtagatttgtacgtgtgtctctgtttgcgtctatggactcacgtcaggcacctactctgtgacagtcattcatcaagtgcctgccttggactaaatgcatccgtctaggcattctggatgccagcatgaatcagacacccacacttccctcaaggagctgaccgttgccttgctgggtaggggaactataggagaggaagggtctttcagctcagtctaggcaggggatgggaggagggggcctaggggcagggaatccaggataggaggctggaaaagcgagtaacagtttgctgataaacctgaggggaaggtggtgtggtcagaagaacagtctatgcctaggggctgaggccggaagcagtttgctttcgcgcttggtgctgtctgttgggaccacagtgtggagaagagaccaaggcgatccagccaaatcctggggaccgccctgcacatgagctgaggagcttggctggatcctcctggcccccgggaacccccgtggcaaaggtttaagctctgcagagtggcataatcagctttgcattttggaaagctcctagagccccagtattgccacatatcttgcggcaagagcgcccgccagatgttgggctctgtgtgatagtgagggatcgctgcatatggtgggcggcgtgctgggatttgcacgaggcagggagagggggatggggaggatggagtgggagtgaagtacgaatcaggatgtgtggagcgggggaaggtagcagttctcgagtgactatcacgtgtcagcttttgctacatcacctcatttaattcttataacatcactgtgaagtagatatcatcccattttatagatgaggaaagcaagactcagggaggttaaagagcttgaggttactcctaagcaaagagcagagcgaaactcccagcccggctctgccctctccaaagcctacgtgtctggaatttccaccccctcccctctgcgctggaagtgtggacgaattcagtcccctggcttgacagatgaggaaaccaacctgggctcaaatctgttcccgcgaactgtgttactgttcttcagctgtcccatgtcaccacggatcagattcttttctttcagagtattagcaacgcctcctaattcagggctacttgcagatttgataagcatgttttacattcctttgctcaacccatcagcaaagatatggaataaaacagaccccgtgacagatgcgagaatgaaccacttgctacgcttccttggagccattgaggggcacggcccaagccatctgctcacccttcttggcgtgttagccccctccctccgcagagggaacctcaggcaggcccagagcagaagcatcgcttcccgctttgccctaagactccatcgcggaagagttggcagaagagaacatgcttttcacaacgctgctcttagggccaaatgtcaaggggaagggccaggagttgttcagacctggctctcgtggagactccaagcacgacccagaagcatgcggggcctgaatgctgggccagccgtaaagctccttgcaccaggtgtggggtacacacagcacccctgcctggcactccccgccctctctggacctgtcaggacctggagaggtggcctggcaccggggggtccaccccttcctcctgggctgggttgcacggaattgaatccaacagggaaaaaaatgtgccgagcacctacaggccctgcacacgtggtgtaggtgagagatgccagcaggactgcatatggcagaggggtggcctgtcggggcccccaaaggctttctccgcatgcccagtggaagctggtgcccatcgtgaaatgtcaagcgtctttatttttaggtcattgctgtgtggtaactgtggttccctttcactgatcagacgctctggttggctttcacatctctgattagttcagaataaaagaaagtcaaatgactccctctgtcacaaatgcagcttgttaaagaaatagtttaaagcccagaatttatgggcagtaggagtttttccagaatctgccctaggcgctggggattcagcagtaaacaaaacagacaaaggggcagaccctttgtgaagctgtcttctcaaaacctgagaagcagacaggatggacacctccctacccccattttatggatgaggaaactgaggctccgagaggctgctcctgtaactaggaaatggacaagctgggctcatgcccactttgctttttccatgacctcacatgacccctaatgattttacaagtgctgatcaagacggtgcccatttggcactggttgaatcggattagaagccccacctcagcctgaaattcttagcagttgtgtagatccaatcagaaccttcacaaatactaagctgcactggcaatacgtcctctctgtgtgatcctttcttacactcgggggtctggggtcttcagaagatggttcagaagcagcaggcctcccctcccatggggcgtgtgcccttgggcaccctgagccctcatgaaggctcccgcgagtttgctcctcctcccacagcctttctgccctcaccgcccgtctgctgtgctttcctgaaaagagcccctgaaggatctccggcctcggggctggcctctccctccacagacggcacggggctccccagtcccctggcatcctttccagatgcttcctctgctgtgctccttaaattgccctctttggtccttctccagccctcactgccgtgtcactctgcacagcttagtgacagtgctcttgaaggtcagccattctgacccctagccaagcctcctcatcctcagtatctttctcagacagctttggtctccagtgttggcagccctccccttccttgctcccacaacacaccagggtcctcttctgcacctccgagcattctctctctatctcctgtgtgagctctgcctctctccagcttgactgtggccaccacctatgctaagaccgggcacctctgttcaccctccatgtgcatgtctttggaggcctcccagaacttgagccatgatggtctgccctgcagacatgcctcagctggaggatggatggaggggccagggatgctcttcccgctggtcttatgtctacagctggtcacagtaggtttccatttgtgtgtcccactgtcacctaaaaattaatgtggcaaaccacaaggtattttgcaatcacagaaaatgaaagttgagtaggttgttgagggccatcttactcaggtctccaggagagctgtggaccctcttctacctctgtgctgccagcactgccctgggttggcccctccttggggagggcccaaaagccatctttggctcctgacatcgtgcagggtatgaagagcagcctgtggcccatctgcagggtcaggacgtctttctccccacttccccagcatcatttctgctggggattagtcatgccagagcccagctgagtcagaagggacttgtgggtggtttgtggccaggctggagtagtgaggcaggactttctaggtttatgtctctgtgccccaggagacaagcatgtcagggcccagccgtgcacaggtgttatgtgaggggcggggagtaaaacaaacttctgttccaaactcggtgaatgcaagagatggagaggagctgagattcggtccctgagatcctcctgcaggctgacaggcatgcgcggtgccagctccacatggcaggggtgttcacagctgtacaagtcctccactttccatagttgtccatttcgagtattgcctggacttcaaatagatattgctaaatttaatatttcttttcacttgattttctttttttttttttgagacagtctcgctctgtctcccaggctagagtgcagtggcgtgatcttggctcactgcaacctctgcctcccaggttcaagcaattctcctgcctcagcttccctagtagctgagactacaggcgcgtgccaccatgcccggctaattttttgtatttttagtagagatggggtttcatcgtgttagcaggatggtctcaatctcctgacctcatgatccacctgcctcagcctcccaaagtgctaggatcataggcgtgagccaccatgcccagcgattttattttcaataaaggtgattcattaagtagatcacaaatgtaattgtgtatctgtgaacaaaagctttctcatataaataaacttttaactcaaatcctttatcagagcttgtgttatgattttatggttcggaaaatgtggtcataaagagagagagatgccttccggggggacagcagcgtctgccctgaagttccccaaaggatgtattatttatatatatatatatatatatatatatatatatatatatatatatatatatatatattatattatttagatatataaatatatatctaaatatatatctaaataatatattatttagatatctaaataatatattatttagatatctaaataatatattatttagatatatatatgggtgtgggtatttttatagagatggagtctcactatgttgcccaggctggtctcaaactcctggcctcaagggatcctcctgccttggcttctcaaagtgctgggattacaggcatgagccacggtgcctggccaaggatatattttaagtggggctctgaggggtgcctcaagcctcatcaggcaggaggggagttggtgggggcatattacacagccctggaagtgtggctcgtgggacaggaggaggctggggctggaagggaagccagggccagccccagacaggcctggaatgctacactgctgggatccacctctggcctcacaggctgtgtctgtgcctgcaggacggagaggagcaggaggagccgcgtggcaaggaggagcgccaagagcccagcaccacggcacggaaggtggggcggcctgggaggaagcgcaagcaccccccggtgagtgtcacatcctacacaccctcctgtgcacacacacaacacacacgtgcctggcctgtcgatggcagtgatgtatgtcctgcagggacctggagacatttaagaccaggtgatggggtatacccctccagctgcaggtaccccattcttcaaatcccaccctcacatttccctgttttcttatttatttatttacttttttactgagacggagtctcactctgttgcccaggctggagtgcagtggcgtgatctcggcttactgcaacctccacctcctgggttcaaacagttctcccacctcagcctcccaagtagctgtgattacaggcacgcgccaccattcccggctaatttttgtatttttagtagaggcgagatttcgccatgttggccaggctggtgtcgaactcctgacctcaagtgatccgcccacttcagcctcccaaagtgctgggattacaggcatgagccaccgcgcccggccacatttccctgttttctgcatagagaagtggaggccctgagaagccaggggacttggctggttggtcttgggcctaagacttggaccaggcccctgactcctggcctccctctggaagggaagcaagagggcctgccagaatcccaggctggactaagatctccctggactatctatgaaggcccctccctaccacactctcacccagggtctcagcccccacctggaggaagggaactgaggcctgggagtctgatcaaatgggaagggggagatgcaggctgaggagtctctgtgaggcagcaggagaaccagggccctggggaaggaagtcctgcaggcatgtaggctgcagggccagccccagcgccaggcttctgggaaccagtgccgctgaggcctgggggcagctctggagaacaagacccctgggctgtaggagcctaactggtttatgtcttaaactcagatgtggagcagtcattatttcccagcaccagagacagctggttcctttcttttaccccccttccagctgagaatggtaaacttggagcaagaaggtaagggggagtcagaggctctggatttgttactcagcccttcaaagaaatgttctcagcaggcatggagcccaggacttgctccctttgggtagagagccgggttgaaggtgactgaagtgaaatgggacagtagaggcggggggggtggtgagttcctggaggtggggggtgtgggaacctgctgtgtactgagatgcacccctgccagttctgcctgaagatttgaggcggggggcaggggggcggagtgaagtcattttactggtaagtaattttaaaccttttaatattaaagcaaacgtggatatgtaatgaatgaaattcattctggaatgaaaaattcacgtgatgttgaaaaataacacggggcttcagagaggactttctggctggcagcagactccagattcccagggcccctgcaccctcctctgcccacagggcaccttaatggagaaggtgtgggaggagagccaggccggagtcagagcacactggtgactccacatttgcagcgtgccctgcctctctcctgaggcttggcaacgtgcaatatgctaagcaaactccccctgtccccgtccagtttctgaggacaagagccaccacctgtagcaaataaagacccagcaaccctttgactcatctttgtgagtctctggaatcagagggtagccacatcgctgagaggtggagtgaagcactcgggtgaaaaggtacaaggaagtcagggacaggagtgtggggacatcacctagacaatgacagagaagaggggcacagccgagtgaggggagaggggccggcagtcctacatcccctggcctgaagcacgctccagggcagaaggaaaaacactgtctttggggtccaagagacctgagttcaaattctggctccaccactgaccacctgtgtaaccttgaactgctgctgcctgaacctcaggttccccttctaaaaatagaggagaaaaggatgcatttctccttgcccctgtgagaacgaaatggtgcaagcaccaaggagcctcagcaaaggtcgggcctgcccccgcctggccaaacctttcctcttcaggaggccacggcaaccgtagtttgacagaagagcagcaccttgatttaatgcttcccagcatgtgtccttgagcaagtcacctaacctctctgggctgcttcctcattgggaaaatatggctgccagtaaaacctgccctgtccacctcctggggcacttggcaaacagcaaaagagtccaaatgtgcaggctgggccaggcgcagtggctcatgcctgtaatcccagcaatttaggaagccaaggtgggcggatcacctgaggtcaggagtttgagaccagcctggccaacatggtgaaaccttgtctctacaaaaatacaaaaattagccgggcatgatggcgggtgcctgtaatcccagttactcgggaggctgaggcaagagaatcgcttgaacccggaaggggaaggttgcagtgagccaagattgtgccactgcactccagcctgggcaacagagcgagactctgtctcaaaaaaaaaaaaaaaaaaaaacaatgcagagctggctgtgtaaaaaacctgttccactgcagggcccagtgtccaccaggctggggtgcaggcctatggggtgggggcccagcatcagcctctcagcagccctgggaggcggggcgcatcccgtgcccctcgtggtctggatgtgttctagcccaagtcctaggttacacctgccgtcgcctggcctctcaggagaggcccagggtgaggaggagcatggtaaaggtgaagctgattgggaagtcagctgttgggaaagcaactccttgcacattggaggaaccgagaaagactgaccccgaggacagcagccagcatggcccttcctgggagcccatgttgggggattcctgctgcagccaaggctcagcccttgtggtcgcaggtgctggtcctggcctcttcccctcccatgcaggagcacaggagagatggcttctgaggacctgttgcagctgtggccctgggaatagatttgccagggagctttaaagcagctgagtgtgtcatccagctaagcctggggaaggagcttggctcaggtcctgacaggtgtgacagggatggggactgggaagtaagagatgaaaccctggctggaggctgtgagcttccacagccagcgctggacagggagggtccagatatacccactagtgccctcaccaggtcccacccccaagcccaagaaatcctgggctctctcctccttttgcccccaaggactgtctgtcttggccttcaacaggcatctgtccctgggtgccttctgccccctggcattgccctcacccaaaccagactccacctgccccttgcatgcccaggccagtgtgctcccttcacgacaaacactttccacttcctgagcactgaccatgtgcgggaagccaccagaggaagatgccttctatttcagtcaagtgtgctggagagaggaggccacacataatcacagtaaatgtagcaccggcacgtccttcttcaggaaccccccgggactccattttttaggggacaagatctgaattccagcccctcaccatctgaccactcctttccctgctatgcctgatgccacccctttggccacagaaacctcaccttctcactgctccccaaacacactagccagccattcctcctgcccgccccttacacagttccacagcctgccttctctccgcctgccccagcaccacagcccggtagaacctagcctgaaaatgaagcccagttcagccactgtgacccacactggcctttcccttctccaactgtctccaaagttaataaaaatgaactgataaagacaagttccaagcccaccgatcacacacatgggactgcagtggtctgcagacctcactttgagaagcatttgttgtcacctgtcggtacagaactgagtataactattcagaagcttttatgtgtgtgtgacaatgttgccacatccacgtgctgtgattttatattttacaaccgtatcatttcttggtgcactgagaatttaaaaatctggaaccaaagcttgagctttcatccctgacagtttgaaaatgcaccgccattgcatagtcgctgagctgtgccacactgagtttgcctgcctcactgaggacagagaccagcagtaaaggctcatggttatgcagcatgttactgcctacaaaggcgttttacaggggttaccttatttgatgctgacaaacctgcgtgaggtagttggggaccgctttcattatccccacattcagaagaagagactgaggttcagagaagatgatcaggacttggacttcacacctcccaactgcaaagcctatggtgctctttcctccttatcactgcatccgtgcagtttggctactgggtacctacccaggggttctctgtcagttaatatgtgaattcagggctgctggaaaaagtgtgtggaactggtccaaaaagcatacaaagaaaatggaactgaagagagttattgacacaaataccagggagcctgtgtgtaagtgccgaaggctgcagggggctgagggcaaagaggtgcgcctgcttaaaatcggcaacggcctagttgtctgcagcactggccagagagccaggatgctgcagtcagagccggcaacaggaaacaggaagtcagctgcagggcagggcggggcttaggaagggagctggcacctgggccagggcctgcgggctggccagtgcccatgatagggaggcgggggctggtgcgtggagctgagcccgtgtgtgggtgtgaggtggggaggaaggggcagcctctaaggatgttgcagggtgacaagtgtgggtgtctgaggctggttcctgcctggttgccagcccagacagtcctgcagaggcctttgtccttcatgggtggttccggttggagccctgaagggagagtttctgcctttgcccacaaggccctggggggtgagtggccctgtggggctccatcccctatttactagcgatgcgtatttattcattgacgatttatacccaccaacttcctccagggttatttttgctcagagggagcactctgaggtcacctcctgctaagaataacccagaatgggagggaggaggaggagggggctgggcgctggtgctgagctcagctgagcccaagctggtgttagggctgaccctccaacaccgcaggagtgagccagtccaagagggcaaactgccccacttcagagcagatgacttggggtaacctaaatggggaagtcagggaaccattctgctgcaggcaaaagtcagctaggtacagcttatggcagctcttggaggtttgcagcactggctagctttccagcctgataggacctttgattcttctccaagttaagacagagctcagcagccctgcttgttttcagatcagagccgctcctgggctaaccctccggacctcggctgggtgatatggtgcccctaccttttatgcatataacttcctgacatcgtgtctcccactcctgggctgcagcccttggcccacccctagaggtggcttttcttcctgcgagcctgcatcagtacacgctgctgcccactctgtctcctcatgttttggaaggcatgaggaagacaggccgacacactgcttgtgaggtagagcctatggttatgaaccttgcagctccacccacccactccccgacctgagcggtgaaatcaacgcccgctactggagtgctgcatggaagggcctagcccatggtggggacttaagtgcagtgggactcctaaggatgccggggaagccagggaagatggaggaggtggggcttgacctgggccttagaagggaggtgacatttggagaagtcaaggggaaagtgagaggcacccccgaccccagctgagctgggtgtggaggaaggaaggcatgcagggcagggtgagtgtggggaggcccaaaggcaggggccatgagcacacaggcacaggcctggctgaggggactagggattcattgaaaaatgtatacggggctgggtgcaatggctcacacctgtaatcccaccactttgagaggctgaggcaggtggatcacttgaggccaggaattcgagaccagcctggccaacatggcaaaaccccatctctactaaaaatagaaaaaagccgaatgtggtggcacatacctgtggtcccagctgcccaggaggctgaggcacgagaatcgcttgaacctgggcaggcagaggttgcagtgagccgagattgtgccactgcactccagcctgggctacagagggagactctgtctcaaaaaaataaatttttatatatatatataataaatagtagctgggtatggtggtacacctgtggtcgcagctacttgggaggctaaggcaggaggatctcttgaccccgggaggttgaggctgcagtgagccataattgtgtcactgcactccagctgggtgacagagcaaaaccctgtctcaaagaaagaagaaaaatcttctcaatcttgattggaaaggtagtttgcaggcagcctgaggggcttttactctttatcctctctgaaaaatgtgttatctggaaggttgtgaggaggcggatatgacgtgatgggagtcgtgtttgagaaattggctctggcacccatgagcatggcgttcatccctgagcatgagcacatgctgggagaggggagggggcaggggcgccagcaaagtcccagggcctgtggggggtggcagtggcggggcagaattggccaagcctatgctgctggccctgggaacgctggagaggaaggggtgaaaggaagccctagcaatggcacaacagggacagcatcagcgttgttgtcagagacaaggcttctggaagggagcagatggggaaggggcccctgtcaaggccaccttacatagctggtcttacttggggagcatttaagtagaatctcagaatctcctttttttttttttccaaaaataatccctctgtttattttcatctctccacatcccagggccaccttcagcgagtcattcattcccacaggtcctggccttggtggccttcctggggccccatgagcttttctagggcccgggctccacctgcattgttcctcctgttgcaggtacgcgccggcacacccattggaggagagctgttgacatgggctttctttcatcatccccacctagagaattttgtacattttggttctcctacctcaagaaagacataagcgagttggaaaagctccagagaagggcactaaattgatcaaggggaaggcaaggctgccaagagaggacaggctaaagagattaggactccccagtctgcagagacaaaggcagaggatgtgattaaggctatgaaatcatgtcgggtgcggcgggctgagcacagcacagctggggggcagagggaggacacccattagaaatttcagaaagataaatgaagcacagaaaagatagcaccattttccatggtgggttcccttcttaccaaacctgtcaccctaggattggcagaaattgaaaacagaagcaacttcaaaagagatttacttaacgtatgggatctctcatacgtaatggctttttttgtttttgtttttgtttttgtttttgagacagattctcactctgttgcccaggccggagtgcagtggtgcgatctcggctcactgcaacctttgcctcccgggttcaaacaattctcctgtctcagttttctgagtagctgggattacaggcgtgcaccaccatgcctggctaatttttttttttaagggtcgaagggatttattgaaaatgaaagtacactacacaatgtgagagcaggcctgagcatagggggctcaaaggccattttttttgtatttttagtagagatggggtttcaccatgttggccaggctggtctcgaactcccaacctcagatgatccgcccgcctcggcctcccaaagtgctgggattacaggcgtgagccactgaacccagccagcataatggctttttaaagggagcctccagctcactctgaaatcctttgaggttgacttcaaagagcacaaccaggcccttttccaggtggaccagtgtccccgctggaaacagaaccctgagtgggccggccactcaccacgcatggtcattctggcatccatttgccatggcttgtgacggcctagccaggccatggactgagtgacctctaaggctttgcagagcccagagttctctcctcagacacacgggtggagagatctctggggagaagggagacaggcagctcccagcagatgtggtaaggggaatggtaggacctggagggggtgcccaggctgggaccagagtttccagagtcggccctctgtggcctctggtcccgccatccctgaccccactggctgcgcttttagccaccccacagggaaaagggaaattggaggagtcagggtcctagctccatagcgttggagcttgctccatccaccccaggaaggccgagcaagggggcatctggggaccccctggcccctcctggagtgggcatggagcaggcatctctggctctccctaccacaggccacttccagctgctatcagatctctggtgagggccgccaggggaaagggggctgccggaagggcgggggcgggcagtcatggccccgtgccaatgcttcgggggcagagctgccctgcttacgtggacatgcagctgtttcctgaaactagagtcaacctggaaaaattcaggctggttgaggggtgatgggcctgcagagctgaggaactggaaggagcgaggagttgcggtgggagtggaggggctcactggcgtgtttgtgttggcatcacgcagcttccttgcacccctctgtcctgtggctggcagtgggtgtctgcaggcgtgggggtgcctccttctcaagtctgtgcacgtgacggtgaagagtccgtggctggcctgatgccagggttcccggttggcatggaatggatctcacagaggcattaaaagcaccatggcaggcccggccctctgggtgaccaaggtgaggtcgcccctgcatctgctgggctttgggggagaaggcaagaggagctcccagtgggaaaggaagaggcaggggaggagggagaatggcatctacccaccccccacccccacaggatcccccttccccggtgatcgctgcaggtgggctgcagcctggggacagggcttcttgccccaggtgccagggcctcatctcccctgtgccatgaccttcacctctcctcctcctggattttagtttttctgtgtaaataactatagtaaagcaacacttaaaaaccacttctggccgggcgtggtggctcacgcctgtaatctcagtactttgggaggccgaggtgggtggattacctgaggttgggagttcgagaccagcatggccaacatggcgaaaccccatctctactaaaaatacaaaaattagctggatgtggtggcacacgcctgtaatcccagctactggttgggactgaggcaggagaatcgcttgaacccgggaggcagacgttgtaatgagacgagatcacaccattgcactccagcctggagaacagagtgagactgcgtctcaaaaaaaaaaaaaaaaaatcacttccataaaattagctggatgtggtggcacatgccagtggtcccagctccttgggaggctgagataggaaaatcgttgaatttgaggctgcagggagctatggtcacacccctgcactccagcctgggcagcagagtgagaccttgtctcaaaaaaaaaattttttttaattacttccataaaaataaaaagttaggtgtggtggcaggcacctgtagtccctggtaaaactgaggcgggaagattgcttgagcccaggaattcagggccacagtgagccgtgatcgtgccaccgcactccaacctgagtgacaaagggatatcctgtctcttaaaaaaaagaaagaaaaaatcacctccccatattgggagtttcagacattgccccaccctgggacgcaaggctttgcctggagtgaaggaggggcaggaggtctgggtctggcccagactttgggcaaagggggaagaggaggcggcggcagggctggggaattggttccggctgggcaggccacagcagagggctcctccttgccttgcctctgtcttttgcttgaattgagcgctgctcaggggagggccagcagcctctgcccggcctttcttgggcactgcactgccacctctgccttctccaaaccctgggggcatttgtgagcttgcgcttttctgagactcaacccagagcgggagtcggccttcaaatgcctcgcctgtacctgagccatcctggaagctttactgatcagaggcgtgtgacccaaggcaggcctcagaaaagcgagtatcgctccagctcccctaggatccctgtgaaaattacatttattaattttataagcatatgtatgcccagtcctgtgtgggatgctagagagccataggtggagaggcctacaaagttaaataagattggccgggtgcggtggctcacgcctgtaatcccagcactttgggaggctgaggcggatggatcacctgggatcaggagtttgagaccagcctggccaacatggagaaaccccatctctaaaaatacaaaaattagccaggtgtggtggcaggcacctgtaatcccagctacttgggaggctgaggcaggagagtcgcttgaatccgggaggtggaggttgcagtgagccgagatcgcgccactgcactcgcctgggcaacagagcgagactcggtctcaaaaaaaaaaaaaaaagttaaataaggcaaggctcacagtcgaggagggaggccacagtaaacagataattagattcagaggtatgaattgtaaggagattgggtgagaaagggctttgtaaacaaacgtggcatgcattggctgggacaggagtgacagctgcggtgggttcacccccacccttcagggcaaggctgggccagcagctgtccacacctgccaactacagggggctgaggggtccagatccttccatttaaaggagcattcctttaaaatgctccttagaagacagaaagtagaagagtggtgactgggggagaggcaactgggaagttagtgttgaatggagacagtttccgtttgggatcgtggaaaagttctggagatggatgggggtaaaggttgtataaccatgagagtgtacttaaagtatgcttgaaaatggttaaaatggtaaatgttatgtatgttttaccccaggtttttagaagtttaaaaaagtgtttctaggccgggcacagtggcttacgcctgtaatcccagcactttgggaggctgaggtgggtggatcatgaagtcagaagttcaagaccagcctggccaagatggtgaaatcccatctctactaaaaatacaaaagttagccaggcatggtggcagtcagctgagataacaccactgcactccagcctggacaacagagcgagactccgtctcaaaaaaaaaagtgagcgatcttggctcactgcaagctctgcctcctaggttcacaccattctcctgctgcctccccagcagctgggactacaggcgcctgctaccacgcctggcgaattttttgtatttttagtagattcggggtttcaccatgttagccaggatggtctcaatctgctgacctcatgatccacccgcctcagccgcccaaagtgctgggattacaggcgtaagccaccatgcctggccaaaaaaagtgtttcttaagccaggcatggtggctcatgcctgtaatctcagcactttgggaggctgaggtgggtggatcacctgaggtcaggagtttgaatccagcctggacaacatggtgaaaccccatctttaccaaaaatacaaaaagttagccaagtgtaatagcagacacctgtaatcccagctacttgggaggctgaggcaggagaatcgcttgaacccaggaggtggaggttgtagtgagccgagattgcaccactgtgctccagactgggcaacaagagcgaaactccatctcaaaaaaaaaaaagtgttcctttgataaagacagtccctgtccttgcccctaccctcagcccagggtttttcaaagacattattgccattttgtctggactaaatctctgttgggggcgggggtgtcctctgtctggtggggtgtttagcagcttccctagcctctactcactagatgccagcacctcaccctaaccttgtggcagccaaaaatgtcttcacacacatgccaaaatgtcctggtggggagttagcaaaatcgccccagttgagaactacggccttaacctgcctcaaaaagctgctcacaggagaggactgaggaaaagccggaggcaaggtgggggttctgcccagaaggaaagcgaagattggcaaactcggagggaccccaggaagtggtgggcaggatttgaagacccaaagtgtggtataccgggtgggggtaggtggacagtgggtgataggaccaagctgtactgaaatctagaatgatactagattcacaaccctcctgtggcccccatctggccccccacgggaggagaagtgatgggtagttactgcggcttttcccattctgaaattgtcacctgcagctgcacctgctggtggtgccttctagaatggtcggcacccccaacccagccctctcccagataatgggcaacatgagtatctcgggggtgtgctgtgtctctttcaagatgactgtcaaaagcaaacaaaagcagattgtttaacagacatggcagcaaagctgaaactccctctagcttgagtgcaggaggttctcacttttagcctcgtctttgccattcacgaatcaattgtgaatattgtctgagcacctgtcaggtgccccactccctgctaagtctagccggatcgagcaaagtctctgccctccagatcgagcaaagtctctgccctccagaggtcaacaagctgtggagacaggggaaaggaggggaggagagtggctgggaggaggacatggagttgggggcagaagaggccaggcctggctggggtgcccctccctcctgggcttccaggctgaggtttctgcccttctcaccagggatggctccttcagtgagcacccagctcttccccagagacttggacagttgcttcaggccatttgactgggacctttttccttcctctggatggttttgtcctgctggtcaccctgagccccagagagccccattgaggggccacacttagcttgagggagttcttgatgggtaggatgaatcaagggttgagcgtggcagaagtcattgccctcccccccaaatcccagaactttggctgaatcatctcatccagaacctgagagagcaggtgagcaggaggggatggttgggcagaggatggtgtggaccagatgcgggagtggggatttcagcgggtgagctaacccggggctggaaatgtgcaacatgccccgctcgcccatcatctgcccagcatgcccggctcccacctggctttgggggcatctctgggagaatggcgcatctggtgctgttgcagactggggtccgcgggcaaggccagggcagggcttactcttgtgcatgaaggactgggttggtcgggagcctggggctgtgttgggcctcagagccggccctcataggtctctatgctacggagccagcagtcaacaggaagtgagtcccagcatgagaggaggccatgtggtcccaggcaggaagtgagtgacagtggagttgcctccagcagctttgcggggctcagagcctgccccaatctgaggtgtctgcagggcagcaaatgatgagaagaaaggaagacgcagccatgaaaaagtctggctctgacctaagcttcttagctcaagttagccaggcaggaccccacgcatacctctatggtagtgccatgttgggggctttggggtacctggaacagctggatcccagaaggtggctgcagtgcaggggtacactccacttttcaggctgctggggctcatgactgagccttgaagggtgacagacagggtttacaccagggaggaccattctaggctggaaatgccctctttgcttaagcagggagggaggagaggtgttctgagttctggggagggtcctgacacacaggatggaggcaggggtctgccgttccactttccttcccggatttggccttcctggctctgctggcatcagcccaagcccccaagcacagtggaggacttctcgcttttaccagtcacctcctcttcctctcgttgcagccccctgcctccaactgctgaggagtacacacatgcagctttctgtgggtgcttccctcctggaagcaggcctgcccagctgccccgcacagcaggaaaggagcttggcaggccctgcagttccttctgctccctctccggctgctccctgtgcccgggtccccatccagggaggaaagcatacctgagaagactctggttttgtttttgtttttgttttcccaaggcccttgtccctaattgccacctctgggcgttccttgccaaataacagtgggttgagagttcaggtcctagaccgttagagcaaaagcagccttggaaatagggctttgcaggggaggggctggggacccagtgctgtctcaggctgtgcacttgagtggtgggaaactctggagggaatttggcagtatctctgcacatttagctggacaggccctttcacccggcctgaccacttctggaactccgtcccatggaaacacgcagctgtacagagctgtaagtgcaagggtgttcactgcagtgaaaagcaaatcagcccaaatgcccaccatcagggaacggcatgcatacattttggagcatccagatccatctgcactgggatggagggatgcccactctgcttgttaagtgaaaaagcaagttgcagaacattacgactagtatgatacccttttggaaaaaaacatttctggatctgaatgtgtatatgaatacatatgtataaacatgtaacttacacatgattgtcaatgcataggaaaaaatctggaaaggatgctcactgaactgttaataatgggtaccttaggggagagattttgggggtagggagctgaaataggagctttaaccttttagcattatttttttttttttgtatagtcttttttttttttttttttttttttttttttagatggagtctcactctgttgcccaggctggagtgcagtggagcagtttcagctcactgcaacctctacctcctggattcaagtgattctcctgcctcagcctcccaaatagctgagatgacaggcatgcaccaccacacccagctaatttttagtagaaacagggttttgccatgttggccaggctggtctcaaactcctgacctcaggtgatatgtgagggagcctgcctcagcctcccaaagtgctaggattataggcgtaagccaccacgcctggctacagtttgatttttttatgatatatgtgactgacttttataagtagaaaaaaaaagacaagaaagacaaaatctccattggtctagaccagtcatctccacctgacaggtggggaaactgagatgctgagagcagtgccttccttatctgaggcttgccgtgctgatgctgtccctgcctttctactgggtaggtgttctgcatacggagccagcgaggcagagttgctgagtgcagcctggtgcctgtgggtgtggcagagtgccgagccatggggacccctgcctgttcctgtcccacactggacacctccattcttcttgaggtgtcctggcccacttgcctctggatggctgaggactgaatatatatacacagcagagcacacagcaggcacccggctctcctgctcttgctttgtccttgcaaagcctgtgacctggagcaaatcacaaaattccagagagcctcaggttcctcacctgcttcccagggaggctgagaaactctgaggaggtagtaagtgaggagctttgtccactgcgcagtgacacacacagacagccaggattattctttatttgatttttattacctggaaccttagcgagttcagttcttagactctaggggataaagaaatgatttcccttgaagggagaatgagagaccccagtgctcaggaagagttgttgcagagcctgaaaactgtttgccagagccttggctcttctgggtctgagtgcctgaaagaatctggggcggcccaggccaggggagaaggctacgctaggctatgagcctacagaatggttttttgggggtcctccttcgactctcatgcctcaagagaccaagaggaaagctgggagggcactgaccacagaagtcagaccagcctgagtcccagacttgatctggtggcactggaatcttctaagggagaggggggtcttattggattatgggtggcacaaggagcttttttttttttttttttttttgaggcggagtcttgctctgtcgcaaggctggagtgcaatggcgtgatctcggctcaccgcaaactctgcctcccaggttcaagcggttctcctgcctcagcctcccgagtacctgggattacgggcatgcaccaccatccccaactaactttgtatttttagtagagatggggtttctccatgttggccaggctgctcttaaactcccgacctcaagtgattcacctgcctcagcctcccaaagtgctgggattacaggtgtgagccaccacgcctggccggagcatttcttttttaacaggggctgcagagaaggcttggacgtcctatgtaacctcagtgagtggcggtgggatgggggagccgatcccttgagaagtgcaaccaggctggcaaacctgcttggattcttttcctgtttctgaagctggagtccacagactcgtgatttcaaggggtcaagcccaggaatcctttgatgttgaaagcaaaattgtgaatgtatctgtttgcatgaaggtttatatgaacacagtttctggacagaaagtgcataacattctctagaatttcagagcggtcaatgatccaaaacaggctaagcccactgatctaaccctcctcttgtgtatctttcttacatttgcaggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggtgagtcctcagcaccaggggcagcctcttctgggcccaccagcataccctgagagtcagggacttggctctccagcaggtcccaggaaggatggtctgggtcgtggctaaaggtctgcttgccaaggctatggcctggaggctactggctggatgcagcctgcgcatatgttttatttggcccatagagtgttttaaacatttaaaaaattagttgccagtatttaaaaatcaaaaaatttcacataaaaatctggagttttggcttctcatgaaaaaaaaaaaagctagatctggcaacagcgggctttcataacgccaacgattgctagactgggataatggcggtccctccatcgccttctgtggctggttgtgggccttagttttctgcagctctacctggcctgcttactctcccacgtgccatgcagttcctgggggttgctgtatttgtagcccctggcctgggcactcaagggcagcagataccctgtttgcctccctgagtgcagaggtcctgagcccaccctagttgggctgactcaactggaaatttggttgtgacagtggcgtggggagagggctgggtgattgtattctgtgtactgcccagcccaggcctcttcatctggggactttttggcctaaccctggaagcctggaaagttgcccacttttctctttcaggttaagccagcaatttcagggccaaccgagctgtaaacatgttagtaatgaggacaactagcatttgtacagggcttcacagtttacaaagcgctttctcatacattatcacatttgatcctcccagggccctgccaggttgttttgcatatgtgcattttaatttcaaaaagtcttccttccaagcgtgtatgatgaaatgagtaaattgattaattggcgtaacttattttgcatggatccaacctaatgttcatgcaggatagagaacatttccagaatacaaatttccaaacttattaagaagcttttattcatttattattcgacaaatatgtatgatgctcccactatgtgcagaacactacagagggaatctgtattagttgttttatttactcctctaacaatcttattcaagagatatgattatcctcatttcaggagaaaaatattgaggcttaaaacaatgatgtgacttgctcatggagccccaaccttaagtggcagatccagggctcattcagtcctggtcttgtgcctttctactattttccgctgtcctggaatgggacttggtgcggagacgggtcctgaattgtctaggcctcttgcacatattctgatacttcccgagagtgggaagggcctgaggggagtgaaacagatgaggaaagggagaagacaccatccttcccattaacaatgccaggtgtgtgagagaacccatcagccctgcccttccaaatgtggtgcttcccatgggctgactgcagttgacttcgcttcccaggaccccaccatgactcctgccgagctgctacccagggtcacggcattccttcctgccctgaagcatggagccatagccccaaagctgttctctggccttgagggtttgttttcactttgggatttccctacttccatttctagctgttctcaagggctcctgcatcgatcagtcaacaggtgtttcttaaaacacccattcagggtgtgctaggatccataccagggactggggaaactattatgaacaaaaacctgcttcttgccctcaaggacctcacagtcagctggagagacagacattaatgagaaatccacccttaaaaagatgagattacacatggagataagcgctctaagggaagtgaaacatgtccaggggaactgcaacgaagaacctgagttggcctgggctgtccagcgcgagctgaagggcgaagggagtccactgggtgaggggtagagggagagagtgagaacagggaagaaggctcccaggtgggagggagcagggcacccagggaactgatagatgtcacaaggctagagtgcagagagcaaaggagaagggtgtggaaagtgctggggcagacaggccaggccttcaaaggtggctgccggggtggggggtgggggggcggggaggcggggggtggggaggcggggagtgggcagtgtgtgtgatgtaatcagctttgcaatttgaaaagctcatgtcatctggatcctgggatggaatggaggagctgggaagcaggatgacatcggaggggctgatgagcaggatggcagcagagatgaagggaggggacaaatgccaggaacatgagaggtgagggagagatgctgtcaaggatgattcctgggtctgggactttcagacaggtggatcttccttagaatcttagtgctgagtgtggtgggcaaaaactctttaagtctccttgacctccaaaatttggccttgaggctgggcacagtggctcatgctggtaatcctagcactttgggaggcccaggcgggcagatgacttgaggtcaggagttcgagaccagccgggccaacacagtgaaaccttgtctctactaaaaatacaaaaaaaaattagtcgagcttggtggtgcatgcctgtagtcgcagctactccggagtctgagacaggagaatcacttaaacccgggaggtggaggttgcagtaagccaagattgtgccactgcactccagcctgggcaatagagtgagactccatctcaaaaaaatacaaaaaaaaaaaacaaactttggccttggtacactcctgtgtggtcccagctatccaggaagtatagattctggcagggatcagagctggtaggatccctgaaggccactgcactgcctggaagctgaaattggtgagatgttacctcgcggggtgggcagtggcctaggctctgccaatatgcaacggacagtggcttaatcagggtgtggaggctcggtcacggttccagcagtcagctgacatagaacacatgctcagctggttgattcagatgaggaaggagactaaggccccggagaggatgtggctcggccgtggtctcctttcagttccccttattccaggtctcttttcaggggatgctctcctggaacccctttcattagctcctgtacagcaaaaacagactttttaaggcagcgtttcttgtagcctgagcatgtgctgaaagtacagccaggagaaagaatcaggaagacgggagatgatgagatgagtgatagggacaggagccaacacccgacaggcctgtgcttgtcttgggtctgttcctgcccccgccagcattaatgccataatgatgatgattcaggtggcagtggcgatgtggctttcccttattgagtattgactgtattgggcattctctctcctcgctactcacagtggtccatagaccagcagtgttggcagcacccaggaatttagaaatgcagaattccaggctccactgaagacctgctggatcagaatctgcattttaaagagggcctcatgcttcccatgtatatgaaagtttcagaagctctgctgtgtattatgccccatgtgtgatcacgggaggaagatttttttttttttttgagacagcgtctcattcactctgtcgctcaggctggagtgcagtggcacaatcttggcttactgcaacctccacctcccaggttgaagcgattctcctgcctcagcctccggagtagctgggattacaggcgccagccaccatacctggttaatttttgtaattttagtagagacagggtttcgccatgttggccaggctggtcgtggactcctgatctcaagtgatctgcccaccttggccttccaaagtgctgagattacaggcatgagccactgcactcagcctggaagactcttattatcctcatttttcagatggggagactgaagcttcagaaaggttaaattacttacccaagattatgcaactggtgagagaccggattagggtcagcacaaaacccctctctactctgagcatagggccccttttcaggccagtgactgccctctgaggtaaacctggctacagcctggtcatggagatttgagttcattttaaagagaacttgggtggcctctggctgagtatagctcagactcgaaggcgagtgttagaagcaccctggcctcccctgcccccaagtcctgagatgcactagggtcatgttcgggcctgtcagggccacactggaaggtcacaaccacctggccctggcggtgaccctgggcctgggccaggagcagttgggctgcataggccgtactgagagagcagctgaccctccctgactcacgacaagtgccgcattccgaggcggcccggccaggctggaaagcgtgtgtgtctgtgtgtgtgtgtgtgtgtgtgtgtgtgtgtgtgtgtgatcactcaagtgggcatgcgcagagtctgtcagggagaaaattccagaaagaagccagttggaaactgcatagggaaaaatcgttctgttttggggagcttggggggctgggaaattaggaagggggtcacattgagaaataacagtagtggtgcagcggagagggagcttgccggggcatcaggacccctgaaaagctcatctcagctccggccttaagtcactccaccccccaggtctcagctttctgcaaaatttaagtgctgaacaagtactcaatgatctctgcaaagcctttcaaccctgattttctaatattctaatgaataaataattatccgtaaaagtaattaaatgccatggagggtcccaagtaggaaacgtttgaggttctaaagcgggctggggagaaaatgcagtcggaagccccaagtccaggtccgagagccagcccgggggtcctccactccccgccctagccgcagggctgacacagtggttcccagcgccaccgcgagccccggagccccgccgcgtcagggtccgccgggaccgccaggccgcccccaggccgccacctgatcgcgagcaggcgccccctgctgggagcccggggcctcaccgggcagagcaacgaggtctgggaggagggggaagccctggcctgggaacggggaggccccggacaggctggagggggcccccgcgaaaaaagtggggagctggctgtggtgggagagaggacggaaaggaaacagggaggcagagccagacaagatgggtgagacagcccttaaaaggccggtcacgaacaaagcgctggcgagtgcgcgcccgcccacgcgcacaggtgcccgcgacaagacgccccgtccccgcccacgcggcccccgcgggctgagcccggcgccaggcccgcacccacggtggccctcctgcctccctccccctggtgctcccgggcctagctagcgggagtcgctgtcaccagggacagaaccacggaggacagcgctgtcctgggcgcccggggtcagcccaaaaccagtccaggacccccctgtcacgagggaagggagtggcctcccgggctgaggacccgcggcgtgaacactgtggctggcttccgcttcacagccaggagatgaagtcggggctgttttttccgtttcaggctctttgtctccccccaccccaagctaccatgagccagccttagtgaccttcctgcagtgggcccaggcccgatacgctgtcggttcccgacaaagaagctcaggggcccaggtggaaggagggctcacttccccacctccagcaagaagctatgggaggcagaatcagccagccccgcagcctccggggcgctgggcctggaaggggtgcctggaaagggtggtgaggacctggtggggagtgcagggggatgcctgggaggagcgcagctcccctcatgctgggggcttggagaagcacagggagagggcaaggtctcaatagggaaattgagacttccctgccaggacctggaatcagggcccagtagaggaaatgaatccttagggaaggagaaccacactaacatgtattgagcacctacagtatgccaggccccacagtaggcacttaatgctcaccataacctaatgattttagaacatttatctaccttgattcagtgggaaaacaggccaggagaggttacagaggtcacactggaatccaaactcaagtcttctagattaaaggctgagctcaggggcctgagcatggggcagggagtggagggacggagaatgagattagtaatagaatcataatatttggacctgcaggcggaccttggaaatcgcatccagcatccttattttccagatgaggaaactgagtctcgtagagaggaagttgcttcaccggggtcttgttccagttggcccggtacccaccgccccccagtacatgcccttatgcccagaaggcctctgcaaacactcccttgcagggccagggaggccgctcaggctggccctctggcccaggccttcctgcaccccctcctccttccgcaccccctctgccttcctgctcctcctttggggaaggaggtctcttgactgggagcagcagaatgggggtatttttagtccccttctgtgtacctgagacagagttgacaggccagaaacccagctctgaatttcctcgtgttccctcttgcctgggcctcaaggcctgcccctgcccgagttctttctctgccctaggccctgctgggtcctcagggtcagcactgtgcctggcatgtggtgggccatcatatttaacacatgaacagctaaacggccagaggacccccatcccctgtggcagggaccccaggcacaaggtggggcctgtgagccactccagtaattctgccgaccaacgaactggtccctttgttcttccctctccacaggcaaagaacagaaggagaccaacatcgaatccatgaaaatggaggcaagtgttccacccccaggggctctgtggatcctgattctagaagaaggacacgggtggcgagtgtgtgtgagggggcggagggtgggcccctcctccttctttacacatccctctgtgggaaggagaggaagtgggcgtggaggttggaagtcccccaacaaggcagccggctcatccccctccagaggccagacgcctcccttcgtggcctgtggccgtgtccatgtgcccccccacccccgccacacatgcccctcgctcctccagcccacacggggcctgcccagcactcccaggggtgccctctctggccaggagccagtgtccacattgtgtttgaggcgagtgcctccttggtgggtccgtcagtgcccaccctaatgccctaatgtctgtctctctgtcctagggctcccggggccggctgcggggtggcttgggctgggagtccagcctccgtcagcggcccatgccgaggctcaccttccaggcgggggacccctactacatcagcaagcgcaagcgggacgagtggctggcacgctggaaaagggaggtgaggcgccttctggcctgggccccagcccgtcctgcagaactggaaaaccagaagtgatgggcctcagtttccccttcaggctaacccttagaacttactgggagcctcttaatctctagtcatgatgactggttcatctgctccttcagcagatgcttattgaacacttactgtatacctggctgtgtactagaccctgggcgtatacggttcctgctctcatggagttgacattctgatgggggagacaggcagaacataaagaaacaaggggctgaaatagaagatagcagaggcctgctttagttggggtggtcaagacaggcttcttggaggaggtggcatttcagttaaggtctgaaggaaccgaagagctggctctgtgaggagtggggtcagggagagtgttctaggcagaggaaacagcaccagcaaggccttgacttgtttggcaaactgtatgccagggtgaaatggcaaaggagaaagggccagatgcagccagagaggcaggcggggctccacacatcagttggagacctgcagagtctgcccaaaagctggcttacttctgacaatactgggcagccatagaaggtttctgtgcacaggaaggatgtgatgtggatttgagaggcagcagtggagacagagaagagctttggggtgtattttggagctagaatagctagggcttgctgatggaccggcagggaggggaactggtggactcaaggaggactcacaggggtctggcctgaacacctggtgatggttgtgccatttgctcagatgggaagacccaggggaggatgagtttgggacaggtgggaattgagggctccgtgtgagatgccaggtgcctggagtctgggctgtagggaagtacgggcgacagaggtgggatttgagagcagaaccctggcatttaaatccatgggaacttaagggctcagcaaggggagaaggaggagagacgaagtctcagaacatgtccatgaaaaccccgacatttctgggccacatggagaggaggagtggcaggcagtagggactgagggagaaagcagtacagagcggtgtctggggaagcacggggttttcccagagggtggctgcccagccctagggaaagctgaattcactgagacatgcccatggcgttggacaacctggaaaggagctggtgactttatagagaagcttctgtgggggtcggggaggtgggaacagcccgtggagggcttcagccatgcagagaggtgagaaaatggaggctgaaagactgaggagggaaatcaactcgccacgcacaatttttgagctgtcttgtatgtttgattagtagagaactgggtacaaagagaagtgggtggtgacctagctgcacaggaaaagagaggaaactgccaccacgtggggcacttcacgtgaactcagctctcatttcgtctttaccccaggcctccaggggaaggcggccctgtttgtctgttcgcctttgtgtgagagctttctctagagaaagtgcagaacagaggccaggcgcggtggcttacacctgtaatctcagcactttgggaagctgaaaggaggtcaggagttcgagaccagcctggctagcttggtgaacccctgtctctaccaaaaatacaaaaaattagccaggcgtggtggctcatgcctgtaatcccagcactttgggaggctgaggcgggcggatcacctaaggtcaggagttcgagaccagtctgcccaacatggcaaaaccctgtttctactaaaaagacaaaaaattagccgggcatggtggcaggcgcctgtaatcccagctactcaggagactgaggcaggagaatcacttgaacccgggaggcggaggttgcagtgagctgagatcacaccactacactccagcctggacaacaagagcaaaactctgtctcaaaaaaaaaaaaaatacaaacaattagccaggtgtggtggtgcgtgcctatagtcccagctactcaagaggctgaggcaggagaattgcttgaacccgacaggtggaggttgcggtgaggcgagattgagccactgcactccagcctgggcaacagagcaaaattccgtctcaaaaaaaaaaaaaaaagagaaagtgcagaacagggtgggggttctggctgcttgattgttttcttttcctctgggatccaggaaggcagaaattactttcccggaagagcccggagaaagggcagcctgtctgaggtctgggaggagaggcacttacccctgtgcgtggaatgttgtgtttggggtggaaattcaggaatggggctcttctctccaagtcagtcgctgcactcctgggttccccgcaacccagaaacaggggctttgctttttagctcctggttctgtgagcgaagtgtttctgttctcatttagccctggggcaagaagcttagactatgacttttaggaaccggaagaactttcagaaattccatgattctcaaactctgctcccttgaagaagcagtatggaggcctgccctcagttacagtgaaaaaaagggttctttcctcaactcccaggaaaaattagatcatagatacattttctcaattttaggtctctactaaaatttaaaaagacttttttgggctggacgcagtggctcacacctgtaaccccagcacttcgggaggccgaggcgggtggatcaccttaggtcaggagtttgagaccagcctggccaacatgttgaaaccccatctgtactacaaatacaaaaattagctgggcatagtggcgggcacctgtaatcccagctacttgggaggctgaggcaggagaatcactggaacccaggaggcagaggttgcagtgagccgagattgtgccactgcactccagcctgggtgacagaacgagactctgtctcaaaaaaacaaaacagactttgtaaaactcatgaacattttctaacatatgcagaagcagagagtagtgtaacgacctcccattgccatcacctggctccagcaattatcaaaagattttctagtaagtcttcaacccttggcatttgcatccccactcccaaccaatgaaggtagcagtgggcattattagaaaaaccaaaatgttgatttaatttaaaagactttagtacacttgacttgcaatgtggagatctccaaacaaaatatattttctgctaataacattattccttattcctatgctgtaattaaagaatccatgaattccagtacaagttttagaactactgatctcattttcagactaggggacttggggctaccgagttttataactggccacaagctcccaatgaagaattgggaacaggactagaatggaattagaatttacgtttcctattccaatgtggggcccaggccctgactgtcacacagtctcctcctgggctatgcctggggttaggaaggggctagatgcaggtatcaagaatggggaaaacacaggtagctttcaagattcccaagtcgccctcagactcaagagtagtagctgttgctatcaggcctgttttctttgtttgtctgtttttgagacggagtttcactcttgttgtcaaggctggagtgcaaaggctggagtgcaatggctcactgcagcctccgcctcctgggttgaggcgattctcctgcctcagcctcccgagtggctgggatcacaggcacctgctgacacacccggctaactttttgtatttttagtagagatgggggtttcaccatgttggtcaggctggtcttgaactcttgacctcaggtgatctgcccgccttggcctcccgaagtgctgggattacaggcatgaatcactgcacccggcctatcaggcctgtttttgttttttggtttttgtttttttgagacggagtttcgcacttgttgcccaagctggagtgcaatggcacaatctcagctcactgcaacctccatctcccaggttcaagcgattctcctgcctcagcctcccgagtagctgggattacaggcgtgcaccaccacacccggctaattttttgtaatgttttctaagtggtgtaacaacagcacagacatttctcctcttttctcagtctcattttgctctctgacatttgagcccttctgaaactgcctccctgggccttcccaaggccgctgcaggttcctccccacctccttcagctgatctcaagcggcaacctctgttagctgcatccccagatctccaccccccaccctctgccagtgttgatccactgaccccttacccaccctcctagatccaagtaccctcctcctcccaaaaaaggcacctcccctcctgacaaaccacccagccttcaggacccagctccagctgttccccaggcaatatcacttgaagggccagccagcgcttcctaatctcagaatgcaacgcctgcaacctttcattcagggcacctttactgagccggtttccagaagcctccacagctgagtgacatgtaagatactgggactaccagaggcatagaggattcctagttttcccatgataggatgagggctcatagcacctgcagaaagagctgccctgaatggggcgtagacgaatgctgcctggggtcatctacgcatcagcaggggtggcttctggcttttgacggttgaactggaatttaggggagggctgaggaaggcatttcagagggaggaatacagcaggcacatacctctcgaggcacaagacacttgggcttttcaggaatgggagtttgttgtggtggtgcacagcgggtgtgggcagaaagcagctaggaaatgtggctggaaggtggggcaggggccagaaggaggagctgccaccagaccccaggctgcaccagcccttcccctaggctgtgtgccccacctggggggcttcttccctgggaacgcactccccagcttaacttccttctccttgcccacccttgttggaccacagttactgagtgagcctctcccagattacccccttcaggctttccctcccccaagatttaagacgactcagaatgtcacaaaatgaaagaaatggaaaataagtgaacggggaaaatgagacatttctgcagtccctgaggctgaagtgagaattcctgctggagctgggaaggaagttgttgtcttctgtctgttccatctcctgttgctgttccttcacctgcttactagtcctgtaaggactaagctctgttttaatcatgatcacaccctgccatagaacctccaatggctccccattgtctcccccatcatgtcagataccctctggctatttctcaagtcttcaacccttcgacctccacaatcagcccaccccactttcactacagtcctctaatagaagggtgtcccctaggaagggacattgccatgctccttccttccttcctgtcatcgttcctgctgtgatttccccagtccattcttcagggttgaggccaagagctgctgcctcctccaggaaggtttccaagatttctccagtccacaatgacctcttcccttccctgaatccctgtaaccctcactggggtttgtttgtttgtctgagacagtttcactctgtctcccaggctggggtgcagtggtgcagtctcttggctcactgcaacctccacctcccaggttcaagtgattctcgtgcctcgcctcagccttcccagtagctggaattacaggcacgcaccaccacacccagctaatttttatattttaagtagagatggggttttgctatgttgacctggctggtctcgaactcctgaccccaagtgatccgcctgctttggcctcccaaagtactggaattacaggcatgagccactgcactcagactgtccttacggttaatattacttagcactcaatcatatttcatcagggtatttttcactgtttcttatgtgttgatcacatcttcctctgtctggcctaatgcaatgggcagggagaacaggcccagtgaatacctgctggctggctaagcagttgggaggtaggtgaatgtgggaccacttctctgggaacatgagatcttagatttccaccatgacaagggcccacacacttgatttcgaggtggtttttccaggggtgctgacaggtgtacctggaggatggcatggggactggtatggacagcctccatgggtaaaggggggccagagtctcctccatcctgtattcctgccattctcaccaggcttctgagctgaggagaaccttccttggcctggcagagctggccagggctgtggggagggcagggtagggcagttcctcctcccaaaggaaggcactcccttagctcctcttccttctaattctcagagcaaaatgcactctgatggagggcttgagggttaggaagggcctctcgcttcattagccctccctgtagctctgcaaggcagatgagcacagagacccagggtcagtagctgggcggtcatctcgcctgggaagccactcactccagtttgtgtcgcggctggtggttttaggactcagttacagcacattgatggcatgactcgctcattaatctcttcaccccagccggattctaaattccgtctaggcagcgggtaccatgtcctattgttctaggatctggtctctttcctgcttccgacctgaaggtagggacccttgactcatgcatccttatacttattagatgcttaataatattgaatagtgataatgaaacatctgtgactggaacatcatagtctctcctcccttccctcgccacactgagatacaacccactccctccgcccagggctgcccagcagatagcagagccttcatggaccttaagggttggctgctgtttactgcgacctttggaggcaagttcaaatactgccttaagaaaggtcccacttactgggtgtgggaccttaggcatcacttaccctgtaccccagctgaggttcctcctctgtggatggacttggctgtactctgcatctccatgacagcagcactcgccagcttgttttctgcctctacccatttctgcatttaagctcgttcaatacaggcattctgggcaccccctgcccccagggctggcatgtgggaggtgcagggtgaaagtgtgctgaatgaatgaacaattcttatctcagagttgtgtggacaacacacaacatttaaagctcctagcacagagctagcatataataggaactttataattttttagaaagtattttaaattacataagtattacacaaatacattcttgtgaaaaagtccgcccaaacttgccaccccaaaattttattttgagacagggtcctgctccattggccaggctggagtacaatggcatgatcatggctcactgcagcctcaacttcttggctcaggtgatcctcccacctaagcctcctgagtagctgggaccacaggcatgtgccaccacacttggctaatttttaaattttttgtagagatgggggtctccttatgttgaccaggctggctttgaactcctgggctcaagcagtccttttgcctccacctcccaaagtactgggattacaggcatgagccaccacacctggcctgaaaatttaattccttcaccagaaatacttgatgctatcagtttggtgtaacgtcatgattttttcctatccgtgtgtatgcgtgtaaatatataattttgcttcaggggttctttttttcctgttctttttacacaaatggaaccacactgtagtttcattttacagcttggtttttgacttccaataagtctgtgcaatctagccacgtaattactattatattccaccatgtggattcccatggttttatttagccattctgttattggtggccatttatgttgtttatgatattttactgtttagcattgcagcggtgaacatccttagtcatacctctttgtgcatctgtttctctaagacagatacctagaaagaaaaaggcctagctgaagggaatgtgcattttacattttatgacatctgccaaactgccctctaaaaagggcactccaatttatgcttctgtgaacagggtatgagaagacctttgccaacatcaatattttgctagtcaaatgggtgggaaaatagtatcttgttatgcttttaattggcatttcccagataataaggttgaacatcttttcctatgtttattggccatttgtgctgctgcttctatgagcttagcaaatgttgcctcctctgactttacaaccctgcagtctcaacagactgtgcttcctgtctgccctgaggagagcaggccatgaatgtccagaaggtcccccatgctgtgtcatattccatttaataagcagtcactcagcacctactaagtaaaggggccttgtgctaggggctatgggaaggaggagataataaggtgtgatctcctgggctttagtctagttcaggagaaatggaagtgcaaagtagaccagtaaaggctcaaaacaaattattagccaactgtatgggaagcagttttagagcatttggactggaagacattcctcacttatttgccctacctgaccttttgcagcgtggcctcaaaaataacatcacccttgaaggagatgatgatgtttggagtaggcacagcaagtatactttacccaaggctgaacaagttctagaggctcccatgacagcctgtaagcagtagtgacttcgagggctggcctttaccataggctactaaagttgccacagagggactccccattgtcctttaagtagatgggttggttgcgcaccagagtcttcacttctggggcacagagagaacagctcttagaacaacccctactttttagtggtgagaaaaaaagaggcagggtggggtaggtgactagttgctggcccagggccaactcctggccacagcaaagcctgaaccagggaaagggtgttgaacttcactgagcacctactttacatcaggcactatgccaggagcattagctttcaccacaatcctaaaacatattatctgcatttaatgtcttttttttttttttgagatggagtcttactctgtcacccagtctgaagtgcagtggcatgatctcagctcactgcaacctctgcctcttgggttcaagcgattctcctgccccagcctcccaagtagctaggattacaggcacctgccaccacacccggctaattttttgtatttttaggagagatggggtttcaccatgttggccaggctggtcttgaactcctgacctcaggtgatccacctgcctcagcctccaaagtgctgggattacaggcatgagccaccgcgcccggccagaagtatttttttaacctgtattttgctattagaaaattaagatttatctaggtaagtaggttgcggccacatgggtatcctgatggagtggcccaatgggcaaaactagactccaaatctcgtgttgcttctgttgtgtacccaaatgactaacctacactcccagttttcctggtttcctggcttggaaattgtcatgaaattgaagaatgcatttagaagagagggagagagctggcccggctttccagcctgccgcatgctcttcccgcccccggcctgcagttgttggggctgctccccatgcctctttgccaggaggctcaggtccctttcagcctccccgaccacccctccctgctctactcatgtggtcctgaacccttgcccttgaattgctgttttttcatgaccatgtggatgatgttgtgaaactggattaagaaaatgttggaatgagatttgggaggcctggtttctcgtctcagcttctgcccctccttagctatggatgtgaggaggtctttctgctttcggcattttagtttcctggtttataatgcgacagaaatagatgagatgaccccagagattctaaaattccatcacggcaggttggggggtgagactggaatcatgatctgcactctctttattacctagattaccttgtacataaattctaaattaaatatttgagtgaattctcagacctcgcatatacgtctcttctaaaggcacatacgtgaagcatccttataaaaactctttttggccgggggtggtggctcacgcctgtaatcccagcactttgggaggcgaaggcaggcgtatcatgaggtcagcagatcgagaccatcctggctaacatggtgaaaccccctctctactaaaaatacaaaaaattagccgggcgtggtggcaggcacctcccagctactcgggaggctgaggcaggagaatggcgtgaacccgggaggcggagcttgcagtgagccgagatcgcaccactgcactccagcctgggcgacagagccagactctgtctcacaaaaaaaaacaaaacaaaaaactctttttaaaaaaaaaacctttggcccagcacagtggctcacgcctgtaatcccaacactgtgggaggccaaggcgggtggatctcttgaggccaggaattcgagaccagtctggctaacatggtgaaacgccgtctctactgaaaatacaaaaattagctgggcgtggtggcacatgcctgtaatcccagctacttggaaggctgaggcacaagaatcgtttgagccagggtggtggaggttgcagtgagtcaagattgcgccactgcactccagcctgggcgacagagcgagaccttgtctcaaaaaaaaaaaaaaaaaaaaaaaaaccaaagggttgtccagcctttactgagcagatactgtgtgctcagtctttaagataccaccttgtgtgtctgggtgtctactatgttccaacattttctagacactaaggaagcaaatttaaaggcaagtcagaattctaccctcccctgtgagaggttggtaggtaaatgaatgaattgtgctgtagccagggcagggttgttttagaaatactaagggaaggactgtgcaagcaactccctagccctgcctaagggcctcagggaaggcggtgcctagggagaggcagtgtaccgccattgacctgaacaggactgcttggcagggacagtgggggaagggcatggagtggggtcctaagggaggtggtcaaagaaatcctctaggagtttggagtctagagtgcctcaaagtttttcccatagcactgtctatggggctgacaaagcagaaaatataatagccaatgttctctctgctttaaaaaccagactgcttgtcagggaggccgtgggatgttcacatatggggtggtctgccagagccctctgccgctttattaagaggccagtacactcgttaaaggagttaatggacctcagtacttactactgattgcttagtaattcagtacttggtaaataaacatgatactatgctagtggcggggtagggggagaccaattggatatagtccctgtcccagaggcacacattgtcaagtggaggaggcaggaatgtttccaacaagcctcagtggcagtcagagcgtggggcttgctctgtgctcctgtgtaaagagcccaacatgaagcataaaggagcatgagagcagcttctggaggactccaggaaagcttcctggaggaggcaggcatcctttgagaatttcagtaagtgcaaaagaggctgggtgcagtgtaatcccagcactttgggagaccaaagtgggaggattgcttgagcccaggagttcaagagcagcctgggcaacatggccagacccactctctacaaaaaaaatatttaattagctgggcatggtgttgtgcaccaatggtcccagctactcaggaggctgaggtcgaggctgcagtgagccatgatcatgccactggactccagcctgggtgacagagtgagatactatctcaataaattataaacaaacaaataaaaaagtgttaaagaggaggaaggcatttcaggaaaaggaactactgagggcaaagagaaggaggcatgacttctgggttgtttagggagtagtaagagatctggtaactagggtgcaggggttagggaggatgggcaagaagagacagaactagaaagcaagatggtcacatcctcagtggtcttcctgccatagtgagaaatttaggcttcgttgtccagtggagagcaggggtggaagagaattggacacatagtttaggaagataacctggcaggacaataagtattccccagagagaggaagtttagaagcaggaccaggtaggagaacattacatttggccttgactgagggttgaggtaatgagaggcaaacttgaaggaattgctagaactggtcctggttggaggggagggaaaaatttgtctggtattgggagctgggctggggatagggttcattatctgaggtagagaatcttggaggaagagcaggtttgaggcaggatgatacagtgaacttttcattttcttatgtattctgatgagcagaagtttttaataaagtccaacttacccatttttttcctttattggttagtgtttgtgtgtgttatctctaagaattctctgtctaccgcaaggtctttaagatattttctcttaggagggggtggatttttgagctagggtttggataagctgcttttgagaagccagcagattggtgtcaatggggacaggttttttttttttttttttttgagacggagtcttgctctctcgccaggctggagtgcagtggcacaatctcagctcactgcaacccttgactccctggttcaagtgattctcctgcctcagcctcccgagtaggtggggttacaggcatgtgctgccatgcccagctaatttttgtatttttagtagagacggggtggggtttcaccatgttggccaggatggtcttgatctcctgatctcatgatctgcccaccttggcctcccaaagtgctgggattacaggtgtgagccactgcgcccgaccccaggggacaggtttagtcagattccagggtggtcctcatggaggggacgcacaggcaggagggcagggagagaaagcacagaaagcagacctgctaggagaggaccaggatagggaggggcagggacaaggacatgggacttctgggagcaggtggccgggggtcaacgtccaggaggatttcctgaaccagttagaggactactgaacccccctttcacctatctatgcagccctttacttttacaatatatacatttctatttattttatttatttatggtttttttttttttttgagacggagtctcactctgtcacccaggctggagtgcagaggtgtgatctcggctcacttcaacctccgcctcctgggttcaagcaatcctcccacctcagcctcccaagtagctgggattataggtgtccgcccccacctctggcttttttttttttttttttttttgagacagagtctcactctgtcacccaggctggagtgcagtggcatggtcttggctcactgcaacctctgcctcccgggttcaagtgattctcctgcctcagcctcctgagtagctgggactataggcacacgccaccatgcctggctaatttttgtaattttagtggagacggggtttcaccatgttggccaggctagtctcaaactcctgagctcagatgatccacccacctcagcctcccaaagtgctgggattacaggcatgagccaccacacttggccacctctggctaatttttgtatttttagtagagatgggttttcaccatgttggccaggctggtctcgaactcctgacctcaagtgatctgcccacctcggcctcccaaagtgctggaattacaggcgtgagccatcatgccgggcctacgttaatttttgagacagagtcttgctctgtcgcccaggctggagtgcagtggcatgctctgatcgtagctcactgcagccttggactcttgggctcaagtgatcctcccacctcagcttcccaagtagctggaattacaggcacgtgccaccatgccctctgtggagccctttctgacttccatcaactccctgcgaccaccccgcatccttcacctctcagtccgcactccctcccaggccaactgcatggtgagtgtgtgtgtgtgctcccaggccaactgcatggtgagtgtgtgtgtgtgcaagtgtgcgtgtgaatgttaccatttttctgacttgccattcttcagtagcaactctacagatgcgacagctcttccccgttcctctttagtccatgaatggtgggcagtataaatattattttcacttgcaactcagatggattgaacaaaaacaggaataacagctcagcctcctgtatatttgacgctagatgctgttggaaaggcagctttgtggggcaaaaaaaaacaaaaattgagtgagcttgcttatatataccaaagatgctcttagacttcaatgtttctcaaagggtgagattgtgtgttacctcaccagaaccacctgggcagtgcattaaaaaggcacactcctggagcagaagctctgggacagtacttgtaggtttgattttctagccagcccccagatgattctggggcttactacagcttgaaaaccaccaagcctcagatgattagaggtaaaccaaatctggcacagatgagattcccacacagatgtattggtagatgcatttattgtatcgcctgcattggcggcgggggtctctctacacattttgtttaaacctgggccaggttaatcacgttgggaatttagtgatgtacccagcaagggtggtttcccaagctctccgggtaattgatagggcttgggttgccagtctgattgtggtaacaagcaaaggaagaagtagggactgtaaggcgggcagacctagggaacagtttccaaacagggttgggggggaaagaccggaaaggacttggtacaggaatttaaatgtttcctttcggatggagatggcaggggtcctgctgttttcagcaatagttaccgcagtgcctgtctgctgggctcctctggagtcttctctttggaccctgaaacttgagagggtctgcatgcctgaatctgaatgcggctcctttaggcccctccctttgacccctggagcctggagaaggggtgggggaagggtggggaaaacaggcctggctgttaaggtttaccaggttccttttggccacaccccccttagtataaacttaacacttctctccagggtccagtttctaaagcaaagtgacagcatcgacacgtccctaaagcaaacttgtcatgaagtccctcacctctcaaatgtgagtccctcctggcttcctttgattttatctgtaatatttagcagatacgtgtgattctattaggctagttaggaaaccaaggcccaaagaagctaggggttatttaaggtcttgtcatcagctgtaaaacaccctgctcagcgccctgctcagtgaaagacctggcatcccctcccagttgaatttcctggtaaggggcacctgagatccggtatcagtggggtactgctactctcccacccctgccctggcatctctgctccctggccccagactccgtcactggtagagctgggcagacccagcagttccaggatagcgggtggaagtgagcaggaaggaagagctgggtttgtggagaagccttccccttatggggcaggctatagggaaagggaaggcagccaccaaagctccagagcctgatggcgcatctattgggctgggacttcccagcatacaccattagggaagtgtagggttggctgggagagaacgagggagccaggggctctgggatagaggtggggagagaaggggaggtgagggtatcagcaagagggaagttgggctttcctttccagcagttgggctcccccatcctgcatcttcctccttgatttagaagaattcatctagagtgtcagtgcaaaaagccccttaccactccccctccctaacagataccctaaggctgtgcttgtgcttttcattcctgccattcatctgccaggaagctcatgcagttacagatgccccctagatctggccattcctgtctgaaaaacctgggaggttgtgcccattcctagtttccgctctttcatcttcccttgagccttcctgccttgactgatcccagctgcctctgagttccttcggcacatgtagaaagcatccctaccacataatttaccatctaattgcatgctgtcttgaatgagggctgtggtttcatgtgtggtgggtcttgggtacacaattaggctgtgagtaacagaggcctagagccccatcatgtctactagctcttgcacctctttgcttcccttccccccagcctacctactgcaggtgctaaggactgactgacctgatccggtgtttaggtgagcttcaggcaggctcccaggataaagcacaattcgcttacaggttctaaaaccagactgctcagtgcaggggttaggggggtgcccctgcccgacagcatttgcccaaatgccgctcctctccccagactcagggaggaggttctggggcagggccgaggtttggggtgatggcatagagcccaaccaggcagcacactgcagttgccatggtgacccttttgcttttcttcaggaggatggatttctcttttagcccaaatcatctgcctgggctactctgagaaccagattcctagccccctaccttccacattctaccacagggggcagcttgtcagaggctttggtttgaaatcacagtccagcatcagacagcaacaggttgtgcttgatcagggagcaggtgggaaaggcccagaggagggctcacctgggacgcatgccccgagggagctgacctccatctccttctgcttcccctcctctctccagaccacttctctccagtgaattgctctgacctttcaggtgggctcgggcctctctaggcctcagtgttctcatttgtaacagacacagccagggtctccgtgtttccacacctggctagtattagaattacccagggaggttttaatagattcctgggccccaccttaagcccgctgatctacgtaggtagaccccaagaatctgtatttttaaagccctccttgggtgatccagatgcaattaaaccaaagtgaatctggggttctcctggcgaggtgattagcaaggccctcgtgaaaagaaaggaagttctgcctgtgctaaggattgactggcctctccgggcttccaggagcttctggagagccccttacagcctagtcttgtcaggaagcgggtgtttcctggtcatgccggctaagccaagaccagacccccacttctctccctccagagaagccaggagagagagaatgacaggaaggggggatgttgccataacaacaagctccaagcagaggcctcccttccaggggctggtacttcaccccagtcctggtggtttccatggagatgggttactgagggacagggggaatgtctgccccacttagagcatcagccaggagctgccagaaggcagaacaccgctaggtgcgagcctttggacccctctgctctccgggtgcacatctgacaccttccccctgcttagttgctctctctgcgactggtctccttagcaacaagtcctgctaactccacgctgttggcttcatggctatttttagcttcactgccagcagcccagtcctggtcatttgcttgcaacctccatatggattgcaagggaataggaaaacaaagattcccagcctcttttggtagactgggacctcgtagcaacgattagaattcctgctatgaaatggatccattccccaacttggaacttgggcagtggatttgggataaggttcctgagacaacactgcattcagttcatttttaatcctctactatgggcaagatgctctagcccacactgcttccatacgaaggtgaatgaggtccagtccctgccctctaggaatatttaatcttgtaaaggagaaagatgagtacacaatcaccttcgcacaaggcaaagactggcaggtcctaacagaggcttacaaagtacaaggggagtgtggcagggatggggtagaagataagggacagaagggaaagataagggacagaagggaaagataagggacagaaggggaggataagggtgaggggccagggcagcactgtcaaaagaactttctgggatgatggcaatgttttaaaatcagtgccatcgaattcagtagcccccagccacatgtggctcttggacacttggaatgtagccagtgtgactgaggaactaaattttaatttgtatttaattcaaatagacacatgcagccagtaactgccatcctggacagtgcaaggctagaggaactttcatggtggaggtcactggcggactggtgtagaaggatatgcagggtttaggtggtgaaagttgaaggggaggaaccttccaggaagaaggaacagcatgaggaatagcaagtgggcgggcaaagcgtgggcttcatgatatcacacggaggcctccagattcgtgggggcctagaatgtcacgcagagtgttgactgattggcattgaggcttttgagctgtaggtgggaggaggacgggttcagaacagacatccatcctgacctctctttttgtctggatcaggtctccctggtcttggggttggtgggaatacgtgtggggtggaaatacgtgtgcagtgacttcatatgtgttgctttgcagatggctttatagggaccatggcttctctgattcagagtagagagactcgcatggtaaatgtcctggtgggatgacagcaacatcatctaccctcatcctgccggaactggattttatgacagagcatgccacagagcactgaactttgagtaaaacgtcaggcatgagctccgggcgttgctgctctttggagaggtatcatgatgggctagtgtctcgtctcaattctttgaggctggaaagtcacattaacatacttatgatcatgactcttcaatgacccaggagaatgatgcaccccagaatgttagatttaccaattgataacaggacatgtccttgccacacaaatcataatgcttgtcctgctgttttctggcaacttactggtatcagagtcattggaaaatgtaattaaaattggaagtttgcttgatgaatacaggatcagggcagagaggtgtccttcagaggtatttttgtcgggcttaggaggcctatgggatcactgttgtctgcccaccaaacaggaactgtgctctcattctcaggtcactctctccattggaccacactggatacttcctttccagcacctcttttaggctcttgacatccagcatattttcttctcatccaggagttcctcctttcaacatacttttataacaaatccaagaacatggcttattttacatggtgctgctatttgagtttggctatctttaactcaaacaaaagacctatctggtaaactaccagggaggtttactttggaagctcaagagcatgtgtgacctgaggatccttgaagattgccccattttagtaaggggatttttccttttactaaaatcacccctgctctagatcttggggtctcttctgctttggggtctgactgtgcattgaccacaaagaagacacatctctgtttaagcgactcagtctagaaaaggcagtgtttcctgtctgcatagttacaggagagggaagaaagatgctctgggttgttaaggggacacctttgagcctaggcttccttttgcttgttgggaagttgtgtggaagaggtaggatttgactttaatgttaaaggatgagaataaggaagtttgacggccggagggaacagctacctaatatgtggccaagagaaagtgaacagttctggccagttctgacaccttgagtccttgctgcacgcacggcatcattgcagtcttatgtcccaagaccacagaatgtggagtctaaggtgtttagccgcattataatggaagagcgggtatcgtacatgagacagtgtctgcaaaaggccaccaagccagaaccagaaacttcttttgagaggaggcctgaagtaagttctcaggcccgttgaccggcatggatgcagaagaggtatcagtgagagaagcagcgaagcaagtcatgctgcattccctaaaggttgggagcaggcaagagggacaaattctgggcttctcacttctgcttggggagcccctggaaatgtgtaccaggcaaataccttagttgggttgaaagatgggttggaacagaagagggcctgtgtttttgtttgtttgcttgtttttcgaatagcttcttttcctagctgtatggattctgggtctcagggttgggggggagtctgtacccaagtcttctaggtagaaggctctctggtttggggtccttgctcttcagggtttgcttgctctgatggccctcagggggagggggggctccctcactgcacatctcgtgacttcattataccagcacctgtatgtcccaggtgactgtggctgggagccaggcacctagagaattgtctcattgtcattaggagatggtggcgttccatggccaaagagggctgatgtcatcactcgttttgcagatgagacaacagatttcttgggggttaagtgacttgtttaaggtcatggtggtggaaacagaactgaagtccagatcttttttttttttttttttgagacggagtctcgctctgttgcccaggctggagtgcagtggcatgatctcggctcactgcaacatccgcctcctaagttgaagcgattctcttgcctcagcctcccaagtagctgggattactggcgcacgccaccacgcctggctaatttttgtatttttagtagagacaaggtttcaccatgttagtcaggccggtctcaaactcctgacctcatgatccgcctgcctcagccttccaaagtgctgggattataggcgtgagccaccgcgctcggccaagtccagatcttctaacaagtgccgctgcccaaatagccctctgctgtggggtgcattttcctccatttcctcagttcttccttctaattcatcttgccaacggcaactaggctgatttttccaaaatactcattcatcttgtcagaaaacctgcggttattcttccctgctacagaatatacccaaggacgcacctgaaggcttgccattaccttgccctgtcgtgtactgggagggtggaggtgggcgagggtctcctccctccccagcccggcagctcttgctcatcctacccatctcacctcattccaagtccgatccagcctccaggcccagtcggctcacctggaactgacctctgacctcttttgtcatccatgccgcccatttttttctacttggtatttgtggcatagttacctttacatatgtttgttttacagtgatcctttcatatttctccaagtctagtggaatcttcaacccctcgagggcagagccaacagggtctatttctttatctgatcctacagccaacgtaatggagggctgtgggtggggactgcgtctgccttgggggtaggtgcctttgttcaggaggaggaagcttgaaatggcggaggctgcacctggaggccgcacctggaggccccaggagaggagtcaggtcttctcgatctgcagatgtttgagcctgggaatgaaggaattgctgaactttctgaaggagcgccctcgccgcgaccaaccttgcaaacaggaaaatgagaaatccagggaaggcccagagtgacgcaggggccctgggactcgaagcctgacctcctcacgccgcgctttttgaggcccccccgcttctctattcacctgtagtgtggaggcgggagaccccccaaacaatccccgatctggagcgctcccaatgcctgcgcgcgcctgctgtcactctccgtctgtgtgctgagttttcctacagcttcctgggcctcctatctgtaagctttttctttttttttttttggttgtgcttcagagaaactcacttttcacaactttctcccggctctcccaggccgtccgaaagctccggcttgctttcgcccggacccccggctccctccgggcaggcggctcgggagcagccccttccctcccctcccggccccccggccccgcgctaatctcttccagagctgggggaggggccaggcggtcttcccgaaggcggggcgctccctgcagcccggcctgggcgggccctgggaacgggcggggaacggcctcgccccccggccccgcgcccctcggaccggagaagaggggctggcccagcgccagcgtcggagcgccggccccctccccgggccgctcgcagccaaccaggccctccagcggggcccacgtgacctggagtcctagacaaagaaaatgttccctccctcccccccgccgcccccctcccctcccagtggccccctccgcccccagccccatcgcccccttcccctcccccaagacgggcagctacttccagagcttcagggccgcggctcacacctgagcgcgactgcagaggggctgcacctggccttatgggtaggttcctggacggagccccggggagaccgggggcggggaggcggcagggccggtgggaggatctccgcagccctccgggtttgaaaagagtgaagtatgcctccccggcccgcagcagccgcggcgtcccggcctggcccgagttctggagatgctgacttagtgctggcccgccgccttcctgcccacgagccccaaggccccactcctcgagggctccagtggcacctggcgccctcgggggacaggaagagcagccggccgggagcccgggcagtggtaactgcaggctggggcacgcggagctggtggcccaggagttgggttggaggcagcttcagctgagctggcagatgggtgcctggcacttccctggtgacctgcgtttctggctgggggaccacgcggtcctcagggccctgccagcttgcggcctcttaagagggacagcttcctctcctgcgagttcctggaagcctccaggcaggccgccagaaagaagtggtggggggcgggagagcacggtgcgaccctgacagggggccggcctgtgacaccagggtagcccaaggccgcatgggcttgggggcggtggcgctcagacctctgacctttggaatgggaagcgctgcagtcgcagggggaggtggaggcgggacaactgcctgaggagatgggactggaccggaggcagtagtcaccggagttcccggtggctttcccagtctggacggactccaggcccaggggcttccgtgagccagcagggagtggggaatagtgtggtgtctcttgaatgccgcctggtttctccgcccccctgggggtgagcagcccgcagcccaaatgcgagagcctcgagttcacccctgtttctcttattgtttgcctggggaggaaggcaggagtgcagagggaaaggggtgctttccctacgccccagacaggcctagtctagtgaggcccctcccccacccagctccggctgccccttcccctttcgtcaggccccctgctgctcacccacccccccccccgcttcttccttcccccaccccactgccccagtcctgatggaggccccttcctcctccgccagcccttaccgggccgggtgcgtgttctgcaccaagtgggctggggatgctgaacagctcgcagcgcggcatctgacaagctgggggaagttgtgggggaagaagaatgttcaggaactcgctgtgaaggggctgagagacaaaggggccgtgggggagtgctgagggtggtgtcaggcacgactcctagacctggggagccagaggaggagcccagcccacctgctgctgccgctgccaggaactgatagcgggtcctttccccttcccgcccactgctacctgcacacctcgtactggctcaggggcctcgccacgcctggaaacggggtctttggatcatccgaggcatcaccctaggtcagccacccatgcctgaagcttgagaaggcgtggtgggcgaggagcagcggcgtggtgtgggctcctctgcgagggtagctgacgcagctgaggatcccagcaagggaggcaccctcccccatagcggggtccacacttcagtgcaccttctcgtaggggaggaggcgatggctaattgatagtaggaaggggagggcagagttctggccaggtgagagtggggctagggagcgatgagcaattactccacattcttccacccccgcagtccgatccgtgagctcattccgccatccatgagctcattttcccagctgtttgggggttgggggcaaaggatgcattatcctgttttagaatttgttgagtcagaggaagaactaggacttaacatcttcagtgtttttcggaatcgccataagaaacagattggtctgtggcttggaggcaaagacttgaagactcagtgaaattaaatgaatctgtaagacaatacattggaccactgagcttcaaacaaaggttcttaacctgggacccttggatctggggggtccattgacaggctttgggagagtcctgggaatcattggtccatatatacaccagatttttaaacaggtcagaaaagctttccagaaatccttccgttcaggtgcctttttatgttggagcaactcctagaaaggactgctccaaagtcagctgctggttgtgaggctccgacaggaatgtagaggctgcaacttcagattagagctcttcttcatactccaggttgcttgctcatcctctttacccttgaggatgggtttgcattcgcagctctaggcccatgggggagctcttttgaaaggcatgtagaaaatggggttttctcccctctaaatctggtatggtggaaatgggtgcaaagccttgaagtcagagggccgtgtttcctagattcagtaaaaggctaatggttctcagagctaagtatcaaggatttcattctcctttgtagggatcctggagcgggttgtgagaaggaatgggcgcgtggatcgtagcctgaaagacgagtgtgatacggtgaaaggatggaggctgtgcaatgggagaataactggggtttggcttattaatttgttggtgggatggcagagagcacgcctggtctatagtgcctgctcagtaaataactgaatgaatacatgtatttatggatctgaagggtgcacatagaaggggtcgaatctggtaacaatacacactgggtgtgatccataagaactccagcatggcgtctgagcatgggaacagacaggctcttggggagcttcctatggtgtttgagcatgtgctggggggagatgggctctttaaacccagagcagtgatttcttcgtcagcagacatcacaaaactagaattgtggcctggaaaccaggaataggaaggattgattgcccaggggaattgtggagaagaagagagggtcccccttgctttttaaatttttggtgacagggtcttgctatgttgcccaggctggtcttgaactcctgggctcaagtgatcctccctgctcagtaactgggattacaggcacatgcctggctgaagatcctagtcttgattaagaagataggagagggtggaaccaatatctgtgtctaaacaatagtgcctgattaatatttggatagaattaacatctccataggtagggggagggatagcattaggagatatacggtataatgctaaatgacaagttaatgggtgcagcacaccaacatggcacatgaatacatatgtaacaaacctgcacgttgtgcacatgtaccctaaaacttaaagtataataataataaaattaaaaaaaaaacattaacgtaggctagacacagtggctcatgcctgtaatcccagcactttgggaggctgaggcgggcaaatcacctgaggtcaggagttagagaccagcctggtcaatatggcgaaaccccatctctactaaaaatacaaaaattagccaggtgtggtggtacatgcctgtaatcccagctactcgggaggctgagacaggcgaatcgcttgaacctgggaggtggaggttgcagtgagccgagattgcgccactgcactccagcctgggtgaaagagtgactctgtctccaaaaaaaaaaataaataaataacatttccgtatcatagggtaggcaaccgatatgctgatgagattgaggaagattagatctgggaggaaggtttcctaaaaggcagaaaagaaggctggaattgtgtgacttgagttcacctctctacttcaagtctttccatttttcacggcaaggcagctggttggctctctccagcccttgaccttctgtcactgttccgggttttgacaaacatggtccccttgagtgtcaggtggccttgctaattcctggagaggtcaaggtgactttttgttttttgtttccccaggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggtgagcggcttcttcaccagccgtggccagctctttccgggggccttccactgccagctcctcccgtcctgctctcctctctccatctcccacgggccgctcgctctctccacctgaccccgggcctcttcgtctccccgattttgctctgtcttgcctcattcagatggagctccctcctggctgtcctgccccccagatgcccagggctggggttgccatggggggatcagggtggcagggcctcgtgaccactgtgtaatgatttctgctccttggggctccaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtaagttgtggggtttggcagtagcctggggtgggggaaggttcttgatcccagggcctgtggttttggctgggggcagtctggaattggggtgatggaagtgggttctgctcacccctactctgcctgtggtgggtggaagaagctgtagggataattaggttgcacctgtggctcagagagctaggatgctggcccagttttagccttccaggaacctattcactagaattcctttccacctgcactcaggtacttccctactcttggtctggactcctctttgcatcgggtaattatttatcactgtatctggtcccctccagggctgagactgactctcgaggctcccagcccctgccagttgcaaggcatggggtgggtgcttgcaagtgtaagcctcggcaaacaaggcctggcttgtccccccaggtgtgtgttgagaagctgatgccgctgagctcgttttgcagtgcgttccaccaggccacgtacaacaagcagcccatgtaccgcaaagccatctacgaggtcctgcaggtgagtgtccctgctgggagctcggaggaggagcctagagcactaggaggcctggaagttggagtgcaggtcgggcttgagcagaacggaggcccaggaggcagcgactcctttctctccacctcgctcgcccgtatgcacatcccgctcctcctcagcggttcctccccatcttgcctgtgccaccctcactactcagagtctggccttgagcctgacccatctgccttccagcctgtcctgacaaccccaaccctggcgtgtcaccctccaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaaggtaaatgagggcacccagctttctgggacccctgcccgccaggcagatccacaccagggctgggaaagccatgcttagggagggctgccaaggcctccacagaggggcccagtgggaacagccggggtctctcttgccctggggtcaggacttgaatgacctgctctctgttggttggctagaaccgtagagttgggaggtgggaacaagttggagaccaggccgcaccccactgtaaggagggtgggggaaggggctggagtttcctgtcagcctgtaactgaccttggcacctgctttcctcctccagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagaggtagttggcctgcttctggagagggtggcaccaggaggcctgcatccgaggagcaggcgcggcctcctctgacgccagctctcctccccttgcagagcggctggtgtacgaggtgcggcagaagtgccggaacattgagggtaagtttgttgcctgggaactctggcactcctagtgtggcatggggcagagtagcagaggcacttaggaaggtttgaatgacatgggaccaggggccacgtcctgcgtgtcacaccaccttcaccgtgcttcgtgcactgtctgtctttctggtgccatgattctgctctcctgtcaatggcgtgggctggtcttcagggtgtgggactccccagtggccacagaagggcagcccctcttgggtgtgactgaactcacctacaggtcccctttcagcctttcacctttgctcacctctctcatcctggggctgtcgccctcagcctggtgctggtggatctggctcaggaccctcggcctgggaggattggggcaggggtcaggacttgaggattaccccgggaatccctcctgagtaacctggaaccagggctgcttctcatgagcaggtttctgtcctgctgtcactccatccctgtttgtggatggtggcattggtgctctgagatgatggcgttcgagactggggtcacagtgcctcccttttccgtggggcaccctggactcttttctggctgccgctctggcacaccccttttttgtggctggtctggtgctgggctccggcccggggagggagaggcccttcggtggtactcaccccatcccctccctctgctttccagacatctgcatctcctgtgggagcctcaatgttaccctggaacaccccctcttcgttggaggaatgtgccaaaactgcaaggtaggagcacacccacccaggagaggtgccgttgaggccggcccttcggccttctggctgactgtgtccaccgcttctgggcatgtacagggtgacacccggtgtgcgtgtgatgtagacttcagagcttcctctggaagcagagtcctgatgacacgggcccttgggtttcaagcttttgacttgctctcatttttctcctctgaaagattctttacctctctcaagccccattttagtccccagatgtggggatgggagcagagcagcggggacaagctcaggcagagtgtagcagggttgccgtgccctggggagcccaggagaaaaacccagggctgcaggaaggcccaaggtggtggtgcagccccctccctggccttgttctcaggctgctctttggttctgtccatggcggtgcacaggcagatgaggtttccccttcccagatctgcagctggggcctgaccctggctaaggtggtggcggggtcatgtcttcagggcttaggctctgtgaggccaggtgtggagcctcccttcgtcctggccgtcctgggacccgtcctggtggtttctgacccttcccgctgctgtctagaactgctttctggagtgtgcgtaccagtacgacgacgacggctaccagtcctactgcaccatctgctgtgggggccgtgaggtgctcatgtgcggaaacaacaactgctgcaggtgaggctgttgtggcctccagtggtctccttagctgggccccaggctcccgccgcccaccgcctcccctcctctcccttccccacagaccctccaccccctagccatgctccagacccggtctttccattccaggtagcacaccttggcctccccggaccagggctgagagtctcctctgctcactgggtctccttccaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgtgagtgctgggcctggggcgcggtctcgagctcccttggctgagccttggttgtggggcctgagctgtgcgcagggtgtgtgggtctaggagcctggctggagggccagcgctgggtgggagcttgggacaccgctgggcctgcatctgacctgttgtgctcactgcttaggaccctccaaaggtttacccacctgtcccagctgagaagaggaagcccatccgggtgctgtctctctttgatggaatcgctacaggtgaggggtgcaggcccaagaggtgctggccgccttgtcccaggatgggggagccaggtgtgtgggcaactctggcaaaacgaaatgatggttagcttcacagctgttaaaatattatcccaaaggggaagcttgtgtgtgaagtggaaacgtatgcagtccgtggacttagatttgaccaaaaataaaaaagtaaaacaggtggtaaagcagcctgcctgctgggcgcttccttagtaaggaagattcagccacagaggccgagtgatcattagtcagattcacagagatttcacagagcctcaaaggacagattcagcaagacagaaacatacacagttctgttgtgttttgattatacacaagatagtctaaagaagacttatttaaatcacccattatccttttattaatattttggcatatgtccttttcagatttttttctgtgcgtttgtgtttatataaccatacatccatagatagataaattaagtgggtagtgaatgcacacggttttgtaacttgccttgcatttattatattatgattgctttttcatgtcaagcgatataaagctacatttttttttttttttttgagacggagtctgttgcccaggctggagtgcagtggtgccatcttggcccactgcaacctctgcctcctgggttcaagcagttctcctgcctcagcctcccgagtagctgggactgcaggcacccaccaccacgcccagctaatttttgtgtttttagtagagacggtgtttcaccgtgttggccaggctggtctcgatctcttgaccttgtgatccgcccacctcggcctcccaaagtgctgggattacaggcatgagccaccagacccggccaaagctacattctttatttgtaagtgttgcgtaaggttccattgagtcaaagtgccgtaatttatttaacgaacccctattaggtcatttactattttttttattattattataagtagtgctgtgacaagcatcctgatagaatatatacttgaaccattccctccttaggaaaacctttaggagatgctgggtcaccctgcaggcattgtttgcttcatgtgactctggacactgcagaaagcgtcctaggagattgtgaagacacatccacccctctgccctgcccccccagccaagggcttcccggcttttaagaaaggaatagctcttcttagaattcatgtctttggccagagggagataggcagtctcaacccagggaccctggttagagggtgaggcggaggggatggcttccccggccagatgtttgtcttctgttctcagctccatgctgctcttctgtaaacctagagaagttcatttacagctttttgtatcccagactggaaccttgaggccttcttaaatcatgtcacaccccaggaggctgtaaaagctccttggctgggggacatgttgggagcagcagaaacagtgccgtaccctgtttctcccgagaccctctcgggatctgctctgattgtttccttgcctcagttcggctagcatttctggagggggagggatcgtggtaaggaaatctgtgtggttagctcgggggaagcacaatgaaggaggcctcctctgtctggaagagcctttgcaatccacgagagggctgagtgaggaatggaaactgctctgatggggaggcaagggatgcacagagctggggactcatagcggagggggcggtgattccaggggctactgggtggggcagggcatttatgaaacctttgtggagcagggaacattcaggttgagtcaaagggtggggtagaattgtagcaggaggtgataagagggtatggaaggaccaagttgtagacggacaacaacaaagaaaaggcccccaaaaagtcagctgtgattcttcctggatcctggaaccatgtactctccggtcagttttctgcagctttgactgccttccagataggccaaacgaggcatgagacagagcctcgatgtccttactatggatactccagttggatccagaaaggatttaaggcatcttcagggaaaagataggacttgggcctacagctgaccccatgggtcctgttggccagcaggctcacctgccgagaccagggtgccagggagatggctccaagtaacggtgctgtctgctggctggtgcagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatgtatgtccatgctgtggggcgcagcccgtcttcccctccctgcacactcagcacctggaggcagcccttgcctcctttcattttgttcatttgccctctttctcttcaccctttgccccagcctctttttctccttatcttccatttgtcctctgtcttgagagggtaaagggcaaattgaaatgggaatttgggctaggcatggtggctcatgcctataatgccagcactttgggaggccaaggtaggagaatcccttgaggccaggggtttaagactagcttgggcaacatagtgcctacttgagctctggctactccctgggctggtttctggacagtttctagtagagctctttggattagagagaggttcctgtcctcccaatacttttggcaaatagctttatcagcttccatatctgggaggctcaggcagggtcttcccttaagcatgactacaggccctctggagtcctgaagggcaactggaggacattcacatggcccagacaccctgcctaagacactcagcatgccaggtaccttcagactcagccgtccacttgggtcaccttcccagtgggtggctgtgttgcctgcctgggtgatactagtttccccattggcctaagaaatgatccaacttggtcccgttcttgtttaggacttgtccatggttgcagctagggtcgtgagcctgcagagcagctgggtctcctctctttcgtgtcaaaggacttctttgccaagttcacagataatttctctttcttcctgtctgcctctgtccctggacagcaggcccatcacgttgcctttatcctcccagatccaggagtggggcccattcgatctggtgattgggggcagtccctgcaatgacctctccatcgtcaaccctgctcgcaagggcctctacggtaggtaccatcctgtcccctccaccttctcagctggtgcttccgcacataggcttcctccttggatatttctgccctgggacagctattcccgatgaccctgtcttcccgtgccctccccgtgcccgagccacaccactgtcctatgcagacagccccagctgatggctttctcttccgacctctcagagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgaggtatagccagcaaccttggtttggccagctcactaatggcttctaccttggactgctgcttcatcctgtctcatctgcattgtggagctggggactggtgacttctgctttgcaaggggcctgtttgggaagccccaggcttcctggatagaaagggaggaagcagggttagaggcctacctcggaaagaaccagtggtctcactctaagaggtggcattgtgggtgtggacctccggggttgaaggacctgtcactcaatcaggtggggtgccagactcctggggttgggtattctgtatggtgtttgttacacaccaggatctttgagaggacaaaatcagttttccctgagcaagaagggcctatcctcactccctggtaccatgacaggcacctgagaagaaaacaagacttagctcagttttcattccttatcatgtgagtccccgtttaaccctgagccaagtctgagccgcgtgcagagggcttctgctgccagaggcgtgcttggagtggcccttctgggagcttgagcccacctgtgctcctgagtcttggggtgggtcacactgtgagctggtgttctgactgtaagtgtgagcaggcgtctgtttgagttctaatcagggctcatggtaaacacacccatactcgctggagcctgggctctcccagcctctgggtagtctagagcagcactgtgcaatatggtagccaccagccacatgcggctgtttacattaacattaaccattcagctcctcaatcacattggcctcattttaggtacaaaatagccacctgtactagtgggctagtggccactgaattgaacagtgtagaggacaacccaccatcgcagaagttcatttggacaatgctgatctagaacgttccagtgacagcttgtggaatgtggctaggtgtaattccagcttctcctggaggctgcaggctagcccagtgtgtggctcctgagagagaagtaagaggctttgaggctttaaggctcgaccccagcagcatgccggcgctgtttcatgctcctccttggctcatcttcaaaccgtctcctgttttgtagtccaaccctgtgatgattgatgccaaagaagtgtcagctgcacacagggcccgctacttctggggtaaccttcccggtatgaacaggttggtgaaagctcctgggcctggggggctgtgggctgggactgcaggtgggatgacccatagtggggaagcccctgagccgggccctctctggccggcctctgctgaatgactagctgctctgccatgttccttatttccttttccttgaggaggaagaggagtcagggtatagaaaatactcgtctgaaaccaacaactagtggtcttaaagcaggacctttaaaagtactctctttataccaaagaagagtcctgtgctgctgagttaggtagttggttttctgacattggtgatgtgtcccgagccctctggttcttagattctcctgaggcaaagccatggggcatggctttggggctgggaaggggagttgtggccacagcctgttccaggagagggaatcttgcagcggctgcatgcaagttcagggttctgcttaacttaattttactaatcacacactttgttggcttcaggtccttttttggagagattttcttcagttaggttacacagataaagattttaatatctaggaatgaagaaaattcatttcatcagttataaaactggcattataaaaaggctttttaggcgaaataaattgtccagagttggcaattcaaaagcatgctgagagccgctcagtccctgtccattgtggagggaagcgttttccagagtgttctgagtcatgggtgttaaacctccatggtgggctgcttggtatacttggtataactaggagacatcatttcacctggcctcggtcaaataatctccctccttgctattcccatgaggttgtaggaccttccccatctcatacatgtttggtagagttgctatcgagggcttgttgaggggaaggagatggggcattggggcagctggcctgaaactgtcccccgagagttcccaagatacatgtgcatattgggaaatggatgggctaatgaggtccagtattttcatggaaccactcagtgtctcccgacagaccttggtcctgtgggatttctcgcctgcttctttcaactctaaaatactaaactctgagcttctaggaggagcagaggtcctgcatgcagggccgactgggcagtgcacgcataaagagtcggggacatctgtcgagcacctgatttgactgcaggccccgtggttcccacactgagatcctggagccagtgctcagggaagccttccttaaatatccttacatgtcagaaaaggtcagcgttgaaggggacagccagtcagttatgtggcctgcaccctgagaagtgacatatttaatgtcacagaatttttggtctttagggaactgcagaatttaagtctggttttttttttttttggagacagagtcttgctctgtcgcccaggctggagtgcagtggtgccatcctggctcactgcagcctccgcctcccaggttcaagcgattctcctgcctcagcctcccgagtagctgggattacaggcacctgccaccacgtctggctaatttttatattagtagagaccgggtttcaccatgttgatcaggatggactcaaactcctgacctcaggtgacccgcctgcctcagccttccaaagggctgggattgcaggcatgagccactgcgcctggcccagaatttagtcttttaaagcagtacatttaaggcttgcggtttgattaatttcatccagtggacacacccgtgtaacaatcaccacaaatcaagatacagaacattgtcacccccaaagcttgcttcgcagtgggacgcttaaagctcagtgtattttagactaactgatgtaaagatagctgtgctttttttttctttttctgctaggtgttatctgcctgttcacaatttgagaccacttttgaatttacctttaaggattaacgcttggtggatttgtgtctttgcatggctgcagtgtgtgttcgtgtgtttgcggggagtttgaagagagttatgacgtgtgtgcgtgatttggggaaggggctcttaccctgtgaactagtggctgctgggggagggaggggagtcgtgcagctggggcccttcccgctgttatccaggtttctgttgttacagtctctcttctgcctcctaggccgttggcatccactgtgaatgataagctggagctgcaggagtgtctggagcatggcaggatagccaaggtcagctccagcgtctagaacctctgctggggggcagggagaaggaagggcaggatgaggtggagcggtggccagggccttgtcccgcctgcttctccagctagtgtggagaatgcaatttaggagacggtgcagggtcatgcagcagggcagccagaggagggcccagggctgctggccttcagcccttcctccctggtgagtgtcgcccatcacccccggtagacactgaggagtctttaccaagacctggttgtgctgtggcttttttttcatttttaaattttttggccaaaacctattacaaaagccctcaaagcagcctcctgtgatgtggaagctccatttctctctggagctggaatctcccggagtgactacctggggcactgggttctgtgtcagcaggttccagcattcagaaccactggcagctccgagggcatctttgagcccctcagtaacttggcatcctttagcctacaagttactccatctctgaaagaactaattctttggaagacttggaaagtacagtggctacagcttctacttctaaagtctgtggctaggccgggtgcggtggctcacgcctataatcccaatactttgggaggccgaggcaggcggatcacctgaggtcaggagtttgagaccagcctggccgacacggtgaaaccctgtctgactaaaaatataaaaattagccaggcatggtggcgggcgcctgtagtcccagctactcaggagtctgaggcattagaatcgcttgaacccgggaggtggaggttacagtgagctgagatcgcaccactgcagtccagcctgggtgacagagcgagactccgtctcaaaaaaaaaaaaaaaagtctgtggctagatggatctaagattggccaggtgacctccaagtaggaagaaaagtgaggctgtggggtctgtggaggctcctgccctgcgaactctgctcactcagagccatacccagccccctgggtggaccaggccctgtcagggtttggcgagtacctggcatatttggtagacgcatgaccagtgttggctggtgaatgaattcgtgcttattcctcttttctcctcttcatctagttcagcaaagtgaggaccattactacgaggtcaaactccataaagcagggcaaagaccagcattttcctgtcttcatgaatgagaaagaggacatcttatggtgcactgaaatggaaaggtacccgggttgtgctggcatctggctgcggtgtggctgttctgattgctgtgcttgctgcgggggtcctctgcctttcccacctgacttgttttccacaaacgtcctgggaacagtgggttttatcaagcatttcccaacatggcagactcctggggtggactcctgacaccccaggctcctgggtattttaaggctggtcccaagtaagaggagtctaggggccaggagcagtggctcatgcctataatcccagcactttgggaggccgaggcgggtggatcacctgaggtcaggagttcgagaccagcctggccaacatggcgaaaccccgtctctactaaaaatacaaaaattagctgggtgtggtggcgggtgcctgtagtcccagctactcaggaggctgagacaggagaatcgcttgaacctgggaggtggaggttgcagtgagccgagattgcgccactgtgctccagcctgtgcaacagagagattccatctcaaagggaaaaaaaaaaaaaagaggagtctaggaaatcccctgctgccttgtctcccagagaccctgctgatcccccagggaaccactgcagtgacaggaccttaacttgtggtgccaattttataggtcttttgtaaaccaagccagcatagggccaggcagacctcgcagcctcaacaccctgatttgatgcaaatgggactttcggggctggatgaccttcttgttctctgagtgtaactaggatttagccagacaaagagaccatttacacaaacaaccagagcaaacaggaaatccttcagcttctgaagtcaggcaggagttcggctcctttttcgccttccctgctcttgatggggaggatgctagggctacgtgttcgttacatgcagactccaggtgtacgttgccgagctaaccctgcaggagaggagaactagagagtgtgtcctggaggtatttctgagggattgttcaaaattactcttatttctgctgggttgtgaaactctaggcagtgatgaccttactacctttaaggtcacagaaaccagcacagtgcctggcacatggttggtgatctgagtgccgggttgtttataaaggacagaagattcggcagaactaagcaggcgtcagaggagttggtgggtgtgagtgcccctgtccctgcacttcgggtggctgctggtcctccgggtcctgctgtgtggttagacggcttccgggcagcctggtctggccagcactcaccctgccctctctgccttttctcccccagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtactggtgctttcctgtctaagtcgccttttgtgtgttcttttatgaggcccccaccatccacccctctgtgcacaaggcggctcccggcccctggaatgtgatgtttttggtcatctccaaaggctgcagttttatactgggaggctgatgacaccttttattataattattcttatggttctggttataattatgtttgttttaagattttctttaagaaaacaaaaacccaacacccttccctttaggtttcaaaccaaggtgcgggagcagggtgcttctgagttagcggtggctctggtgccctggctcccacccctcgggccaggtgggccactgggcatggtgacagctgggcggccagggagtccccaaggtctgccttctgggcttgtcactcttctcttctacttcccctcctcttgagtcgggtgtgtcagggctggagggaggaccggccgtctctctccttgtgtgtgtgattggagtggtgtgtatttcttttctagtacttggtctgatgatagctgtgctcttacctcgagtcagcagcacctggagccccaagtgctcaacactttgttctacttcctacaaggcaggcggcctggcgtcagtagtaggaagggatggcattgttggctccagggtgagggcccgacgggcaggtgcttgcctgggtagatggggtgtagatatgtggcatatagagatatatattttataatgggaggggggatggcacctcctgggactggtagagctgggaggtgtgtcggcaagcacatggccccaagtccacgtccctagaaagatggggcctacagtgataggcgctctataaatacatagatagggtcatgtttaagaaatattatggggtggggagtgggggaagcaggcttagtgtgctgagcctcggtccccactgcagacctaaaccccacaccaatcagtcaatctcacacgcatacacaggagcatatttctgtactgatgcataatgaatgcactcacacacacacacacacgccagggggctcccagcactcttctcccagggttcaggactttgtggagtcagcccgtgcagctatttacctctgttgccctgattgcaggtagactgtcaatcccagaagttgctaggtgctgaggcaggaaaaggaggaggtttcttttagcaggtgctctcctctagcaggtagtgttctatctcttttgcccctggtgatgtcaaaagattcttctgttagcaacctgtgatactgacctgcaactctaggcaccagtcaagtcagccctcaactccccctacctattctccccaactgaaatcaaattgacatccaggtctgtcttggccagggggcttccattggggaaaaaaaatgggacagtgaacctcacctattgaataaaccaaattagtaattagtgacaaaaccaaaccaaggctttggaccccctgctcattactcaggagtaaattcacacagctggtggccggtctcacccctttttggggtgcttggtcctggaccccaatctactgtctcctcgacagattggtgtttaagatcatgcccagttgcttcctgtcacctcttgacaggtggtgactgggctgctgtggggctggagcagcgaggaagggagcctctgccccctcagtggagtccctgtgctaactgtggtgctcacccagctcctccttgctcccgcggggctctgtccttcgggcctggagtccaccagcgccactccaggagggctgggggaggaggggctgcctcaggcagtccaaggtgctccctcccccgctggccaatgctaaggtcttcccggctcccagggtcaggacagagtcttctaaggctcaggctctcccaagccactgcttagtgacagttagcctttcccagcggcccaagctccaccggaactgggcctggtgctccaaccaaagggttggtgttgggacttgggaagagacttggtagccaagggcatggcaagcctgggggattcctgtgggccggcttgctcgaggagggagtgctgggcacagaggagcccttatggctgacagcttgagagcacttgttcctccagatctagccacttgaccaggccctagaatggaccagatgccccaggggtcatggcccccccccccccccccttgcagggacgagagttaacaatcaccagcttttcttccggcccagcctgtgtgcttggtctcacagtagaatttaggagagtgtggggctttgggattgtgccaacttggccaagctgagatatgcttaatccacctcaacgctggggcctctggggacaggtattgggtcaccttccaaggggtgctgaacttcagcaggctgtagataaggcagaggttagaggcagtggagtgggggaaccaagaaggcctagggaaaattgccaggcatattccctgtggaattttctttccgaagagtaggcccatttcccccttagaaacaagggttctgggggctgatgacagcttcccctgcaggtgccaggtggcgcatgatgccaaagagatccgtggctcttgcctgggggaggtagccaggtgctcgtgctggggacacagtccttggcctcctgcctaagcaggaagctagtttccagcaggcggctggtactaaccccccgccccacgggagcgacaggctggtgcaccggcagactggggggccgcaggcgggcactagcaggggtctgcagtccatagatgatgacatgaatctgttgctgtccttttgtctctaggccccagagacttcttgctattgaacatcctattggaaagcaaactttccaagagagcacccgggacctgttagcttttgaaacaggcctgggtgtgattgactggtagagatgcctgtgtgatttttggcgcattgcaggagtgtggattgggacaggcaccccagcaggaaggaaggctgacaccaaaggggtctcacagggcccagctccccttgcttcccgataattggccaccaggccatggcgactctggggaagcctagatgggaacacccaccttctccagcagtagaaggaatatttcagcagagccatctgggagatgttcttataggaaaactgcagtcattcatttcatagttttgcttatataaatagagtacatttgggaggcagttttgaattcctgttgctactctgcttggtgcggagttgcagtggccccaagtgtctttcagagctgttgcttttaggatccccctcaagctgtgggttagggccagggcagggagggggtgctgcctggcactgtgccctgaagagggctgcttgcttcctttggtcgggagcacggcagagccaacattctggtgggaaccgagtttctagccaggcccctgttttggtcccgggaggtgagtaatctgagtttgtcattgcgtattctccacgctgcctgctctcagtgctggagactagagagcgaccctggagttgtcctctgggagtcctgcctgtttgccagggagcagctgaaacctccccgctcccaatcccgggggtggaaaaggtgtagtggaggaatggtctggagctgggatggcccttctggccatgccctgctgagccaggattaaggaaagtgtcagccgcctccttaccaggccctgtgctggggtcaggaggaccgggaggagagggaaggggtgagaggatggactctgctgttgtagtgtgagctctggaatctttacgttttggtaaaggcaggccagggtctttgaggatcgagttggggaaccttgcctctgtgtttccgagacccagctaaggtgggcctttggtgggtgacctgtctctgacccgaggcagcagagttttgggggagggagaaacaagcaaggagcagggaggacaaggacacaagatccctttgctggtggtgacttactcacttcccccaccagagggcctgggactcccgaaactcccgtaactggaaagctccctggctcagctccactggggggtgaggagggcggctccctcagccccagcctagcggcctccaaccctgtgcctgagaacatgagcagcggtctaaagcatcccctagtcccatttcacttcacagaccgatttggttgcacagccctagaattcaacagacaacaaaaatgctgaaattccttagaatacgcagagggaggaggtgactcaccaaggtgctaaaacattttattaaaaatatatattgttaaattaagtctgctgtctggacaatgactgtttgttttgttttcttgtagtggagtttaaaagagactattattttactctgatatattattattaaaaaggcattttaactttgtacttgaaaactaaatgagcgatttcatgtgttttagctgaggcattgaagtagcgggtgcttacttatttgtgggaaatcatgtattcatactgaagaataaactccgtagctgatttggtaataacttttactgttacagacgatttcgctttgaccccggtggcagagcacttttactccacattccacgcagctaaatgcaggacttcccagactcttccctccccagccctctttcctttctcttaagttcctcttttctcctttattattattatttttttttcctggttccagcatcctttcaccactgtattttttttttttttttttttttttttttttttttttttttttttttttttttagggttgcttctctatttattgacaataataatgtacatttcacagtctggttctaggacattcagggaagggcgagtgggggcccgaggggtccctgccccacccgcctggtgtcctgttgtatctctgtgtaagtgatgctcgtgacatagctgtttatgaaataattaaagaggtcaatgcgtacaacagatgtagaaagtctgtcagttccgccttcatcacatttttttttgtgcccagaagaatataataaagctcctttctaatgtacttgtgctggagaacacttgaataaatggactgtttttgtgcaaaaagaaaaa

SEQ ID NO: 3 - DNA, 46972 bp, Homo sapiens
acccactcccgctgccccgtccggcccgcgccgcttcctcgcagcagctgctcccggctccgcggccgcagcccgcgtggacgctccgagcgccccccgacggacgggaccggctccctggcggtcgggcgagcgggcggcaacgctgcccggccggcagcgctggggttaagtggcccaagtaaacctagctcggcgatcggcgccggagattcgcgagcccagcgccctgcacggccgccagccggcctcccgccagccagccccgacccgcggctccgccgcccagccgcgccccagccagccctgcggcaggtgagcgccccggggccccggggaggctcggccgccagctgcttcgagggctgctaggggaggggacggcgggggctcccgaagctgccggggagagaccctctccagggacctcccgaggaggggctctcgccgggttggagacccggctgggtttgcggggtgaggaaacgtccacggaccctgcttggggtggaacggggacagcgggtgggacgcgggggacatacgtctgacggggaccggagatactagaggaagggacgcgtctgctgcctccaggagcccgaagaggagagaagcttgctcgcagggagggggacgcgctggcttcccggggtccagactcgaggaggaggaggtgcccgtcgagagcatgaacaggggctttgctcatgggcagggggtgtaattacctggcctctgccttgggaaaagttaaggggcagaggaggtgaggaggggtttctgccatgcaggtgggcgtccgccccttgcaactgggatgtggggtcttcaggcgaggcgctgtcccttcccccaatcccagctttaggaaagagctcgtgagtgtgggcttagactcgcgcgggctggggtgtgcgccgtggagggcgtgaggcagagggcgacctcccggggtgctcctgagctgagccatcaagcgcggggagcagcggggctgccctctgcctgtgcacacctggcccgggaggagccggccggaggggcggcgcctccctgtcccggccacgccgcctaccctggggtcttaccttccattcttagcctaactcccgtgtgcggctggccccacttcccagactcgcacactaaggcctcaaaacctctagcaccagctcctggaagataagcttcccggctcgtggttgcatggggagggtgtattgagctccactttatcggggagaaagtggaagcggagaaagacctaacaagttgtaagactgcgctgcccttgggcccaggcttcccgctggaggatctcacctgtggatggagttccgagcccaggtcttgcccgcgcctccagctttccctgctcccttttcctcccctggtctaggagagaggctcgctgggcctccgtgtttccaccctcccccgaagctgggaagtgtttgaattaaagggaattaagggcagagtatgaactttgagttgcctcctgagttctggatgcggtgggttgtggcagacgggtggcagtttttgggaaacagctgttatctcctctaccaggcctgctggggcttcttgttcacccaccttggaaacgccaggtgcggacttgatttttccagagttgagatgatggtacttagtttatagggttggtatacagtaagcactcaatacatgtttgccattgttggaatcactagctagcacacgggctaagcaagcttccttccttaaggaagcatagttcttagttcatcatgtgtttaaaccctgtctcgccaagttcattgctctccgtgccttaattattacatctgtgaattagcaatagtcctactaatagggttgtgtaaggatgaaaccagtcttatgcgtgaattaacctcttcagcccttgatcttcctcagatgtttaggcataatagcctctaccacattgggcctgggtaaagaggcagtgggatagtgcctgtgtagggtcagcacatggtgagtgttgatgtctgtacttaataacgaatagcccctcattttagggtggccattcctgagatttggggatggagggaggcacccctgccagcctctccttttcctgccttcatgaaggcagctctgaaagcttttctcaatcattgtccacgattagggagttggcaggaagaaccgcctttgttccctttcgaacggagtcttggttttgttttataagggttggggccgtttttaaagccctcccggccacgcatgggggcctactgcaaaggtgtaattgcagaactaagtgagatcctttaccttttcccattggggtggagagtgctgagacaaagggacaaggactggggccagtggcatcctgctcttaaagtcatagacataacccccaccccttgcaaaggatggggggaggggaagacggtagctgggagctgcaggggtctaagggaggagcaagggaggtggggttgagaggctgcttccttcccttcctccagccacgtggggcctgtgatccagaggcgcctgtcgcccctcattattgcagccccatggacctgctgccagtgggggcttgggcaagttcttcaaggaaaacttaccttgggattccttttcctagaccagctcttatgccggctgcctcacctccaggctcagacgccttttccagtttcagctaaatgtgaaacttttcagaggtatgaggttgtgtgctcttcctcacacaccagtccttagcccaggcacatactactaattgagtggagttttttcttttaaatcatgaattttcaggatgatttccagtttcacggaggaccagggctggtgaagtatggaattcatttcatgtgcaggccactcttgtaaaaagagactgagcagatatgtctcccctttgttggggggctcatgttctcactgggacccttttacaggtgatagaactgaggctctggaagatacaggtactattacgtcttggtggtgagcattttctttctctttttttctttcttttttttttttttgagacagttttgctttgtcacccaggctggagtgcagtggtgtgatctcagctcactgcaacctctgcctcccgggttcaagcaattctcctgcctcaaccttccgagtagctgggattacatgcgcacaccaccacgcctggctaatttttgtatttttaattgagacggggtttcatcatgttggcaaggccggtctcaaactcctgacctcaagtgattcacccgcctcggcttcccaaagtgttgggattgtaggcgtggctgttacacccggctgctttttatttatttatttatttattttttctttttttctttttttttttttttgagacggagtctggctctgtctcccaggctggagtgcagtggcgcgatctccgctcactgcaagctccgcccctcgggttcatgccattctcctgcctcagcctcccaagtagctgggactacaggcgccggccaccatgcctggctaattttttttgtatttttagtagagacggggtttcaccgtgttcgccaggatggtctcaatctcctgacctcctgatccacccgccttggcctcccaaagtgctgagattacaggcgtgagccactgtgcctggctatgcttttatatgggtgctgcctcacacccataatcccagcacatgggagactgaggctggaggatcacttaaggccaggagttcaagaccagcctggccaacgtgataaaaccgtgactctacaaaaaatacaaaaagtagccaggcgtgtttgtgcatgcctgtaatccgtgctactgggaggctgaggcacgagaattgcttgaacctaggaggtggaggttgcagtgagctgagatcctgccactgttctccagcctgggcgacagaccgagaccctgtctctatgtgtatatatctacgtagatatagatagcttaagtttttttttttataaggacaaggtcttgctgtgttgccaaggctaaccttgaaagcctgggtccaacgatccttctgcctcagccttccaaaggcttgggattacaggtgtgagctaccacacatggcctctaggtcacttttcactcagattttttcattcattcaaacaaaatagcataggtatgacctctgctctagtttattttttatttacttatttatttatattttgagacagtctcgctctgttgcccaggttggagcgcagtggcggttgctcactgcaacctctgcctcccaggttcaagcaattctcatgcctcaacttcccgagtagctgagactacaggcatgcaccaccatgcctggccaatttttgttttgtaaagatggggtttcacctcattgcccaagctagtctcctgggctcaagcaatctatcctcctcatcctctcaaagtgctgggattatagacatgagccaacacacccaaattcaatttttttttaattatttttattttttgagatggagtttcgctcttgtcgcccaggttggagtgcaatggcgtggtctcggctcactgcaacctttgcttcccgagttcaagtgattctcctgcgtcagcctcctgagtacctgggactacgggcgtggtggtgcatgcccggctaattctgtatttttttagtagagatggggtttctccatgttggtcaggctggtctcgaactcccaacctcaggtgatctgcccacctcagcctccctcccaaagtgctgggattataggcgtgagccaccgtgcctggcccaaattcaatttttaatggggtccttgtcttgttttatgggtacaacatcatttatctgcctgagaatattgtagggtttagtgttccctcgtgttcccgccaatattaagggatactcaattttccagtaccttgccctgtaggtctggctacagggccaggagactggtgaggtctcagtctggtatctgttcctccacttgtcccagtctgtaagctcttgtgtgtgttttttttttaattatttcttttgagacaagtctcgcattgtcacccaggctggagtgcagtggtgcgatcatagctcactgcagccttgaactcctgggctcattccagtatcctcccacttcagtctccaaagtagctgggaccttagtcacgtgccaccacgcccatctaatttttaaaattgtttgtagaaacattaaagtctcactatgttgccccagctggtctcaaactcctcggctcaagcaatcctgcctcagtctccgaaagtgctgggactacaggtgtgagcctgtgcgcccagctgtctcatgttcttgtccagagcctcagcctgcatgtctgaagaagagtggttgtctccagagcagcaggctgagggaggggacttgggagtcaagggtctaactcttcctttggaagaaggcctctctcttagtaggcttttctggattccagcagcatttatgtcctgttgtatgtgtgtgttctggggccagagctggggctggaacccaggctgccactgggccccagctcctgcagaagtgctgatgcctttgtagggcgtttgctgcagccagaacagtgatgggccctggcagcatggcaacatggctcactctgttgcttaggctggagtgcagtggtgcgatcacagcttactgcagccttgaactcctgggtcaagtgatcctctcacctcagtcacctcctgagtagctaggactacaggtgagagccaccaggcttggctaattttaatttttttttttttttttttggagatggagtttcactcttgtcgcccaggctggagtgcaatggcgggatctcggctcaccacaacccccgcttcccgggttcaagcaattctgactcagcctcccgagtagctgggattacaggcatgcaccaccattcccgactaattttgtatttttagtagagatggtgtttctccatgttggtcaggctggtctcgaattccccacctcaggtgatccacccgcctcggcctaccaaagtgctaggattacaggcatgagcctctgtgcctggctaattttaaaattttttatagagataggctctcggtatgttgcccaggctggtctcaaattcctaggctcaagtgtaatcccaaagtgctgggattacaggtgtgagccaccatgcctggtcccttgcctgcctttgattcctaattactatcatggttcctgaagcctagtagaactcatctgaatggatacaggaggctctggagatgggaatggggaggcactaactagggtcccactgagccagctacaggcccccgctcttttgtgaaaagatggttgtgatgaagacttaagtgttcattatattccaggcaagccattttctctgactctgggagttgtttggcaccatgggtaaggaagccctaccccgacaatggcactggaggaagagcctcaagcttaggcactggctttcccaagtccttgggtgaatttgcaaacctcacctgtgtcctcatgttgccctaggatgttacatgctgagctgtaagcaggatgctgaattaggggaattgctcagtactaaatatgattgttactacaaccacaaaactagcaaggagtagaactgcattaaaacactgttgcaaatttttttattttttgagacggagtcttgctctgtcgcccaggctggagtgcagtggcgagatctcggctcactgcaagctctgccttccgggttcacgccattctcctgcctcagcctccctagtagtagctgggactacaggcgcccgccaccttgcccggctaattttttgtatttttagtagagacggggtttcaccatgttcgccaggatggtcttgatctcctgacctcgtgatctgcccgcctcggcctcccaaagtactgggattacaggcgtgagccaccatgccgggcccactgttgcaaatttctttccaaaacctctgccagcacatggctgaacccacagaactggcaggacacacaagatgcgctgggttaagatgctttggtgctctctgtgggttcagctgactgttttacaggcctgcttcttgaccgttggggtgggcagatctgagcccccactaggtaattgtggacagtttttgaaaagtggtttttatgtgagatcatgggcttcttttaaagccatttgtatatatgcagttcccttatctgcaggccttaattttgacatttctgttttccttctcgagatttgttttctgtccctgcagtactgctttttccagaatgtcatataaatggaatcatacattattattatttttgagacagcatctcactctgttgcccaggctggagtgcagtggcacgatttggcacattgcaacttccgcctcctgaattcaagcaggaaccatacattatatagcccttcgagtctgggttcttttacttattgtgagatatttgagatttatttgtgttatgacacgtactagcagttatttcttactttttaaaaaattttttaagacaggtctctggagttcagtactacaatcacagctcactgtagcctcaactccaatgctccagcagtcctcccactgcagcccccactgcccagtagcttcgactacaggcacttgtgcaccactatgctcagttaatttaaaaaaaaattatttttgagacagggtcttgctctgtcactcaggctagagtgcaatggcatgatctcagctcactgcaacctccacctcctgggttcaaacgattcttctgcatcagcctcctgagtagctggaattacaggtgcccgccacctcatcaggctagtttttgtaatttttttttttttctgaggcagagtttttcactcttgttgcctaggctggaatgcaatggtgtgatctcagctcaccgcaacctctgcctcccaggttcaagcgattctcctgcctcagcctcccaagtagctgggattacaggcatgtgccaccaccaccagctaatttttgttggtttgtttgttgttgttgttttagtagagatggggtttctccatgttgatcaggctggtctcgaactcccgacctcaggtgagccgcccaccttggcctcccaaagtgctggaattataggcttgagccaccatgctccgccttactttttttttttttttccttttgagacagagtttcgctctttgcccaggctggagtgcaatggcatgatcttggttcaccacaacctccgcctcccgggttcaagtgattctcttgcctcagcctcctgagtagctgggattacaggcatgtgccaccatgcccggctaattttgtatttttagtggaaacggggtttctccgtgttggtcaggctggtcttgaacccctgacctcgggtgatctgcctgcctcagcctcccaaagtgctgggattacaggcatgagccaccatgcccggcctaaattttttttttgtaatgttggggtctcactttgttgcccaggctgatcttgaactcctgggctcaagcaatcctcctgcctaggcctcccacattgctggggttacaggcatgagccactgtccctggcatctttttttttgctgtgaatataacatttctagcttttctttgtgttgtgatttttaccttttcaatacatagtgtgcaaggacatattttctggttacgatagaaataatttatttgaaagtctatccatgttgaaacaactaaaaatgctgtgcctttatctgttggtagaagaactaagctcccttaaagaaatctagagagcagaagagaacaggaaatgagagaccacaaactaaagggagaaaatgaacttttcaataaggcttgcaggcttgctctagcagttgtagtgattaaaagtagtggcaatggctgggtgcagtggctcacacctgtaatcccagcactttgggaggctgaggtgggcggatcgcctgaggtcaggagttcgagaccagcctggccagcatactgaaaccctatctctactaaaaatacaaaaattagccgggcgtggtggcatgtgcctgtaatcccaaggaagctgaggcaggagaatcacttgaacccaggaggtggaggggttacagtgacccgagatcatgccattgcactccagcctgggcaacagagcaagaccgcatctcaaaaaaaaaaaaaaaaaaggagtggtaaaggataaataccaaacaaaagtgagcctcaaaacatattacatatatatgggaagttcaaataagagatagagttgggatgtattcataggtgtctggctttacttacggggaaaaatattaggtccctatctcataccatatacaaataatcaaataattatgttgattaaacctgagtggcctaattcaaataaatgtttctcagacaggcgagttatgttttccgattgtaaaagttacatgggcttaaggtagttaccccccaaaccttccttctgagtatgaatttttatagttagagaagaaatgctgcagagaaatggcttaaacatcaataattgatgttgcattatgcaggtttcttgttttcactttacataactttgtctaacaataagtactgcactctttgccccctattgtggaagcaggtaatttagtcaaccagtcctcttgaccgtgatttgattgcagtgtttgctatgacaaatgggactgcagcaacccccttgcacagaggccttggcgagagctttgaactgggtggtttctgagcagtagaatcctgtgtaacaggatatagacattaggcattttagatgacacagtgaagattactatcccaaagactgaaccagtatccctgctgtgtaggtatttaggcttaagatgactaaatactgtgtagataggtagacttgagatttaaggcgttcttagttgaagccacaggtgcagcctgctgtgagggaggggccaaggttgagggggtctctttccagaatattgacctcctttgcccccttgggaggggacagggctgttggctctgattttgccaaggggtaggtggccacaggaattgggcatagcagtgcctaggattctgctccaatgctgcccctcattctctgagttaccattgaccactccttggggctgccacacccttcaaggggttctcaaagtggatataggggaaggggctgggtgtttgtgatttctgtataaagggcaaaaagtttagactctaaagttactgtcatatcttttcagatctttatggtcctagttttttttttcctgttacttatctaaaaggtaatgacattattttattccagtgcgttgacaaacatctgagtatattttggtgtcatgcacagtctgcagatagccacttgtctggtttttccctgtttggacacattttttttttttttttttgagatggagtttcgctcttgttgcccaggctagagtgcaatggcacgatcccgagtagctgggattacaggcatgtgccaccaccccggctaattttgtatttttagtagagagcggggtttctccatgttggtcaggctggtcttgaactcccgacctcatgtaatccgccctcctcggcctcccaaagtgctgggatcacagacgtgagccactgtgcctggctttttttttttttgagagggagcctgagcctgtactgttgcccaggctggagtgcagtggctcactgcgacgtccacctcccgggttcaagcgattctcctgcctcagcctcctgagtagctgggactataggtgtgtgccaccacgcctagtttattttttgtatggacacactcttctgataattctgcttactatgccaagggcccagcttctactgtaggatgacagtggtacatgtactcatgacctagaggcactgagcaattccccaaacgcattctgaattgtggagggtgatggggtggatacttcttgattggccctgtggtgtggtgctgtggtgggtagccagatggcacatgagaataagtgacttcaggtatcatgactgtgtcatctatgtaggggtccttggtaccaacttcccagtgaattgggtgggttactttatttcacagattataaaactggggtttgagagaaatattgggctttttttttttttttttataacagggtcttgtgctgtcactgggcctagagtgcagtggcatgacgatagctttactgcagcctcaacttccgaggcttaggtgatcattccccatcagcctccgaggaggtgggactacaggcgagtagctaggactacaggggcaggccgacacattcagctaattttaagaaatattttaatagagaagaggttttgctgtgttgcccaggctggcgttgaactcctgagcccaagctcaagccttggcctcctaaagtggtggtattaacaggcatgagccactgcgcccggcagtggttttttttttttttttttttttttttacgctttcctgtaaatctgatgttttctggaatttagggtaagttccttatcagacagagcaaaagcacagtactgggaccttgtgttctgtcatgtgttctgctgccgccgccagtgtggacagcctgagtctggccccctttcctttggatgtgctgtccctgctgcctggatccttcccgctctctacttcacctaggtgtggtgtccctccctgggcttacttactgcattgttttatggtgtcttttttttcttttttttttgagacggagtctcgctctgtcacctaggctggagtgcagtggtgctatctccgctcactgcaagctctgcctcccaggttcacaccattctcctgcctcagcctccggagtagctgggactacaggtgctcaccaccatgcccggctaattttttgtgtttttagtagagacggggtttcatcatgttagccaggatggtctcgagctcctgacctcgtgatctgcccacgtcggcctcccaaagtgctgggattacagacctgagccaccgcgcctggccttttttttttttttttttttttagattcaagaagtacatgtgcttgtttgttgcatgggtttattacttactggtggggattgggcttctggtgtatccattaccgaaagagtgaacgttgtacctgataggtgatttttcaacccttgctccttcccatcctcccgtctttttggggtctcccatgtcttgtcttgtcttgtcttgtcttttttcttttctattttgagatggagtctcccccttgtcgcccaggctggagtacagtggcacgatctcaacctcactgcaacccccgcctcccgggttcaagtgattatcctgcccagcctcctgagtagctgggattacatgcgcccaccaccacgaccggctaatttttttttgtatttttagtagagactggttacaccatgttggtcaggctggtctcgaactcctggcctcaagtgatcaacccgcttcagcctcccaaagtgctgggattacaagcgtgagccaccgcgcctggcctaatttttttatttttagtagagatggggtttcaccatgttggccaggctggtcttgaactcctgacctcgggtggtccacctgcctcagcctcccgaagtgctgggattacaggcttgagccactgcacctggccctagatttctctaacataggctgtggaggtggaatttttatttattttttctttaaatcaatactctccactgaatgtaggattttagtttttaattttttactgcaccccaggggttggggcagataccaaagtgaccccccttcctgcattgtgaggtttaattaatgcaattaacatttacgaagtgctctgaaacagctctgctgctggtatcagcctggaggaagtgagtgacatcagttctcagcattactgctgggttggggcgagtccctgagcctcctctgagtctaattgggggttgccacctacccctctgtctgccacattgcatttgagtacatctgacctggttcagtcggggcacaggcaccctgctgcactaatgaggaaggcccctcggggagaagcttgtgggtgggcccacccagcactgctgcccactctgaatcacacgctggaccccagcctctgggaaagggtcagagttcacctctcatcacaaggttaggcagccacctcctgtgaagggaaacgctcagctggtatatcatgtcccagccacgaagctcccttcatccctgggatgggtgatgcattcattgttcataacttctgtaattcttgtctgtcttgtctcagttacggcaattccaggcacgtgccctggattgcctgggcatgagatttggcactgccacttctctgggtgacttaactcatctgagtctgttgaaggcagggggataataagacctgccccaggatggggtgggggttaaaggaggtggcagacaggctgggtgcagtggctcacgcctgtaatcccaggccaaggcgagtagatcacctgaggtcaggagtttgagaccagcctggccaacatggtgaaaccacgtctcttctaaaaatacaaaaattagccaggcatggtggtgcaagtctgtaatcccagctactcgggaggctgaggcaggagaatcgcttgaacccgggaggtggaggttgcagtgagccgagatcgcgccactgcactccagcccaggtgacagagacttgatcttaaaaaaagaaaaaaaaaaaaaaatccatggtggtagacagtgttcagcctacagtaagtagggccctgggaactgttagctacttaatgttacagtggcagtgtgggatgatgcagttctaaatccagcatgtctgccactctggataggtcatattagataaagcagttggcttgtgatgggggccatgttatctggaaatgccagccttgaatcatgaagtcagtgagatacatagaaaagaggtgggggagatttgtatccactggtacacacctgatgctaatttaatttctcttgtcctgattctaagaacatgtagttaaatttgtatgttatttactttccctatatattgatcaaggaggtagtatgtcagtctctgtcttggtttctccatctgcaaaacaggaataacagtacttaccacatttaaaggttaaatgagttaagataagtaaagccaaaacagtgcctgacacatagtaacactgaaaaaaagatgaccttattggggggatttttatatatttatatatgtgggaagaaaggcctaggagagaagcttttattcctggattttgagctgtgggttttgaggtctgagaaaggaagcaggaggtgtagggagttggggagggcttccaggctgagagaacacagcagcacatgcctcgaggctagattgtcttggcctgcgtgtagactgaagcggggtgggggaggaactgagctgggcatctttagtcatgggtgcaggttctgaagttggtgtgcgctgtctacacagctgtggctataaggcaatgagggtagttaatttcactacaaacgtagggtcgtgataaaaacagaaggaattttaaatgaaatcacattgaattatgcaacgagaagaaagcatatataagtgctcagcttgctcctgggggacacccaggtagctagtgccatttattgtgggttttcttcctggactgtctctccagtacccacaggtcttgttctaggggcttgaggcttttcctgaatgttgagatatgggttttggaaggtgtgctgcccacacgttgggtaggctggtggccagttggcactgctgaggccctgggggaaggcccagggtggttaatcattgtcagcctgggtctgtctaggaggtgagactcctgtctggcttcttgttgacccagttagggcctgtctgaggaatggggttcaatgttgcctgcgaaacccagcacacattagaaccccaggccctggatgaaggcgaaggaggtcacatccttcactccaggcctctggtctgctaccagctcctggccacaaacaaaccctgtgtgtcctccccccagagcactttggggaggtggaccctcctggagacaccaggtacaccaggtagccccacctcccactctatgccaggcccactaggttacttagttcactgatttggcctgaggtgaaacgcttgaaattaatgattattctgaaccgacaccgcagcagctttcctgggattctgactgctttgtctcataggaactggccataggtcagccagcctgttcttccgggtcagggcttgggagaaaaagggctgagaagcagctgattggtctgattcaggcacgtcccttgtccttggtaggggaaattccttaacaggtgctgatacacactctggatcatgtgaagcacagaaagcctatgtgttatgtatcagcgcacatgtttataatctgaacaatgctgctttaacatgcatattaatcacctaggcatgacttgtgtgtgttaaaatgtcttgtgaaaatcccgcttcaggccgggtgtggtggctcacgcctgtaatcccagcactttggtaggctgaggtgggcagatcacgaggacaagacatcgagaccatcctggccaacatggtgaaacctgtctctactaaaaatacaaaaatcagctgggcgtggtggcgcatgcctgtaatcccagctactcaggaggctgaggcaggagaatcgcttgaacctgggaggcagaggttgcggtgagctgagatcgtgccattgcattccagcctggcggaggtcgcaccactgcactgcagcctggcgacagagggagactctgtctcaaaaaaaaaaaaaatgaaaaccctgcttcaggacatctagcttagtggtggagatttctccagccagctcccacttccaacagttcttccaagcagctctcagttcatggtactgtagcctgcgtggctcctgggccacacagggagtagcagcagaggtccagttgccccatgccatgggcaggtgcagggtctggaattgggagctctcaccacagattgcacctctgcctgggggcgccactgggctcatcagactgagctcacggtgccccctggtgggcgatgccagtggcgccagtgcagcttggagcccgcaatacaaggtgtggcttctagaaggtaccctggtttgagatgctgaggtgggaggatcccttgaggccaggagtttgaggccagcctgggcaacatagccagactctgtttctacaaaaaattagccacacctggtggcacacacctgtgggcccagctggaggatcactgaagccctgggagttgaagcctacagggagccgtgatcacgccactgcactccagcctgggtgaccaagtgagaccctgtctctaagatagatggatggatggatggatggatggatggatggatggacagatagaagataaactgcgaggctagcacgggttgtctggcaggggctggtatgtctccacgatggagcccgaggcagggaggcccggtgcatatggcagcagcttattggggaagcttttcccaggcacggttccaggacacaagggtgtggtgtgagtgacctggagctgtttgtggttcagggatgggggcctggaggtctcattatgcctaggcttttataatatttttgccaaccaaaggtggaacaaaaagcaaaggcaagtgacttggaaaactcggtttcagcggggatttatgccctgaagttttttactcgttctctatctaatcctggccctacctcttagatgggctatatgaccttggagaataacctagtctggatgtcagttttctttttctagaaggtgagctccccagtctccctagggggagatacaggttgttgcatccaacaaaatctgtgtgcccataatgttgcaggcaatgggccagcccagaggttatagcctgccaacccatccgtatggccctggccctgtgtgttagctgatttacctgaccaggaggggcagcagtggggaggccaagtcccagaggtgagggtgaggctacaatggatctggggagtccccctcctggtaactgcccccaggctttctgcctgtgcctccatccttccctccttgggaacttgggactctgagccccagctggtgagggaggaggggagtgagcgacaggccagcagaccaatgggccctggaggatggggtgaggcggggcttgtgctgccatggagaggagagaagcggttctgtgggggaggagggggtgggtgggaagaggacctggagattgaggggaagggagagagcaaacaaaggggtcaggagggaaaataatgcactggcttcctgagcccctgcagaggctgagcagggagagggggccagggccagaggggacagaggctggcggcagacgggccgggacaggcaggtcctaaatggcattgtttgaaggggccggctaattgcacagagcagtctgagcctgagaccccagccctggcctccccactctgtcctgggtgctggcgtctgagccttcgggacagcctgtccacatggaaccaagtcctgagcctccaagcttggtgagggggaggctatggggggcagcctgggtggggtacttgggcaagagcatcaccctaagaatgcatcctggggccttggcctgagaacacagagcccatggcgggggaacactgtccctctcatgtccctgcttccctttcaccccacccattctggcttctcccacaggaaagcatgaagggagacaccaggcatctcaatggagaggaggacgccggcgggagggaagactcgatcctcgtcaacggggcctgcagcgaccagtcctccgactcgcccccaatcctggaggctatccgcaccccggagatcagaggtggctgggcagtggggactggggtggtgtcaggcgctgacatagtgagcggtcactgcagacaactggaggctttggggagagtctctgacaacctccaccacaattccccgggagggaagagagctctagcaaggagggatgcagggtcgagcccttcacacctgcccgcagcccttggcctcccctttgggactctcatctcagctgggactctgagcgtgacacaagggtgatggttccctgtcctctgccagtcatgacaggggtggtctcagcatgggcccttggagagcccttctgcagtgggaccctctccccactcagagctgggctggggttgggagggggaggtctggagtgtgcttcctttccaccctgccctgagcagctccagccagctcacttgggatcccgccccagctgggttggaaagccctgcattgtcctctcagctgtgccatcccatggaacttcctgcgagcgtgaaagggttctatttctgcattgttccccacaatagccacactctacatgggagcacttgagaagcggctgacacagcaggcagtgaattgttcctggaaatgtagttagtggtccctggatgggcagcaccctgctggaaggaatcggagggctccccttggtaacttcagtgttggtttgggtcctgtcacgtgcctggcccagggctctgttgtcttaggaataaaatcagtggagaagatgaacgatactgacggactgagagcaaatccctgtggctgacaatagtgtaggggagatcccaggccacgccacggagaaaagcccctattagcaaggccgttccccagactggtgcctgcccccacaaaacagactcctggctgtttcctctacaggccgaagatcaagctcgcgactctccaagagggaggtgtccagtctgctaagctacacacaggtatggtctctgctctccctttttcagggctcagggactttgtctttggctttcatcacgtgggctgcctgaggcccataaaaactggcatctgcaaatgtatggagggttgccgagctagatgctttctgcatatatttggcattattttttcaatctggacgatcagttacattgctaaatctctaatatggtagtcccctcttatctgtcattttgctttccacgatttcagttgcttgcagtcaactgccgtctgaaagtattaaatggaaaatattaaatggtaaatcccagaaataaacaatttatacattttaaattgagtgccattctggtagcatgatggaatcttgcactgtcctgctccgtcctgcccaggatcagtcctgtctttgtccaacatctctatgcttaggtgctacctgcctgttagtcacttgtagccgtctccgtgatcagatcagttgcagtatcacagtggttgtgttcaagtagtgcttgttttacttaataacggctccaaagtgcaggagtgatgacgctggcaattcagttatgccaaagagaagccatgaagtgtttcctttaagtgaaaaagtgaaagttctcaaaaaggaaagaagaaatcatatgctgaagttgttagatctgtggtaagaacaaatcttccagccgtgaaatcatgaagaaggaaaaataaatgcatgctagttttactgttgcacctcaaactgcaaaagttatggccatgttgtgtgataagtgtgtagttaagatgggaaaggcattaaatttgtaggtggaagacatgaacagaaatatgttctgattgacagcagtgctttgtgccagggagcattgagcctatagcaagggatcccctgacaccaaaccatttacttcaagtaagggatggttacatagattcaagaataggtttctactgaaaactataaaaatgactggagaggctgcatttgccaatgaaaaacctgctgctacattttcagaagagttgaagttgattgagaaaggaaacaatccaaaactaagactgcagtgaaaccagactcctccagaagatgatgtctaatagaacctaccttcataaaagtgcaaaggaggaagacactaggtcataaaccatggaaggacagattaaccctgaatagagtacaagaagatattttgagagagaaaccacattcatatggcttttattaacagtatattgtaattgctctattttattattagttgttaatctgtgcctaatttataaattaaactttatcataggtatgtagatatagacaaaaacagagcacatatagggttccatcttccaaggtgtcaggcagccatggggggtcttgcatgtatcccccctggataaggggagactactgtatagcaaactgtctttttattcctggaatgagtccaacttggttatggtgcattttgaagttgattttattcatgtattcatgaatgaaatgggtatttttttttattcatttatttttgagatgaagtctgttgcccaggctggagtgcaatggcatgatctctgctcactgcaacttactgcctcccaggcccaagcgattcccctgcctcaggctcccgagtagctgggactacaggcaatggaccaccatacccagccaatttttttgtatttttagtagagatggggtttcaccatgttggccaggctggtctcgaactcctggcctcaagtgagccacctgtcccggcctcccaaagtgctgggattacaagtgtgagccaccatacctggcccattttatttctttaaatatatctatggttttgatgttaggattaggtgggctcaaggaattttttttacaagctggaagaaacttgtctaagcttattttaatgaatttcaaacaattatatcttctaagacttgctacattatcaagctttctacttcatttacttttccaaggttggatctttcccatcttgttttgtgttccttcctttactgacaccgtctgtttcctaattattcttagtagagtttgtcaattgggttggttttgctcagagccagtatcagtcataaaagtcctgggcaatgaaaactcaccctcagcttccattctcttggtggtggccaacctacttaggggactcagaaggtggggaacctaagccctgggaggaattagggcagctgcaggatcagaggctcagcactgctgttttgtttctggtagctagttaccagaaacagtaatgaaatctcttgcccacacgtttctttttgtttgtttgttttgttttgagacagagtctccagtctgtcgcccaggctggagtgcaatggcacgatctcggctcactgcaacctccgcctcccaggttcaagtaattttcctgcctcagcctcccaagtagctgggcttacaggcgtgcaccaccatgcccagctaacttttgtatttgtatttgtatttttttttttttttgagatggagtcttactcttgttccccaagctagagtgcggtggcgcgatctcggctcgctgtaacctccacctcccaggtttaagcggtactcctgcctcagcctcctgagtatgagtagctggaattacaggcacatgccaccatgccctgctaatttttgtatttttagtagagacagggtttcgccatattggccaggctggtcttgaactcctgacctcaggtgatccacccgcgtcggcctcccaaagtgttgggattacacgcctggcctaatttttgtattcttagtagagatggggtttcaccatcttggccaggctggtctggtgacctcaggtgacctcagtctgccccagactcccaaagtgttgggattacaggtatgagccaccacaccctgcatattatttgattcttgatgtaaccctgttgtgctatgagggaagtactgttattccaattttaaaatgagggaaatggactcaaagccacacagttggtgagtgaaggcaagtgcttaacactccagccagacctagctgctcgctgcttcagcttttgtgtttggcttatgctgggttttggtgtgaggtgtgaggcctgcacgaggaagccctgatgacatggtgcaggtttcatgccattgcttccggggcatctgtcgtggttggtctgtgtctgcacccatatgcagtgtgttgtgatgagtgacccggtctccctgccaggcacaggcgatcagtctttcatattgcaggtgccttgtttctttgacttgctgataccctggggtcttcatcatgttcatcatgtttccttataaaggacttgacaggcgatggcgacggggaagatggggatggctctgacaccccagtcatgccaaagctcttccgggaaaccaggactcgttcagaaagcccagctgtaagtagccacacctcgagccaaagcacttgtggccaacactctacatagcatacatagcatgctacatacatagcatagctccttaggggagcttttaagagaaagtgatgaaaaaatgttttgaaactagaaaatataagcaaggctcttggactattgttggagggttttcttttttctttttttttttctgagacggagtctcactgtgttgcccaggctggcgtgcaatggcgtgatcttagctcactgcaacaacctctgcctccttggtccaagtgattctcctgccacagcctcccaagtagctgggactacacgcacctaccaccacacccggccagtttttgtattttcagtagagatgaagttttaccatgttggccaggctggtctcgaactcttgacctcaggtgatccacccaccttggcctcctaaagtgctgggatttcaggtgtgagccactgcgcccggctggaaggttttctctatgtgccatttctttcaggtggtttttaagttctttcttgtaacactcttggaggtgcctaggggtgatagagtttgcagggtggatggctcagctgctaatgacctagggagtccaccatttctgtgggtggattttgtgcctcagagttttgaggtatggcaatgggaaggcagttattgcttatggtcacccagccctttagagaccagactgacagttactatcccaggtgtgggttacagtcttcccttcccaggatatggtctgtatcatcccctttgtggagatgagagaagattccagcaacttaactgaggttaaacatcaactcttctatgcataatttggacacaggttctgcaacaaccaaattcatgccatttttgacaatgaacatggagcctaaggaattctattccatcttgatgatctggtgccttcctgaacccagtaattggccttatgcttctttgacataaaagtttattttagagtgaacacgaatttttctttctttctttctttctttttttttctttttttttgagatgcagtctcactgtgttgcccaggctggagtgcagtgacgtgatctcagcacactgcaacctctgcctccctggtccaagtgattctcctgcctcagcctcctgagtagctgtgactacaggcacccaccaccatgcctggctaatttttgcatttttagtagagatggggtttcaccattttatccaggatgatcctgaatttctgattgcaagtgatctgcccgccttggcctcccaaagtgctgggatcacaggcatgagcgagccactgcgcttggccagatttttatttttcaatcccaattggcaggtgccatttcagctggccatgtctgtctgctgtgacaggcagagcaggacctgctgggaattgtcgggcatcctagctggccaccctaccacctctattccaagaactggggaattctggctggactcagtccagagtcccacctcatcacctgttcacttccagttgtcctgaagctggctaccaggtctccttggccacctgaaggcctaatccttctggccccgccagaccccaggcctccagtcacctaaggcccagtgagtgtcctctcttgcttctaggtccgaactcgaaataacaacagtgtctccagccgggagaggcacaggccttccccacgttccacccgaggccggcagggccgcaaccatgtggacgagtcccccgtggagttcccggctaccagggttggttccccagatgcccagacccctgcccgcagtctctaactgggagatatgcctcactcactgcactactggttgtggctggtagataatctgtgtccttttttcacactgcttttcaggttcttgctttttcttttctctcctgggtaagcttcccgtaagcctgttggcttctctcctggtctgatctcagatgacactctgttgggaatggaagctttccctgttgacttggttcttgctctaacttggaaacaaggtagaaaacactgacatccagaactgtcttctccctcatgtcttcttcacctgtcttgaggcaaccctagcgggtatggcaacatttcactctctgagagtccctcgtcattacccatcatttgtgcctttgtcatctttctgtctctgggacagttacaatgactttctccccccttaagggatacgtgttcctggaaaagtttcttcagcggtctctgttctctttaacttcagtctttcctctttctttttgcctaggagccatatgggggtgccgttggtctctggtcaccgacatcctttgctctggcccaaactatgtgtccttctgtccacagtccctgagacggcgggcaacagcatcggcaggaacgccatggccgtcccctcccagctcttaccttaccatcgacctcacagacgacacagaggacacacatgggacgccccagagcagcagtaccccctacgcccgcctagcccaggacagccagcaggggggcatggagtccccgcaggtggaggcagacagtggagatggagacagttcagagtatcaggtatggccgagaggggctcctgcccagggtgactgaggaccctgaacacggggaaaaaccagttacctgctactgttggtaacagagcgacaacagttgttagaagtttcgtaattgtaataattgacatgtttgaattctaagtatcttccatatgcaccacggacctttctttaccttaatcctctcccagacataagtcaaatgccagtatttaccacgtgaatactgtggtttttcccaacatgacatttatgtaaggactgcaactgcgtacgttatacatttcatgtacgcccgtgtgaagagaccaccaaacaggctttgtgtgagcaataaagctgtttatttcacctgagtgcaggtgggctgagtccaaaagagagtcagcgaagggagataggggtggggccattttctaagatttgggtaggtaaaggaaaattacagtcaaagggggtttgttctctggcgggcaggagtgggggtctcaaggtgctcagtgggggagttttttgagccacgaggagccaggaaaagggatttcacaaggtaatgtcatcacttaaggcaaggaccggccatttatacttcttttatggtggaatgtcatcagttaaggtggggcagggcatattcacttcttttgtgattcttcagttacttcaggccatctgggcttaaacgtgcaagtcacaggggatgcgtgcaagtcacaggggatgcgatggcttggcttgggctcagaggcctgacaatacatattgtgattctgttcatcagagtgcacatactgtatatattgtacgtatgatacctttgtagacttgcttttttcttctattaagaactagagttgcctgaggttcatgtctggataataaatccagtggtctgggtcatagcttaattaaccgattcaggttgtttatgcttattcaagcattgctgcacgaagactccttacacatgtatgtatttatgtgttgttaggatcccttttttctatgtccattattacattttactttgtatagaagggatctcactatggtgcccaggctagtctcaaactcctggcctcaagcgatcctcctatctcagcctcctgagtagctaggactacaggcgtgcaccaccaagcccagctactttataaattcttttgtggaaatggagtcttgctgtgttgcccgggctggtatccaactcctggcctcaagcgatccccctgcctcggtctcccaaagtactgagaattacaggcatgaaccacggtgtctggctacatcttgcattttatggcaagctttttgttgccctcagggacagtggagtggacaggacttggcccaggatggcctctcctcactgggatttcttcatgtgggttttcttccaggatgggaaggagtttggaataggggacctcgtgtggggaaagatcaagggcttctcctggtggcccgccatggtggtgtcttggaaggccacctccaagcgacaggctatgtctggcatgcggtgggtccagtggtttggcgatggcaagttctccgaggtgagtccggggaagggcaaggggttctgcaggcctgaggctgtgcctgcctgcctccctttgaagacaaaggcctgggattgtattctgcagatgtgtgagcctatgccttcacactgtctgggaggaaggacaaaacttcctgtgttaaccagctacagcagattccactcacacgggggacaaaagtgctagaaagttctggatccatgggcagttggattttatgttatttcttagatttatcttttgggggcagaatcatcagtgtggactctgaggggccaggagtggccacgtgagcaagtacccaaaagtgttcagtggagaagcctggaagagaccggaagcatggccgcggcctcttgtcatttagccctggggagagaaaggcatggagttgggtttagggtcacctggtatcagttgtgaaaagggtttttagtagaattgggtatttcagagtctcttgatgattgattctagccttttaaaatctttcttggtaatgacatttagctggggaagagctgtgttgggcataactaggctgttttgattcttacactctttcaacaaagatttttttgggggcggtgagaatggagtctcactctgttgcccagactggagtgcagtggtgcgatcttggctcactgcaacctccgcctcccaggttcaaacaattctcctgcctcagcctcctgagtggctggggttacaggcacctgccaccgcaccctgctaatatttgcatttttagtacagactgggtttcaccatgttggccaggctggtctccaactcctgacctcagatgatccacccgcctcggcctcccagtgctgggattacaggcgggagccaccacgcctggcctcaacaaagattttaatcagacagacctatcctgtgatggtctcttagaagtaaatgttagtcctaccctcagagccctgagagaggctaagttaggtgcctaaggtcattgggcaagttgcagcagaagcaagccaggcaggattgaaggaaggactgtacattcctgggattcagctcaagcagagattctgtttttcccaggaattgagctaccatggggccatttgccatgagctaattgaagtttcccaagggctacaataatatattgggtgtggagtctcctttgtaggagagcccctgcatgcagttgcctgctgtgaccagcagagggaggacaagtcctgggcccatgctggcagtgaggcagctgcttccagggatgggagagcagtaactcaagtgaaggaagatggattgtttgcaattgctttgccccatcgagacataaaatgcactttgcaagatagagtctgaggctaatacaatgcagtgtggggctgtcagcattaggggatgagatgacaatatgggacctgggtctggcctgggcaagtgcctgctcagcatgtgtacgctgtttgggttgtattggagctattgggcgctggaaccagcacttgtgttggagcggtttccccccaccagagccacagtgttttctccccagtggtggtcacacccccatataggaaatcaggcattccgtgataaaatggaggtttgcatgagtgtgagcactctttacggagactagaacattccctgtgtctttcagttcctttttttttttaagagatgaggtcctcactgtcacccaggctggagtgcagtggcactgtcttggctcactgcagcctcaatcacctgggctcaagcaatccttctgccttaacagagcagctgagactgcaggtgcacaccactgtgtttggctcttaaaagtttttgtagagatggggtcttgctgtgttgtccaggctagtcgaactcctgggctcaagcgatcctctcaactcccactcccaaagtgctgggaatataggcctgagccactacgtctggcctttcagttctgattagcatccctgaatggccattttctctctcactggcaactcagtcagttcatgcttggtccagggacttggtacatcaggagacttgtggaatatggattaaatcacaagaactaaagttaaataatcacaacaagtgaaaggtgctgtgtcgagcactgttttctaaacacattcattaggtgaactcattttaataaaagggtaaagagtaaacaattatcatgcttaggaaagaaggaatatgtgcaaaccctcaatgctggagcagccacagagaatattaggactgagcctcaggggtccacaccagggacacctgcttggaaaatgtatacaaacacagggttcgcctaattgccttcattcagagtatcctccttttccacagtggaagtgtcgtacctaacaaggttctggatatctgtgtgttgagactctgccatttctatttttctgcttttttctttccttcctttcaaatagatgtctgtggactttatccccaggaatggtctcttggttaaagtgtgtgaaaatcttctgcatctgatggacaacattgtgatagacatggcacctgggacacacctgtaggtggatgttgatgatgtctttctcttttaggtctctgcagacaaactggtggcactggggctgttcagccagcactttaatttggccaccttcaataagctcgtctcctatcgaaaagccatgtaccatgctctggaggtaacatgggatgagggaatgagggctaagccctgagagcagggatgaagcaagagacccacagaaacctagctcagtgttgccaagggtggtttggaggggacagggtggccagggaggaggtaaatgacaacataacatgatggccaccggattgggcctcccaacaaagggatttgagacatcccagctgccacttattgggaccactaactgaatcagaaggaaagagcacgggcagttaggggtgcaatattcgtatcagtgaatgtgcttttccagagacctcagagccttcttatgagggttggagttcacagcaagacagcagtggaggccagctgtcttctatctgtgggccgtcttatcccctatgaaccattataccctgtttctcccttcaccttgattttttccccccacctttgttcagcatttaagagccaacctcttctcattgctaaaggcttcattagacactgaaatccttggtccttcatgtaggacacaagttaatgtccccatctttagggagataaaacaagaaatctagtgccgccttatataagatacagaggagatgagatagtgctggctggggcttctatatcttggtggtagagttaattctgatatgtctgccttttgagtgggaggtgaccactgacattccctagggggaagagatcctatttggtgcctgcatgaaaagatttaaccacaaccccccttccgaggcctctccctgcacaggcccctccttcctctgtcacataccacagcccaggacagctgtctggctatgaaagggcccagtgtgaaggggaatgtaggccctggctgggggaatccctgggtgtggcctggcgagcacctcctccccacccccccattcatcacagactctgcctttgcagaaagctagggtgcgagctggcaagaccttccccagcagccctggagactcattggaggaccagctgaagcccatgttggagtgggcccacgggggcttcaagcccactgggatcgagggcctcaaacccaacaacacgcaaccaggtgggaatgagtccccatggcagcacccgctgcctctgctggtgggaccacttcttgggagagtcagccacccctgctgccccacaccccacccagctttctagcagctggtctcaactctgaatgttggaaaaataggagcaggcatacgaaggctgtgccagctctatctggcttccccggccactttcttgctgatggtcacgtgcatataatttgtattgaatctgacatctgtcttgaagtcaggatgcagtgttgactagcaccttttttattaaaggaaaaaatagttaagctatgctcatttgagaaaattggaaagtgttaaagaagaatatgccacctgctaacattttgaccagtaccatatttctcccattataagataactctggtttctagaaatccaggacttgtcttacaatccatagatttaaaggccaggcctagtggctcatgcctgtaatcgcagcactctgggaggctgaggcgagtggatcacttgaggcgatcacctggacaacatggtgaaaaatagaaaaattagcctggtgtgcttggcatgtgcctgtggtcccagctacccgggaggctgaggtgggagaattggcttgagcccaggaggcagaggttgcagtgcgccgagatcgcaccactgcattcaagcctgggtgacagagcaagaccttgtctcaaaaaagaaaacagtccatacatttaatgtaatgttttttctgttttgttttgttttcccctcaaaagtggttaataagtcgaaggtgcgtcgtgcaggcagtaggaaattagaatcaaggaaatacggtatttccttcctgtcttttgactgtgccctgttttctatgcactttcttctgatttctttgcatataaaatggtcactggaaaagaatcaaatttcttgaaaagtcaatctgaactccctccccactctcccctcacatcccaccctcacaaataccctccaggttaacgttgggttgcacatccttccagatttttctgaacactagttggattcctgctttcccctttattaatattaatactgtaagcactgtctgatgtcattttgtcactaaagggtccttaagatttttaatggctgtacaattttctttcctagggtcagagtcaaatttaaccatgtccattaagattttctgtgtagatggaatagaaatacaaatgggttttacaaaatcccatttatagaatgggagccatattatgtaaaattaaaaagtgctgggctcagtggctcacgcttataatcccagcattttgggatgcaaataaccatctcttggatttcccagcattttgggacgctgaggtgggcagattgcttgagctcaggagttccaagaccagcctgggcaacaggatgtaaccctgtttctaagagaaatacaaaattagctgggtgaggtggtgtgtgcctgtggaggctgaggtgggaggatcacctgagcccaggaagtcaggctgcagtttgctaagatcatgccactgcactccagcctgggtgacacactgagaacctgtctcaaaaaaaaaaaaaaaaaaaaaaaccaaaacagaaacaaaaaagaaagaaaaaaacgaggccaggtgcagtggctcatgcctgtaatcccagcactttgggaggctgaggcaggcagatcccttgtggtcgggagttcaccagcctggccagcatgatgaaaccccgtctctactaaaatacaaaaattagccaggtgtggtgctgcatgcctgtaacaggctgaggcaggagaatttcttgaatccaggaggcggaggttgcagtgagccaagatccacgtcattgccctccagcctgggcaacaagaaaggaacaggtgaaattaatacaaatcatgatttttttagcgctaaatttccaagatgttagcattttaatatccagctattataaaaactgagatagtctacatttatagtgctggtttcaaaatttcacttattttatatattttacatattacatattttacagatctcaattttgatgctaaatttgcaatgaaaatacttgatctgtattaactatatacagattcttgactaaatacagatgtagatggttaaacacaaattaactaaattcataaaacgtgcatttgagaaagcagactcatatgtctaggctagtcttaatgagccagttagttgacttacttgtcagtgttttaattaaccttaaatgcagaattcatttcctcaacagtttcaagttctcagtagccaccccctatgcctactgcccagcatgtcagatatcacagacatgggtatgccaggcttttagctttgaatcgagctttgctctctgagctgagtcatttttacatcctagttgtagccatttaaaaccttgtaaacatgtgttaaggatcctgggtgcaaggggttttgaatgagctcctgttgtgagttgcccatcaaggggccatatacattcagtgtggaccccctgtcatgccccaattgcagctggtacccaggcatagcatggtcttgtcctgggcccgcattctctctggaccctcctaccaagccacggctgcagtctaattacctttcacagagaacaagactcgaagacgcacagctgacgactcagccacctctgactactgccccgcacccaagcgcctcaagacaaattgctataacaacggcaaagaccgaggggatgaagatcagagccgaggtgattgttgggtacctgggatcatgggacagatgggaggaggacgctgcagatcaggaattgatctgtacccggctccctgacctcatctcatgccttcttcttttctcaatagaacaaatggcttcagatgttgccaacaacaagagcagcctggaaggtaacgttctctccctcccagtcatccccctcacaccctggctagggctctaggcctgccttgtcctggctcatccaccctgtcagggtttaacccagagctctgttccttaacctcaggccaaggcctatttctggaccctgcatcttagttgctagtttatggaatgagttgccaattgtgccccttaagagaaaggtacaggaagggtatggtgaggaacaagggaagctgggccattttggccgggagagccataacctaaaaccaagattactggtatcatgaaccttcccaaagaagaactgcaactaacaagcttttagtcacttgcctactgtcaggctaaatactctgcttgccctggctggggattgccactggattggatactctgatcagaatatacctcatggaatacagaatgtgtgaggatgaagtctcccttgcttccttaacccctagttactcaatgttgaataggaaaacaccacatccctgacccagagaacccggggaagagcttcggtttagaaatcctattccccaagactcactcccccatctggctaggtagggatgcttgtttgtgggttttataaaggtcctatctacccagccagggatgcaactccatgctgacctggtgatatgaagggaaggcagggttgcctactggttaaaggacctggattctgaaattacagccatttgctgtttacagggtgtgggcatgggcaagtcctatcatgagacaacaagccaattggagtcaccacaaaatccttagtgtcccagtatttaaagcttctcaaggattgtcacaccagggaggtccccctgctcagcgttctgcctgacatcatcctactggggcagccccttgcccatcttgatgttccccatctgtgaagtagggatgccacctggaagcattgttgagtggtagaagctgtgaggcatggcctgaggtgcaaagagtccttggcaagctgctggcctggagggaaatcttaggaactgagagaccccaggctttagcagctggtgtcagggcctcaactgccaaaagccacaaccctgtttttcttacagatggctgtttgtcttgtggcaggaaaaaccccgtgtccttccaccctctctttgagggggggctctgtcagacatgccgggtaagtcctcctactactgccctggaccttcctccccttgcctcctctaactcccctctccttcccaacagcacctgtgtggagactcagtgaagcccactcatcccagctgccttgcactctgcctctggggcaggatgggggacagctgtcacagaggccaatggcacgcaggtcacagccagttgtccttttaaaggacaccaagccaccagcagtgtgttctgcagctgtatcctagggcctccaccagcaagccggcagggcctgcccttctctggtctccgatttcactggtgtttctctctggctgccaggatcgcttccttgagctgttttacatgtatgatgacgatggctatcagtcttactgcactgtgtgctgcgagggccgagagctgctgctttgcagcaacacgagctgctgccggtgagcactgggccctgtggggtggatgtgggtgggcccccaaggctcctacgttcctgcagtctgcagacagctgtctgttgaatggaatcctaggcatgggaatagggagctaatttgccctggaagcagcacacagggtttatattttgtggtggctgtggttgtagcataattggagtagaactcatgtatagggaacggctcttttttttgagacagggtctccctgtgttgcccagggtgtacagtgacatgatcataggtcactgcagcctccaattcctgtgctcaagcaatcctcccacctcagcctcctgaacagcttgattataggcacatgccatcacacccagctagttattcttttttttttttgaggtggagtcttgctctgtcacccaggctggaatgcagtggcacaatcttggctcactgcaacctctgcctccagggtacaagcaattctcgtgcctcagcctcccaagtagctggaattataagtgcggagcaccacgcccagataatttttgtgtttttagtacagacagggtttcaccatgttgaccaggcttgtctcgaactcctgacctcaggtgatccacctgccccagcctctcaaatcccagcactcccaaagttctgggattacagtcatgatccaccgtgcctggcctattttttgtaataaataaaaagtgtaataaacgctgcctcggctggtctcaaatccctggggtcaagccattctcctacctcagcctcccaaagccccagagttgtaggtgtgagccaccacacccagctagaagtgctcttaataggccaagagcatgagggaaggggatgagtagggtgtcagggaaggctaattccctggtttgccagttggcctgtaatccaaagtgtccatggaatgaagtaggttgtcgggagagcatttctgatcagagagcctgtgggattaagggggttggggcatccaaggaagacgtcagggaagcccgtactgcacagggccccgcaggctatgctgttaagcagccgatcctaggtaagctttcaggagggggttggcatttccctgtggaagtggtaagggggtggcacaggagaccagctctgacaaaggcatcccttctccctgccactgggtccaggtgtttctgtgtggagtgcctggaggtgctggtgggcacaggcacagcggccgaggccaagcttcaggagccctggagctgttacatgtgtctcccgcagcgctgtcatggcgtcctgcggcgccggaaggactggaacgtgcgcctgcaggccttcttcaccagtgacacggggcttgaatatgtaagccacaggctcccgcctctaccaccacagatcccaggggcacagggtgttggaaagctctggaattctcagaaagagtaatagaagtaaagacacgttgtacctcttggagcctcaatggctgagagacctggcgaattgccagctcctctgcacggttttcagccgtgccagggttcattcactccctccttgccggcttctcagcgtatggattttcaggggcctgatgaagaaatggttgtatgtagccttctgagttagcagagctgagagggaaggaaacagtagaaatgaattttctagttcttagagggaagccctaggtcattcctgtctggggtgataccttttcatgtgtgcctctgtgcatacatatctggctcatgtccaaggataaattgaccattccttgccaaatgcaggctcgtgtggtacacacttgtctccccaatccccatcaagccttcagtgggctttttgcagtggctacggcaaggtttgaagccctctgagcagggtcagcctgcccctccctcagagcttgagtctttgccctgtgccttcaccaccatgacctccttccttacctggcaggaagcccccaagctgtaccctgccattcccgcagcccgaaggcggcccattcgagtcctgtcattgtttgatggcatcgcgacaggtgagttcggggaacacctggagacactgctatcgtgtcacaacagggtagccagggagtcagaaggcatggttaaggtgtctgacatcagtggcaagaaaggtccctggggattaccagccctgaaaaaaaccctgtctccttgtttctctccaccttctcaaccaccttgcagtcttggtcattgagttttatttatttattttttcgagatggagtctccctttgtcacccaggttagagtgcaatggcgtgatctgtgctcactgcagcccccatctccgaggttcaagtgattctctcctcagcctcccaagtagctgggattacaggtgctcaccaccacgcccagctaatttttatatttttcgtagagacggggtttctccacgttggtcaggttggtctcgaattcctgacctcaagtgatctgcccaccttggcatcccaaaccgctgggattagagacatgaaccaccgtgcctgggcagtcttggtcattttaaaaggacatacatcttagctgttacactttattttttatgtgcagttatcttgaacttgattttcattccacatgcataagtataattggagataaactagttaacaactactgctacctctggttttggttcccttcttttcctcctatatggagtttacacagccaatatgtgtggtgcgtgtacagcctttgctggattgaacgcatgtgatacagtcatgaggaactgttcattttaccatagcagggagtggaggaaatgagctgctgtgtgctcagcatcatttatgcttctgtgtctctctggcccccacaggctacctagtcctcaaagagttgggcataaaggtaggaaagtacgtcgcttctgaagtgtgtgaggagtccattgctgttggaaccgtgaagcacgaggggaatatcaaatacgtgaacgacgtgaggaacatcacaaagaaaaatgtgagggcagtctgtaccttgcgggcctcatctcttcctgtctttttccccagtcctccacaccctgaaacccacatgtaggccccatccctgagaccccagaaaaaaggattgaaatcctgtgggaatcttaagcattaggttagggtcagaagtggagttggtgaccaagcaagattctatttttttctggagacagtctccctctggagtgcagtggcatgatctcagctcactgtaacctctgcctcccgggtttaagcgattctcttgcctcagcctcccaaagtgctgggattacaggcacctgccaccatgctgggctaatttttatatttctagtagagttggggtttctccatgttggccaagctggtctcgaactcctgacctcaggtaatccacccccgccgtcagccttcctgggattacaggtgtgagccacctcgtccagccccacgcaagattctagaagtgggtccagctctctttccctctgtccacaccctcatcctgactctgtctctctctttcagattgaagaatggggcccatttgacttggtgattggcggaagcccatgcaacgatctctcaaatgtgaatccagccaggaaaggcctgtatggtgagcatccttctctctggcagtccctggagagcctatgtcacctgaccactggcccaggtgcagcagcctgagaaggagccacttgcttctggccaagttactggcagcatcaggggcctgttggtgctgcctacgctccatagtaaatcctcagcccacaagggaaataccctagtaaatagtgccctgctgctgcctgtgtccctgctgtcattcaggtggacatagactggtaggcatcaccctgaactgtcaggaggccattgggaacctgctggtctcagggaataaggtgggttgggctggaggtttcaaatgaaccctgcgctgtcatcttttctgagcacagagggtacaggccggctcttcttcgaattttaccacctgctgaattactcacgccccaaggagggtgatgaccggccgttcttctggatgtttgagaatgttgtagccatgaaggttggcgacaagagggacatctcacggttcctggaggtgagggaatctggggacctgattgtcacagacagccagggcagggaaagcgctgctggcagtgatgattggtgggtgttgccaacattgggaatgactttcccgttcttggtctggctagatccaatagtgagggattcagtgggttctcttagtacatggaaaatattttttttgagacagggtcttgctctgtcacccaggctgaagtacagtggcattattacagctcactggagcctcaaacagctgggctcaggcaatcctcctacctcagcctcccaaatggctgggactacaggggcacaccaccgtgcccagccaatttttgtatttttggtagagacagggtcttggtatgttgcacaccctagtcttgaactcctgggctcaagcaatccgcccacctcccaacgtgttgggattggagatagagatgtgagccaccatcccctgccagtacaaggaaaataattgatctctaatctcagcccaactctttgaagtcagaaatgatgaaacaaaccatcatctgctgttaaggactcatgagatttgactcaattttttactctagagagttttctcatttttgatatgaaaactgctttagaccaggcacagtggctcatgtctgcagtcctagcattttgggaggctgaggttggaggatcacttgagcccacggtgtatcatattttgtaataatctggcgtatcccaggggataccgtctcagaatctgcaggtttgaccttatctctttagctaccattagcatagtatgagggcatttaccatgtcctctagccagcctcctgatggcagtaggtctttcctgttttggaggatccgtgcctcatccatagtcagggaatagccctgtcacctgcaaaggtctggttgacactgaaactctaataataggctcctaacagtaaccttctttctccccacagtgtaatccagtgatgattgatgccatcaaagtttctgctgctcacagggcccgatacttctggggcaacctacccgggatgaacaggtaacaaagggctcttagtgggtcaggtaacagccaagttaaatatgtgataacaagctctgacattcaagccttcctagaaagacctggctctgctgagaaatagttatacttggatttcagaccacctgtggtcgtgcgggttgatctctgttaagatgttcagcaagtacctgggaatctctgtaggagtcccactcttacctgttgtaagctgctgatggacctgatcctaaaagtcccacgtggctgggtgtggtggctcacgcctgtaatcccagcactttgggaggccgaggctggaggatcgcttgagcccaggagttctagactagcctgggcaacaaagtgagaccccgtctctacaaaaaaataaagaaaaatacaagtctcatgagctgtggccagactgtgtacctccttcctgccagggatctctcaagacttgagcttgttgatgctcctgcccttcacctctcctccccgttgatgtagtgacagccgtctggaaatggaagtagattgttgttaccggtgcctttcctcctgccctggagatgaagcgatcttggccaggctcatggtgaccaaccaggtggcagagctgctatgacaaacaatagcccaaagactatgtccctcagattctcatcaccaggagcatttcatattttgttatcgtatactaaaaacagtcttcttcagacactctgaaagccacaaagcagctttgctgaggattaattatttcttctatatgtggtttcaaggagaagaacttgctaattaagatgtaaaatgttatggtaatgtgacatcccagacttgcaaatgagttgagcttaaaacattctcctgtagaaggaggcatggaaacataataaccttctgccacttagtgaagtgtggacagaggtgagggtcctggggtagcttggaggatttttgttggtcaccttgcccgtgtacttggctaagcagccagtcttagggtgccatgtctgtgtctggaattaaatgggttaatgctttcaacatttgaactcccagtaggccaggcactgttacgctgttgccattttcatggttaaacacatcatgtcgatagtgacatgccgaagggggagggagagaagacaatgatttggggtttattgctggcggatggacattggaagggttgatggggaaggcttctcattctagcgaaggtcctaagtgaagggatggacccatgcagttattagagtactccaggtgggaagagggtctgggatgatgggaaacatccccagaccagctcagagcaccctaggtggggtggctgggagagaaggaccaagtaacttgggccttaatgtggtaccgctgaaatcactttggcatgggaagttacagatgagatgaattgtgctttggttcatggtagtttctgggtggattatagacaataacaggatggaggcgggaatggggagacaagtgaagggactagactgctcaaagaaggagagatgactgattatgatgttatgccccaggaggtgccatccatagagaacacagtgtgagtggtggcccctggggtttgcagcacagcaatcctgtgacttagcaaggacggatcagattcaacatatgtggagggtaggacatttgctgatgcattggatgtgtaggctatgaggtggaaagagcagtgagacctgatgcagatctgatgctgtgactaagctctgcaggcttataggatgggtgtgtcaggcatcatttaggggacagacccaagtcatctggcttgtttagagcttaatcttatagtcagcacaaggcggggctgtgcagtgtccttttcttctgggccctcagctcccctgggtccatgtcggaggtaggaatggggatagaggtagcatttgaagcctggcaggtgaggtcaaggaaatctatcagtctaggggaaatatgtgttgtgatttcctcagcccctcttgtaggcacggttcccgttctcatgtctgagttgtttggccttgacgttcctaacacaggttactatggccgtccctgcgtccagtcctcttgcaccagtttctgttctggagctgctcaactactttcagttctagcccttgcccttcttagtcatctgtttgcctctctctgcatttgcaaaataattttcaagggtgaaagttaaaagggtgcttagtctttttttttttttttttttttggaaccttaagagacagggtctgtctcccaggctggagtgcagtgttgtaatcataactcactgtagcctccaactcctaggcttaagcgatcctcctgctttgaactcccaaattgctgggattaagggtaggaaccactgcaggcggttgccattcacttttaaagcctcctgaggctgtatccccagcagtgagccaccaaaccgtgggtgtgatatgataattgtatcaggtagacccacaaaaatctattccaagctcctcactttaaaatagacaggaagcccaaaggggtagtgtgactcgctcaagctcagtcagtgagcagtgtggagctgggacctgaggtttctcctgccctaccccatgcagtgttcctgctcaatgggaacctgactttgaagtccaggcaggggagcattacctcctggatgctgggctgtcccctctagcccatccttggctttggcacacaggcttgtgctcatgccaggatcattttcatcatttatttgtagccaagttcactgccagggcacatctctgcaacatagaccctcactcccaccttgtgcctagcagaggaccctctatagctagtaagaagtaatgggttttggctgttcccaggcccgtgatagcatcaaagaatgataaactcgagctgcaggactgcttggaatacaataggatagccaaggtaagacgagctgtggccctctggaaaaatgcacttggtgacctccaagtggggacttgggagatgaccttggtgtttgattggttcctactcctccccccacgtgacttcctggtgttgggcttccctctcccacatgattgttctgtcccatcctcaaatacagagactaggagacattcgtgataagtaatcacgacgagaaaaggcaactagtctttttaattttagtactggatacataaagacaaagagtttcagctggccctctcagggagaaaggaatcattaatccttttgccaaaaaattaatttgctgtcaagtcatagaatatgaagacctccataaacctaaatctcaatgtttgcatcaagctaagatccattttctaaactccaattgagcattctctgtatctgggtggtttttacttttttacttaatcttgcttgatcaggaactctggtgtcttcttggccccccacgtgatctcgttcatggtcacttttttgtttatctcattttctctgaggctggtccttcctgttaacgtcttggcatttgtgggaagcacaaaatgttcttgtctctccaactctgcttttcgctccctgccctgccattcctctcccgcgcctgccctctcccttccatctttcccaggtacttttctctcccagccctgccactcttctgccgcacctgcgctctcccctccatctttcccaggtacttttgagccttgactccccaggtcccttcattctgtgctcactccatgatgtcattttgttctccagttaaagaaagtacagacaataaccaccaagtcgaactcgatcaaacaggggaaaaaccaacttttccctgttgtcatgaatggcaaagaagatgttttgtggtgcactgagctcgaaaggtgagcaaggctgcacttggagagggaaactgtgtagatcaaaacacaaatgggcagacatgggcaggtgcttaccttcattcttgatggcctcactgccctttggtgttactggggcgaggagtaataataccttttcatagttcgcccttatttcctgacaaaaatggcggagggggtgtggtggtggtgaggaatctgagacatgaggtcattaggccacaccatctgtctgttggctgcactgccgtgacatctgcctgtcccaacatgctgggacactacgatgtgtgagaagccagtgaaagaaaaccccacagacatgccattcttgaagacttcagtttgctcattctataaatgattccttcttagccacagtttatcctctccttccctttctttgggctaagtttgtctgttttgatgcaaataaccatctcttggatttcagttctgttctgttctgttttctttcactcctctattgtttgttcattttgcttatactattttttaaaaatcattatttgctatagtgattatggctttgaacattcctctttacatgtattctgtattctgaaggctttgatatagagtattttcattccagatatttcacttctataactcatttaatttcttctttgacttaccaacagttttctgagtataaaagtaacttctgattagtatttatgctgtagattattctgtatggacctaaacctaaaatggttggtgcagatggctgacattgtgtagacgctaagaccaagcaagacacaggcctgaggtatcacatctttggcatgtttctgaataacccacgcagccattggcctgttgggatgacttttagtattggattgtatacctgagtaacactgaggctcagcctctgtagcctctttccaggcctggtagaatcctatagaggaagcttttggtatatttgatgccatctgcctggagtcatctagcatggtccttcatccggcagctgggtttgaagagggaaggaaaactctggttgcctgagcctgtgatgactgaaaatctaggctgtttttgagtgaccttgttccttattaaggaaattgcttaacttctcccatcatagtgttttgaaaatggatacattgctggctggctgatttagtatgcatgcatttattctaggaaggtcctctgagcaccaactctatgccaagagtatggtgctaggcctgcacaaaacgggggactttcatttgtgagcaatttgtaagcaagtgttctgaattaagggctctgaatttaggtccttggggaccttactgatgggactgagggatggcgagggcagaaagagtgggacctggctggttgaggctgtcaacatcctggaggcacttctgacttgctgtcttttcactccggtacccccaggatctttggctttcctgtgcactacacagacgtgtccaacatgggccgtggtgcccgccagaagctgctgggaaggtcctggagcgtgcctgtcatccgacacctcttcgcccctctgaaggactactttgcatgtgaatagttccagccaggccccaagcccactggggtgtgtggcagagccaggacccaggaggtgtgattcctgaaggcatccccaggccctgctcttcctcagctgtgtgggtcataccgtgtacctcagttccctcttgctcagtgggggcagagccacctgactcttgcaggggtagcctgaggtgccgcctccttgtgcacaaatcagacctggctgcttggagcagcctaacacggtgctcattttttcttctcctaaaactttaaaacttgaagtaggtagcaacgtggctttttttttttcccttcctgggtctaccactcagagaaacaatggctaagataccaaaaccacagtgccgacagctctccaatactcaggttaatgctgaaaaatcatccaagacagttattgcaagagtttaatttttgaaaactggctactgctctgtgtttacagacgtgtgcagttgtaggcatgtagctacaggacatttttaagggcccaggatcgttttttcccagggcaagcagaagagaaaatgttgtatatgtcttttacccggcacattccccttgcctaaatacaagggctggagtctgcacgggacctattagagtattttccacaatgatgatgatttcagcagggatgacgtcatcatcacattcagggctattttttcccccacaaacccaagggcaggggccactcttagctaaatccctccccgtgactgcaatagaaccctctggggagctcaggaaggggtgtgctgagttctataatataagctgccatatattttgtagacaagtatggctcctccatatctccctcttccctaggagaggagtgtgaagcaaggagcttagataagacaccccctcaaacccattccctctccaggagacctaccctccacaggcacaggtccccagatgagaagtctgctaccctcatttctcatctttttactaaactcagaggcagtgacagcagtcagggacagacatacatttctcataccttccccacatctgagagatgacagggaaaactgcaaagctcggtgctccctttggagattttttaatccttttttattccataagaagtcgtttttagggagaacgggaattcagacaagctgcatttcagaaatgctgtcataatggtttttaacaccttttactcttcttactggtgctattttgtagaataaggaacaacgttgacaagttttgtggggctttttatacactttttaaaatctcaaacttctatttttatgtttaacgttttcattaaaattttttttgtaactggagccacgacgtaacaaatatggggaaaaaactgtgccttgtttcaacagtttttgctaatttttaggctgaaagatgacggatgcctagagtttaccttatgtttaattaaaatcagtatttgtcta

SEQ ID NO: 4 - DNA, 1538 bp, Spiroplasma sp.
gatcttgataattcaactttatttggatgttgtgaaatgtagataaatcttacattttttattgtaaaatcttattagattgtttttttagaggagctaaaaatgagcaaagtagaaaataaaacaaaaaaacttagagtatttgaagcttttgctggaattggtgctcaaagaaaagccttggagaaagtcagaaaagatgaatatgaaatagtagggcttgctgaatgatatgttcctgcaattgttatgtatcaagctatacacaacaattttcatacaaagttggagtataaatcagtttctagagaagaaatgattgactatttggaaaataaaacactatcttgaaactcaaaaaatccagtatctaatggttattggaagagaaaaaaagatgatgaacttaaaattatatataatgcaattaagttatctgaaaaagagggtaatatttttgatattagagacctttacaaaagaactttgaaaaatatagatttattaacatattcatttccttgtcaagacttatctcaacagggtattcaaaagggtatgaaaagaggttctggtactagatcaggtctcttatgagaaattgaaagagctttggattcaactgaaaaaaatgacttaccaaaatacttgttaatggaaaatgtaggagctcttcttcacaagaagaatgaagaagaactaaatcaatgaaagcaaaaattagaaagtcttggctatcaaaactcaattgaagttttgaatgccgctgacttcggttcctcacaagcaagaagaagagtttttatgatatctactttaaatgaatttgttgaactaccaaagggagataaaaaacctaaaagtatcaaaaaagttttaaataaaatagtttctgaaaaagatattttaaataatttattgaaatataatttaactgaatttaaaaaaacaaaatcaaatataaataaagcttcactgattggttacagtaaatttaattcagaaggttatgtttatgatcctgaatttacaggaccaaccttaactgcaagcggtgcaaattcaagaataaaaatcaaagatggatctaatattagaaaaatgaattcagacgaaactttcttatatattgggtttgattcacaagatggaaaaagagtaaatgaaattgaatttttaactgaaaatcaaaaaatatttgtttgtggaaattcaatatcagtagaagttttggaagcgattatagataaaattggaggttaatacatgtcaataaaagataaaacatatttagtaaatgttcctataataagaaaaaattttttttctactaattcagctaagtttaagggaaatttgcaaattatttcaaatgattttggaataataaaagattgaaaaattgaattcaaagaagtttgaatagatatatttacaaccccaaggtgatttagtgataagcctagcttatttaaatatagggaaaggctttttgaagaattaaatttaattcaggagcacagaccttcaaaaagatc